(19) United States
(12) Patent Application Publication
(10) Pub. No.: US 2024/0188429 A1
(43) Pub. Date: Jun. 6, 2024

(54) COMPOUNDS FOR ORGANIC ELECTROLUMINESCENT DEVICES

(71) Applicant: Merck Patent GmbH, Darmstadt (DE)

(72) Inventors: Amir Hossain PARHAM, Darmstadt (DE); Christian EHRENREICH, Darmstadt (DE)

(21) Appl. No.: 18/279,891

(22) PCT Filed: Feb. 28, 2022
(86) PCT No.: PCT/EP2022/054903

§ 371 Date: Sep. 1, 2023

(30) Foreign Application Priority Data
Mar. 2, 2021 (EP) 21160186.9

(51) Int. Cl. H10K 85/60; C07D 471/04; C07D 471/14; C07D 487/04; C07D 519/00; C09K 11/02

(52) U.S. Cl. CPC H10K 85/6572; C07D 471/04; C07D 471/14; C07D 487/04; C07D 519/00; C09K 11/02; H10K 85/622; H10K 85/624; H10K 85/626; H10K 85/654; H10K 85/657; H10K 85/6574; H10K 85/6576; H10K 50/12

(57) ABSTRACT

The present invention relates to compounds that are suitable for use in electronic devices, and to electronic devices, more particularly organic electroluminescent devices, containing these compounds.

The present invention relates to compounds suitable for use in electronic devices, especially in organic electroluminescent devices, and to electronic devices, especially organic electroluminescent devices, comprising these materials.

Emitting materials used in organic electroluminescent devices are frequently phosphorescent organometallic complexes. For quantum-mechanical reasons, up to four times the energy efficiency and power efficiency is possible using organometallic compounds as phosphorescent emitters. In electroluminescent devices, especially also in electroluminescent devices that exhibit triplet emission (phosphorescence), there is generally still a need for improvement. The properties of phosphorescent electroluminescent devices are not just determined by the triplet emitters used. More particularly, the other materials used, such as matrix materials, are also of particular significance here. Improvements in these materials can thus also lead to distinct improvements in the properties of the electroluminescent devices.

Document JP 2000/156288 describes quinazoline derivatives that can be used as matrix materials for phosphorescent emitters.

In general terms, in the case of these materials, for example for use as matrix materials, there is still a need for improvement, particularly in relation to the lifetime, but also in relation to the efficiency and operating voltage of the device.

It is therefore an object of the present invention to provide compounds which are suitable for use in an organic electronic device, especially in an organic electroluminescent device, and which lead to good device properties when used in this device, and to provide the corresponding electronic device.

More particularly, the problem addressed by the present invention is that of providing compounds which lead to a high lifetime, good efficiency and low operating voltage. Particularly the properties of the matrix materials too have a major influence on the lifetime and efficiency of the organic electroluminescent device.

A further problem addressed by the present invention can be considered that of providing compounds suitable for use in phosphorescent or fluorescent electroluminescent devices, especially as a matrix material. In particular, a problem addressed by the present invention is that of providing matrix materials that are suitable for red- and yellow-phosphorescing electroluminescent devices, especially for red- and green-phosphorescing electroluminescent devices, and if appropriate also for blue-phosphorescing electroluminescent devices.

In addition, the compounds, especially when they are used as matrix materials, as electron transport materials or as hole blocker materials in organic electroluminescent devices, should lead to devices having excellent color purity.

A further problem can be considered that of providing electronic devices having excellent performance very inexpensively and in constant quality.

Furthermore, it should be possible to use or adapt the electronic devices for many purposes. More particularly, the performance of the electronic devices should be maintained over a broad temperature range.

It has been found that, surprisingly, particular compounds described in detail below solve this problem and are of good suitability for use in electroluminescent devices and lead to improvements in the organic electroluminescent device, especially in relation to lifetime, color purity, efficiency and operating voltage. The present invention therefore provides these compounds and electronic devices, especially organic electroluminescent devices, comprising such compounds.

The present invention provides a compound comprising at least one structure of the formula (I), preferably a compound of the formula (I),

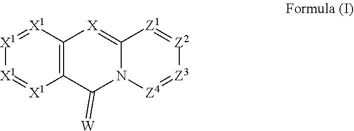

where the symbols and indices used are as follows:
W is O or S, preferably O;
Z¹ is X², or the Z¹ group together with the Z² group forms a fused ring A¹ bonded via two adjacent carbon atoms to the further groups of the 6-membered ring;
Z² is X², or the Z² group together with the Z¹ group forms a fused ring A¹ bonded via two adjacent carbon atoms to the further groups of the 6-membered ring, or the Z² group together with the Z³ group forms a fused ring A² bonded via two adjacent carbon atoms to the further groups of the 6-membered ring;
Z³ is X², or the Z³ group together with the Z² group forms a fused ring A² bonded via two adjacent carbon atoms to the further groups of the 6-membered ring, or the Z³ group together with the Z⁴ group forms a fused ring A³ bonded via two adjacent carbon atoms to the further groups of the 6-membered ring;
Z⁴ is X², or the Z⁴ group together with the Z³ group forms a fused ring A³ bonded via two adjacent carbon atoms to the further groups of the 6-membered ring; X is the same or different at each instance and is N or CR, preferably N;
X¹ is the same or different at each instance and is N or CR¹, preferably CR¹, with the proviso that not more than two of the X¹ groups in one cycle are N;
X² is the same or different at each instance and is N or CR²;
A¹, A², A³ is the same or different at each instance and is an aromatic or heteroaromatic ring system which has 5 to 40 aromatic ring atoms and has R³ radicals, where the aromatic or heteroaromatic ring system is bonded via two connected carbon atoms to the further groups in the structure shown in formula (I), where two adjacent A¹, A², A³ groups together may form a fused ring system;
R is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is possible here for an R radical together with a further radical, preferably a R², R³ group to form an aliphatic, heteroaliphatic, aromatic or heteroaromatic ring system, preferably an aliphatic, heteroaliphatic or heteroaromatic ring system; more preferably, the R radical does not form any such ring system;
R¹ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R¹ radicals together to form an aliphatic or heteroaliphatic ring system; the R¹ radicals preferably do not form any such ring system; with exclusion of the formation of an aromatic or heteroaromatic ring system by two R¹ radicals;
R² is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R² radicals or one R² radical together with a further radical, preferably an R, R³ group, to form an aromatic, heteroaromatic, aliphatic or heteroaliphatic ring system; the R² radicals preferably do not form any such ring system;
R³ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R³ radicals or one R³ radical together with a further radical, preferably an R, R² group, an aromatic, heteroaromatic, aliphatic or heteroaliphatic ring system; the R³ radicals preferably do not form any such ring system;
Ar′ is the same or different at each instance and is an aromatic or heteroaromatic ring system which has 5 to 40 aromatic ring atoms and has R⁴ radicals, preferably an aryl group which has 6 to 30 aromatic ring atoms or a heteroaryl group which has 5 to 14 aromatic ring atoms and has R⁴ radicals;
R⁴ is the same or different at each instance and is H, D, F, Cl, Br, I, R⁵C═C(R⁵)₂, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁵ radicals, where one or more nonadjacent CH₂ groups may be replaced by R⁵C═CR⁵, C≡C, Si(R⁵)₂, C═O, C═S, C═Se, C═NR⁵, —C(═O)O—, —C(═O)NR⁵—, NR⁵, P(═O)(R⁵), —O—, —S—, SO or SO₂, or an aromatic or heteroaromatic ring system which has 5 to 40 aromatic ring atoms and in each case has R⁵ radicals, or an aryloxy or heteroaryloxy group which has 5 to 40 aromatic ring atoms and in each case has R⁵ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁵ radicals; it is possible here for two or more R⁴ radicals together to form an aromatic, heteroaromatic, aliphatic or heteroaliphatic ring system, preferably an aliphatic ring system; more preferably, the R⁴ radicals do not form any such ring system;
R⁵ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F; it is possible here for two or more R⁵ radicals together to form a ring system;
   where the structure of formula (I) has at least one ring A¹, A², A³ which is formed by at least two adjacent groups selected from the Z¹, Z², Z³ and Z⁴ groups, and
   at least one of the R, R¹, R², R³ radicals is selected from the group consisting of a heteroaromatic ring system which has 6 to 60 aromatic ring atoms and has R⁴ radicals, an aromatic ring system which has 10 to 60 aromatic ring atoms and has R⁴ radicals, an aryloxy group having 10 to 60 aromatic ring atoms or heteroaryloxy group having 6 to 40 aromatic ring atoms, each of which has R⁴ radicals, a diarylamino group having 6 to 60 aromatic ring atoms in the respective aromatic radical, an arylheteroarylamino group having 6 to 60 aromatic ring atoms in the respective aromatic or heteroaromatic radical, and a diheteroarylamino group having 6 to 60 aromatic ring atoms in the respective heteroaromatic radical, where the diarylamino, arylheteroarylamino, diheteroarylamino group has R⁴ radicals.

The R¹, R², R³ groups have R⁴ radicals, where the R⁴ radical may be H. If R⁴ is not H, the R⁴ radical is a substituent, and so the R¹, R², R³ groups may be substituted by R⁴ radicals. This clarification applies correspondingly to the further groups and radicals.

The Z¹ group together with the Z² group may form a fused ring A¹ bonded via two adjacent carbon atoms to the further groups of the 6-membered ring. If the Z¹ group together with the Z² group forms a fused ring A¹, two adjacent carbon atoms of the fused ring A¹ together with the Z³, Z⁴ groups and the nitrogen atom and the carbon atom bonded to the X group form a 6-membered ring, as shown in formula (I). The expression “together with the further groups of the 6-membered ring” refers to the above-identified atoms and groups Z³, Z⁴. This clarification applies correspondingly to the formations of the rings A² and A³ by Z², Z³ and Z⁴ groups.

Two R² radicals or one R² radical together with a further radical, preferably a R, R³ group, may together form an aromatic, heteroaromatic, aliphatic or heteroaliphatic ring system. If a ring system is formed, this is preferably formed by two R² radicals, giving rise to a fused ring system. This is also applicable to the further radicals, especially to two R¹ radicals.

In a further preferred embodiment, it may be the case that the compounds of the invention include a structure of the formulae (II-1) to (II-7), where the compounds of the invention may more preferably be selected from the compounds of the formulae (II-1) to (II-7)

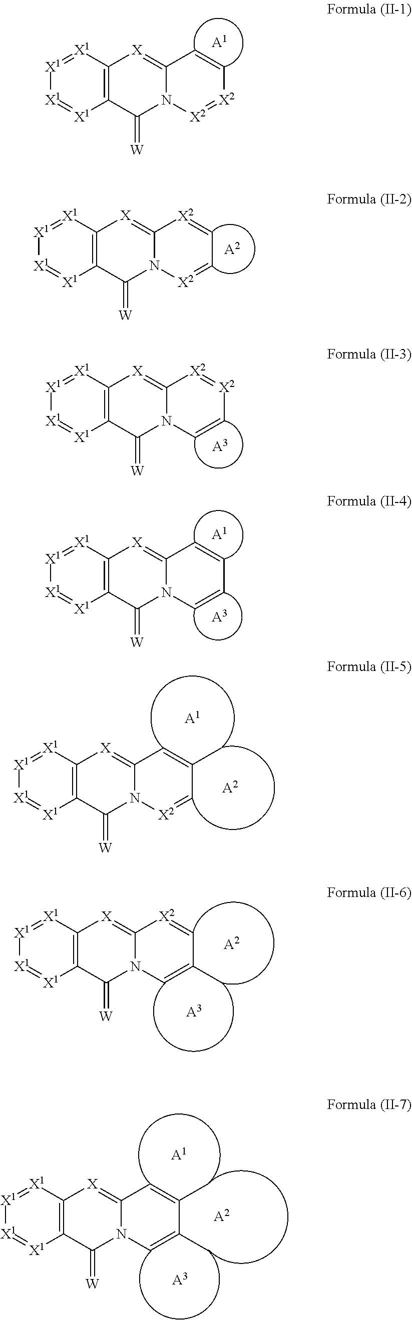

where the symbols W, X, X¹, X², A¹, A² and A³ have the definitions given above, especially for formula (I). Preference is given here to structures/compounds of the formulae (II-1) to (II-4).

It may preferably be the case that the rings of the formulae A¹, A² and A³ can be represented by structures of the formulae (KAr-1) or (KAr-2)

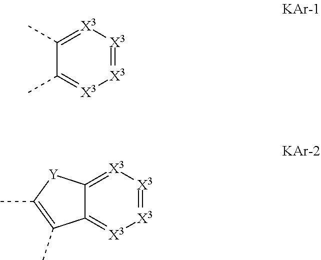

where the symbol X³ is N or CR³, preferably CR³, with the proviso that not more than two of the X³ groups in one cycle are N, Y is O, S, NR³, NAr′ or C(R³)₂, preferably NAr′, where Ar′ and R³ have the definitions given above, especially for formula (I), and the dotted bonds represent the sites of attachment of the ring to the further groups of the structure shown in formula (I) or (II-1) to (II-7), where the carbon atoms bonded to the dotted bonds represent the Z¹, Z², Z³, Z⁴ groups shown in the structure of the formula (I).

It may further be the case that at least one X² group is N, where the Z³ group is preferably X²═N.

In one embodiment of the present invention, it may be the case that the structure has at least two, preferably at least three, nitrogen atoms, where these nitrogen atoms are preferably ring atoms, where these nitrogen atoms are more preferably not adjacent and, most preferably, the structure has no two adjacent nitrogen atoms.

In a further embodiment of the present invention, it may be the case that the X¹ group is N and one of the X² groups is N, where preferably one of the Z¹, Z², Z³ groups is X²═N, more preferably the X¹ and Z³ groups are X²═N.

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatics joined to one another by a single bond, for example biphenyl, by contrast, are not referred to as an aryl or heteroaryl group but as an aromatic ring system.

An electron-deficient heteroaryl group in the context of the present invention is a heteroaryl group having at least one heteroaromatic six-membered ring having at least one nitrogen atom. Further aromatic or heteroaromatic five-membered or six-membered rings may be fused onto this six-membered ring. Examples of electron-deficient heteroaryl groups are pyridine, pyrimidine, pyrazine, pyridazine, triazine, quinoline, quinazoline or quinoxaline.

An aromatic ring system in the context of this invention contains 6 to 60 carbon atoms in the ring system. A heteroaromatic ring system in the context of this invention contains 2 to 60 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a non-aromatic unit, for example a carbon, nitrogen or oxygen atom. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferably, the aromatic ring system is selected from fluorene, 9,9′-spirobifluorene, 9,9-diarylamine or groups in which two or more aryl and/or heteroaryl groups are joined to one another by single bonds.

In the context of the present invention, an aliphatic hydrocarbyl radical or an alkyl group or an alkenyl or alkynyl group which may contain 1 to 20 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, further preferably F or CN, especially preferably CN.

An aromatic or heteroaromatic ring system which has 5-60 or 5-40 aromatic ring atoms and may also be substituted in each case by the abovementioned radicals and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

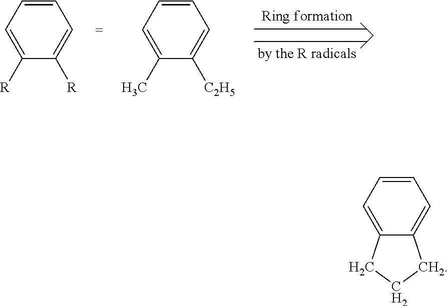

In addition, however, the abovementioned wording shall also be understood to mean that, if one of the two radicals is hydrogen, the second radical binds to the position to which the hydrogen atom was bonded, forming a ring. This will be illustrated by the following scheme:

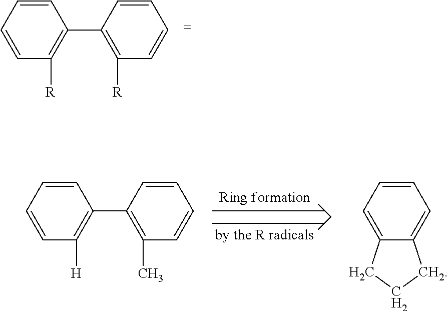

In a preferred configuration, the compounds of the invention may preferably comprise at least one structure of the formulae (III-1) to (III-11) and are more preferably selected from the compounds of the formulae (III-1) to (III-11):

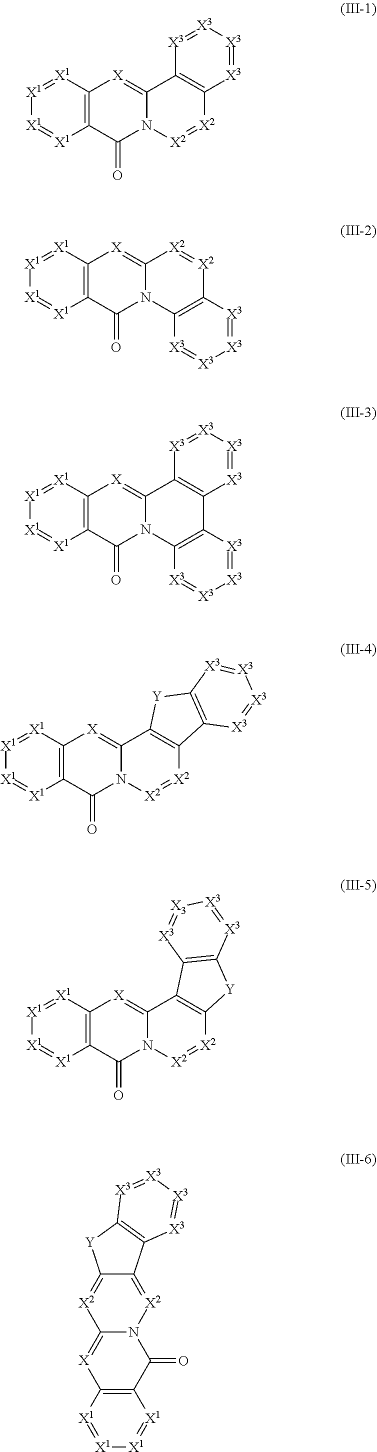

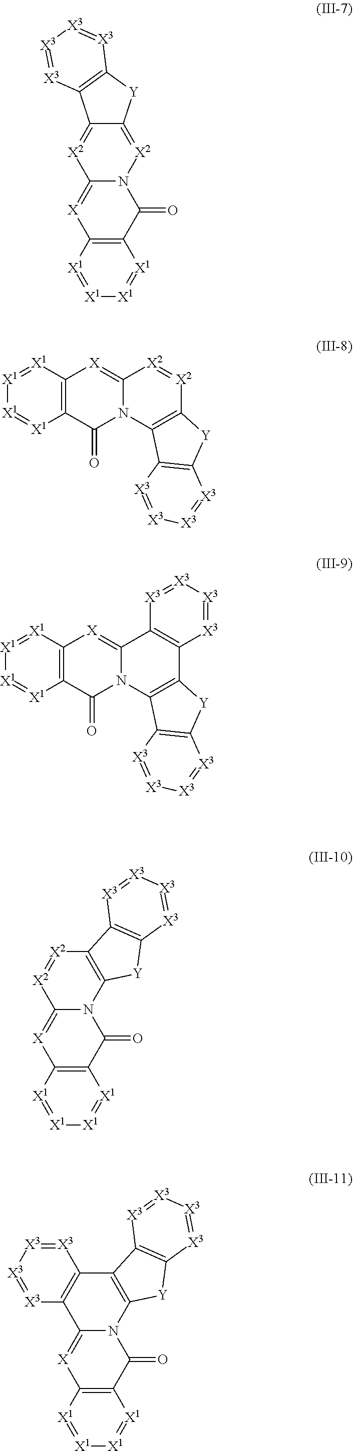

where the symbols X, X¹ and X² have the definitions given above, especially for formula (I), and the symbols Y and X³ have the definitions given above, especially for formulae (KAr-1) and (KAr-2). Preference is given here to structures/compounds of the formulae (III-1) to (III-7), (III-9) and (III-11), particular preference to structures/compounds of the formulae (III-1) to (III-3), (III-9) and (III-11), and very particular preference to structures/compounds of the formula (III-1).

It may preferably be the case that, in compounds of the formulae (III-1) to (III-11), not more than four, preferably not more than two, X¹, X³ groups are N, preferably all X¹, X³ groups are CR¹ or CR³, where preferably not more than 6, more preferably not more than 4 and especially preferably not more than 2 of the CR¹ or CR³ groups represented by X¹ or X³ are not the CH group.

In a further preferred embodiment, it may be the case that the compounds of the invention include a structure of the formulae (IV-1) to (IV-11), where the compounds of the invention may more preferably be selected from the compounds of the formulae (IV-1) to (IV-11)

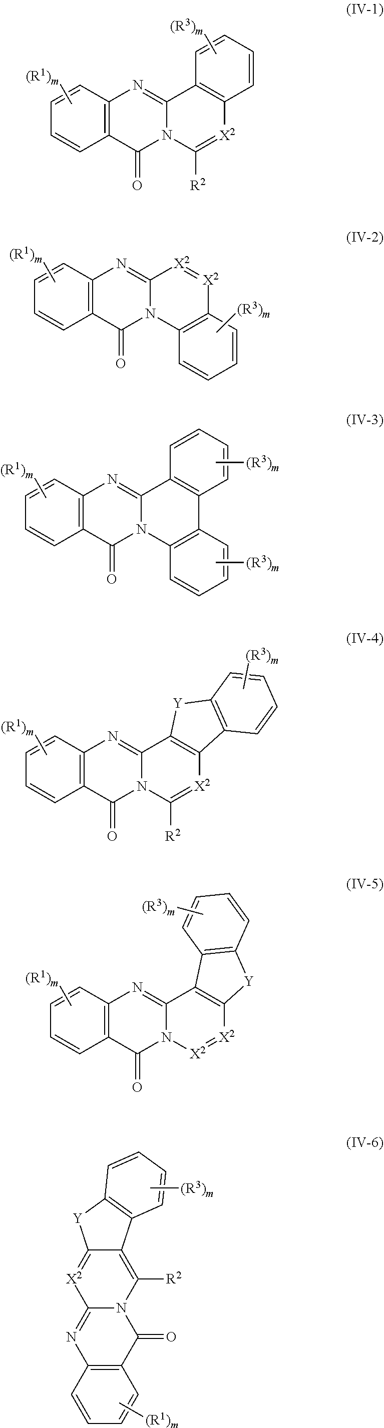

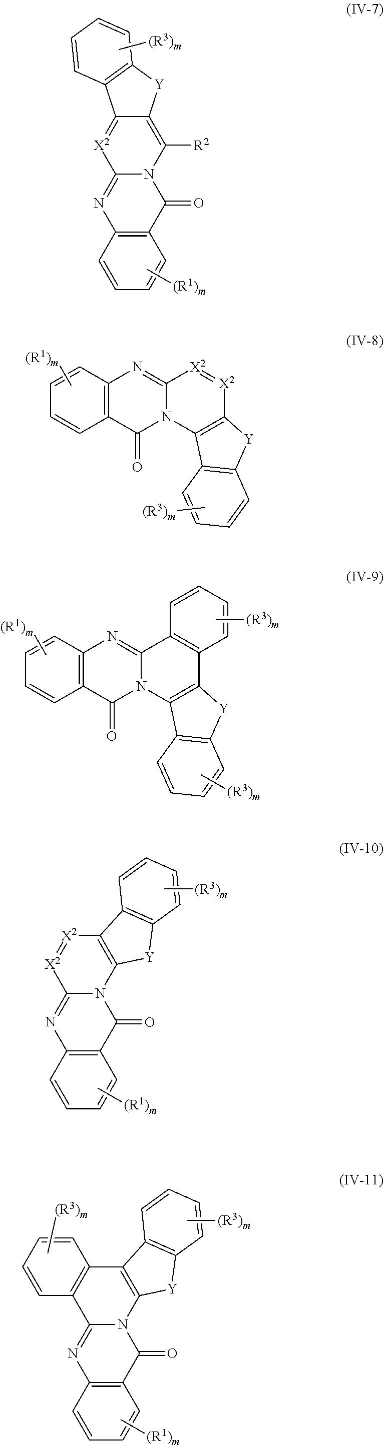

where the symbols R¹, R², R³ and X² have the definitions given above, especially for formula (I), the symbol Y has the definition given above, especially for formula (KAr-1) and (KAr-2), the index m is 0, 1, 2, 3 or 4, preferably 0, 1 or 2, where preferably at least one X² group is N, more preferably exactly one X² group is N. Preference is given here to structures/compounds of the formula (IV-1), and particular preference to structures/compounds of the formula (IV-1) with X²═N.

The index m is 0, 1, 2, 3 or 4, preferably 0, 1 or 2. If the index m is less than 4, the respective rings have a corresponding number of hydrogen atoms. It should be emphasized here that the R¹, R², R³ groups may be H. If, therefore, the index m is not 0, these rings preferably have substituents R¹, R², R³. This means that the corresponding R¹, R², R³ groups are preferably a radical other than H. In this context, the preferences set out above and hereinafter for the corresponding R¹, R², R³ groups are applicable. This clarification is correspondingly applicable to the further groups, radicals, for example R⁴, R⁵, R⁶, R⁷, R⁸ and/or R⁹, and indices, especially for n, l and r.

In a further preferred embodiment, it may be the case that the compounds of the invention include a structure of the formulae (V-1) to (V-5), where the compounds of the invention may more preferably be selected from the compounds of the formulae (V-1) to (V-5)

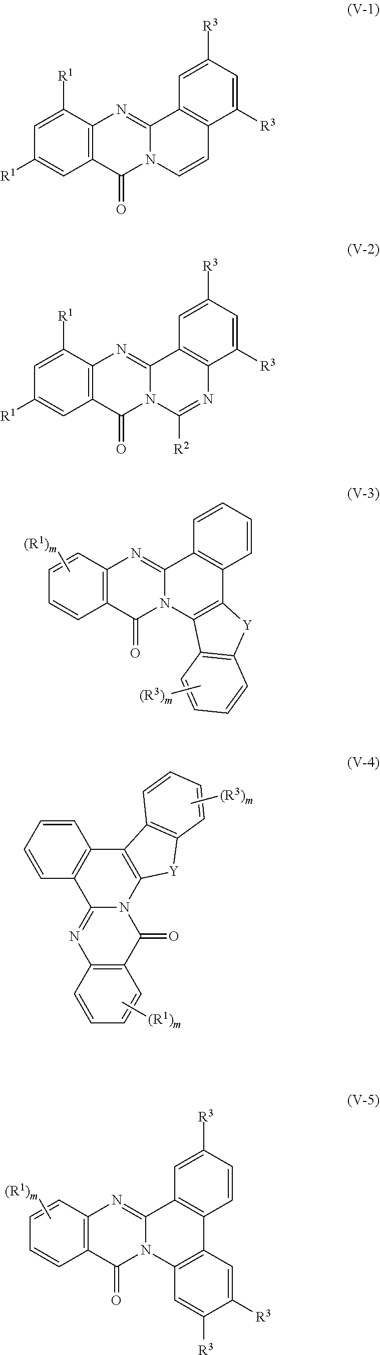

where the symbols R¹, R² and R³ have the definitions given above, especially for formula (I), the index m is 0, 1, 2, 3 or 4, preferably 0, 1 or 2.

Preferably, the sum total of the indices m is not more than 6, especially preferably not more than 4 and more preferably not more than 2. This is applicable to structures/compounds including those of the formulae (IV-1) to (IV-11) and (V-1) to (V-5). It is preferable here that at least one of the R¹, R² and R³ radicals shown explicitly is a group which is not H, and so there is substitution at this position. Radicals shown explicitly are not represented via the index m, but instead indicate the position of substitution on the ring. More preferably, at least two of the R¹, R² and R³ radicals explicitly shown are a group other than H.

It may preferably be the case, especially in formulae (V-1) to (V-5), that at least one R¹, R² and/or R³ radical in formula (VI-1) is an aromatic or heteroaromatic ring system which has 5 to 60 aromatic ring atoms, preferably 5 to 40 aromatic ring atoms, and in each case has R⁴ radicals. Particular preference is given here especially to heteroaromatic ring systems having 6 to 60 aromatic ring atoms, and/or aromatic ring systems having 10 to 60 aromatic ring atoms.

In a further-preferred embodiment, it may be the case that the compounds of the invention comprise a structure of the formula (VI-1), where the compounds of the invention may more preferably be selected from the compounds of the formula (VI-1):

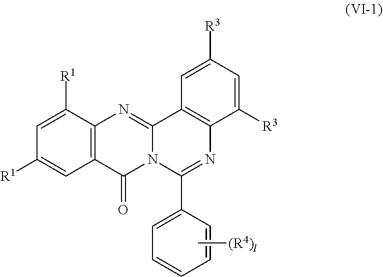

where the symbols R¹, R³ and R⁴ have the definitions given above, especially for formula (I), and the index l is 0, 1, 2, 3, 4 or 5, preferably 0, 1 or 2.

Preferably, in formula (VI-1), at least one of the R¹ and R³ radicals explicitly shown is preferably a group which is not H, and so there is substitution at this position. Preferably at least two of the R¹ and R³ radicals explicitly shown are a group other than H. It may preferably be the case that at least one R¹ and/or R³ radical in formula (VI-1) is an aromatic or heteroaromatic ring system which has 5 to 60 aromatic ring atoms, preferably 5 to 40 aromatic ring atoms, and in each case has R⁴ radicals. Particular preference is given here especially to heteroaromatic ring systems having 6 to 60 aromatic ring atoms, and/or aromatic ring systems having 10 to 60 aromatic ring atoms.

Preferably, two adjacent R¹ groups do not form a fused aromatic or heteroaromatic ring system with the groups to which the R¹ groups bind, where this includes the R⁴ or R⁵ radicals by which R¹ groups may be substituted.

It may further be the case that the substituents R, R¹, R² and R³ according to the above formulae do not form a fused aromatic or heteroaromatic ring system, preferably any fused ring system, with the ring atoms of the ring system. This includes the formation of a fused ring system with possible substituents R⁴, R⁵ which may be bonded to the R, R¹, R², R³ radicals.

When two radicals that may especially be selected from R, R², R³, R⁴, R⁵, R⁶, R⁷, R⁸ and/or R⁹ form a ring system with one another, this ring system may be mono- or polycyclic, aliphatic, heteroaliphatic, aromatic or heteroaromatic. In this case, the radicals which together form a ring system may be adjacent, meaning that these radicals are bonded to the same carbon atom or to carbon atoms directly bonded to one another, or they may be further removed from one another. In addition, the ring systems provided with the substituents R, R¹, R², R³, R⁴, R⁵, R⁶, R⁷, R⁸ and/or R⁹ may also be joined to one another via a bond, such that this can bring about a ring closure.

It may preferably be the case that at least one R, R¹, R² and/or R³ group is the same or different and is selected from the radicals of the following formulae SAr-1 to SAr-18:

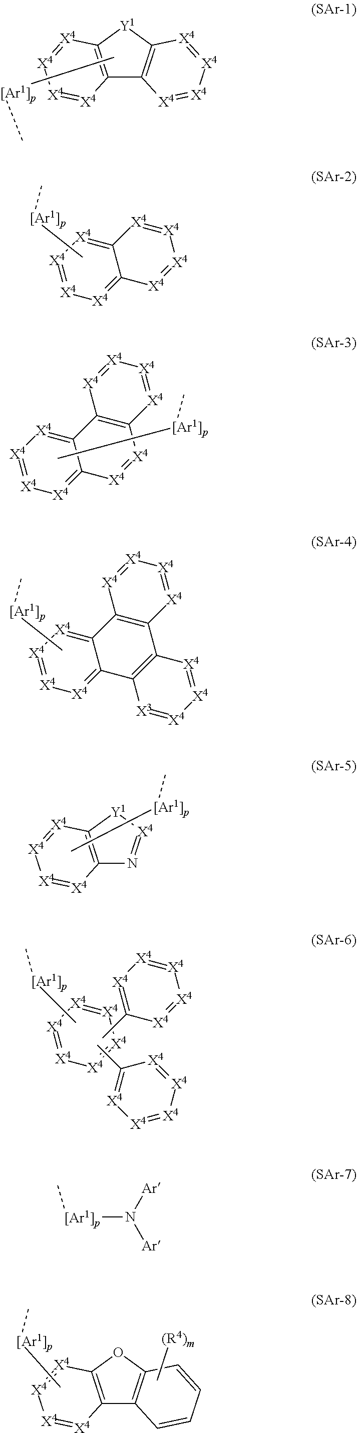

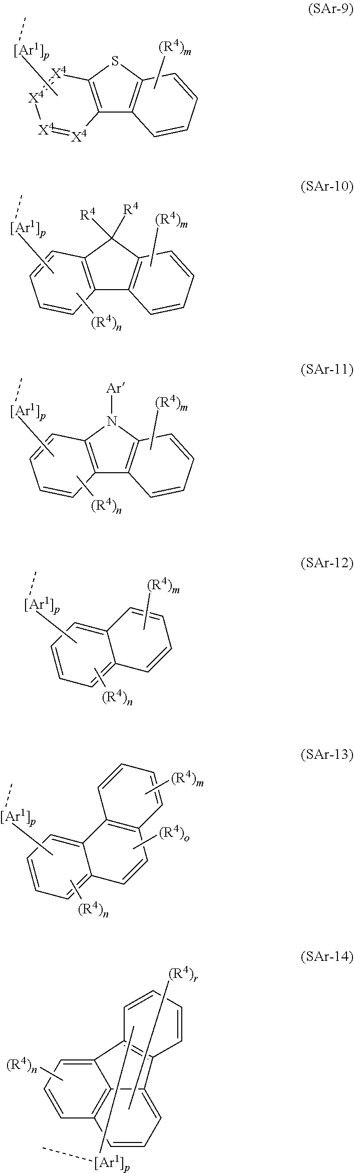

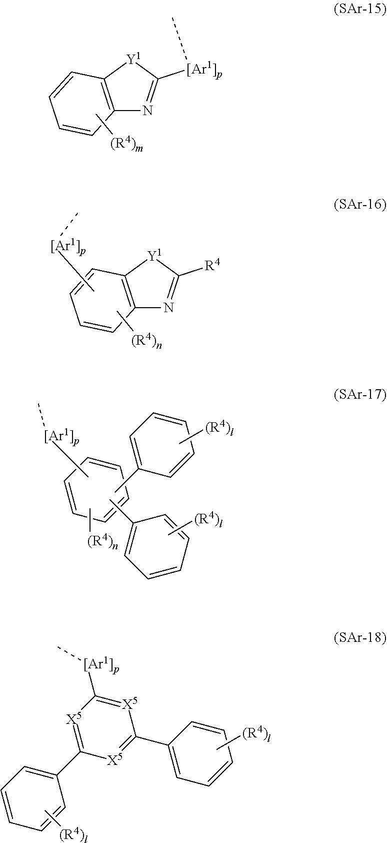

where R⁴ and Ar′ have the definitions given above, especially for formula (I), the dotted bond represents the bond to the corresponding group, and the further symbols and indices are as follows:
X⁴ is the same or different at each instance and is CR⁴, N, or C if the [Ar¹]ₚ group binds thereto, preferably CR⁴, where there are preferably no N—N bonds;
X⁵ is the same or different at each instance and is CR⁴ or N, preferably N;
Ar¹ is the same or different at each instance and is a bivalent aromatic or heteroaromatic ring system which has 6 to 18 aromatic ring atoms and in each case has R⁴ radicals;
Y¹ is the same or different at each instance and is C(R⁴)₂, NR⁴, O, S, or, if the [Ar¹]ₚ group binds thereto, is C(R⁴) or N;
p is 0 or 1, where p=0 means that the Ar¹ group is absent and that the corresponding aromatic or heteroaromatic group is bonded directly to the corresponding radical;
n is 0, 1, 2 or 3, preferably 0, 1 or 2;
m is 0, 1, 2, 3 or 4, preferably 0, 1 or 2;
1 is 0, 1, 2, 3, 4 or 5, preferably 0, 1 or 2;
r is 0, 1, 2, 3, 4, 5 or 6, preferably 0, 1 or 2.

Preference is given here to structures of the formulae (SAr-1), (SAr-4), (SAr-8), (SAr-11), (SAr-14), (SAr-18).

It may further be the case that R, R¹, R² and/or R³ are the same or different at each instance and are selected from the group consisting of H, D or an aromatic or heteroaromatic ring system selected from the groups of the following formulae Ar-1 to Ar-79; preferably, at least one R, R¹, R² and/or R³ group is the same or different and is selected from the groups of the following formulae Ar-1 to Ar-79 and/or the Ar′ group is the same or different at each instance and is selected from the groups of the following formulae Ar-1 to Ar-79:

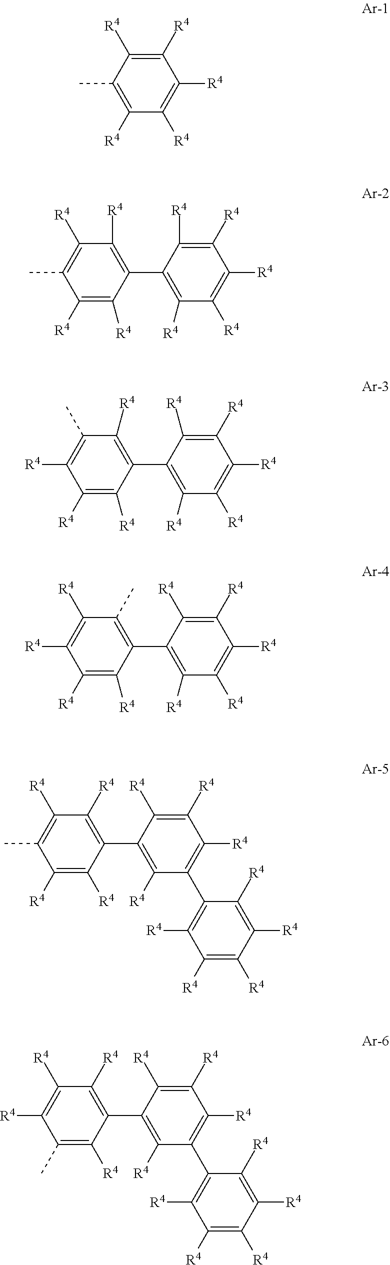

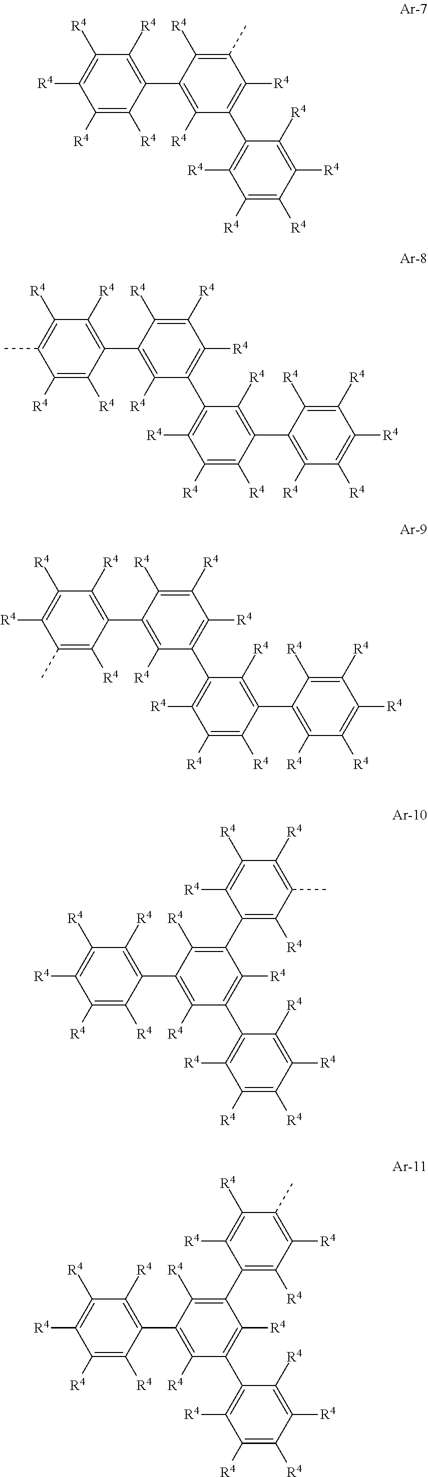

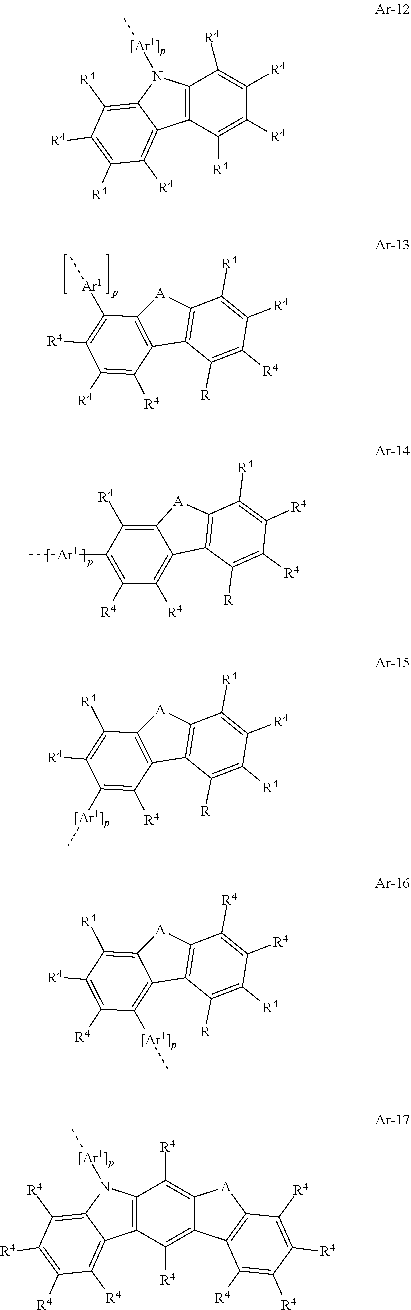

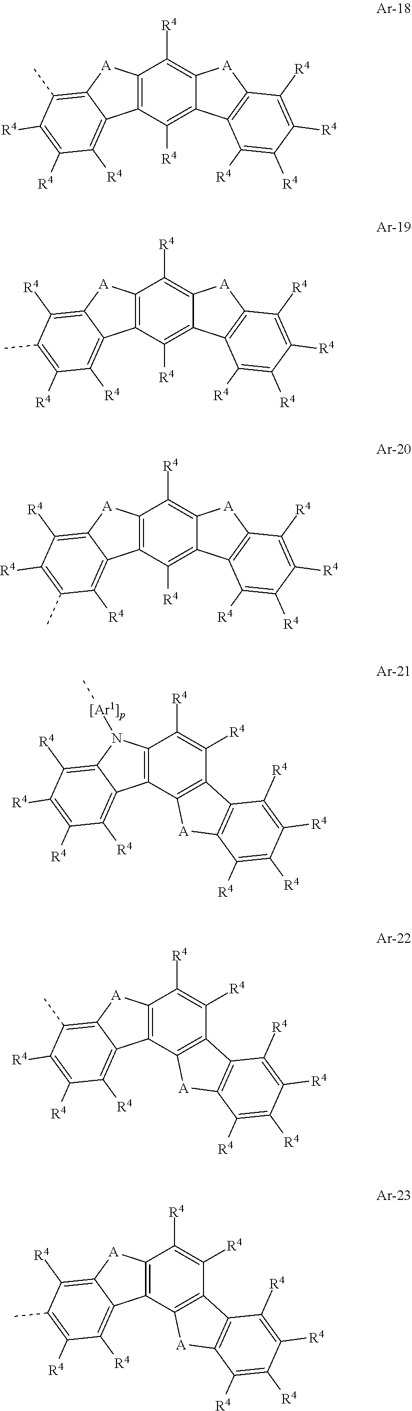

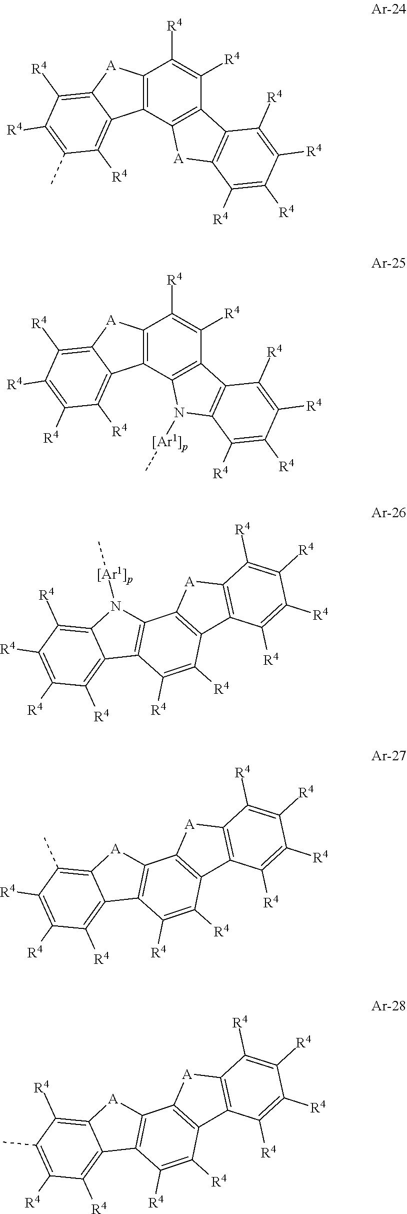

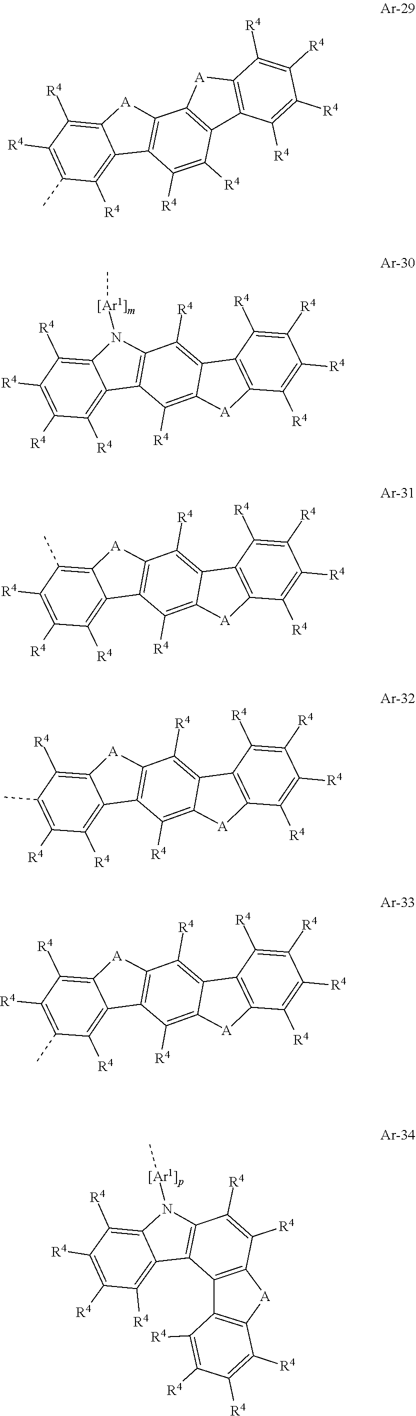

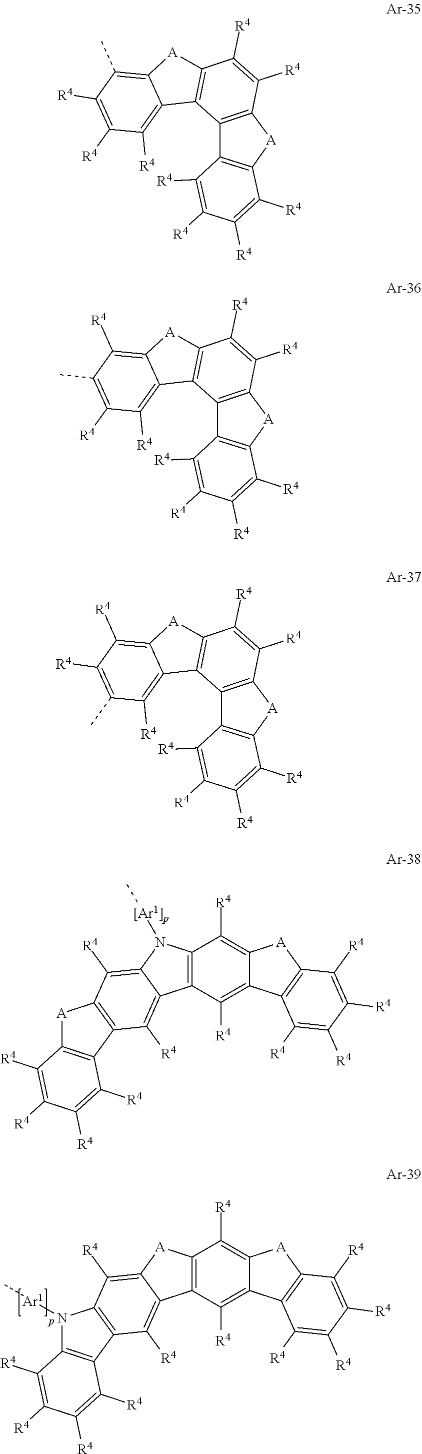

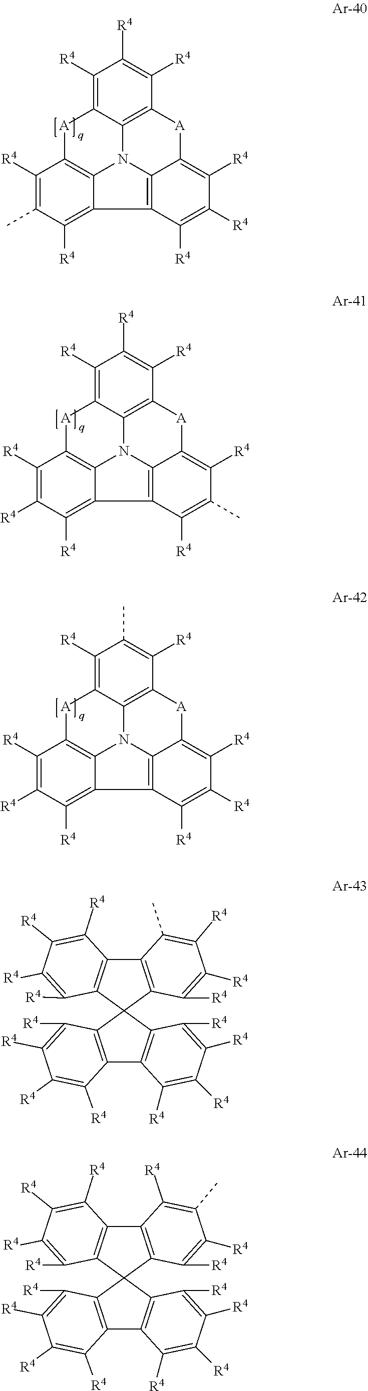

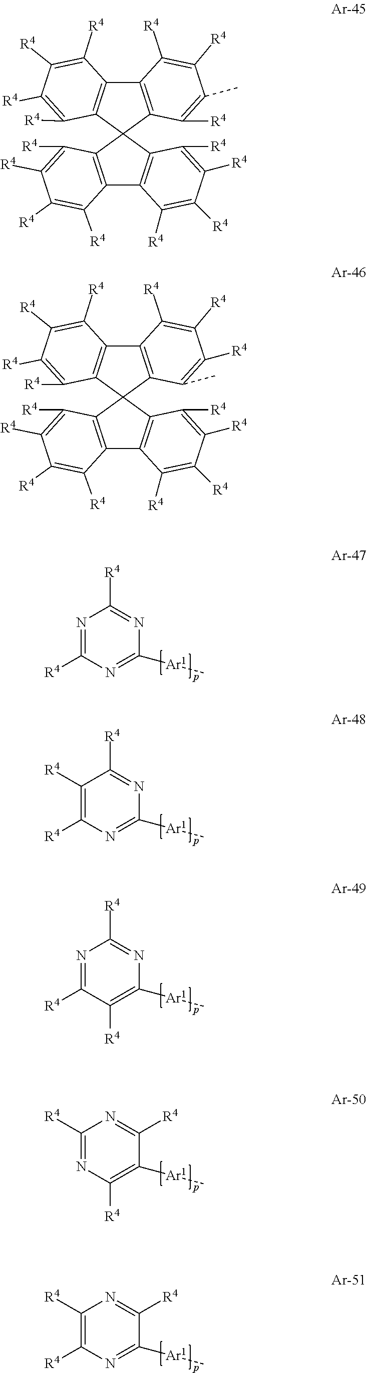

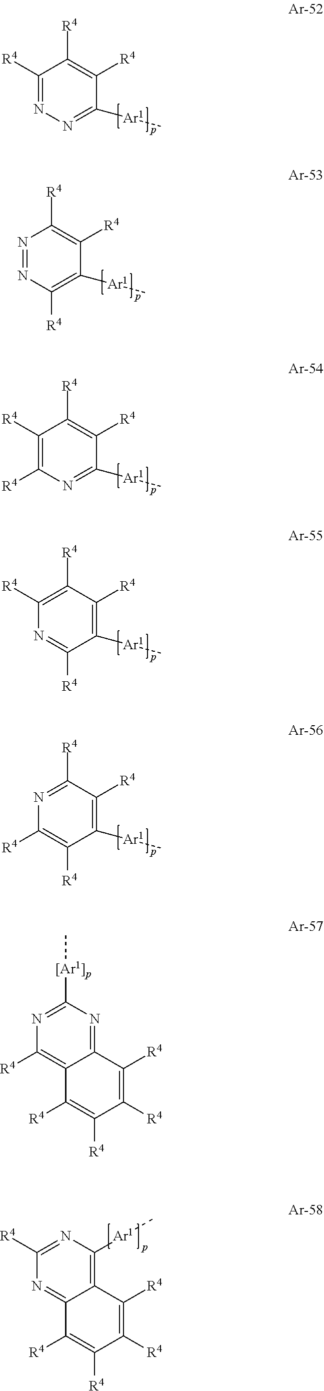

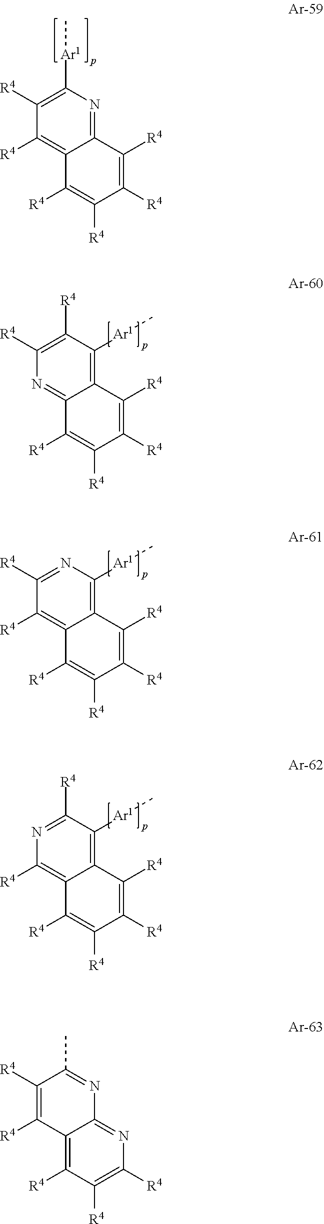

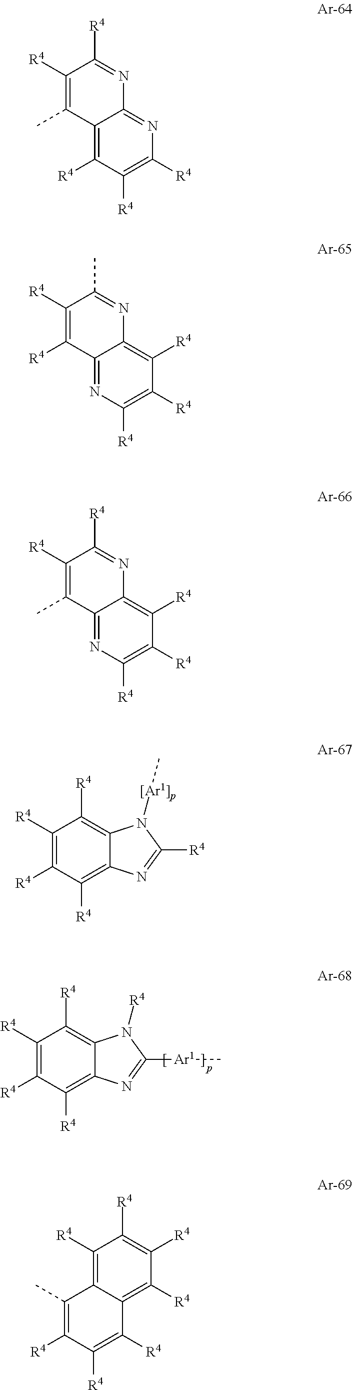

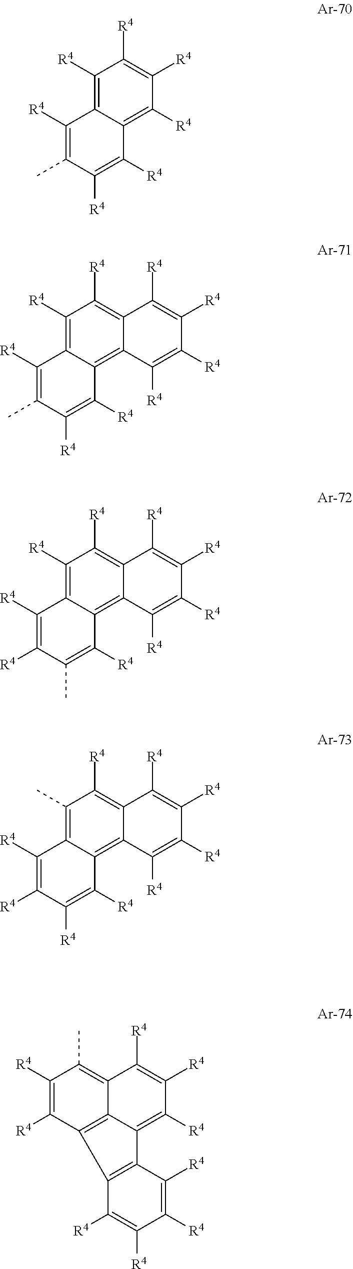

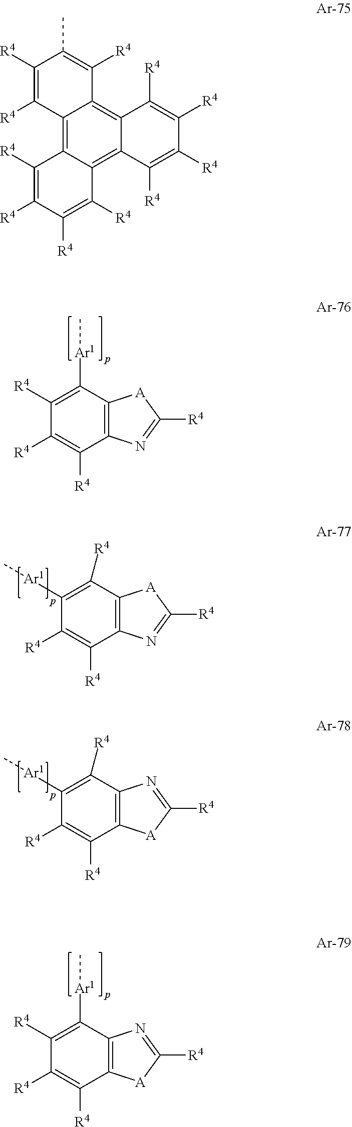

where R⁴ has the definitions given above, the dotted bond represents the bond to the corresponding group and in addition:
Ar¹ is the same or different at each instance and is a bivalent aromatic or heteroaromatic ring system which has 6 to 18 aromatic ring atoms and in each case has R⁴ radicals;
A is the same or different at each instance and is C(R⁴)₂, NR⁴, O or S;
p is 0 or 1, where p=0 means that the Ar¹ group is absent and that the corresponding aromatic or heteroaromatic group is bonded directly to the corresponding radical;
q is 0 or 1, where q=0 means that no A group is bonded at this position and R⁴ radicals are bonded to the corresponding carbon atoms instead.

Preference is given here to structures of the formulae (Ar-1), (Ar-2), (Ar-3), (Ar-12), (Ar-13), (Ar-14), (Ar-15), (Ar-16), (Ar-40), (Ar-41), (Ar-47) (Ar-57), (Ar-69), (Ar-70), (Ar-75), (Ar-76), and particular preference to structures of the formulae (Ar-1), (Ar-2), (Ar-3), (Ar-12), (Ar-13), (Ar-14), (Ar-15), (Ar-16) (Ar-57), (Ar-47).

When the abovementioned groups for R, R¹, R², R³ have two or more A groups, possible options for these include all combinations from the definition of A. Preferred embodiments in that case are those in which one A group is NR⁴ and the other A group is C(R⁴)₂ or in which both A groups are NR⁴ or in which both A groups are O.

When A is NR⁴, the substituent R⁴ bonded to the nitrogen atom is preferably an aromatic or heteroaromatic ring system which has 5 to 24 aromatic ring atoms and may also be substituted by one or more R⁵ radicals. In a particularly preferred embodiment, this R⁴ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R⁵ radicals. Preference is given to phenyl, biphenyl, terphenyl and quaterphenyl having bonding patterns as listed above for Ar-1 to Ar-11, where these structures, rather than by R⁴, may be substituted by one or more R⁵ radicals, but are preferably unsubstituted. Preference is further given to triazine, pyrimidine and quinazoline as listed above for Ar-47 to Ar-50, Ar-57 and Ar-58, where these structures, rather than by R⁴, may be substituted by one or more R⁵ radicals.

When A is C(R⁴)₂, the substituents R⁴ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R⁵ radicals. Most preferably, R⁴ is a methyl group or a phenyl group. In this case, the R⁴ radicals together may also form a ring system, which leads to a spiro system.

There follows a description of preferred substituents R, R¹, R² and R³.

In a preferred embodiment of the invention, R, R¹, R² and R³ are the same or different at each instance and are selected from the group consisting of H, D, F, CN, NO₂, Si(R⁴)₃, B(OR⁴)₂, a straight-chain alkyl group having 1 to 20 carbon atoms or a branched or cyclic alkyl group having 3 to 20 carbon atoms, where the alkyl group may be substituted in each case by one or more R⁴ radicals, or an aromatic or heteroaromatic ring system which has 5 to 60 aromatic ring atoms, preferably 5 to 40 aromatic ring atoms, and may be substituted in each case by one or more R⁴ radicals.

In a further-preferred embodiment of the invention, R, R¹, R² and R³ are the same or different at each instance and are selected from the group consisting of H, D, F, a straight-chain alkyl group having 1 to 20 carbon atoms or a branched or cyclic alkyl group having 3 to 20 carbon atoms, where the alkyl group may be substituted in each case by one or more R4 radicals, or an aromatic or heteroaromatic ring system which has 5 to 60 aromatic ring atoms, preferably 5 to 40 aromatic ring atoms, and may be substituted in each case by one or more R4 radicals.

In a further-preferred embodiment of the invention, R, R¹, R² and R³ are the same or different at each instance and is selected from the group consisting of H, D, an aromatic or heteroaromatic ring system which has 6 to 30 aromatic ring atoms and may be substituted by one or more R⁴ radicals, and an N(Ar′)₂ group. More preferably, R, R¹, R² are the same or different at each instance and is selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R⁴ radicals.

Preferred aromatic or heteroaromatic ring systems that the R, R¹, R², R³ and Ar′ radicals may preferably represent are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁴ radicals. Particular preference is given to the structures Ar-1 to Ar-79 shown above, preference being given to structures of the formulae (Ar-1), (Ar-2), (Ar-3), (Ar-12), (Ar-13), (Ar-14), (Ar-15), (Ar-16), (Ar-40), (Ar-41), (Ar-47) (Ar-57), (Ar-69), (Ar-70), (Ar-75), (Ar-76), and particular preference to structures of the formulae (Ar-1), (Ar-2), (Ar-3), (Ar-12), (Ar-13), (Ar-14), (Ar-15), (Ar-16) (Ar-57), (Ar-47).

Further suitable R, R¹, R² and R³ groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R⁴ radicals. The total number of aromatic ring atoms in Ar², Ar³ and Ar⁴ here is not more than 60 and preferably not more than 40.

Ar⁴ and Ar² here may also be bonded to one another and/or Ar² and Ar³ to one another by a group selected from C(R⁴)₂, NR⁴, O and S. Preferably, Ar⁴ and Ar² are joined to one another and Ar² and Ar³ to one another in the respective ortho position to the bond to the nitrogen atom. In a further embodiment of the invention, none of the Ar², Ar³ and Ar⁴ groups are bonded to one another.

Preferably, Ar⁴ is an aromatic or heteroaromatic ring system which has 6 to 24 aromatic ring atoms, preferably 6 to 12 aromatic ring atoms, and may be substituted in each case by one or more R⁴ radicals. More preferably, Ar⁴ is selected from the group consisting of ortho-, meta- or para-phenylene or ortho-, meta- or para-biphenyl, each of which may be substituted by one or more R⁴ radicals, but are preferably unsubstituted. Most preferably, Ar⁴ is an unsubstituted phenylene group.

Preferably, Ar² and Ar³ are the same or different at each instance and are an aromatic or heteroaromatic ring system which has 6 to 24 aromatic ring atoms and may be substituted in each case by one or more R⁴ radicals. Particularly preferred Ar² and Ar³ groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene or triphenylene, each of which may be substituted by one or more R¹ radicals. Most preferably, Ar² and Ar³ are the same or different at each instance and are selected from the group consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

In a further preferred embodiment of the invention, R⁴ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R² radicals, or an aromatic or heteroaromatic ring system which has 6 to 24 aromatic ring atoms and may be substituted in each case by one or more R⁵ radicals. In a particularly preferred embodiment of the invention, R⁴ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R⁵ radicals, but is preferably unsubstituted, or an aromatic or heteroaromatic ring system which has 6 to 13 aromatic ring atoms and may be substituted in each case by one or more R⁵ radicals, but is preferably unsubstituted.

In a further preferred embodiment of the invention, R⁵ is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

At the same time, in compounds of the invention that are processed by vacuum evaporation, the alkyl groups preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

It may further be the case that the compound comprises exactly two or exactly three structures of formulae (I), (II-1) to (II-7), (III-1) to (III-11), (IV-1) to (IV-11), (V-1) to (V-5) and/or (VI-1), where preferably one of the aromatic or heteroaromatic ring systems that can be represented by the at least one of the R¹, R², R³ groups or to which the R¹, R², R³ groups bind is shared by the two structures. It may additionally be the case that the compound comprises a connecting group via which the exactly two or three structures of formulae (I), (II-1) to (II-7), (III-1) to (III-11), (IV-1) to (IV-11), (V-1) to (V-5) and/or (VI-1) are bonded to one another. These connecting groups are preferably derived from groups that are defined for the R¹, R², R³ groups, but where one or two hydrogen atoms should be replaced by bonding sites. In a further configuration, an inventive compound comprising structures of formulae (I), (II-1) to (II-7), (III-1) to (III-11), (IV-1) to (IV-11), (V-1) to (V-5) and/or (VI-1) may be configured as an oligomer, polymer or dendrimer, where, in place of one hydrogen atom or one substituent, there are one or more bonds of the compounds to the polymer, oligomer or dendrimer.

When the compounds of the formula (I) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. An exception to this is formed by phenanthrene and triphenylene, which, because of their high triplet energy, may be preferable in spite of the presence of fused aromatic six-membered rings.

In addition, it is a feature of preferred compounds of the invention that they are sublimable. These compounds generally have a molar mass of less than about 1200 g/mol.

The abovementioned preferred embodiments may be combined with one another as desired within the restrictions defined in claim 1. In a particularly preferred embodiment of the invention, the abovementioned preferences occur simultaneously.

Examples of preferred compounds according to the embodiments detailed above are the compounds detailed in the following table:

| | |
|---|---|
| 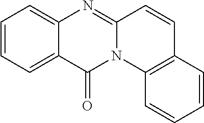 [SMILES: O=c1c2ccccc2nc2ccc3ccccc3n12] | 1 |
| 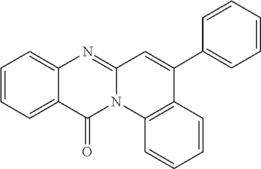 [SMILES: O=c1c2ccccc2nc2cc(-c3ccccc3)c3ccccc3n12] | 2 |
| 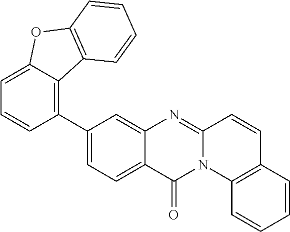 [SMILES: O=c1c2ccc(-c3cccc4oc5ccccc5c34)cc2nc2ccc3ccccc3n12] | 3 |
| 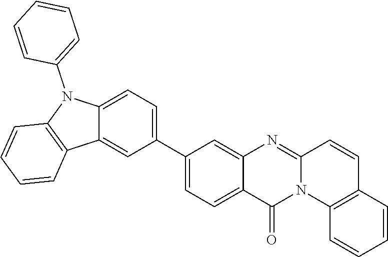 [SMILES: O=c1c2ccc(-c3ccc4c(c3)c3ccccc3n4-c3ccccc3)cc2nc2ccc3ccccc3n12] | 4 |
| 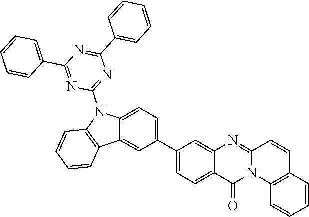 [SMILES: O=c1c2ccc(-c3ccc4c(c3)c3ccccc3n4-c3nc(-c4ccccc4)nc(-c4ccccc4)n3)cc2nc2ccc3ccccc3n12] | 5 |
| 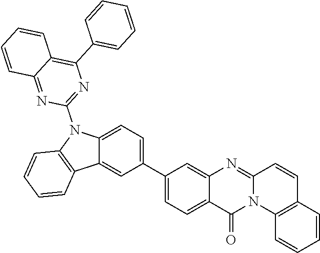 [SMILES: O=c1c2ccc(-c3ccc4c(c3)c3ccccc3n4-c3nc(-c4ccccc4)c4ccccc4n3)cc2nc2ccc3ccccc3n12] | 6 |
| 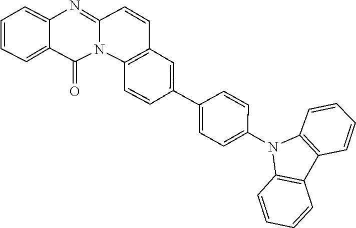 [SMILES: O=c1c2ccccc2nc2ccc3cc(-c4ccc(-n5c6ccccc6c6ccccc65)cc4)ccc3n12] | 7 |
| 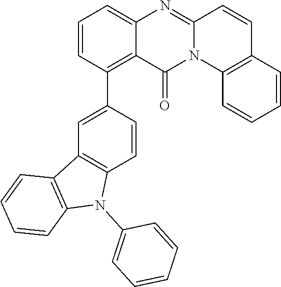 [SMILES: O=c1c2c(-c3ccc4c(c3)c3ccccc3n4-c3ccccc3)cccc2nc2ccc3ccccc3n12] | 8 |
| 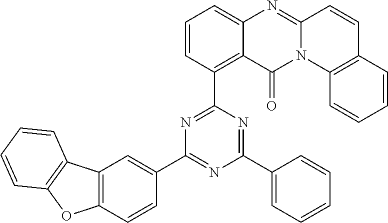 [SMILES: O=c1c2c(-c3nc(-c4ccccc4)nc(-c4ccc5oc6ccccc6c5c4)n3)cccc2nc2ccc3ccccc3n12] | 9 |
| 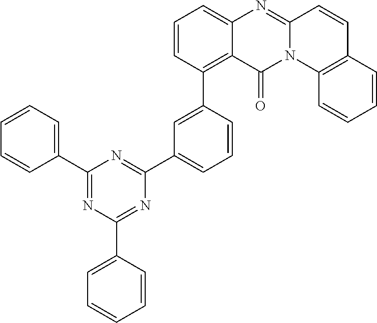 [SMILES: O=c1c2c(-c3cccc(-c4nc(-c5ccccc5)nc(-c5ccccc5)n4)c3)cccc2nc2ccc3ccccc3n12] | 10 |
| 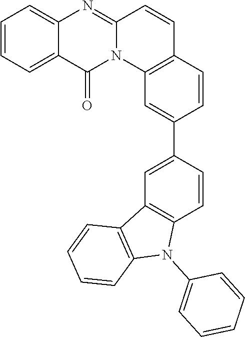 [SMILES: O=c1c2ccccc2nc2ccc3ccc(-c4ccc5c(c4)c4ccccc4n5-c4ccccc4)cc3n12] | 11 |
| 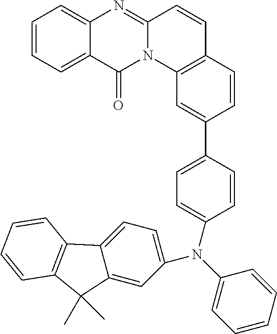 [SMILES: CC1(C)c2ccccc2-c2ccc(N(c3ccccc3)c3ccc(-c4ccc5ccc6nc7ccccc7c(=O)n6c5c4)cc3)cc21] | 12 |
| 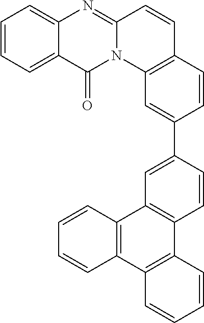 [SMILES: O=c1c2ccccc2nc2ccc3ccc(-c4ccc5c6ccccc6c6ccccc6c5c4)cc3n12] | 13 |
| 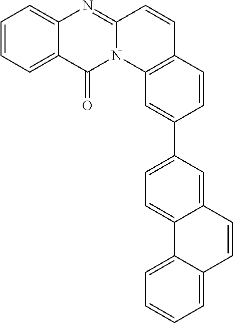 [SMILES: O=c1c2ccccc2nc2ccc3ccc(-c4ccc5c(ccc6ccccc65)c4)cc3n12] | 14 |
| 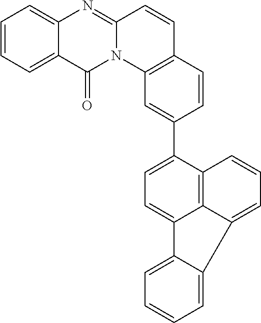 [SMILES: O=c1c2ccccc2nc2ccc3ccc(-c4ccc5c6c(cccc46)-c4ccccc4-5)cc3n12] | 15 |
| 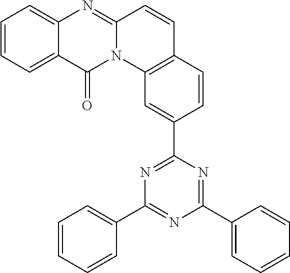 [SMILES: O=c1c2ccccc2nc2ccc3ccc(-c4nc(-c5ccccc5)nc(-c5ccccc5)n4)cc3n12] | 16 |
| 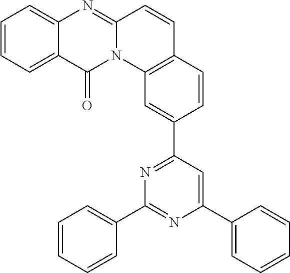 [SMILES: O=c1c2ccccc2nc2ccc3ccc(-c4cc(-c5ccccc5)nc(-c5ccccc5)n4)cc3n12] | 17 |
| 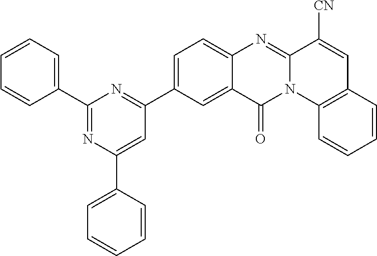 [SMILES: N#Cc1cc2ccccc2n2c(=O)c3cc(-c4cc(-c5ccccc5)nc(-c5ccccc5)n4)ccc3nc12] | 18 |
| 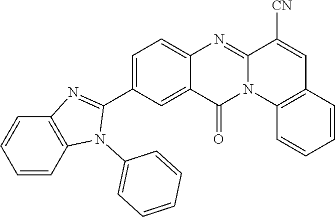 [SMILES: N#Cc1cc2ccccc2n2c(=O)c3cc(-c4nc5ccccc5n4-c4ccccc4)ccc3nc12] | 19 |
| 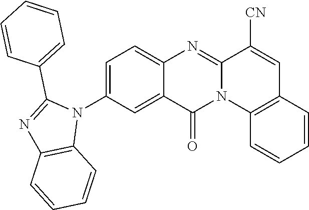 [SMILES: N#Cc1cc2ccccc2n2c(=O)c3cc(-n4c(-c5ccccc5)nc5ccccc54)ccc3nc12] | 20 |
| 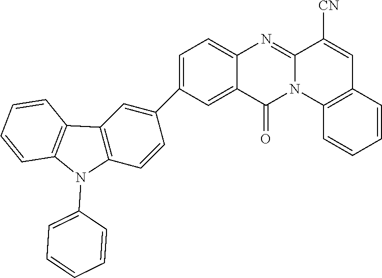 [SMILES: N#Cc1cc2ccccc2n2c(=O)c3cc(-c4ccc5c(c4)c4ccccc4n5-c4ccccc4)ccc3nc12] | 21 |
| 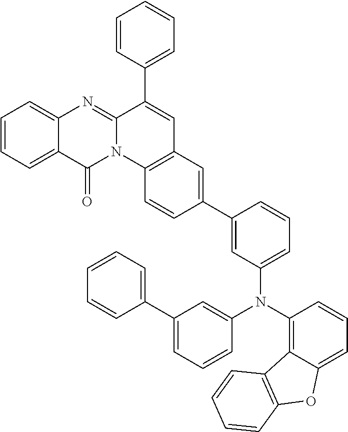 [SMILES: O=c1c2ccccc2nc2c(-c3ccccc3)cc3cc(-c4cccc(N(c5cccc(-c6ccccc6)c5)c5cccc6oc7ccccc7c56)c4)ccc3n12] | 22 |
| 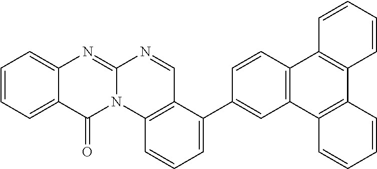 [SMILES: O=c1c2ccccc2nc2ncc3c(-c4ccc5c6ccccc6c6ccccc6c5c4)cccc3n12] | 23 |
| 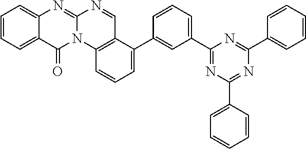 [SMILES: O=c1c2ccccc2nc2ncc3c(-c4cccc(-c5nc(-c6ccccc6)nc(-c6ccccc6)n5)c4)cccc3n12] | 24 |
| 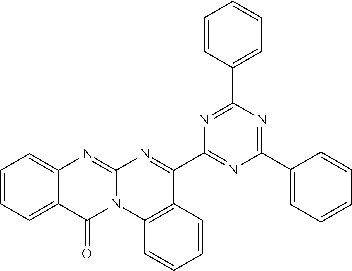 [SMILES: O=c1c2ccccc2nc2nc(-c3nc(-c4ccccc4)nc(-c4ccccc4)n3)c3ccccc3n12] | 25 |
| 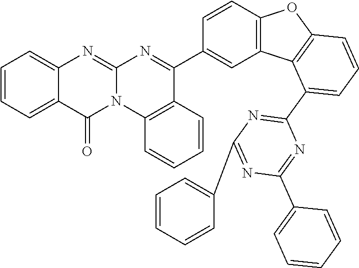 [SMILES: O=c1c2ccccc2nc2nc(-c3ccc4oc5cccc(-c6nc(-c7ccccc7)nc(-c7ccccc7)n6)c5c4c3)c3ccccc3n12] | 26 |
| 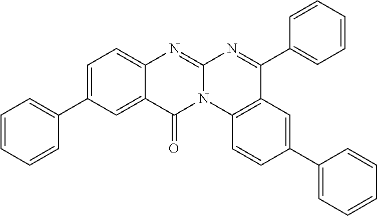 [SMILES: O=c1c2cc(-c3ccccc3)ccc2nc2nc(-c3ccccc3)c3cc(-c4ccccc4)ccc3n12] | 27 |
| 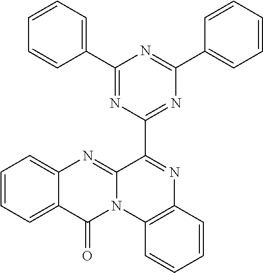 [SMILES: O=c1c2ccccc2nc2c(-c3nc(-c4ccccc4)nc(-c4ccccc4)n3)nc3ccccc3n12] | 28 |
| 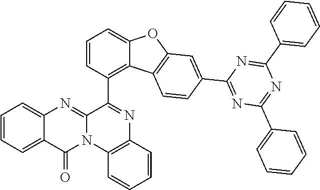 [SMILES: O=c1c2ccccc2nc2c(-c3cccc4oc5cc(-c6nc(-c7ccccc7)nc(-c7ccccc7)n6)ccc5c34)nc3ccccc3n12] | 29 |
| 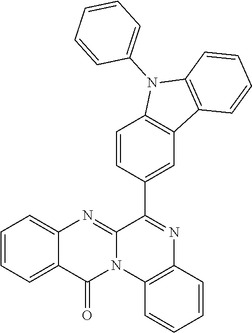 [SMILES: O=c1c2ccccc2nc2c(-c3ccc4c(c3)c3ccccc3n4-c3ccccc3)nc3ccccc3n12] | 30 |
| 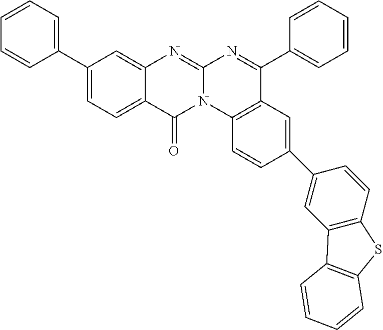 [SMILES: O=c1c2ccc(-c3ccccc3)cc2nc2nc(-c3ccccc3)c3cc(-c4ccc5sc6ccccc6c5c4)ccc3n12] | 31 |
| 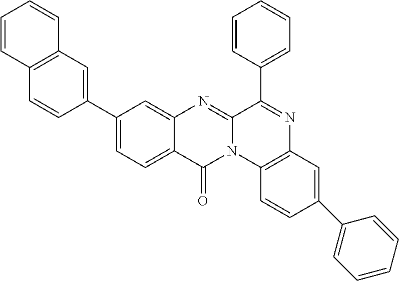 [SMILES: O=c1c2ccc(-c3ccc4ccccc4c3)cc2nc2c(-c3ccccc3)nc3cc(-c4ccccc4)ccc3n12] | 32 |
| 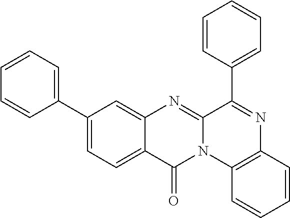 [SMILES: O=c1c2ccc(-c3ccccc3)cc2nc2c(-c3ccccc3)nc3ccccc3n12] | 33 |
| 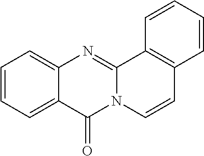 [SMILES: O=c1c2ccccc2nc2c3ccccc3ccn12] | 34 |
| 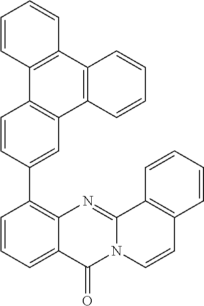 [SMILES: O=c1c2cccc(-c3ccc4c5ccccc5c5ccccc5c4c3)c2nc2c3ccccc3ccn12] | 35 |
| 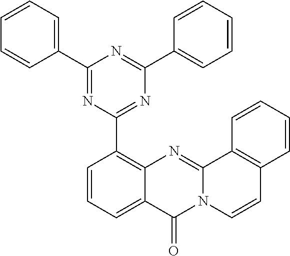 [SMILES: O=c1c2cccc(-c3nc(-c4ccccc4)nc(-c4ccccc4)n3)c2nc2c3ccccc3ccn12] | 36 |
| 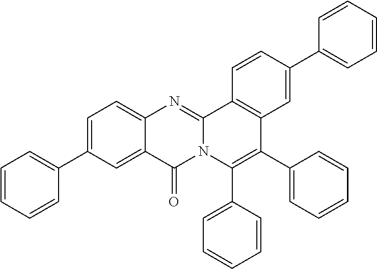 [SMILES: O=c1c2cc(-c3ccccc3)ccc2nc2c3ccc(-c4ccccc4)cc3c(-c3ccccc3)c(-c3ccccc3)n12] | 37 |
| 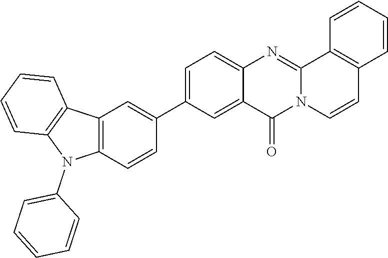 [SMILES: O=c1c2cc(-c3ccc4c(c3)c3ccccc3n4-c3ccccc3)ccc2nc2c3ccccc3ccn12] | 38 |
| 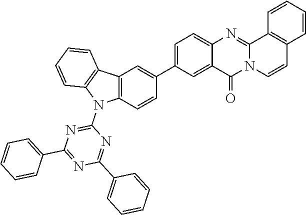 [SMILES: O=c1c2cc(-c3ccc4c(c3)c3ccccc3n4-c3nc(-c4ccccc4)nc(-c4ccccc4)n3)ccc2nc2c3ccccc3ccn12] | |
| 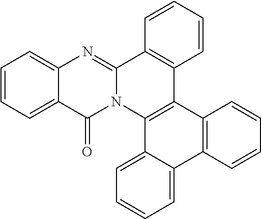 [SMILES: O=c1c2ccccc2nc2c3ccccc3c3c4ccccc4c4ccccc4c3n12] | 40 |
| 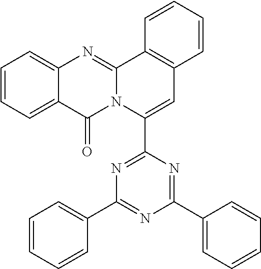 [SMILES: O=c1c2ccccc2nc2c3ccccc3cc(-c3nc(-c4ccccc4)nc(-c4ccccc4)n3)n12] | 41 |
| 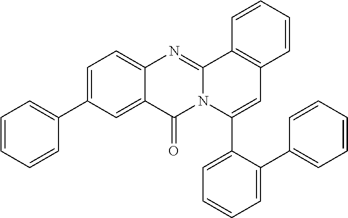 [SMILES: O=c1c2cc(-c3ccccc3)ccc2nc2c3ccccc3cc(-c3ccccc3-c3ccccc3)n12] | 42 |
| 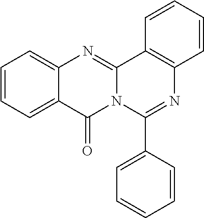 [SMILES: O=c1c2ccccc2nc2c3ccccc3nc(-c3ccccc3)n12] | 43 |
| 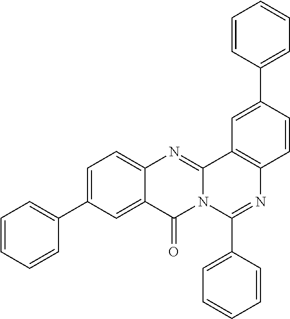 [SMILES: O=c1c2cc(-c3ccccc3)ccc2nc2c3cc(-c4ccccc4)ccc3nc(-c3ccccc3)n12] | 44 |
| 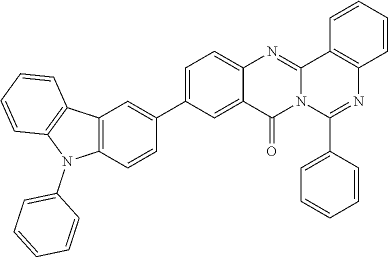 [SMILES: O=c1c2cc(-c3ccc4c(c3)c3ccccc3n4-c3ccccc3)ccc2nc2c3ccccc3nc(-c3ccccc3)n12] | 45 |
| 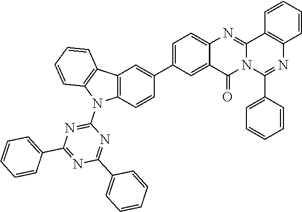 [SMILES: O=c1c2cc(-c3ccc4c(c3)c3ccccc3n4-c3nc(-c4ccccc4)nc(-c4ccccc4)n3)ccc2nc2c3ccccc3nc(-c3ccccc3)n12] | 46 |
| 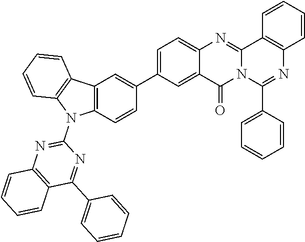 [SMILES: O=c1c2cc(-c3ccc4c(c3)c3ccccc3n4-c3nc(-c4ccccc4)c4ccccc4n3)ccc2nc2c3ccccc3nc(-c3ccccc3)n12] | 47 |
| 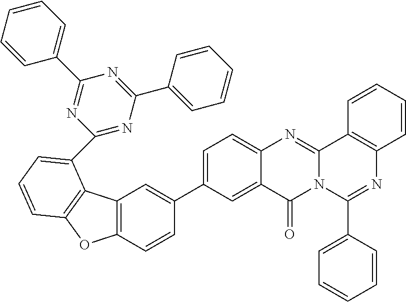 [SMILES: O=c1c2cc(-c3ccc4oc5cccc(-c6nc(-c7ccccc7)nc(-c7ccccc7)n6)c5c4c3)ccc2nc2c3ccccc3nc(-c3ccccc3)n12] | 48 |
| 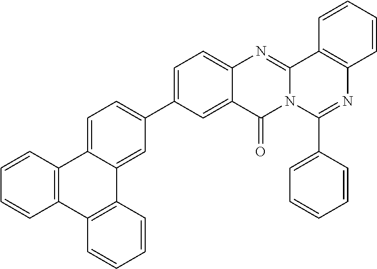 [SMILES: O=c1c2cc(-c3ccc4c5ccccc5c5ccccc5c4c3)ccc2nc2c3ccccc3nc(-c3ccccc3)n12] | 49 |
| 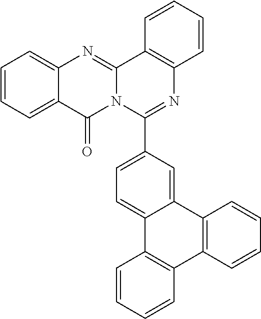 [SMILES: O=c1c2ccccc2nc2c3ccccc3nc(-c3ccc4c5ccccc5c5ccccc5c4c3)n12] | 50 |
| 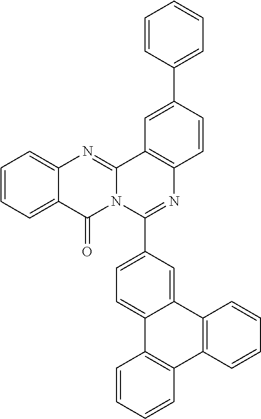 [SMILES: O=c1c2ccccc2nc2c3cc(-c4ccccc4)ccc3nc(-c3ccc4c5ccccc5c5ccccc5c4c3)n12] | 51 |
| 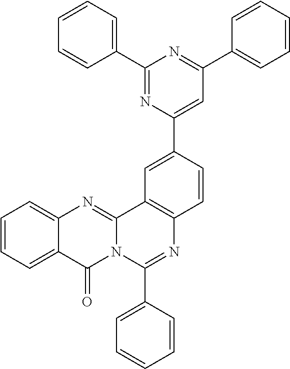 [SMILES: O=c1c2ccccc2nc2c3cc(-c4cc(-c5ccccc5)nc(-c5ccccc5)n4)ccc3nc(-c3ccccc3)n12] | 52 |
| 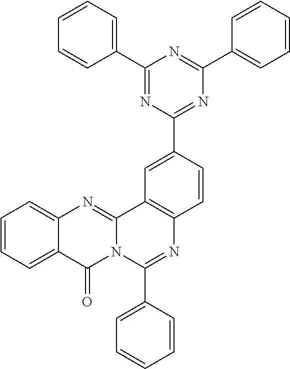 [SMILES: O=c1c2ccccc2nc2c3cc(-c4nc(-c5ccccc5)nc(-c5ccccc5)n4)ccc3nc(-c3ccccc3)n12] | 53 |
| 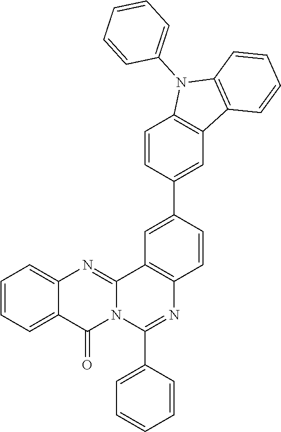 [SMILES: O=c1c2ccccc2nc2c3cc(-c4ccc5c(c4)c4ccccc4n5-c4ccccc4)ccc3nc(-c3ccccc3)n12] | 54 |
| 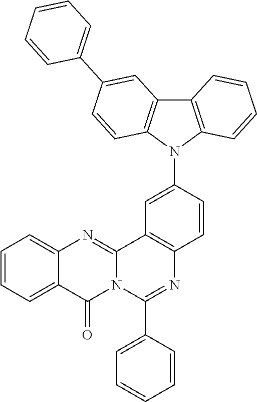 [SMILES: O=c1c2ccccc2nc2c3cc(-n4c5ccccc5c5cc(-c6ccccc6)ccc54)ccc3nc(-c3ccccc3)n12] | 55 |
| 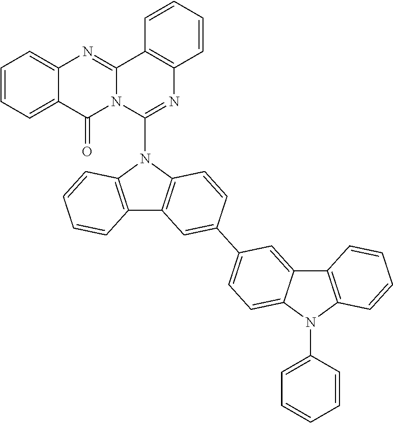 [SMILES: O=c1c2ccccc2nc2c3ccccc3nc(-n3c4ccccc4c4cc(-c5ccc6c(c5)c5ccccc5n6-c5ccccc5)ccc43)n12] | 56 |
| 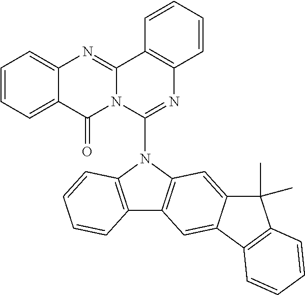 [SMILES: CC1(C)c2ccccc2-c2cc3c4ccccc4n(-c4nc5ccccc5c5nc6ccccc6c(=O)n45)c3cc21] | 57 |
| 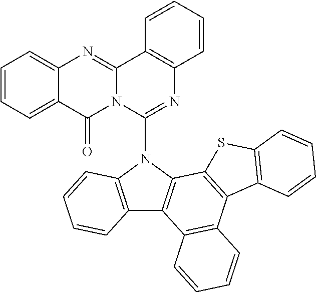 [SMILES: O=c1c2ccccc2nc2c3ccccc3nc(-n3c4ccccc4c4c5ccccc5c5c6ccccc6sc5c43)n12] | 58 |
| 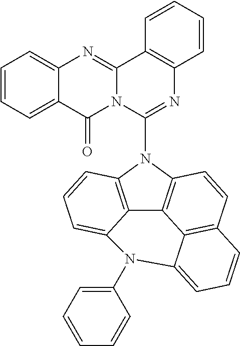 [SMILES: O=c1c2ccccc2nc2c3ccccc3nc(-n3c4cccc5c4c4c6c(cccc6ccc43)N5c3ccccc3)n12] | 59 |
| 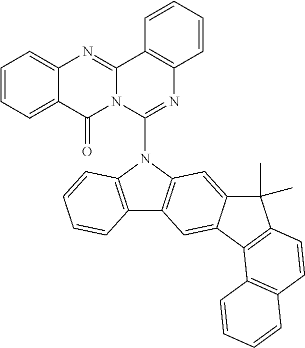 [SMILES: CC1(C)c2cc3c(cc2-c2c1ccc1ccccc21)c1ccccc1n3-c1nc2ccccc2c2nc3ccccc3c(=O)n12] | 60 |
| 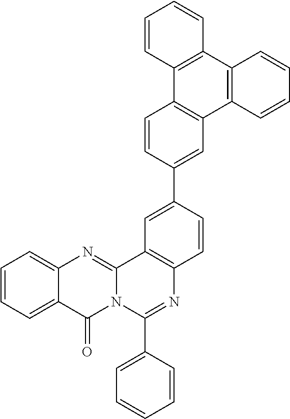 [SMILES: O=c1c2ccccc2nc2c3cc(-c4ccc5c6ccccc6c6ccccc6c5c4)ccc3nc(-c3ccccc3)n12] | 61 |
| 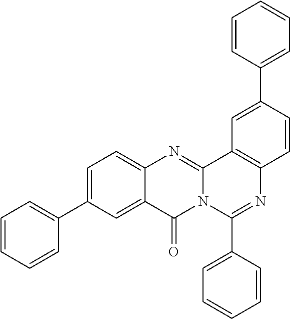 [SMILES: O=c1c2cc(-c3ccccc3)ccc2nc2c3cc(-c4ccccc4)ccc3nc(-c3ccccc3)n12] | 62 |
| 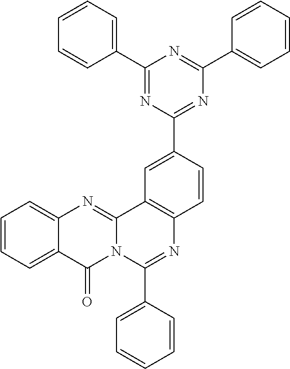 [SMILES: O=c1c2ccccc2nc2c3cc(-c4nc(-c5ccccc5)nc(-c5ccccc5)n4)ccc3nc(-c3ccccc3)n12] | 63 |
| 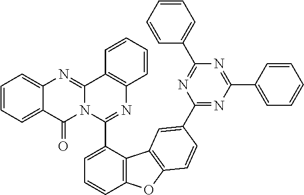 [SMILES: O=c1c2ccccc2nc2c3ccccc3nc(-c3cccc4oc5ccc(-c6nc(-c7ccccc7)nc(-c7ccccc7)n6)cc5c34)n12] | 64 |
| 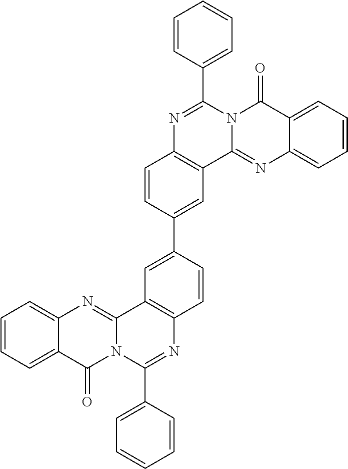 [SMILES: O=c1c2ccccc2nc2c3cc(-c4ccc5nc(-c6ccccc6)n6c(=O)c7ccccc7nc6c5c4)ccc3nc(-c3ccccc3)n12] | 65 |
| 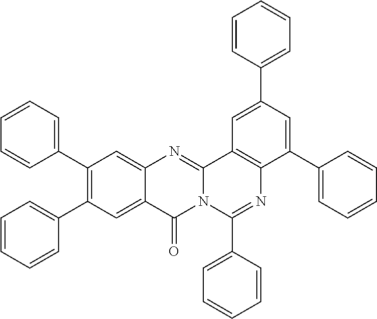 [SMILES: O=c1c2cc(-c3ccccc3)c(-c3ccccc3)cc2nc2c3cc(-c4ccccc4)cc(-c4ccccc4)c3nc(-c3ccccc3)n12] | 66 |
| 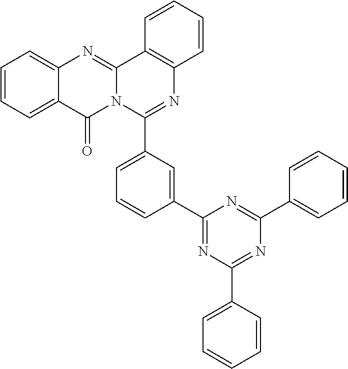 [SMILES: O=c1c2ccccc2nc2c3ccccc3nc(-c3cccc(-c4nc(-c5ccccc5)nc(-c5ccccc5)n4)c3)n12] | 67 |
| 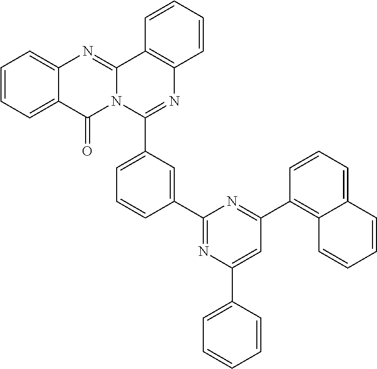 [SMILES: O=c1c2ccccc2nc2c3ccccc3nc(-c3cccc(-c4nc(-c5ccccc5)cc(-c5cccc6ccccc56)n4)c3)n12] | 68 |
| 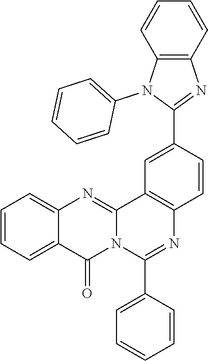 [SMILES: O=c1c2ccccc2nc2c3cc(-c4nc5ccccc5n4-c4ccccc4)ccc3nc(-c3ccccc3)n12] | 69 |
| 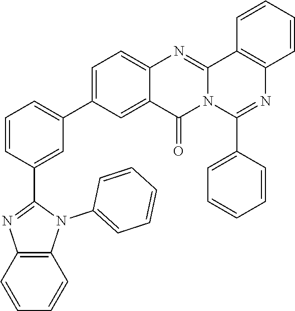 [SMILES: O=c1c2cc(-c3cccc(-c4nc5ccccc5n4-c4ccccc4)c3)ccc2nc2c3ccccc3nc(-c3ccccc3)n12] | 70 |
| 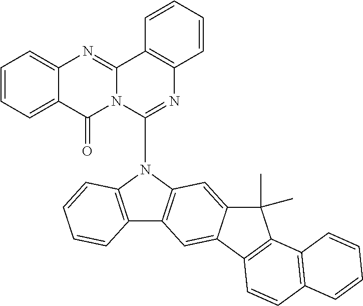 [SMILES: CC1(C)c2cc3c(cc2-c2ccc4ccccc4c21)c1ccccc1n3-c1nc2ccccc2c2nc3ccccc3c(=O)n12] | 71 |
| 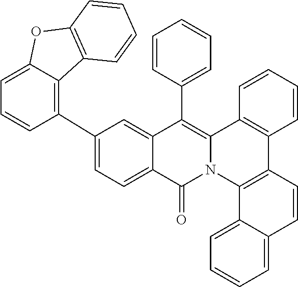 [SMILES: O=c1c2ccc(-c3cccc4oc5ccccc5c34)cc2c(-c2ccccc2)c2c3ccccc3c3ccc4ccccc4c3n12] | 72 |
| 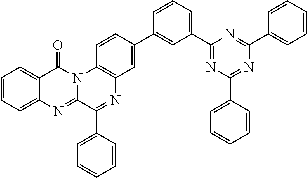 [SMILES: O=c1c2ccccc2nc2c(-c3ccccc3)nc3cc(-c4cccc(-c5nc(-c6ccccc6)nc(-c6ccccc6)n5)c4)ccc3n12] | 73 |
| 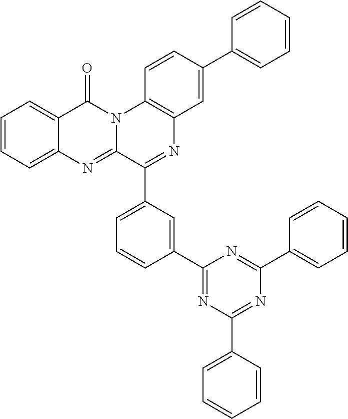 [SMILES: O=c1c2ccccc2nc2c(-c3cccc(-c4nc(-c5ccccc5)nc(-c5ccccc5)n4)c3)nc3cc(-c4ccccc4)ccc3n12] | 74 |
| 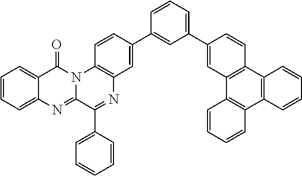 [SMILES: O=c1c2ccccc2nc2c(-c3ccccc3)nc3cc(-c4cccc(-c5ccc6c7ccccc7c7ccccc7c6c5)c4)ccc3n12] | 75 |
| 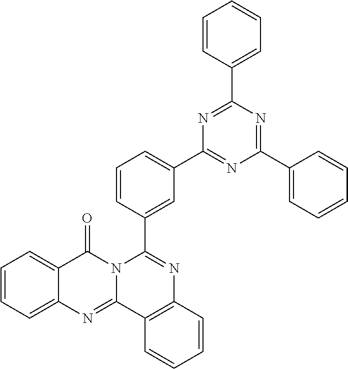 [SMILES: O=c1c2ccccc2nc2c3ccccc3nc(-c3cccc(-c4nc(-c5ccccc5)nc(-c5ccccc5)n4)c3)n12] | 76 |
| 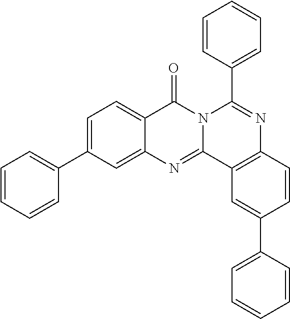 [SMILES: O=c1c2ccc(-c3ccccc3)cc2nc2c3cc(-c4ccccc4)ccc3nc(-c3ccccc3)n12] | 77 |
| 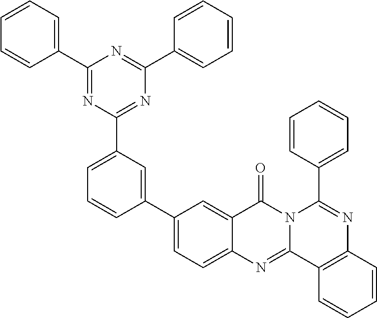 [SMILES: O=c1c2cc(-c3cccc(-c4nc(-c5ccccc5)nc(-c5ccccc5)n4)c3)ccc2nc2c3ccccc3nc(-c3ccccc3)n12] | 78 |
| 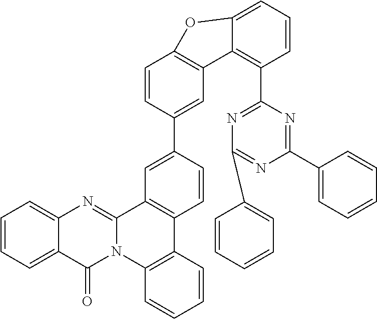 [SMILES: O=c1c2ccccc2nc2c3cc(-c4ccc5oc6cccc(-c7nc(-c8ccccc8)nc(-c8ccccc8)n7)c6c5c4)ccc3c3ccccc3n12] | 79 |
| 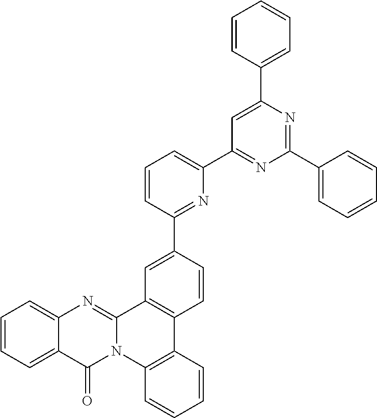 [SMILES: O=c1c2ccccc2nc2c3cc(-c4cccc(-c5cc(-c6ccccc6)nc(-c6ccccc6)n5)n4)ccc3c3ccccc3n12] | 80 |
| 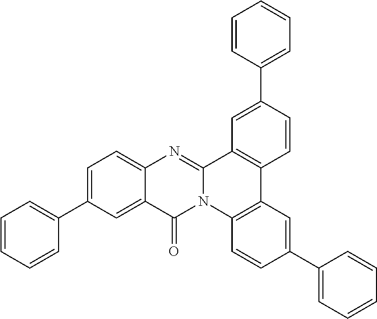 [SMILES: O=c1c2cc(-c3ccccc3)ccc2nc2c3cc(-c4ccccc4)ccc3c3cc(-c4ccccc4)ccc3n12] | 81 |
| 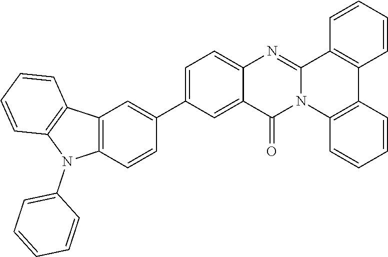 [SMILES: O=c1c2cc(-c3ccc4c(c3)c3ccccc3n4-c3ccccc3)ccc2nc2c3ccccc3c3ccccc3n12] | 82 |
| 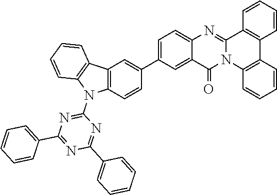 [SMILES: O=c1c2cc(-c3ccc4c(c3)c3ccccc3n4-c3nc(-c4ccccc4)nc(-c4ccccc4)n3)ccc2nc2c3ccccc3c3ccccc3n12] | 83 |
| 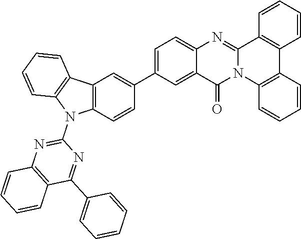 [SMILES: O=c1c2cc(-c3ccc4c(c3)c3ccccc3n4-c3nc(-c4ccccc4)c4ccccc4n3)ccc2nc2c3ccccc3c3ccccc3n12] | 84 |
| 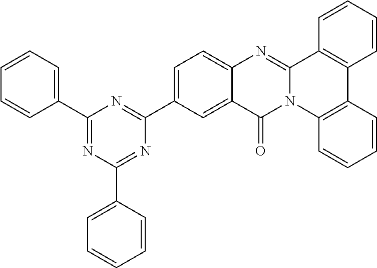 [SMILES: O=c1c2cc(-c3nc(-c4ccccc4)nc(-c4ccccc4)n3)ccc2nc2c3ccccc3c3ccccc3n12] | 85 |
| 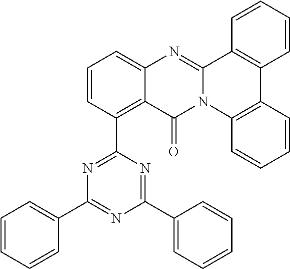 [SMILES: O=c1c2c(-c3nc(-c4ccccc4)nc(-c4ccccc4)n3)cccc2nc2c3ccccc3c3ccccc3n12] | 86 |
| 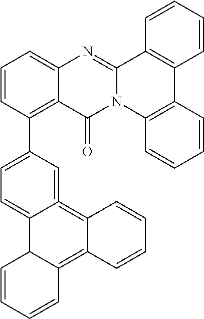 | 87 |
| 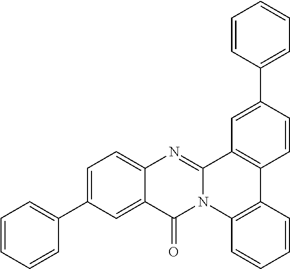 [SMILES: O=c1c2cc(-c3ccccc3)ccc2nc2c3cc(-c4ccccc4)ccc3c3ccccc3n12] | 88 |
| 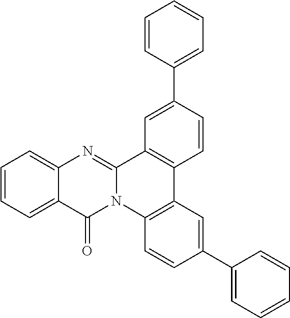 [SMILES: O=c1c2ccccc2nc2c3cc(-c4ccccc4)ccc3c3cc(-c4ccccc4)ccc3n12] | 89 |
| 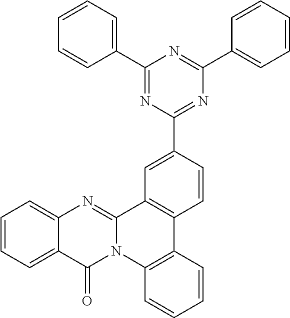 [SMILES: O=c1c2ccccc2nc2c3cc(-c4nc(-c5ccccc5)nc(-c5ccccc5)n4)ccc3c3ccccc3n12] | 90 |
| 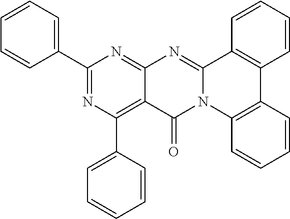 [SMILES: O=c1c2c(-c3ccccc3)nc(-c3ccccc3)nc2nc2c3ccccc3c3ccccc3n12] | 91 |
| 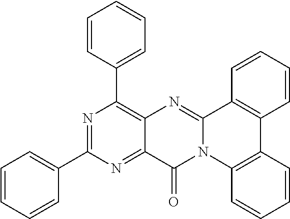 [SMILES: O=c1c2nc(-c3ccccc3)nc(-c3ccccc3)c2nc2c3ccccc3c3ccccc3n12] | 92 |
| 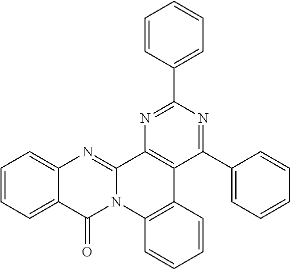 [SMILES: O=c1c2ccccc2nc2c3nc(-c4ccccc4)nc(-c4ccccc4)c3c3ccccc3n12] | 93 |
| 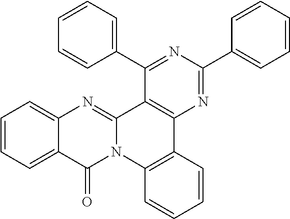 [SMILES: O=c1c2ccccc2nc2c3c(-c4ccccc4)nc(-c4ccccc4)nc3c3ccccc3n12] | 94 |
| 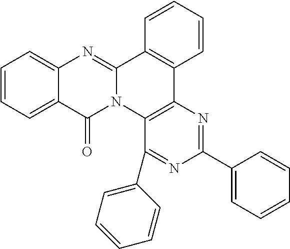 [SMILES: O=c1c2ccccc2nc2c3ccccc3c3nc(-c4ccccc4)nc(-c4ccccc4)c3n12] | 95 |
| 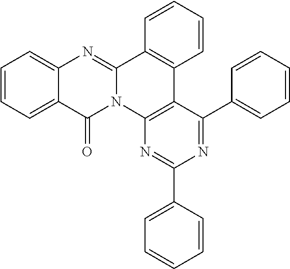 [SMILES: O=c1c2ccccc2nc2c3ccccc3c3c(-c4ccccc4)nc(-c4ccccc4)nc3n12] | 96 |
| 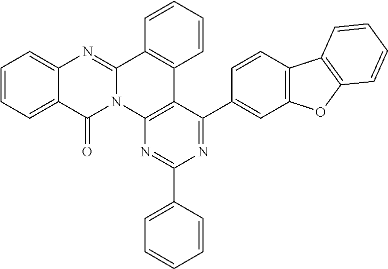 [SMILES: O=c1c2ccccc2nc2c3ccccc3c3c(-c4ccc5c(c4)oc4ccccc45)nc(-c4ccccc4)nc3n12] | 97 |
| 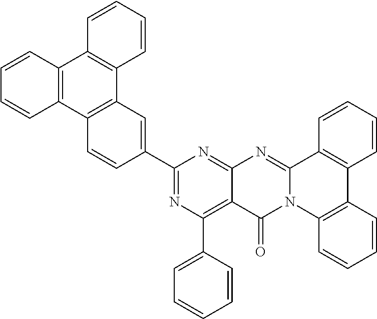 [SMILES: O=c1c2c(-c3ccccc3)nc(-c3ccc4c5ccccc5c5ccccc5c4c3)nc2nc2c3ccccc3c3ccccc3n12] | 98 |
| 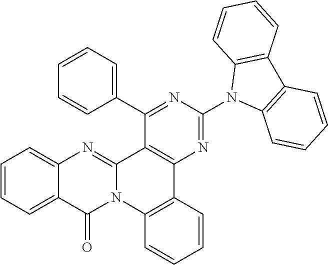 [SMILES: O=c1c2ccccc2nc2c3c(-c4ccccc4)nc(-n4c5ccccc5c5ccccc54)nc3c3ccccc3n12] | 99 |
| 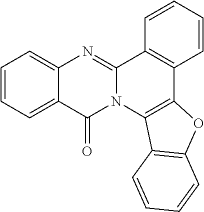 [SMILES: O=c1c2ccccc2nc2c3ccccc3c3oc4ccccc4c3n12] | 100 |
| 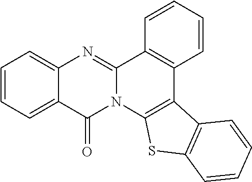 [SMILES: O=c1c2ccccc2nc2c3ccccc3c3c4ccccc4sc3n12] | 101 |
| 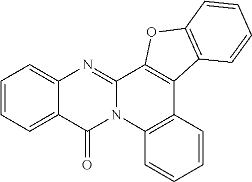 [SMILES: O=c1c2ccccc2nc2c3oc4ccccc4c3c3ccccc3n12] | 102 |
| 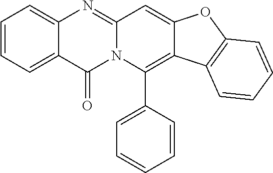 [SMILES: O=c1c2ccccc2nc2cc3oc4ccccc4c3c(-c3ccccc3)n12] | 103 |
| 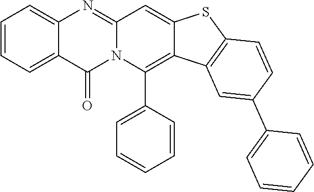 [SMILES: O=c1c2ccccc2nc2cc3sc4ccc(-c5ccccc5)cc4c3c(-c3ccccc3)n12] | 104 |
| 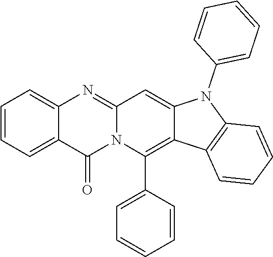 [SMILES: O=c1c2ccccc2nc2cc3c(c(-c4ccccc4)n12)c1ccccc1n3-c1ccccc1] | 105 |
| 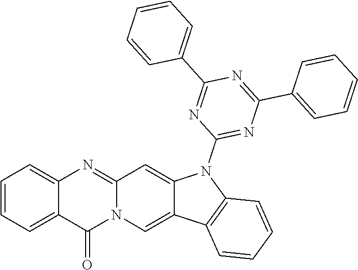 [SMILES: O=c1c2ccccc2nc2cc3c(cn12)c1ccccc1n3-c1nc(-c2ccccc2)nc(-c2ccccc2)n1] | 106 |
| 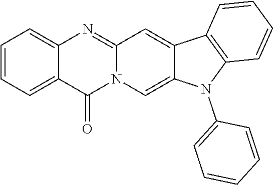 [SMILES: O=c1c2ccccc2nc2cc3c4ccccc4n(-c4ccccc4)c3cn12] | 107 |
| 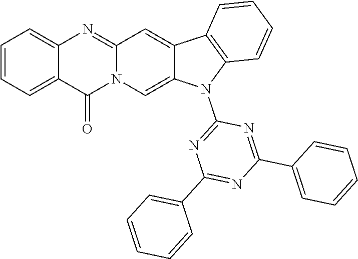 [SMILES: O=c1c2ccccc2nc2cc3c4ccccc4n(-c4nc(-c5ccccc5)nc(-c5ccccc5)n4)c3cn12] | 108 |
| 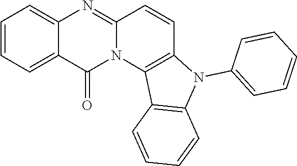 [SMILES: O=c1c2ccccc2nc2ccc3c(c4ccccc4n3-c3ccccc3)n12] | 109 |
| 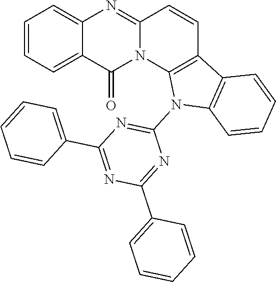 [SMILES: O=c1c2ccccc2nc2ccc3c4ccccc4n(-c4nc(-c5ccccc5)nc(-c5ccccc5)n4)c3n12] | 110 |
| 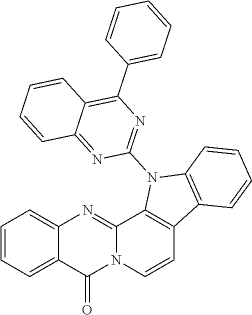 [SMILES: O=c1c2ccccc2nc2c3c(ccn12)c1ccccc1n3-c1nc(-c2ccccc2)c2ccccc2n1] | 111 |
| 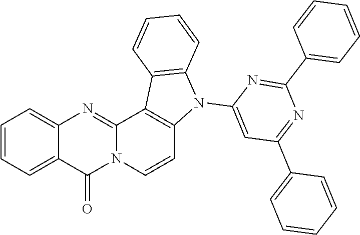 [SMILES: O=c1c2ccccc2nc2c3c4ccccc4n(-c4cc(-c5ccccc5)nc(-c5ccccc5)n4)c3ccn12] | 112 |
| 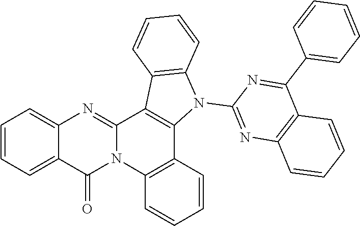 [SMILES: O=c1c2ccccc2nc2c3c4ccccc4n(-c4nc(-c5ccccc5)c5ccccc5n4)c3c3ccccc3n12] | 113 |
| 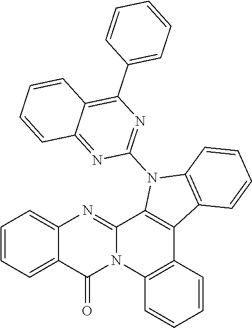 [SMILES: O=c1c2ccccc2nc2c3c(c4ccccc4n3-c3nc(-c4ccccc4)c4ccccc4n3)c3ccccc3n12] | 114 |
| 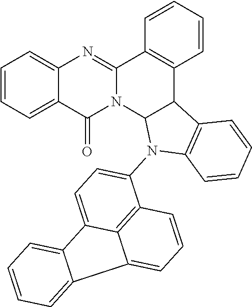 [SMILES: O=c1c2ccccc2nc2n1C1C(c3ccccc3-2)c2ccccc2N1c1ccc2c3c(cccc13)-c1ccccc1-2] | 115 |
| 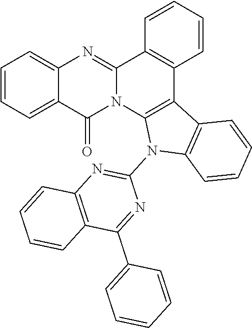 [SMILES: O=c1c2ccccc2nc2c3ccccc3c3c4ccccc4n(-c4nc(-c5ccccc5)c5ccccc5n4)c3n12] | 116 |
| 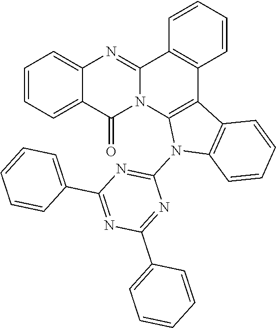 [SMILES: O=c1c2ccccc2nc2c3ccccc3c3c4ccccc4n(-c4nc(-c5ccccc5)nc(-c5ccccc5)n4)c3n12] | 117 |
| 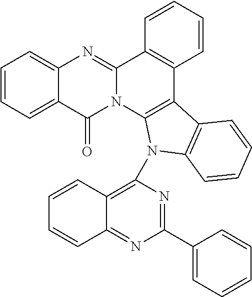 [SMILES: O=c1c2ccccc2nc2c3ccccc3c3c4ccccc4n(-c4nc(-c5ccccc5)nc5ccccc45)c3n12] | 118 |
| 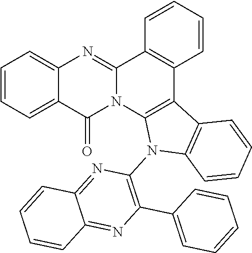 [SMILES: O=c1c2ccccc2nc2c3ccccc3c3c4ccccc4n(-c4nc5ccccc5nc4-c4ccccc4)c3n12] | 119 |
| 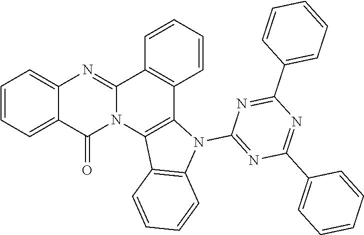 [SMILES: O=c1c2ccccc2nc2c3ccccc3c3c(c4ccccc4n3-c3nc(-c4ccccc4)nc(-c4ccccc4)n3)n12] | 120 |
| 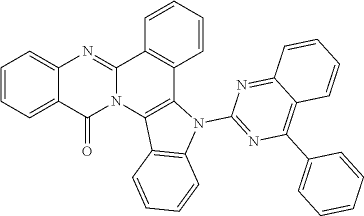 [SMILES: O=c1c2ccccc2nc2c3ccccc3c3c(c4ccccc4n3-c3nc(-c4ccccc4)c4ccccc4n3)n12] | 121 |
| 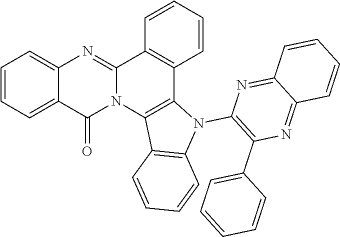 [SMILES: O=c1c2ccccc2nc2c3ccccc3c3c(c4ccccc4n3-c3nc4ccccc4nc3-c3ccccc3)n12] | 122 |
| 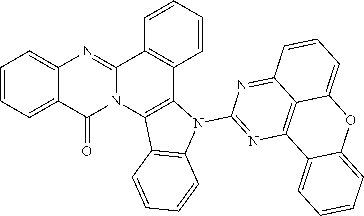 [SMILES: O=c1c2ccccc2nc2c3ccccc3c3c(c4ccccc4n3-c3nc4c5c(cccc5n3)Oc3ccccc3-4)n12] | 123 |
| 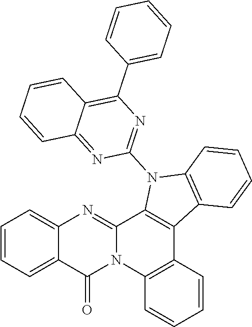 [SMILES: O=c1c2ccccc2nc2c3c(c4ccccc4n3-c3nc(-c4ccccc4)c4ccccc4n3)c3ccccc3n12] | 124 |
| 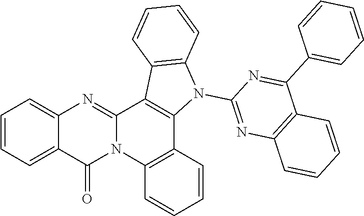 [SMILES: O=c1c2ccccc2nc2c3c4ccccc4n(-c4nc(-c5ccccc5)c5ccccc5n4)c3c3ccccc3n12] | 125 |
| 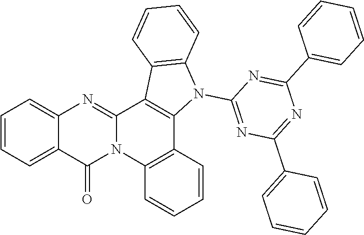 [SMILES: O=c1c2ccccc2nc2c3c4ccccc4n(-c4nc(-c5ccccc5)nc(-c5ccccc5)n4)c3c3ccccc3n12] | 126 |
| 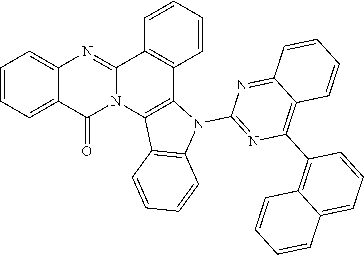 [SMILES: O=c1c2ccccc2nc2c3ccccc3c3c(c4ccccc4n3-c3nc(-c4cccc5ccccc45)c4ccccc4n3)n12] | 127 |
| 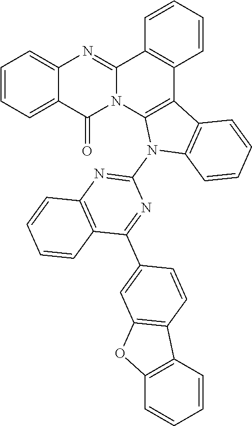 [SMILES: O=c1c2ccccc2nc2c3ccccc3c3c4ccccc4n(-c4nc(-c5ccc6c(c5)oc5ccccc56)c5ccccc5n4)c3n12] | 128 |
| 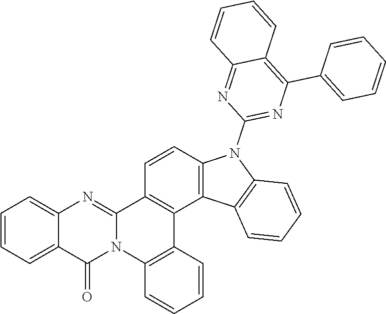 [SMILES: O=c1c2ccccc2nc2c3ccc4c(c5ccccc5n4-c4nc(-c5ccccc5)c5ccccc5n4)c3c3ccccc3n12] | 129 |
| 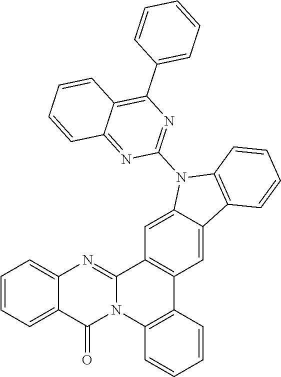 [SMILES: O=c1c2ccccc2nc2c3cc4c(cc3c3ccccc3n12)c1ccccc1n4-c1nc(-c2ccccc2)c2ccccc2n1] | 130 |
| 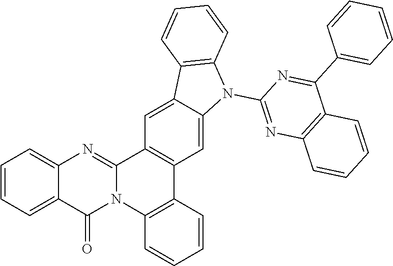 [SMILES: O=c1c2ccccc2nc2c3cc4c5ccccc5n(-c5nc(-c6ccccc6)c6ccccc6n5)c4cc3c3ccccc3n12] | 131 |
| 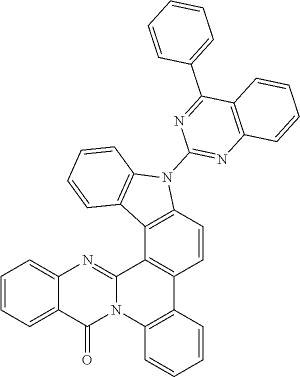 [SMILES: O=c1c2ccccc2nc2c3c(ccc4c3c3ccccc3n4-c3nc(-c4ccccc4)c4ccccc4n3)c3ccccc3n12] | 132 |
| 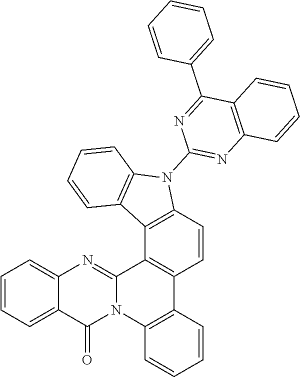 [SMILES: O=c1c2ccccc2nc2c3c(ccc4c3c3ccccc3n4-c3nc(-c4ccccc4)c4ccccc4n3)c3ccccc3n12] | 133 |
| 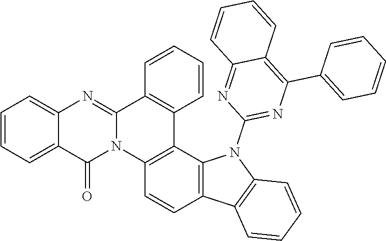 [SMILES: O=c1c2ccccc2nc2c3ccccc3c3c4c(ccc3n12)c1ccccc1n4-c1nc(-c2ccccc2)c2ccccc2n1] | 134 |
| 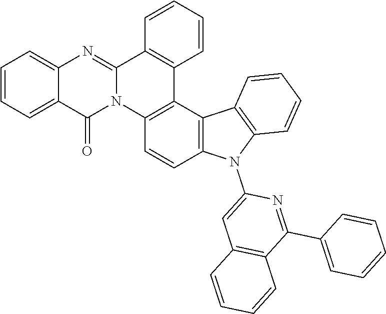 [SMILES: O=c1c2ccccc2nc2c3ccccc3c3c4c5ccccc5n(-c5cc6ccccc6c(-c6ccccc6)n5)c4ccc3n12] | 135 |
| 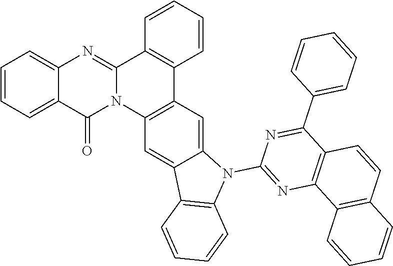 [SMILES: O=c1c2ccccc2nc2c3ccccc3c3cc4c(cc3n12)c1ccccc1n4-c1nc(-c2ccccc2)c2ccc3ccccc3c2n1] | 136 |
| 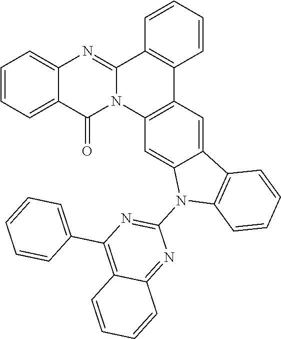 [SMILES: O=c1c2ccccc2nc2c3ccccc3c3cc4c5ccccc5n(-c5nc(-c6ccccc6)c6ccccc6n5)c4cc3n12] | 137 |
| 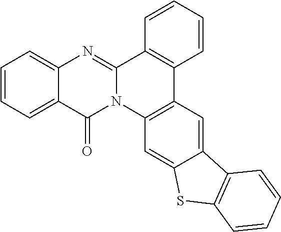 [SMILES: O=c1c2ccccc2nc2c3ccccc3c3cc4c(cc3n12)sc1ccccc14] | 138 |
| 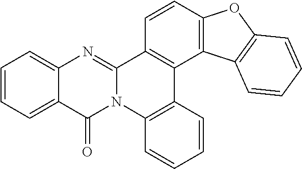 [SMILES: O=c1c2ccccc2nc2c3ccc4oc5ccccc5c4c3c3ccccc3n12] | 139 |
| 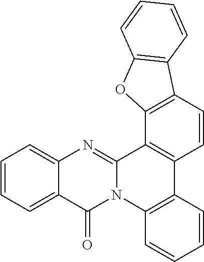 [SMILES: O=c1c2ccccc2nc2c3c(ccc4c5ccccc5oc43)c3ccccc3n12] | 140 |
| 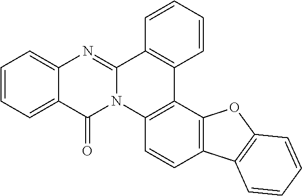 [SMILES: O=c1c2ccccc2nc2c3ccccc3c3c4oc5ccccc5c4ccc3n12] | 141 |
| 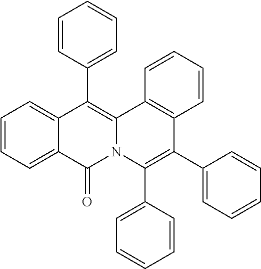 [SMILES: O=c1c2ccccc2c(-c2ccccc2)c2c3ccccc3c(-c3ccccc3)c(-c3ccccc3)n12] | 142 |
| 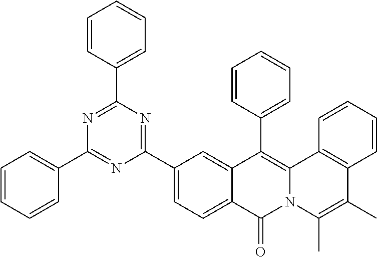 [SMILES: Cc1c(C)n2c(=O)c3ccc(-c4nc(-c5ccccc5)nc(-c5ccccc5)n4)cc3c(-c3ccccc3)c2c2ccccc12] | 143 |
| 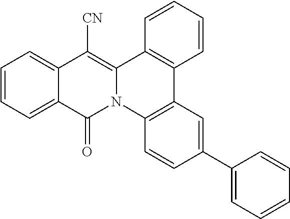 [SMILES: N#Cc1c2ccccc2c(=O)n2c3ccc(-c4ccccc4)cc3c3ccccc3c12] | 144 |
| 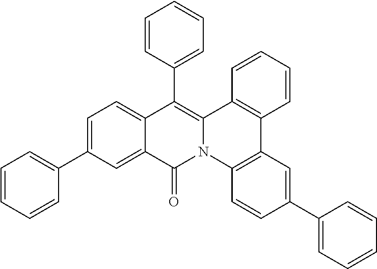 [SMILES: O=c1c2cc(-c3ccccc3)ccc2c(-c2ccccc2)c2c3ccccc3c3cc(-c4ccccc4)ccc3n12] | 145 |
| 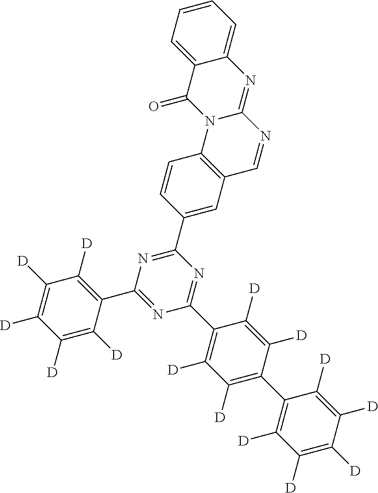 [SMILES: [2H]c1c([2H])c([2H])c(-c2nc(-c3ccc4c(cnc5nc6ccccc6c(=O)n54)c3)nc(-c3c([2H])c([2H])c(-c4c([2H])c([2H])c([2H])c([2H])c4[2H])c([2H])c3[2H])n2)c([2H])c1[2H]] | 146 |
| 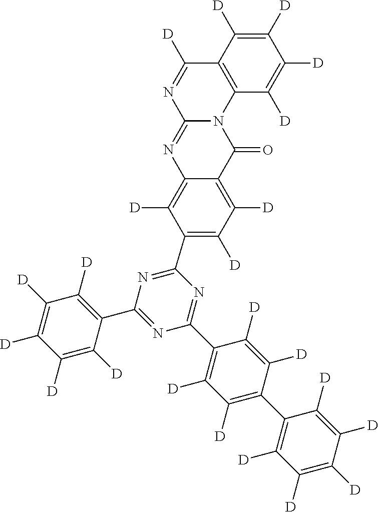 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c4c(=O)n5c(nc([2H])c6c([2H])c([2H])c([2H])c([2H])c65)nc4c3[2H])n2)c([2H])c1[2H]] | 147 |
| 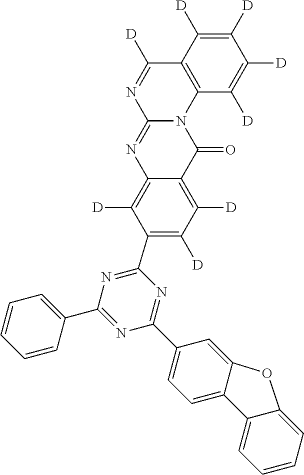 [SMILES: [2H]c1c([2H])c([2H])c2c(c([2H])nc3nc4c([2H])c(-c5nc(-c6ccccc6)nc(-c6ccc7c(c6)oc6ccccc67)n5)c([2H])c([2H])c4c(=O)n32)c1[2H]] | 148 |
| 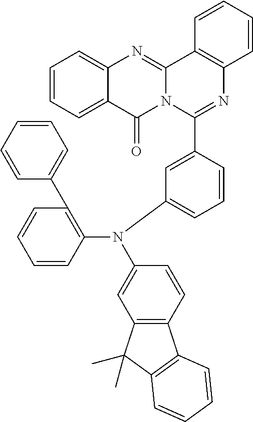 [SMILES: CC1(C)c2ccccc2-c2ccc(N(c3cccc(-c4nc5ccccc5c5nc6ccccc6c(=O)n45)c3)c3ccccc3-c3ccccc3)cc21] | 149 |
| 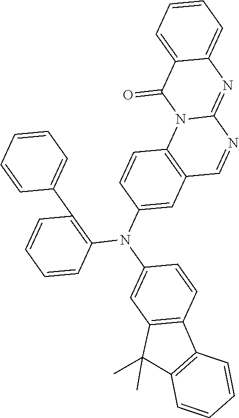 [SMILES: CC1(C)c2ccccc2-c2ccc(N(c3ccc4c(cnc5nc6ccccc6c(=O)n54)c3)c3ccccc3-c3ccccc3)cc21] | 150 |
| 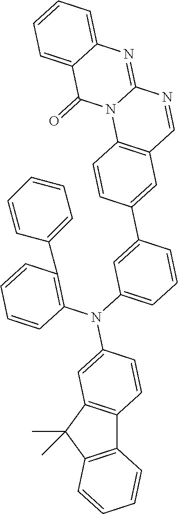 [SMILES: CC1(C)c2ccccc2-c2ccc(N(c3cccc(-c4ccc5c(cnc6nc7ccccc7c(=O)n65)c4)c3)c3ccccc3-c3ccccc3)cc21] | 151 |
| 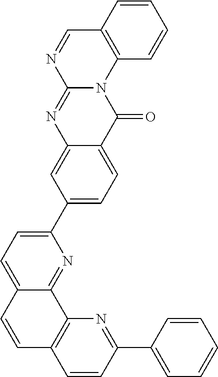 [SMILES: O=c1c2ccc(-c3ccc4ccc5ccc(-c6ccccc6)nc5c4n3)cc2nc2ncc3ccccc3n12] | 152 |
| 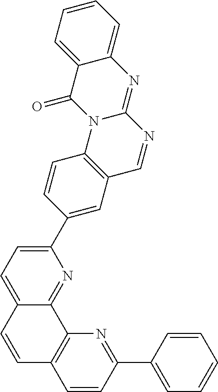 [SMILES: O=c1c2ccccc2nc2ncc3cc(-c4ccc5ccc6ccc(-c7ccccc7)nc6c5n4)ccc3n12] | 153 |
| 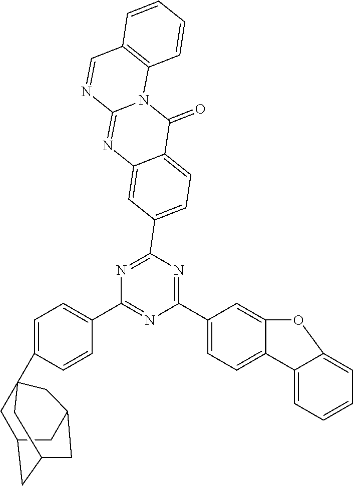 [SMILES: O=c1c2ccc(-c3nc(-c4ccc(C56CC7CC(CC(C7)C5)C6)cc4)nc(-c4ccc5c(c4)oc4ccccc45)n3)cc2nc2ncc3ccccc3n12] | 154 |

The base structure of the compounds of the invention can be prepared by the routes outlined in the schemes which follow. The individual synthesis steps, for example C—C coupling reactions according to Suzuki, C—N coupling reactions according to Buchwald or Hartwig-Buchwald or cyclization reactions, are known in principle to those skilled in the art here. Further information relating to the synthesis of the compounds of the invention can be found in the synthesis examples. Possible syntheses of the base structure are shown in schemes 1 to 13. These can be effected by known reactions, as set out by way of example in CN 108101904 and in the publications Chemical Communications (Cambridge, United Kingdom), 53(75), 10394-10397; 2017; Synthesis, 48(22), 3941-3950; 2016; A European Journal, 22(10), 3506-3512; 2016; Open Journal of Medicinal Chemistry, 4(1), 12-37, 26 pp.; 2014; Heterocyclic Communications, 10(1), 89-92; 2004; Monatshefte für Chemie, 150(7), 1305-1315; 2019; Organic & Biomolecular Chemistry, 16(11), 1851-1859; 2018; Organic Letters, 18(13), 3142-3145; 2016; Asian Journal of Organic Chemistry, 4(5), 462-469; 2015; Organic Letters, 21(24), 9841-9845; 2019 and Chem Cat Chem, 7(18), 2986-2990; 2015. Schemes 14 to 15 show various possible options for preparation of the base structure and for introduction of substituents. By way of example, schemes 16 and 17 show means of preparing base structures having further ring nitrogen atoms, as described inter alia in the following publications: Archiv der Pharmazie (Weinheim, Germany) (1983), 316(8), 702-6 or Indian Journal of Chemistry, Section B: Organic Chemistry Including Medicinal Chemistry (2007), 46B(12), 2071-2073.

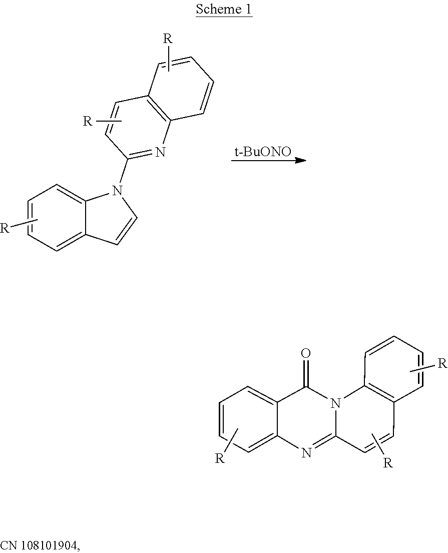

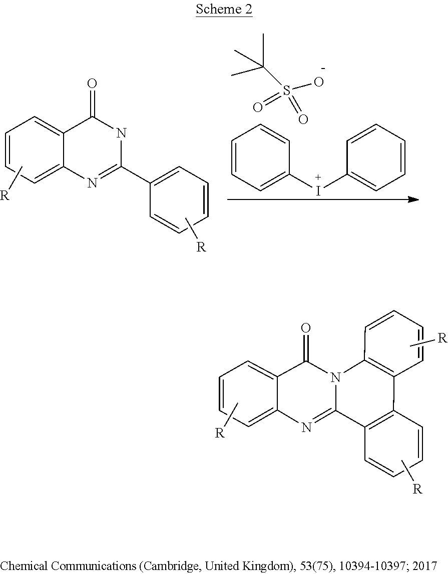

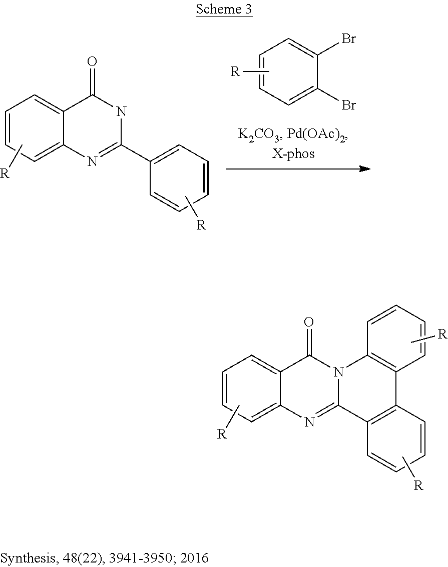

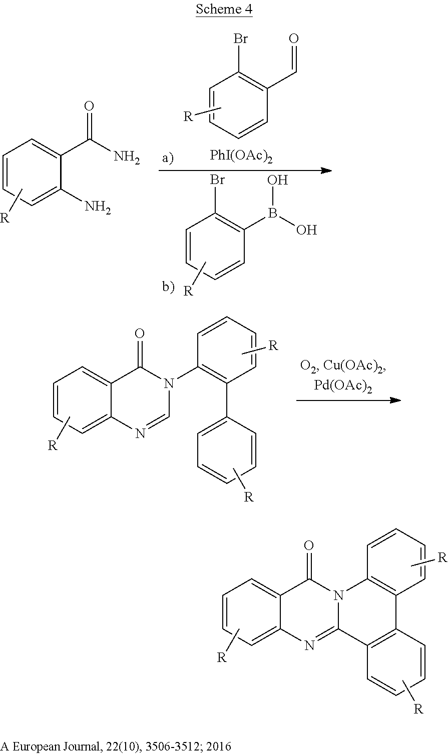

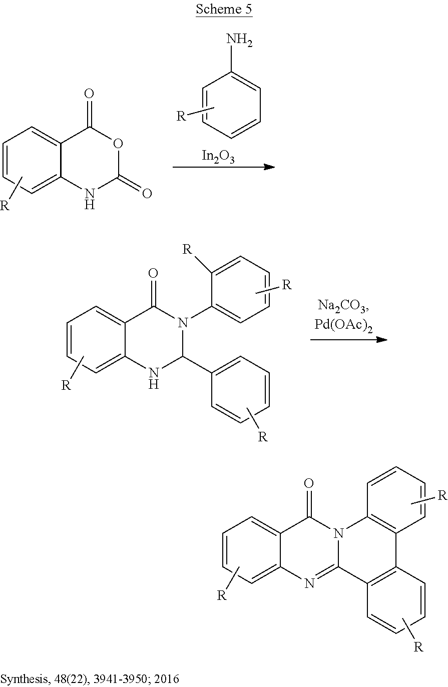

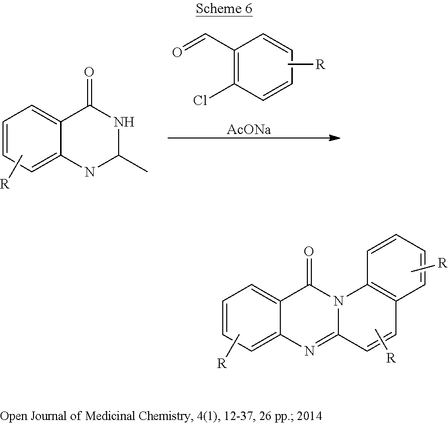

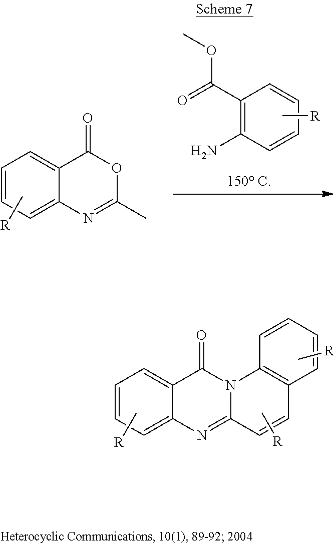

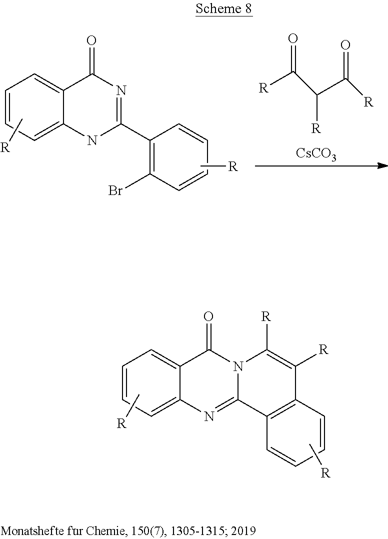

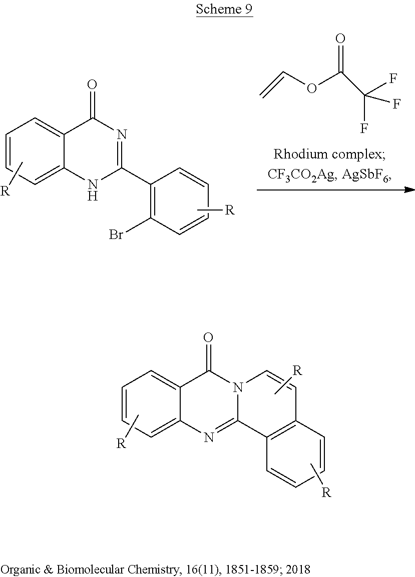

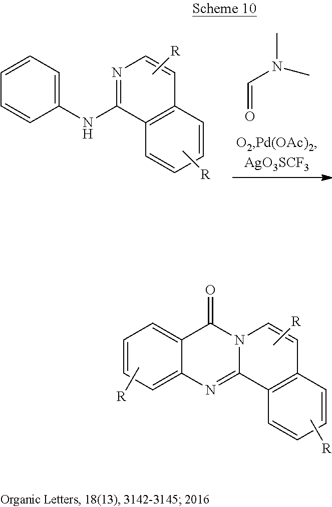

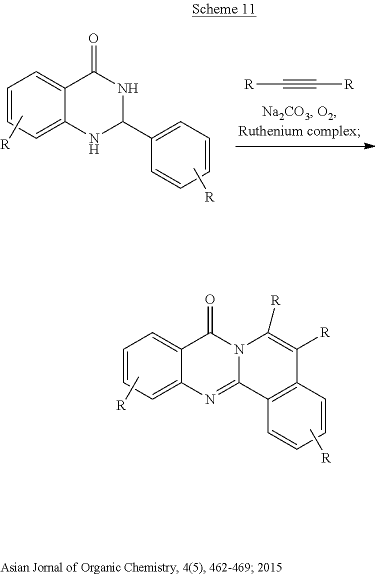

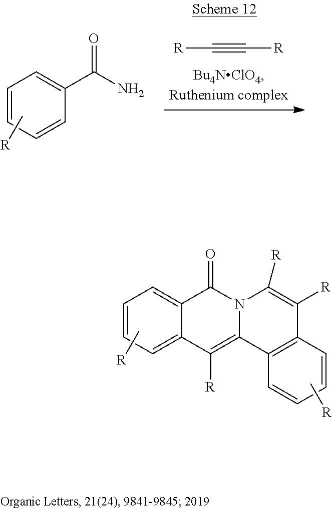

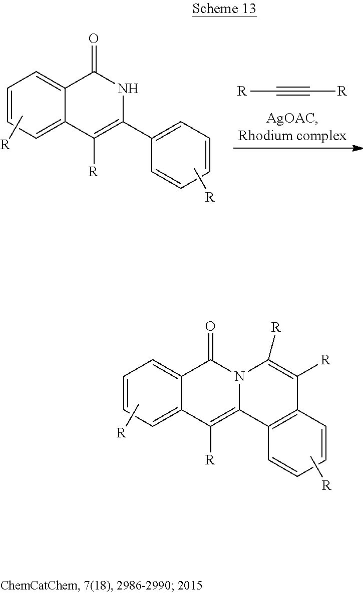

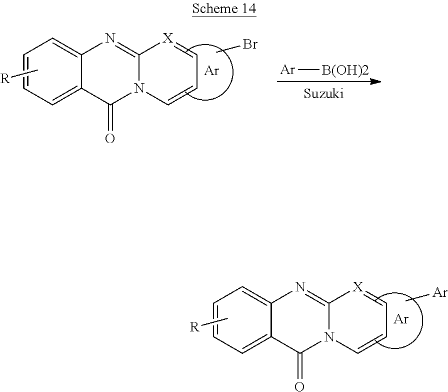

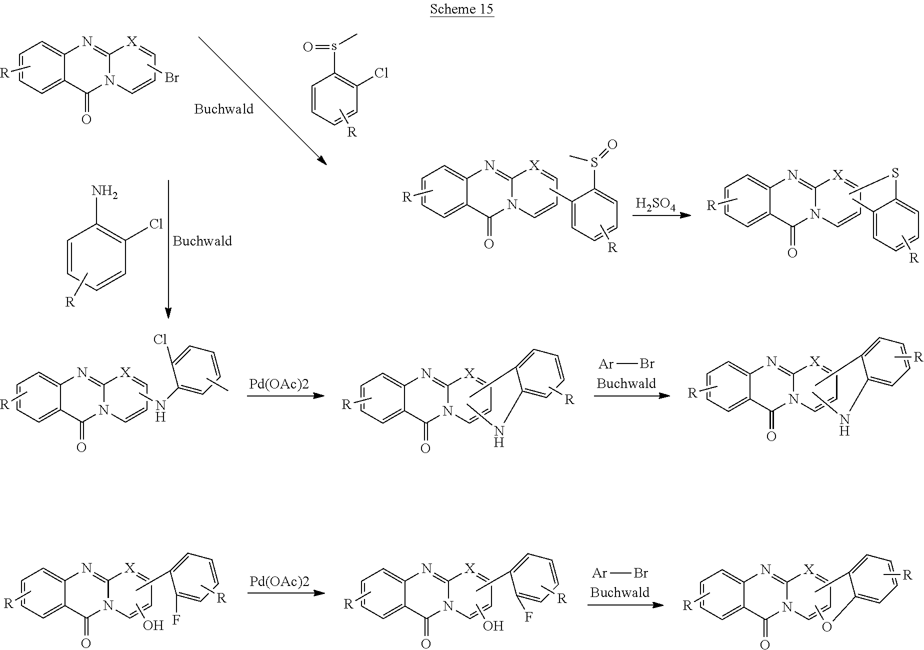

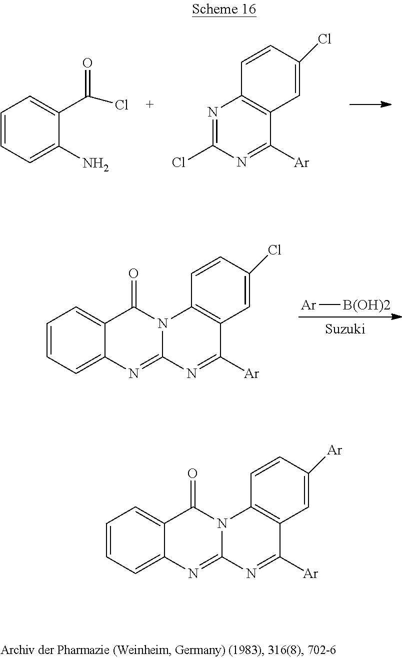

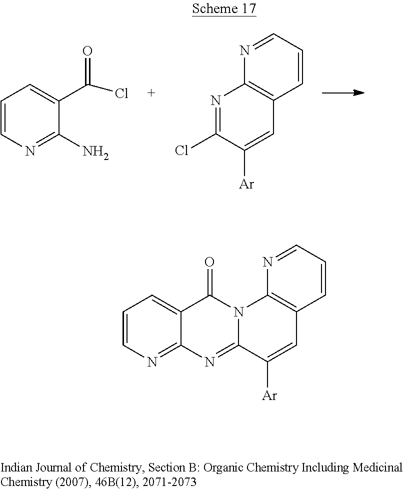

The definition of the symbols used in schemes 1 to 17 corresponds essentially to that which was defined for formula (I), dispensing with numbering and complete representation of all symbols for reasons of clarity. In addition, for reasons of clarity, the use of the symbol X for representation of possible nitrogen atoms in the heteroaromatic rings has been dispensed with in many cases, as shown in particular in formulae (I), (II-1) to (II-7) and/or (III-1) to (III-11) by the symbols X¹, X² and X³. These details should therefore be understood by way of illustration; the person skilled in the art is capable of applying the syntheses set out above and hereinafter, especially in the examples, to compounds in which one or more of the symbols X¹, X² and X³ are nitrogen.

The present invention therefore further provides a process for preparing a compound of the invention, wherein a nitrogen-containing aromatic or heteroaromatic compound is reacted in a ring-forming reaction.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation or a composition comprising at least one compound of the invention and at least one further compound. The further compound may, for example, be a solvent, especially one of the abovementioned solvents or a mixture of these solvents. If the further compound comprises a solvent, this mixture is referred to herein as formulation. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device. The further compound may also be polymeric.

The present invention further provides for the use of a compound comprising at least one structure of the formula (Ia), preferably a compound of the formula (Ia),

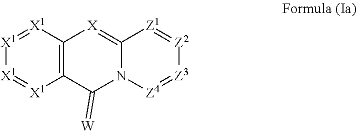

where the symbols and indices used are as follows:
W is O or S, preferably O;
Z¹ is X², or the Z¹ group together with the Z² group forms a fused ring A¹ bonded via two adjacent carbon atoms to the further groups of the 6-membered ring;
Z² is X², or the Z² group together with the Z¹ group forms a fused ring A¹ bonded via two adjacent carbon atoms to the further groups of the 6-membered ring, or the Z² group together with the Z³ group forms a fused ring A² bonded via two adjacent carbon atoms to the further groups of the 6-membered ring;
Z³ is X², or the Z³ group together with the Z² group forms a fused ring A² bonded via two adjacent carbon atoms to the further groups of the 6-membered ring, or the Z³ group together with the Z⁴ group forms a fused ring A³ bonded via two adjacent carbon atoms to the further groups of the 6-membered ring;
Z⁴ is X², or the Z⁴ group together with the Z³ group forms a fused ring A³ bonded via two adjacent carbon atoms to the further groups of the 6-membered ring;
X is the same or different at each instance and is N or CR, preferably N;
X¹ is the same or different at each instance and is N or CR¹, preferably CR¹, with the proviso that not more than two of the X¹ groups in one cycle are N;
X² is the same or different at each instance and is N or CR²;
A¹, A², A³ is the same or different at each instance and is an aromatic or heteroaromatic ring system which has 5 to 40 aromatic ring atoms and has R³ radicals, where the aromatic or heteroaromatic ring system is bonded via two connected carbon atoms to the further groups in the structure shown in formula (I), where two adjacent A¹, A², A³ groups together may form a fused ring system;
R is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is possible here for an R radical together with a further radical, preferably a R², R³ group to form an aliphatic, heteroaliphatic, aromatic or heteroaromatic ring system, preferably an aliphatic, heteroaliphatic or heteroaromatic ring system; more preferably, the R radical does not form any such ring system;
R¹ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R¹ radicals together to form an aliphatic or heteroaliphatic ring system; the R¹ radicals preferably do not form any such ring system; with exclusion of the formation of an aromatic or heteroaromatic ring system by two R¹ radicals;
R² is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R² radicals or one R² radical together with a further radical, preferably an R, R³ group, to form an aromatic, heteroaromatic, aliphatic or heteroaliphatic ring system; the R² radicals preferably do not form any such ring system;
R³ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, preferably 5 to 40 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R³ radicals or one R³ radical together with a further radical, preferably an R, R² group, to form an aromatic, heteroaromatic, aliphatic or heteroaliphatic ring system; the R³ radicals preferably do not form any such ring system;
Ar′ is the same or different at each instance and is an aromatic or heteroaromatic ring system which has 5 to 40 aromatic ring atoms and has R⁴ radicals, preferably an aryl group which has 6 to 30 aromatic ring atoms or a heteroaryl group which has 5 to 14 aromatic ring atoms and has R⁴ radicals;
R⁴ is the same or different at each instance and is H, D, F, Cl, Br, I, R⁵C═C(R⁵)₂, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁵ radicals, where one or more nonadjacent CH₂ groups may be replaced by R⁵C═CR⁵, C≡C, Si(R⁵)₂, C═O, C═S, C═Se, C═NR⁵, —C(═O)O—, —C(═O)NR⁵—, NR⁵, P(═O)(R⁵), —O—, —S—, SO or SO₂, or an aromatic or heteroaromatic ring system which has 5 to 40 aromatic ring atoms and in each case has R⁵ radicals, or an aryloxy or heteroaryloxy group which has 5 to 40 aromatic ring atoms and in each case has R⁵ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁵ radicals; it is possible here for two or more R⁴ radicals together to form an aromatic, heteroaromatic, aliphatic or heteroaliphatic ring system, preferably an aliphatic ring system; more preferably, the R⁴ radicals do not form any such ring system;
R⁵ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F; it is possible here for two or more R⁵ radicals together to form a ring system;
   where the structure of formula (Ia) has at least one ring A¹, A², A³ which is formed by at least two adjacent groups selected from the Z¹, Z², Z³ and Z⁴ groups;
in an electronic device, especially in an organic electroluminescent device.

The groups defined for formula (Ia) in many cases correspond to the radicals defined above for formula (I), and so the details, definitions and/or preferences set out above are also applicable to the formula (Ia). In addition, structures/compounds of formula (I) and preferred embodiments thereof are preferred structures/compounds of formula (Ia), and so the details above and hereinafter in this regard are also correspondingly applicable to structures/compounds of formula (Ia).

The present invention still further provides an electronic device containing at least one compound comprising at least one structure of the formula (Ia), preferably one compound of the formula (Ia),

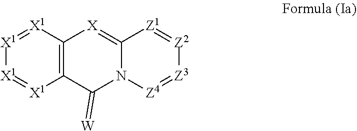

where the symbols and indices used have the definition given above, especially for formula (Ia), and the structure of formula (Ia) has at least one ring A¹, A², A³ which is formed by at least two adjacent groups selected from the Z¹, Z², Z³ and Z⁴ groups.

An electronic device in the context of the present invention is a device comprising at least one layer comprising at least one organic compound. This component may also comprise inorganic materials or else layers formed entirely from inorganic materials.

The electronic device is more preferably selected from the group consisting of organic electroluminescent devices (OLEDs, sOLED, PLEDs, LECs, etc.), preferably organic light-emitting diodes (OLEDs), organic light-emitting diodes based on small molecules (sOLEDs), organic light-emitting diodes based on polymers (PLEDs), light-emitting electrochemical cells (LECs), organic laser diodes (O-laser), organic plasmon-emitting devices (D. M. Koller et al., Nature Photonics 2008, 1-4), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs) and organic electrical sensors, preferably organic electroluminescent devices (OLEDs, sOLED, PLEDs, LECs, etc.), more preferably organic light-emitting diodes (OLEDs), organic light-emitting diodes based on small molecules (sOLEDs), organic light-emitting diodes based on polymers (PLEDs), especially phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem electroluminescent device, especially for white-emitting OLEDs.

The compound of the invention may be used in different layers, according to the exact structure. Preference is given to an organic electroluminescent device containing a compound comprising at least one structure of formula (Ia) or (I) or the above-recited preferred embodiments in an emitting layer as matrix material for phosphorescent emitters or for emitters that exhibit TADF (thermally activated delayed fluorescence), especially for phosphorescent emitters. In addition, the compound of the invention can also be used in an electron transport layer and/or in a hole blocker layer in an electron blocker layer, preferably in an electron transport layer. More preferably, the compound of the invention is used as matrix material for phosphorescent emitters, especially for red-, orange-, green- or yellow-phosphorescing, preferably red- or green-phosphorescing, emitters in an emitting layer or as electron transport material in an electron transport layer, more preferably as matrix material in an emitting layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state>1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

In one embodiment of the invention, the compound of the invention is used here as the sole matrix material (“single host”) for the phosphorescent emitter.

A further embodiment of the present invention is the use of a compound comprising at least one structure of formula (Ia), (I) or a preferred embodiment thereof as matrix material for a phosphorescent emitter in combination with a further matrix material. A further matrix material which is used in addition to a compound comprising at least one structure of formula (Ia), (I) or a preferred embodiment is referred to hereinafter at least sometimes as co-host. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, dibenzofuran derivatives, for example according to WO 2015/169412, WO 2016/015810, WO 2016/023608, WO 2017/148564 or WO 2017/148565, or biscarbazoles, for example according to JP 3139321 B2.

It is likewise possible for a further phosphorescent emitter which emits at a shorter wavelength than the actual emitter to be present as co-host in the mixture. Particularly good results are achieved when the emitter used is a red-phosphorescing emitter and the co-host used in combination with the compound of the invention is a yellow-phosphorescing emitter.

In addition, the co-host used may be a compound that does not take part in charge transport to a significant degree, if at all, as described, for example, in WO 2010/108579. Especially suitable in combination with the compound of the invention as co-matrix material are compounds which have a large bandgap and themselves take part at least not to a significant degree, if any at all, in the charge transport of the emitting layer. Such materials are preferably pure hydrocarbons. Examples of such materials can be found, for example, in WO 2009/124627 or in WO 2010/006680.

Particularly preferred co-host materials that can be used in combination with a compound comprising at least one structure of formula (Ia) or a preferred embodiment thereof, especially compounds comprising at least one structure of formula (I), are compounds of one of the formulae (H-1), (H-2), (H-3), (H-4) or (H-5):

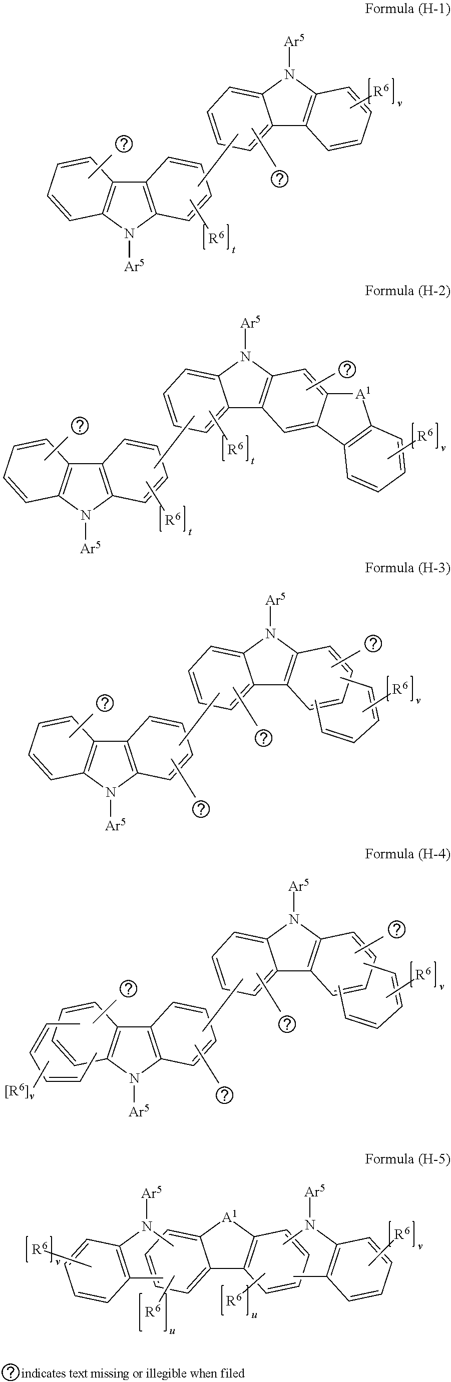

where the symbols and indices used are as follows:
R⁸ is the same or different at each instance and is H, D, F, Cl, Br, I, N(R⁷)₂, N(Ar″)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R⁷ radicals and where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, preferably 5 to 40 aromatic ring atoms, and in each case has R⁷ radicals; at the same time, two R⁸ radicals together may also form an aromatic, heteroaromatic, aliphatic or heteroaliphatic ring system; preferably, the R⁸ radicals do not form any such ring system;
Ar″ is the same or different at each instance and is an aromatic or heteroaromatic ring system which has 5 to 40 aromatic ring atoms and has R⁷ radicals;
A¹ is C(R⁷)₂, NR⁷, O or S;
Ar⁵ is the same or different at each instance and is an aromatic or heteroaromatic ring system which has 5 to 40 aromatic ring atoms and has R⁷ radicals;
R⁷ is the same or different at each instance and is H, D, F, Cl, Br, I, N(R⁸)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R⁸ radicals, where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and in each case has R⁸ radicals; at the same time, two or more R⁷ radicals together may form an aromatic, heteroaromatic, aliphatic or heteroaliphatic ring system; preferably, the R⁷ radicals do not form any such ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
v is the same or different at each instance and is 0, 1, 2, 3 or 4, preferably 0 or 1 and very preferably 0;
t is the same or different at each instance and is 0, 1, 2 or 3, preferably 0 or 1 and very preferably 0;
u is the same or different at each instance and is 0, 1 or 2, preferably 0 or 1 and very preferably 0.

The sum total of the indices v, t and u in compounds of the formulae (H-1), (H-2), (H-3), (H-4) or (H-5) is preferably not more than 6, more preferably not more than 4 and especially preferably not more than 2.

In a preferred embodiment of the invention, R⁶ is the same or different at each instance and is selected from the group consisting of H, D, F, CN, NO₂, Si(R⁷)₃, B(OR⁷)₂, a straight-chain alkyl group having 1 to 20 carbon atoms or a branched or cyclic alkyl group having 3 to 20 carbon atoms, where the alkyl group may be substituted in each case by one or more R⁷ radicals, or an aromatic or heteroaromatic ring system which has 5 to 60 aromatic ring atoms, preferably 5 to 40 aromatic ring atoms, and may be substituted in each case by one or more R⁷ radicals.

In a further-preferred embodiment of the invention, R⁶ is the same or different at each instance and is selected from the group consisting of H, D, F, a straight-chain alkyl group having 1 to 20 carbon atoms or a branched or cyclic alkyl group having 3 to 20 carbon atoms, where the alkyl group may be substituted in each case by one or more R⁷ radicals, or an aromatic or heteroaromatic ring system which has 5 to 60 aromatic ring atoms, preferably 5 to 40 aromatic ring atoms, and may be substituted in each case by one or more R⁷ radicals.

In a further-preferred embodiment of the invention, R⁶ is the same or different at each instance and is selected from the group consisting of H, D, an aromatic or heteroaromatic ring system which has 6 to 30 aromatic ring atoms and may be substituted by one or more R⁷ radicals, and an N(Ar″)₂ group. More preferably, R⁶ is the same or different at each instance and is selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R⁷ radicals.

Preferred aromatic or heteroaromatic ring systems represented by the R⁶ or Ar″ groups are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁷ radicals. The structures Ar-1 to Ar-79 listed above are particularly preferred, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). In the structures Ar-1 to Ar-79 set out above, in relation to the R⁶ and Ar″ radicals, the substituents R⁴ should be replaced by the corresponding R⁷ radicals. The preferences set out above for the R¹, R² and R³ groups are correspondingly applicable to the R⁶ group.

Further suitable R⁶ groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R⁴ radicals. The total number of aromatic ring atoms in Ar², Ar³ and Ar⁴ here is not more than 60 and preferably not more than 40. Further preferences for the Ar², Ar³ and Ar⁴ groups have been set out above and are correspondingly applicable.

It may further be the case that the substituents R⁶ according to the above formulae do not form a fused aromatic or heteroaromatic ring system, preferably any fused ring system, with the ring atoms of the ring system. This includes the formation of a fused ring system with possible substituents R⁷, R⁸ which may be bonded to the R⁶ radicals.

When A¹ is NR⁷, the substituent R¹ bonded to the nitrogen atom is preferably an aromatic or heteroaromatic ring system which has 5 to 24 aromatic ring atoms and may also be substituted by one or more R⁸ radicals. In a particularly preferred embodiment, this R⁷ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R⁸ radicals. Preference is given to phenyl, biphenyl, terphenyl and quaterphenyl having bonding patterns as listed above for Ar-1 to Ar-11, where these structures, rather than by R⁴, may be substituted by one or more R⁸ radicals, but are preferably unsubstituted. Preference is further given to triazine, pyrimidine and quinazoline as listed above for Ar-47 to Ar-50, Ar-57 and Ar-58, where these structures, rather than by R⁴, may be substituted by one or more R⁸ radicals.

When A¹ is C(R⁷)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R⁵ radicals. Most preferably, R⁷ is a methyl group or a phenyl group. In this case, the R⁷ radicals together may also form a ring system, which leads to a spiro system.

Preferred aromatic or heteroaromatic ring systems Ar⁵ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁷ radicals.

The Ar⁵ groups here are more preferably independently selected from the groups of the formulae Ar-1 to Ar-79 set out above, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). In the structures Ar-1 to Ar-79 set out above, in relation to the Ar⁵ radicals, the substituents R⁴ by the corresponding R⁷ radicals.

In a further preferred embodiment of the invention, R⁷ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R⁸ radicals, or an aromatic or heteroaromatic ring system which has 6 to 24 aromatic ring atoms and may be substituted in each case by one or more R⁸ radicals. In a particularly preferred embodiment of the invention, R⁷ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R⁸ radicals, but is preferably unsubstituted, or an aromatic or heteroaromatic ring system which has 6 to 13 aromatic ring atoms and may be substituted in each case by one or more R⁸ radicals, but is preferably unsubstituted.

In a further preferred embodiment of the invention, R⁸ is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

Preferred embodiments of the compounds of the formulae (H-1) and (H-2) are the compounds of the following formulae (H-1a) and (H-2a):

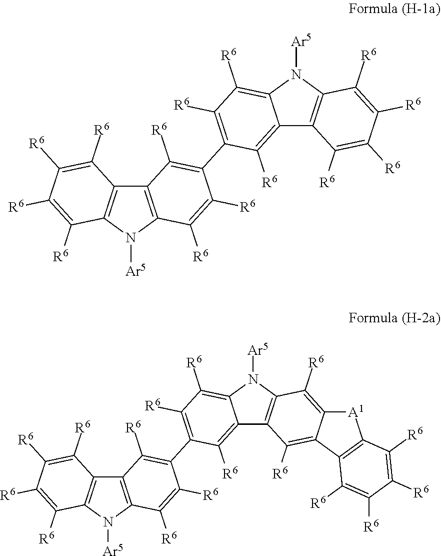

where R⁶, Ar⁵ and A¹ have the definitions given above, especially for formula (H-1) or (H-2). In a preferred embodiment of the invention, A¹ in formula (H-2a) is C(R⁷)₂.

Preferred embodiments of the compounds of the formulae (H-1a) and (H-2a) are the compounds of the following formulae (H-1b) and (H-2b):

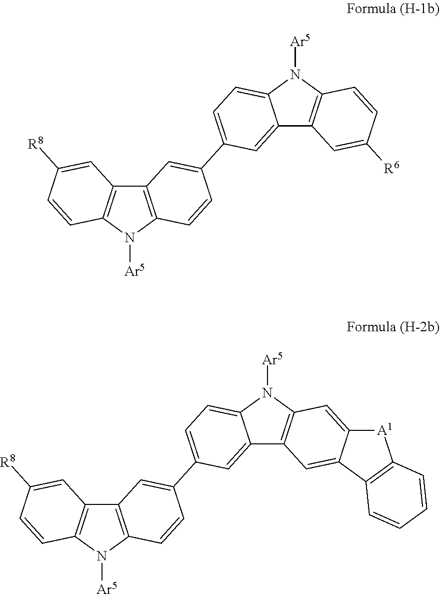

where R⁶, Ar⁵ and A¹ have the definitions given above, especially for formula (H-1) or (H-2). In a preferred embodiment of the invention, A¹ in formula (H-2b) is C(R⁷)₂.

Examples of suitable compounds of formula (H-1), (H-2), (H-3), (H-4) or (H-5) are the explicit compounds depicted in application no. PCT/EP2020/074320, entitled “Materials for organic electroluminescent devices”, on pages 69 to 73 as examples of compounds of formula (6), (7), (8), (9) or (10). These compounds are incorporated into the present application by reference thereto for purposes of disclosure.

The combination of at least one compound comprising at least one structure of formula (Ia), (I) or the preferred embodiments thereof that are set out above with a compound of one of the formulae (H-1), (H-2), (H-3), (H-4) or (H-5) can achieve surprising advantages. The present invention therefore further provides a composition comprising at least one compound comprising at least one structure of formula (Ia) or the preferred embodiments thereof that are set out above, especially compound comprising at least one structure of formula (I), and at least one further matrix material, wherein the further matrix material is selected from compounds of one of the formulae (H-1), (H-2), (H-3), (H-4) or (H-5).

In a preferred configuration, it may be the case that the inventive compound comprising at least one structure of formula (Ia) is used as matrix material for phosphorescent emitters in combination with a further matrix material selected from compounds of one of the formulae (H-1), (H-2), (H-3), (H-4) or (H-5). Accordingly, preference is given to electronic devices in which the compound comprising at least one structure of the formula (Ia) is used as matrix material for phosphorescent emitters in combination with a further matrix material selected from compounds of one of the formulae (H-1), (H-2), (H-3), (H-4) or (H-5).

It may preferably be the case that the composition consists of at least one compound comprising at least one structure of formula (Ia) or the preferred embodiments thereof that are set out above, especially compounds comprising at least one structure of formula (I), and at least one compound of one of the formulae (H-1), (H-2), (H-3), (H-4) or (H-5). These compositions are especially suitable as what are called pre-mixtures, which can be evaporated together.

In this context, the compounds of one of the formulae (H-1), (H-2), (H-3), (H-4) and (H-5) may each be used individually or as a mixture of two, three or more compounds of the respective structures.

In addition, the compounds of the formulae (H-1), (H-2), (H-3), (H-4) and (H-5) may be used individually or as a mixture of two, three or more compounds of different structures.

The compound comprising at least one structure of formula (Ia) or the preferred embodiments thereof that are set out above, especially compounds comprising at least one structure of formula (I), preferably has a proportion by mass in the composition in the range from 10% by weight to 95% by weight, preferably in the range from 15% by weight to 90% by weight, and very preferably in the range from 40% by weight to 70% by weight, based on the total mass of the composition. Compounds comprising at least one structure of formula (Ia) may be used individually or as a mixture of two, three or more compounds.

It may further be the case that the compounds of one of the formulae (H-1), (H-2), (H-3), (H-4) and (H-5) have a proportion by mass in the composition in the range from 5% by weight to 90% by weight, preferably in the range from 10% by weight to 85% by weight, more preferably in the range from 20% by weight to 85% by weight, even more preferably in the range from 30% by weight to 80% by weight, very particularly preferably in the range from 20% by weight to 60% by weight and most preferably in the range from 30% by weight to 50% by weight, based on the overall composition.

It may additionally be the case that the composition consists exclusively of compounds of the formula (Ia) or the preferred embodiments thereof that are set out above and one of the further matrix materials mentioned, preferably compounds of at least one of the formulae (H-1), (H-2), (H-3), (H-4) or (H-5).

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the above-described emitters can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439 and WO 2018/011186. In general, all phosphorescent complexes as used for phosphorescent electroluminescent devices according to the prior art and as known to those skilled in the art in the field of organic electroluminescence are suitable, and the person skilled in the art will be able to use further phosphorescent complexes without exercising inventive skill.

Examples of phosphorescent dopants are listed in the following table:

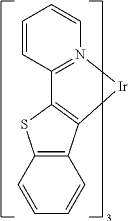

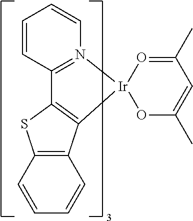

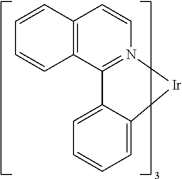

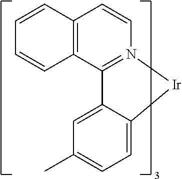

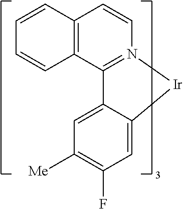

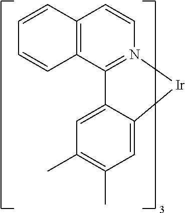

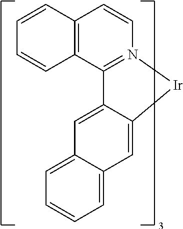

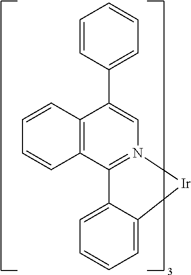

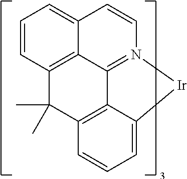

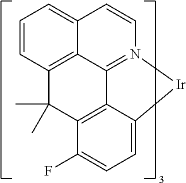

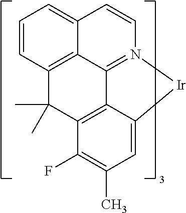

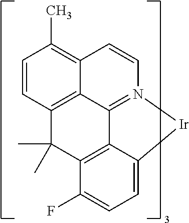

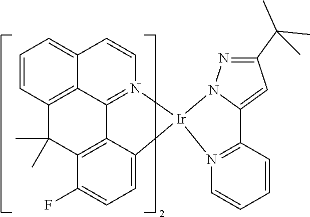

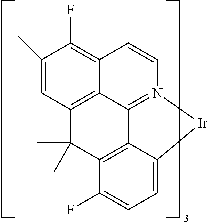

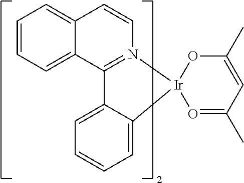

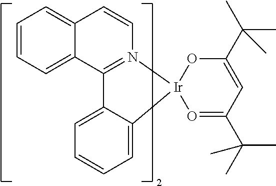

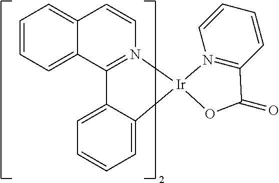

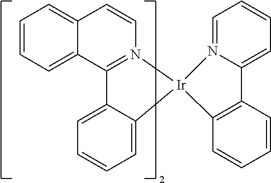

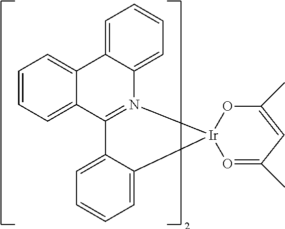

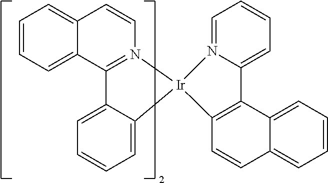

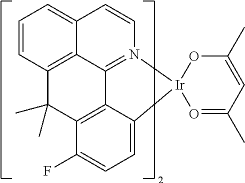

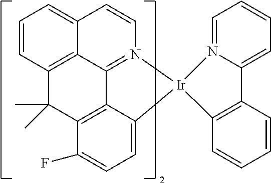

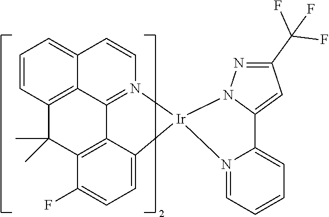

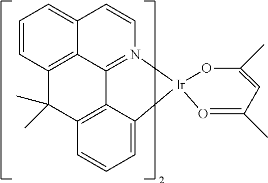

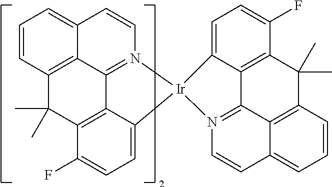

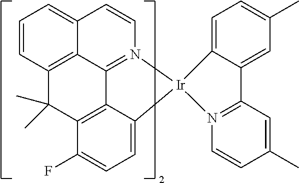

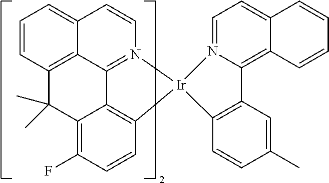

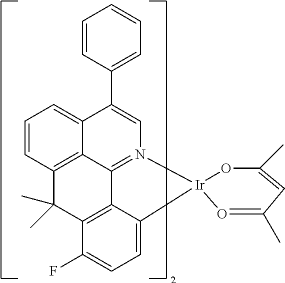

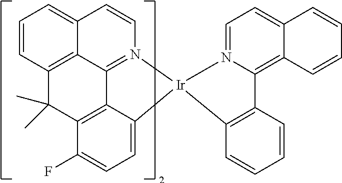

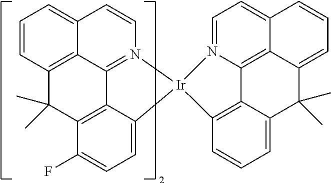

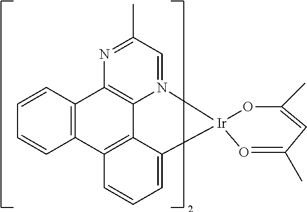

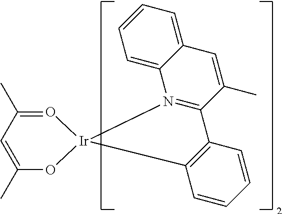

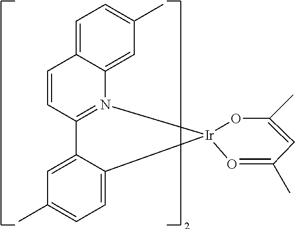

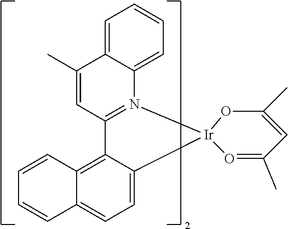

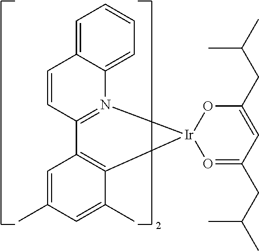

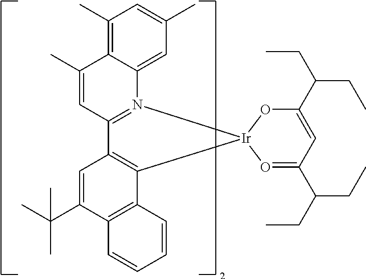

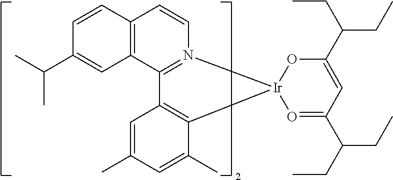

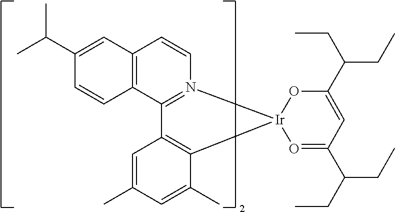

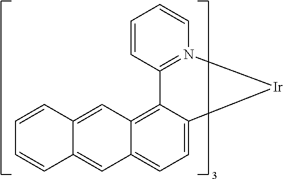

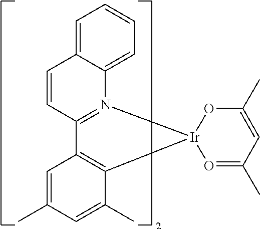

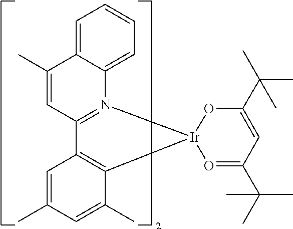

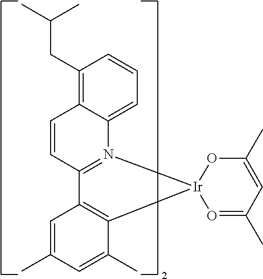

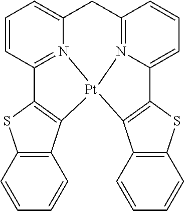

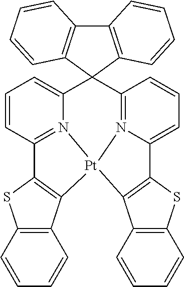

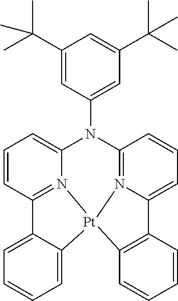

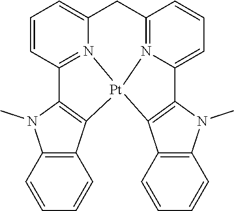

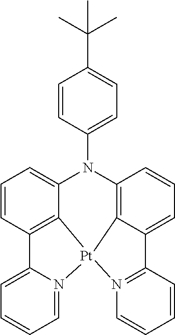

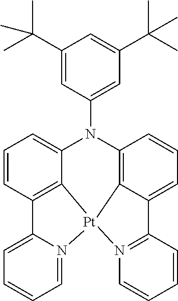

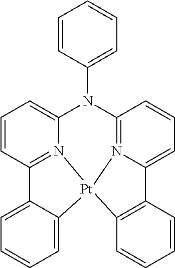

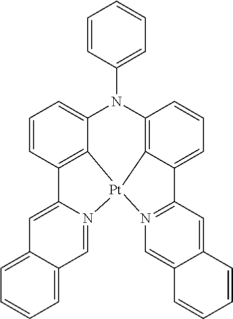

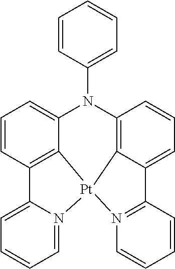

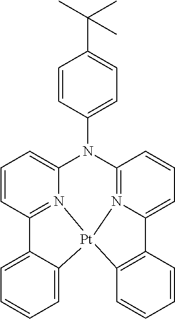

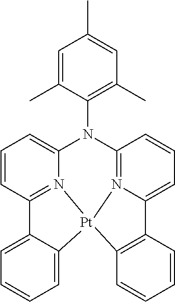

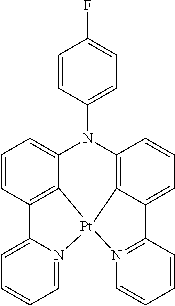

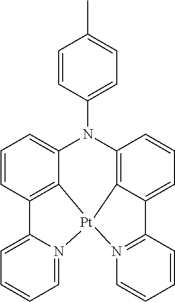

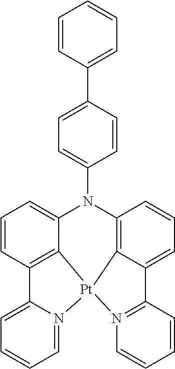

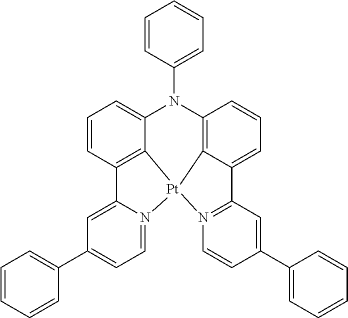

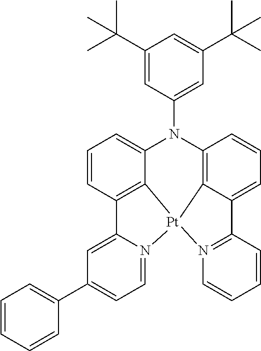

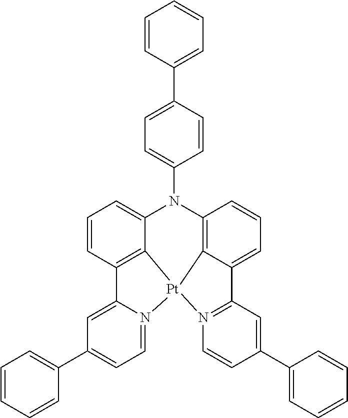

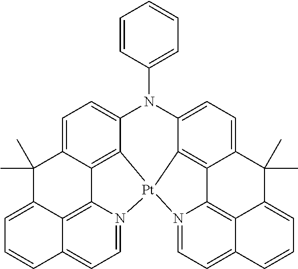

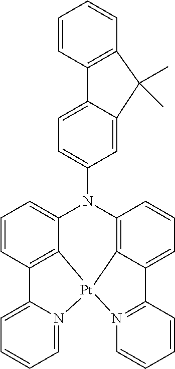

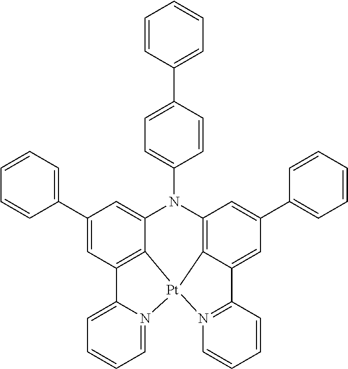

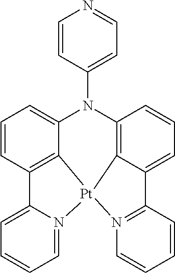

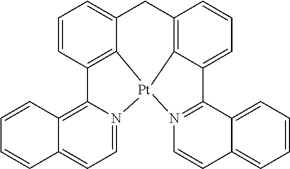

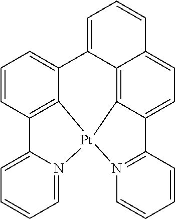

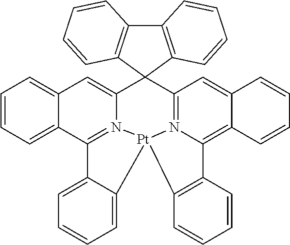

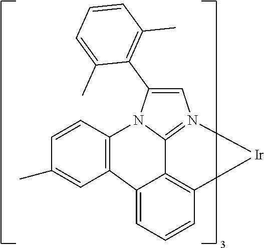

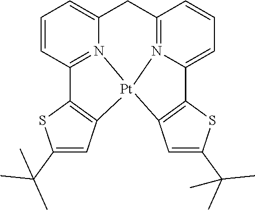

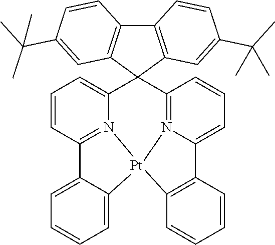

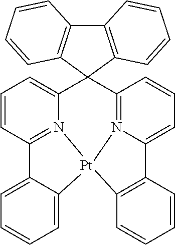

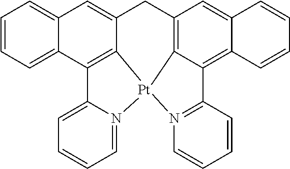

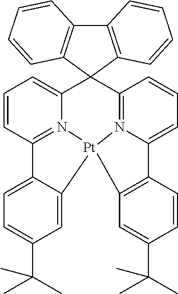

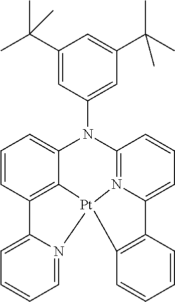

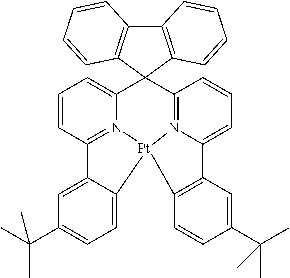

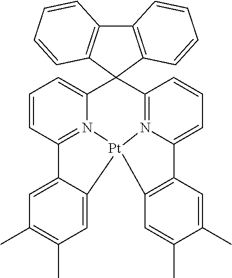

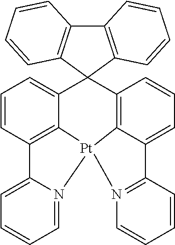

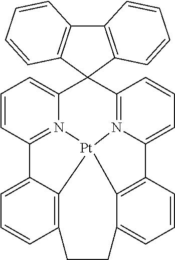

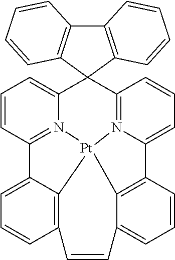

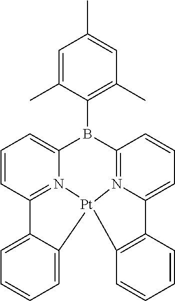

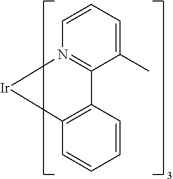

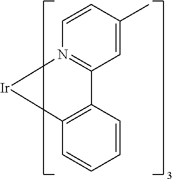

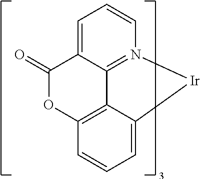

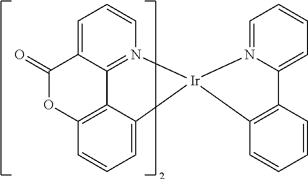

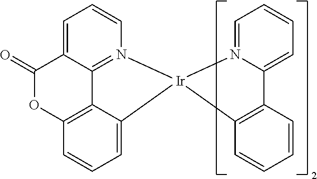

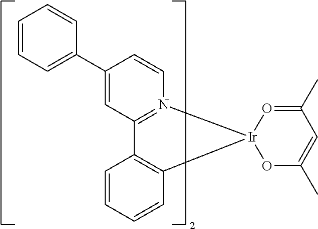

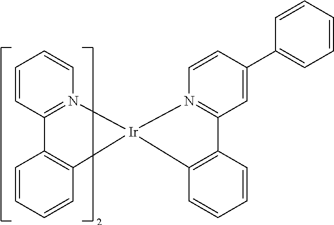

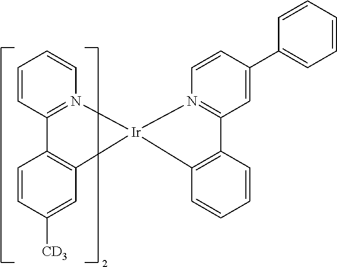

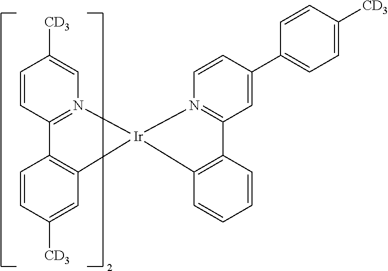

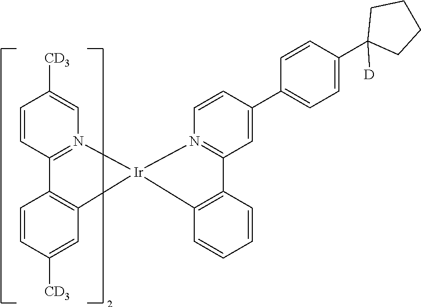

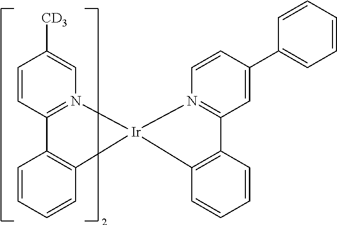

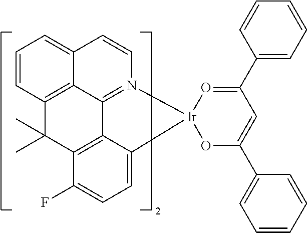

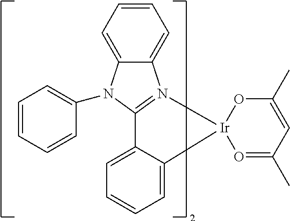

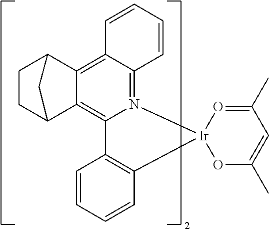

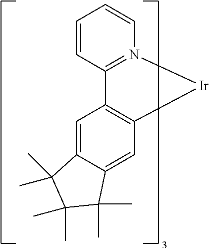

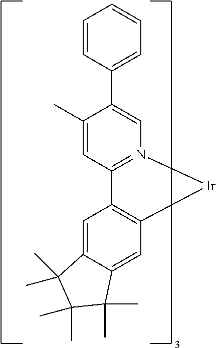

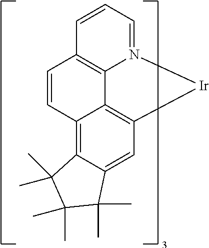

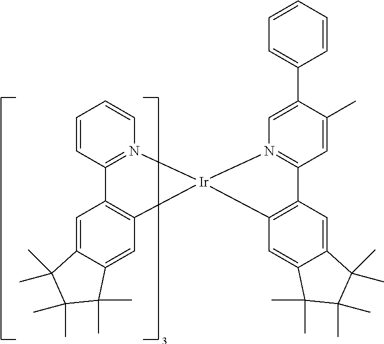

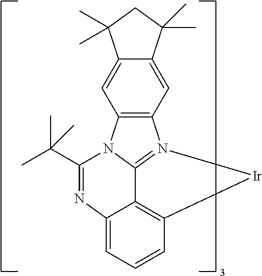

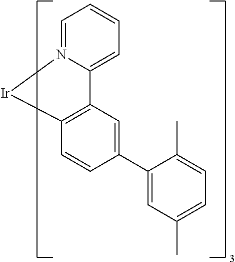

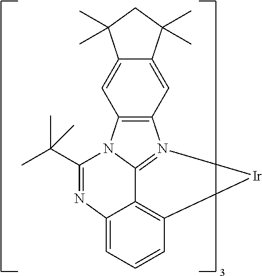

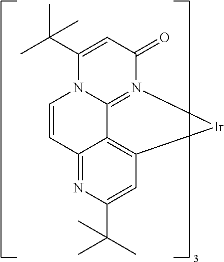

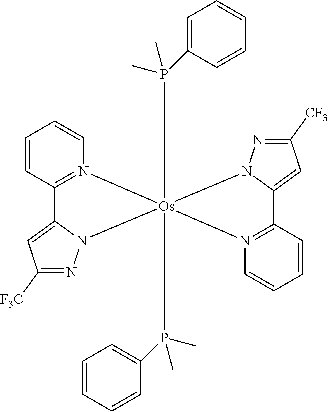

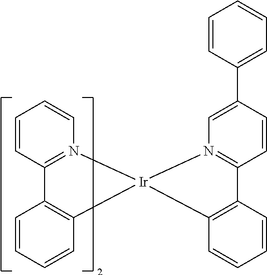

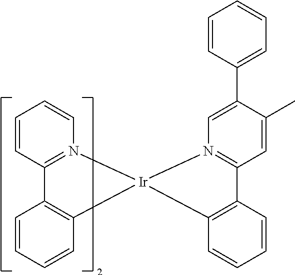

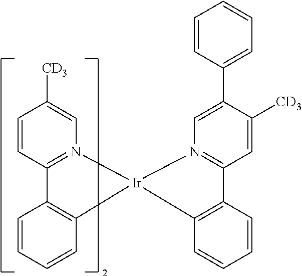

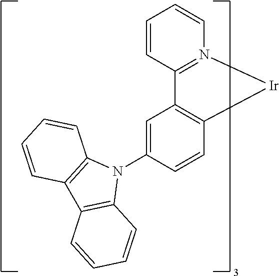

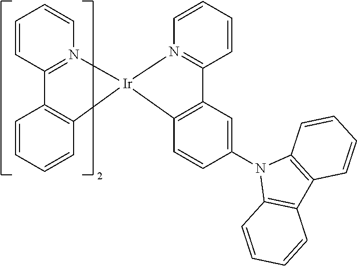

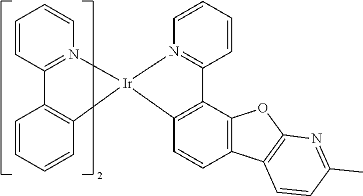

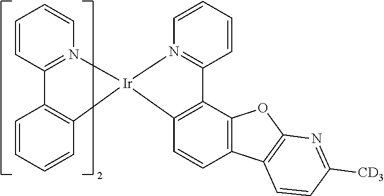

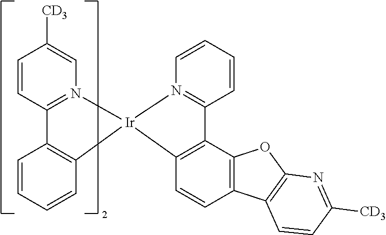

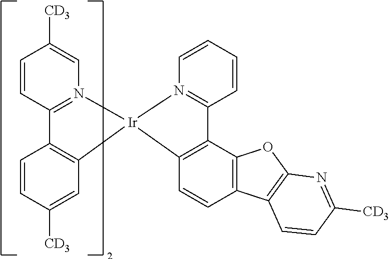

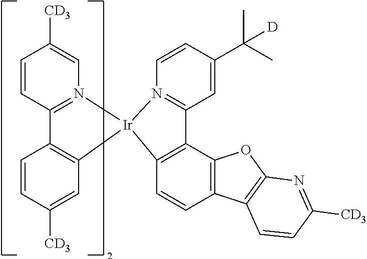

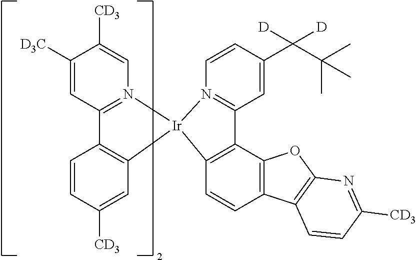

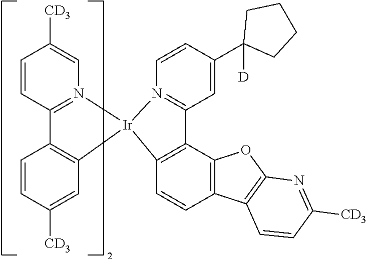

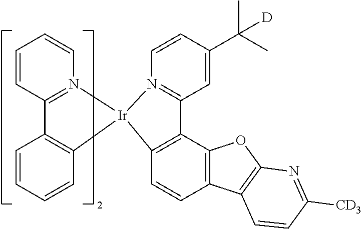

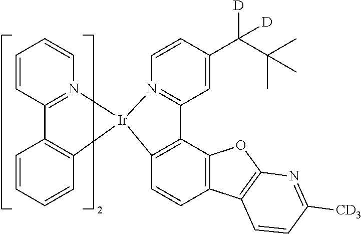

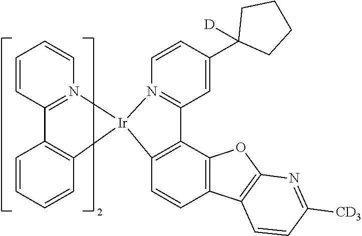

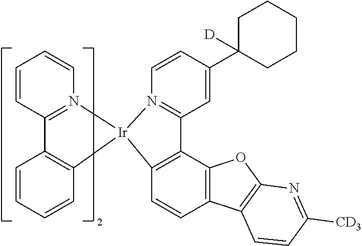

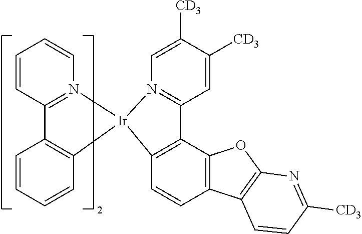

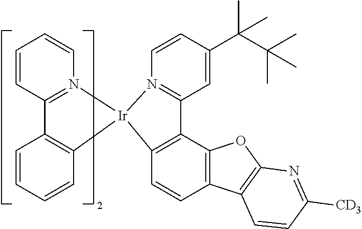

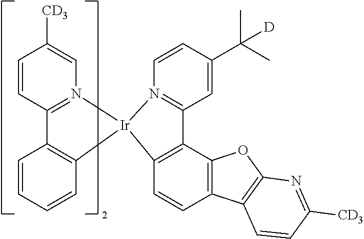

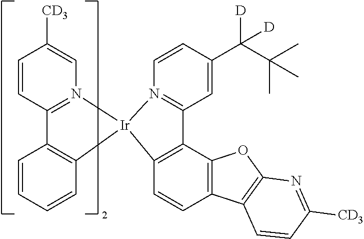

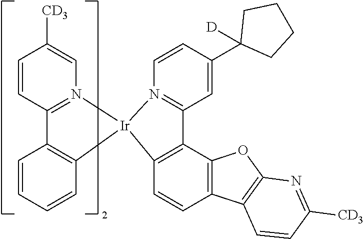

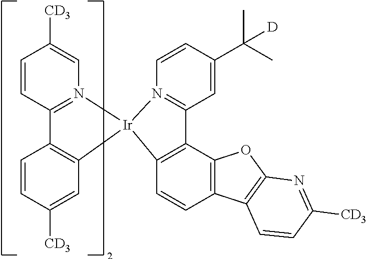

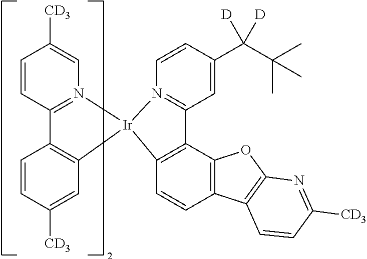

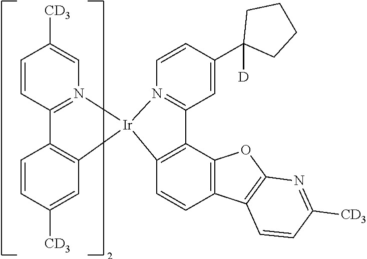

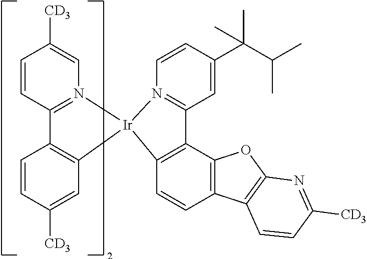

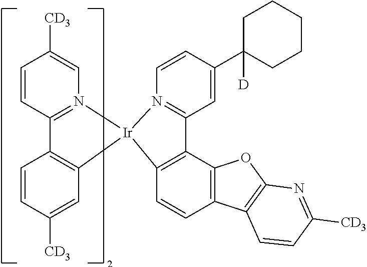

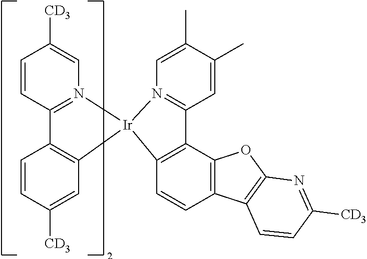

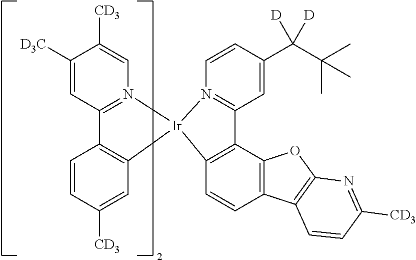

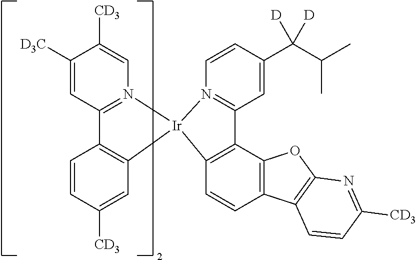

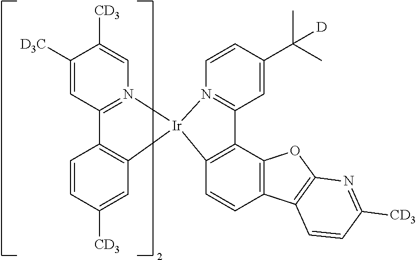

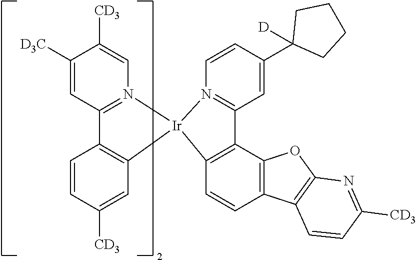

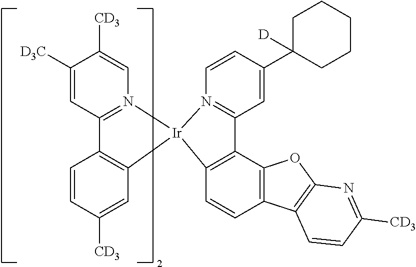

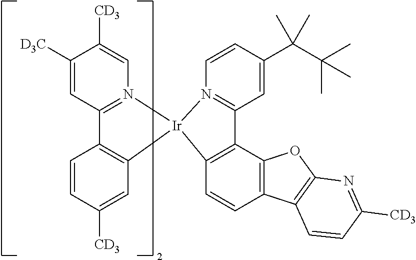

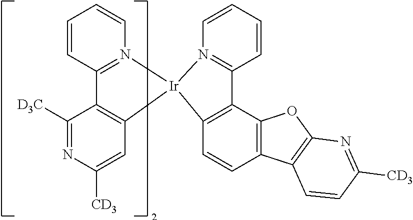

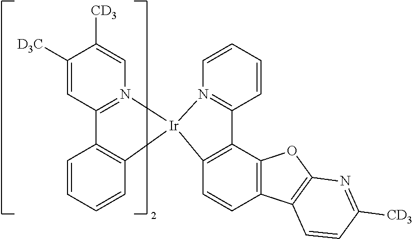

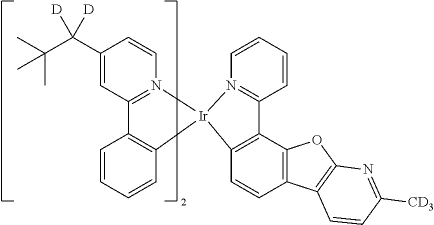

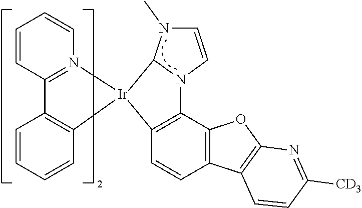

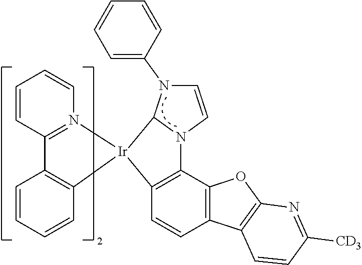

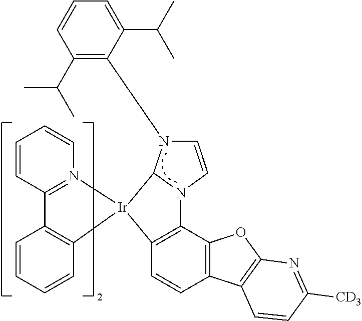

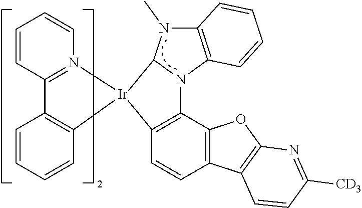

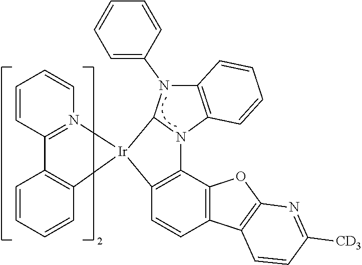

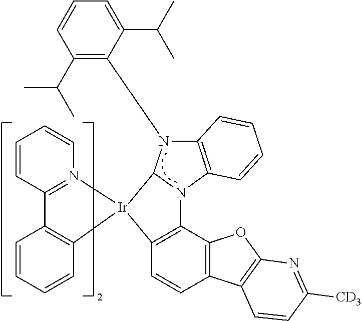

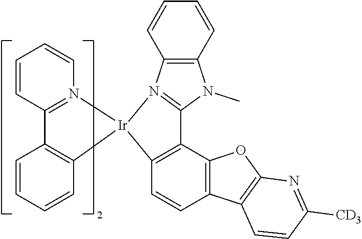

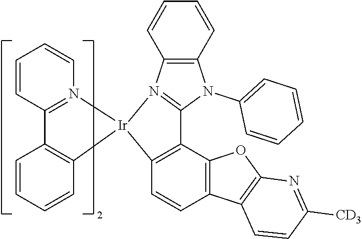

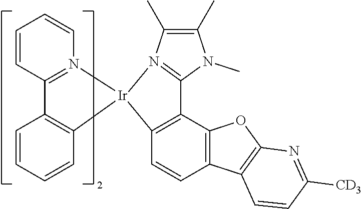

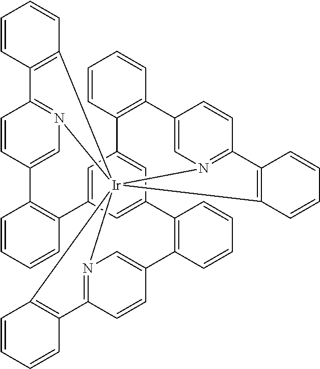

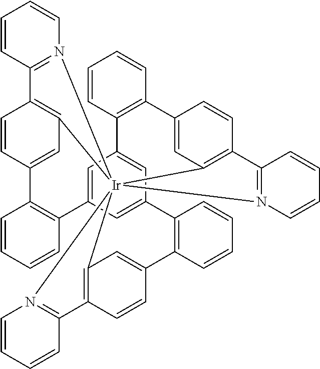

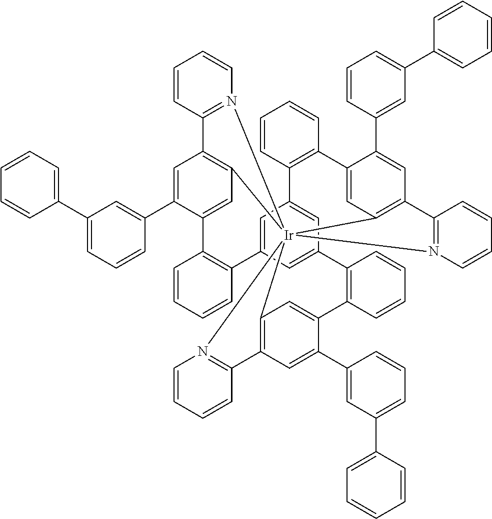

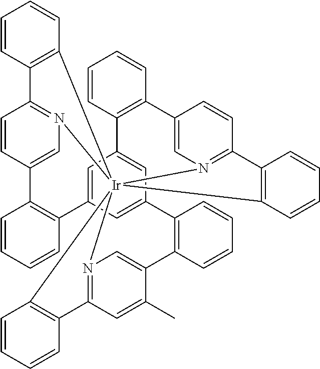

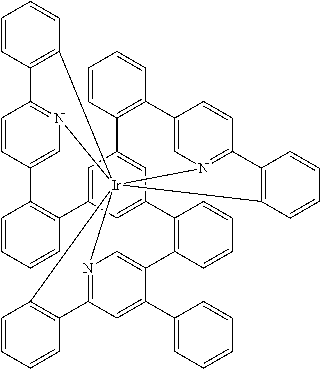

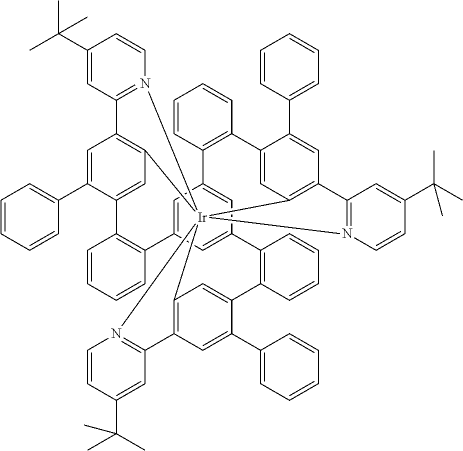

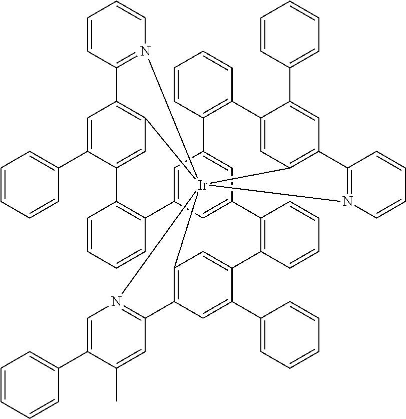

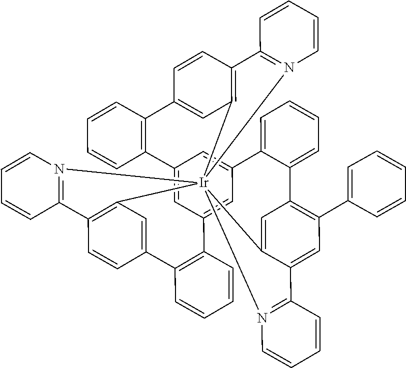

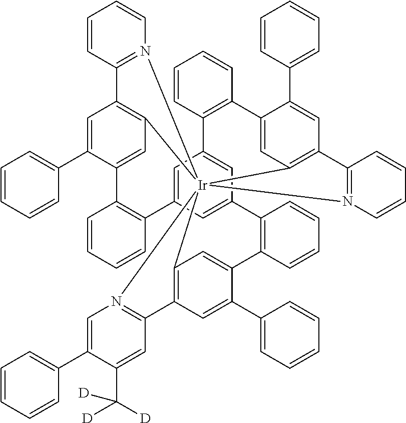

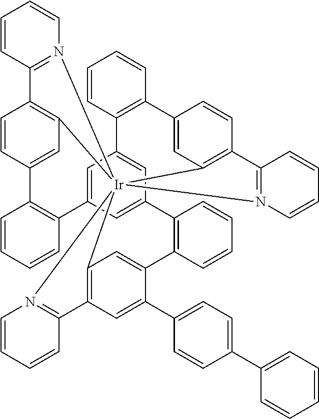

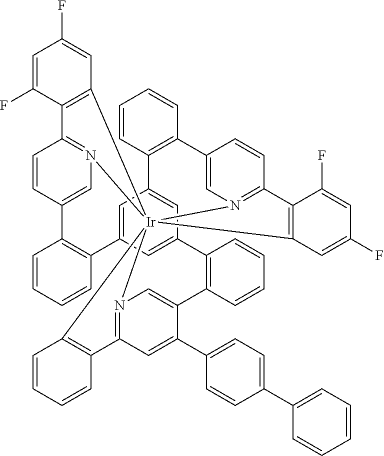

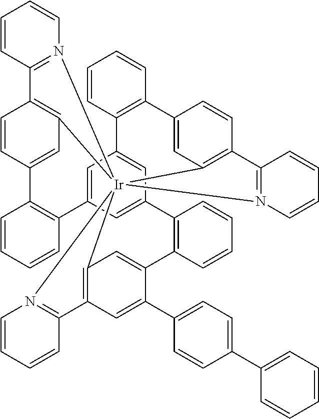

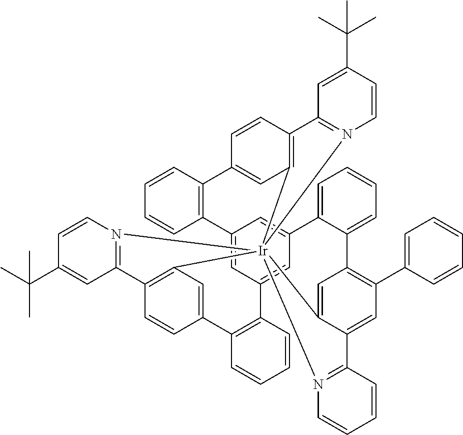

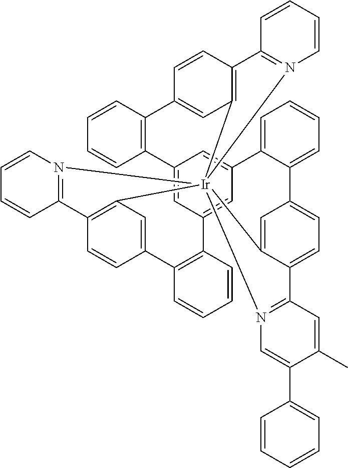

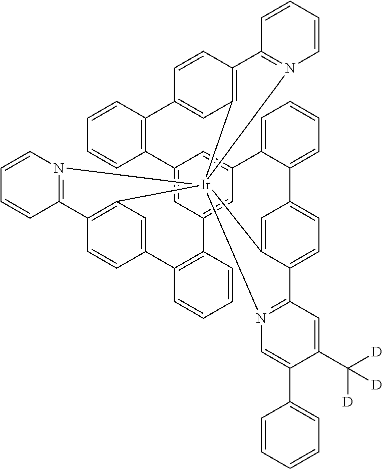

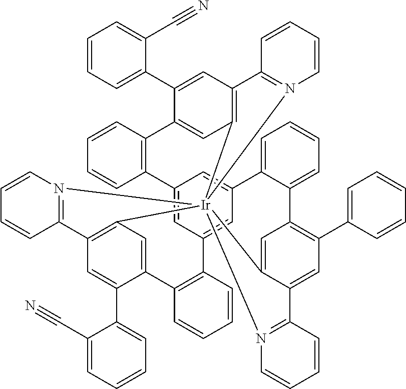

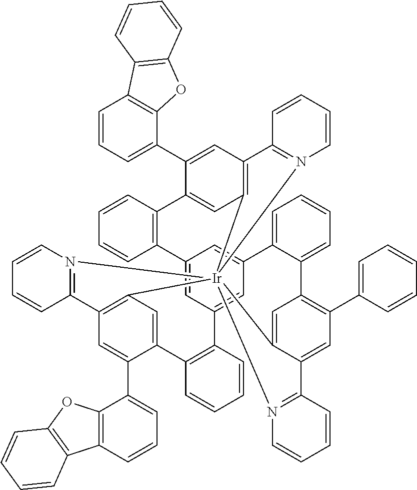

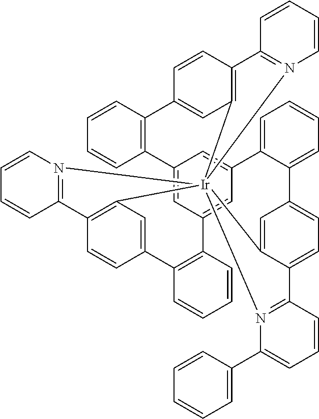

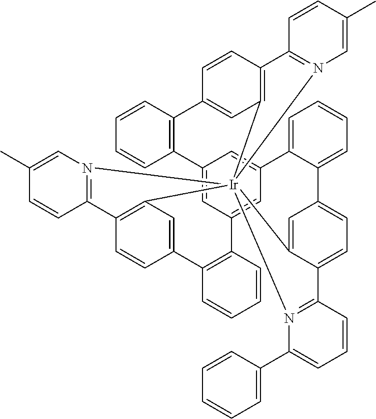

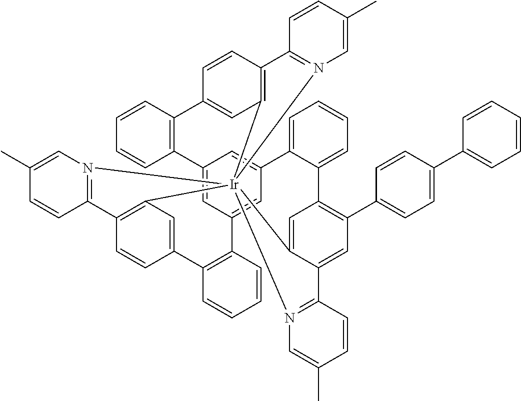

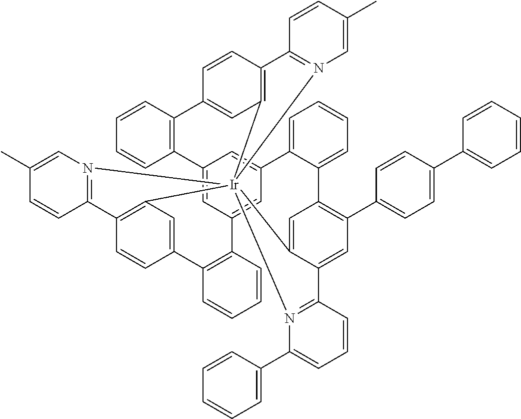

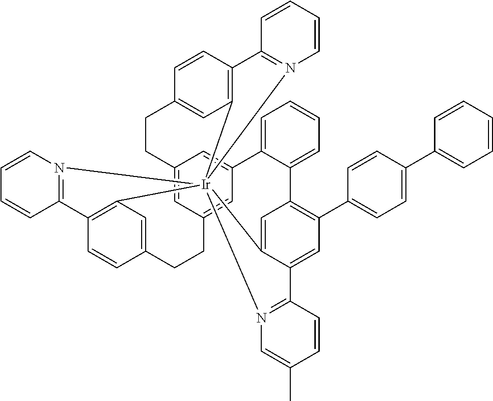

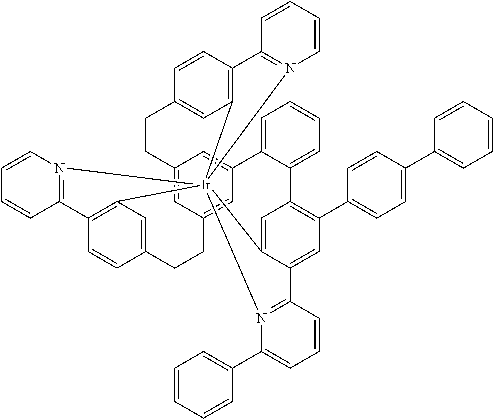

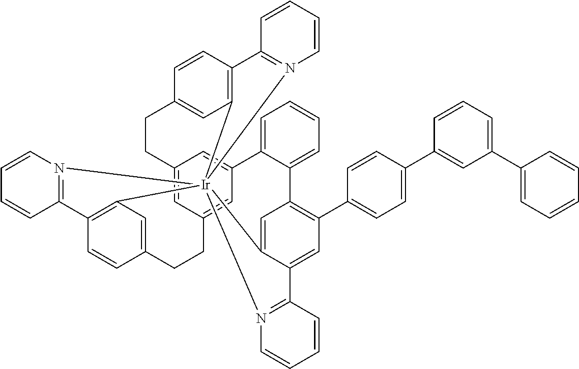

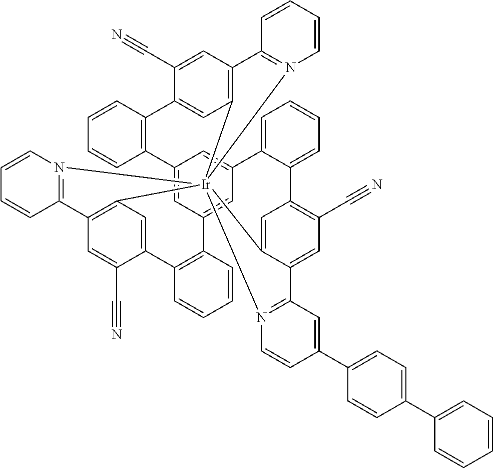

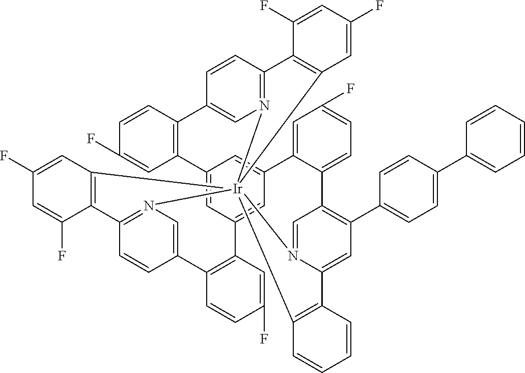

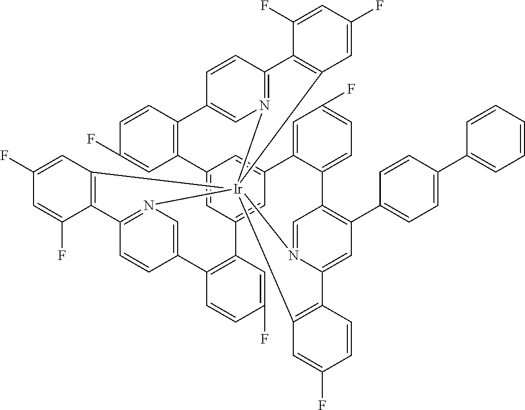

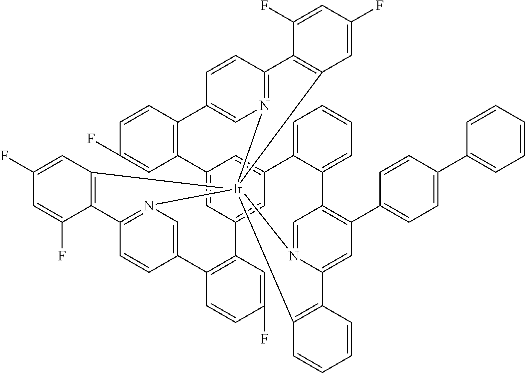

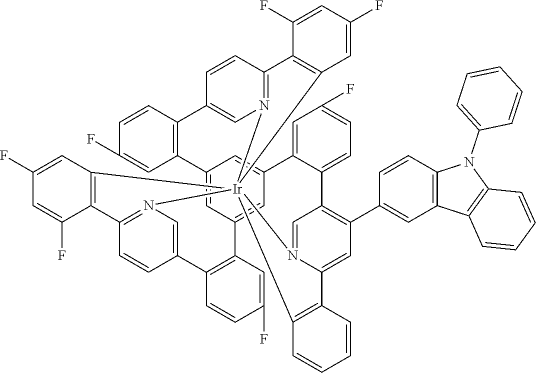

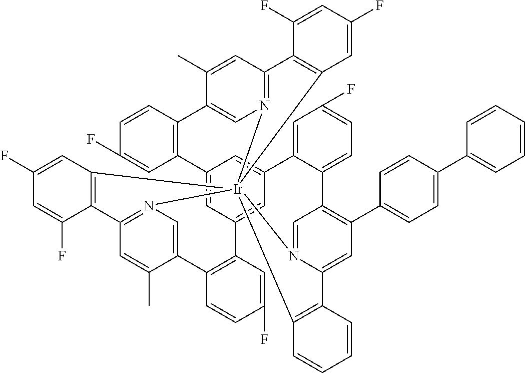

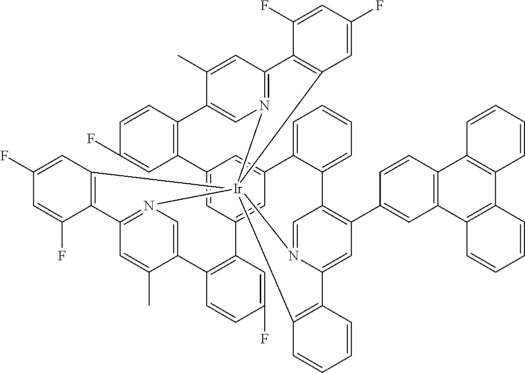

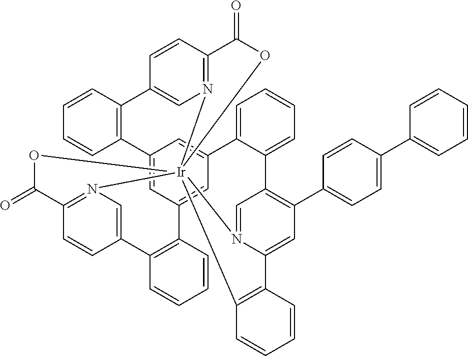

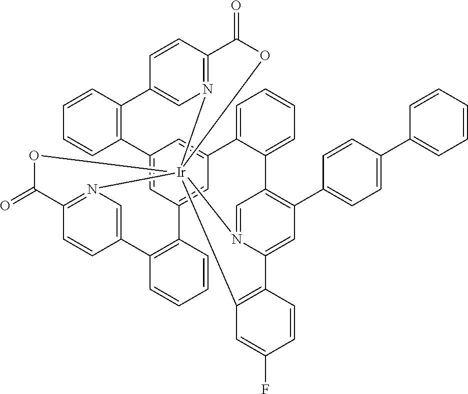

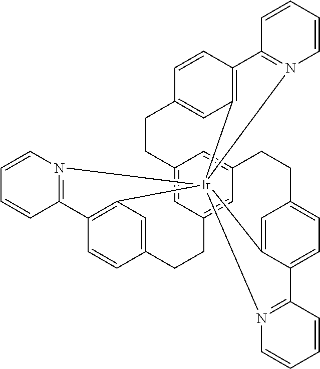

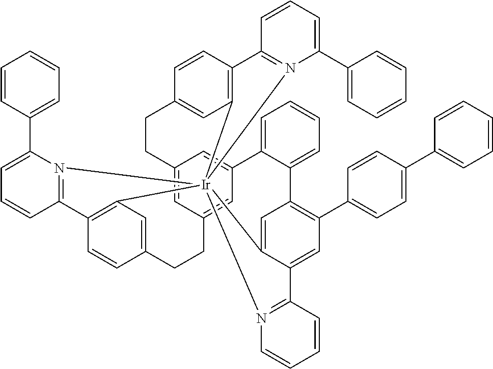

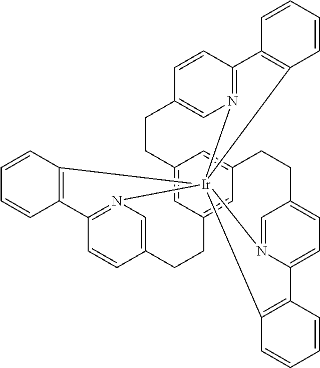

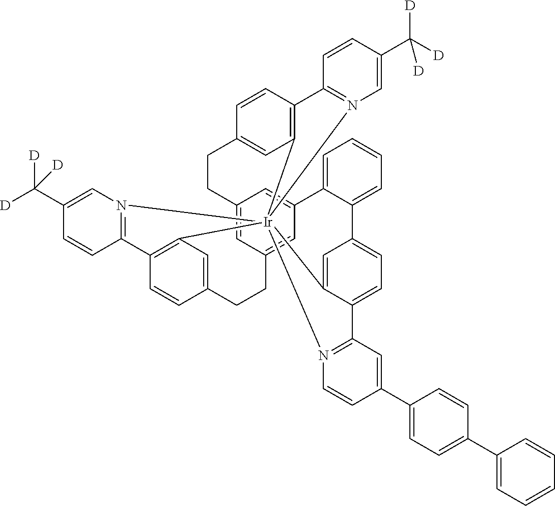

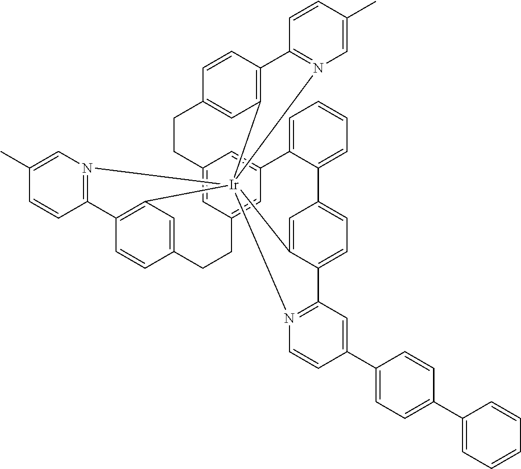

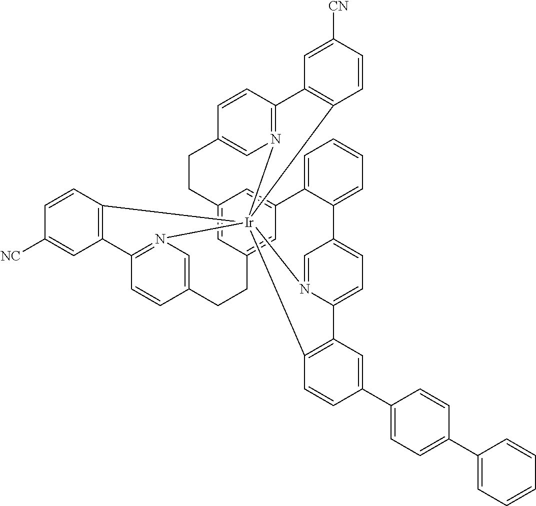

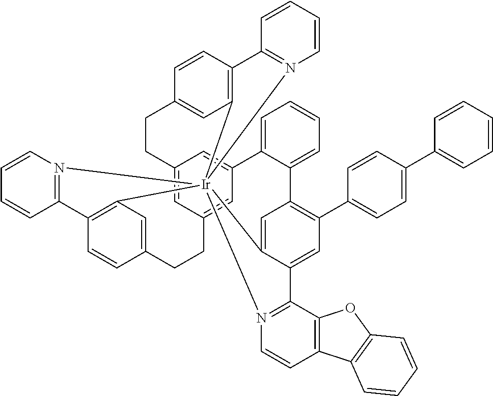

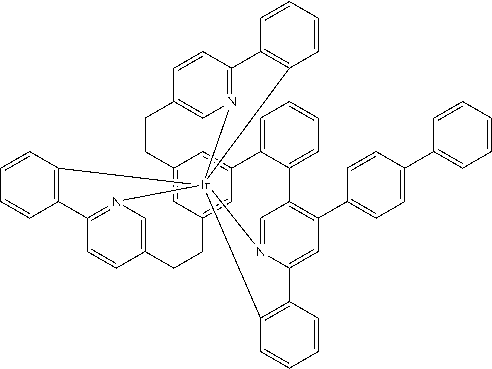

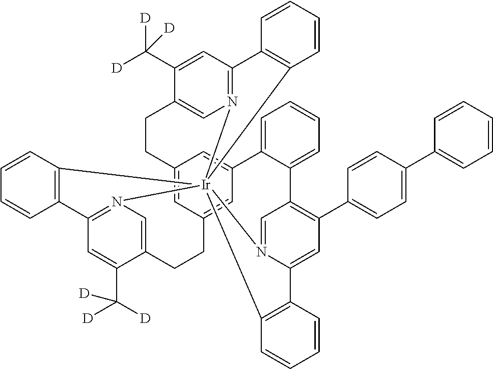

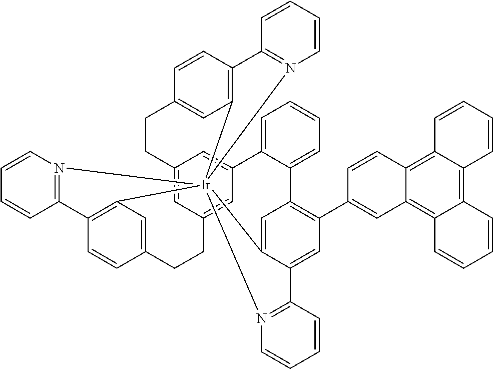

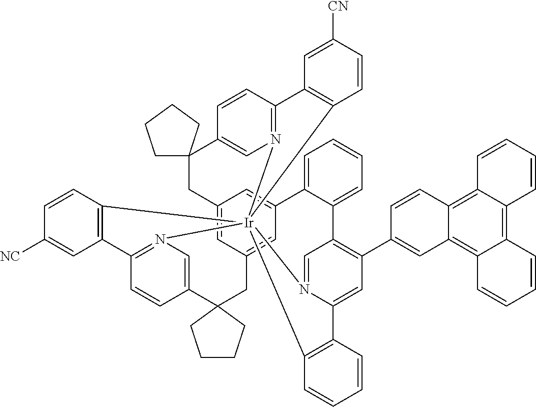

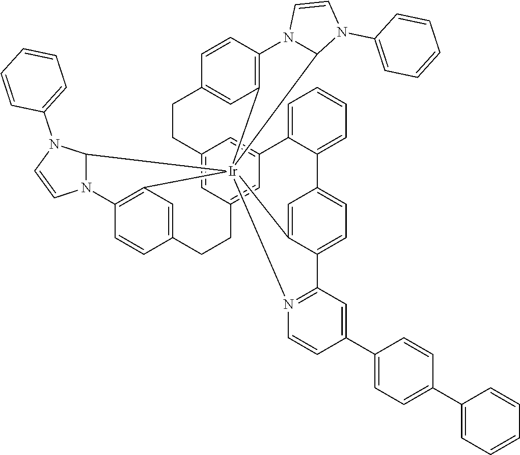

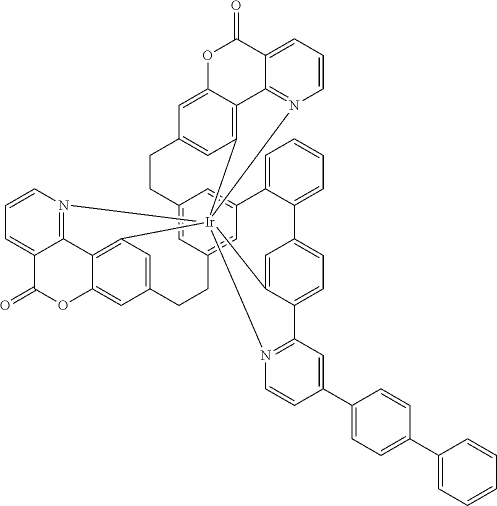

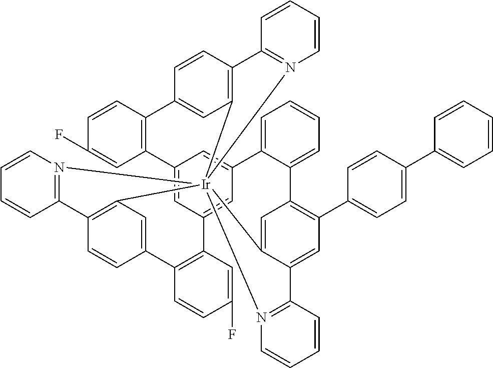

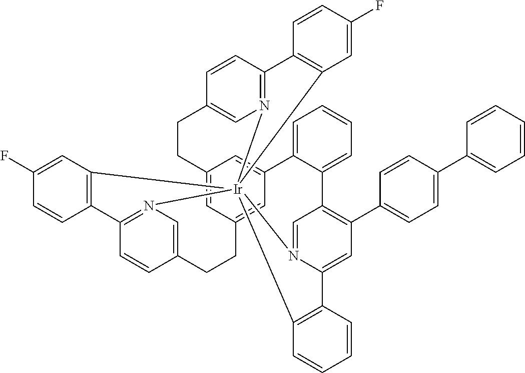

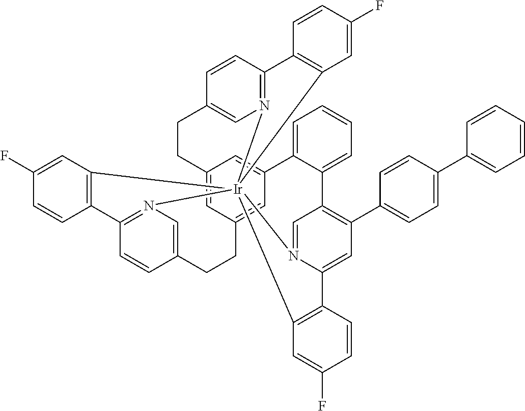

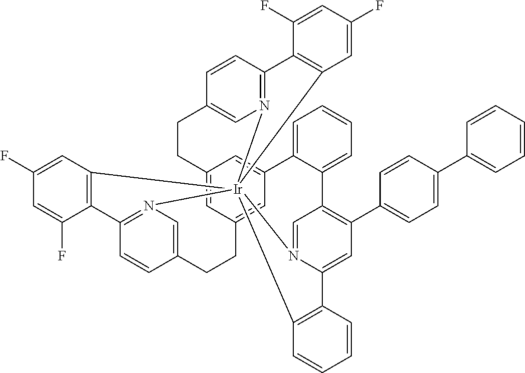

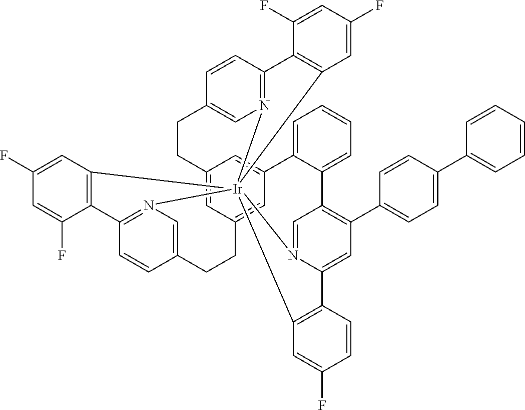

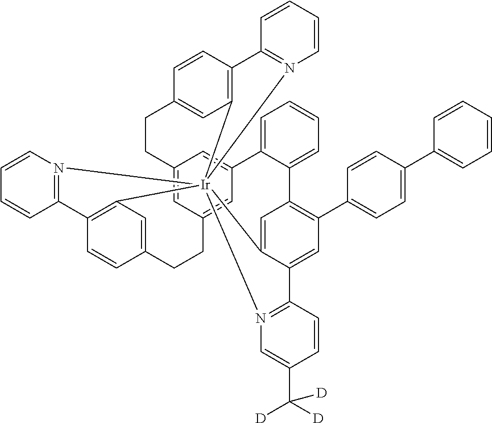

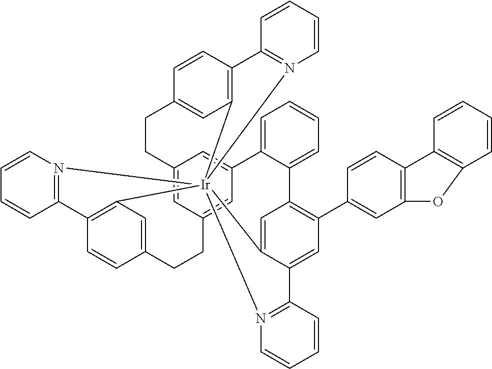

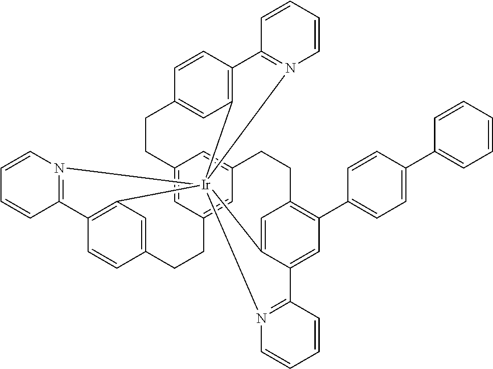

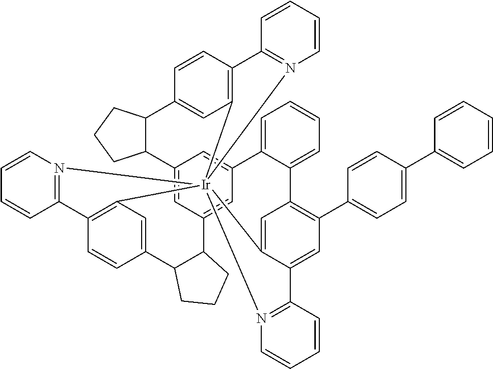

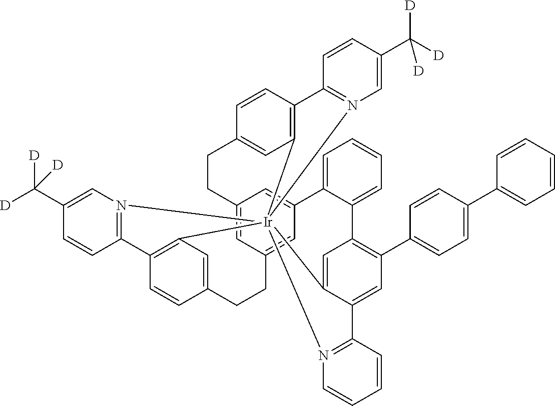

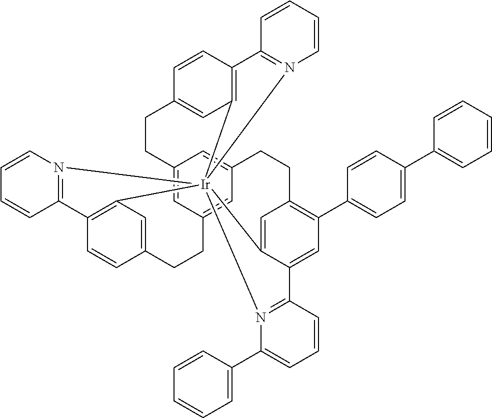

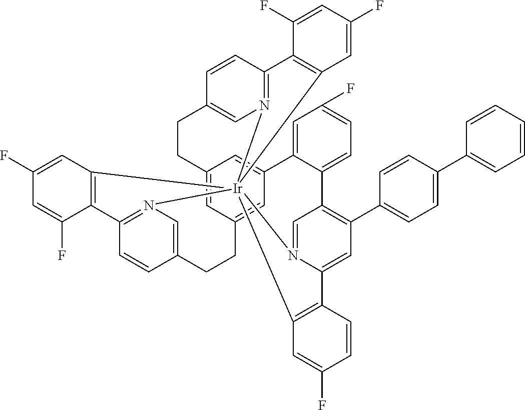

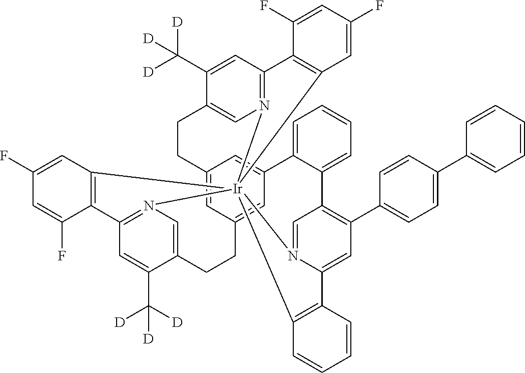

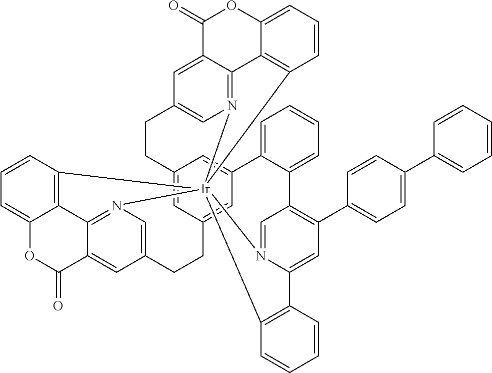

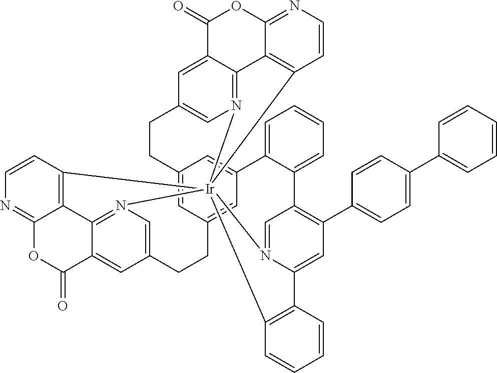

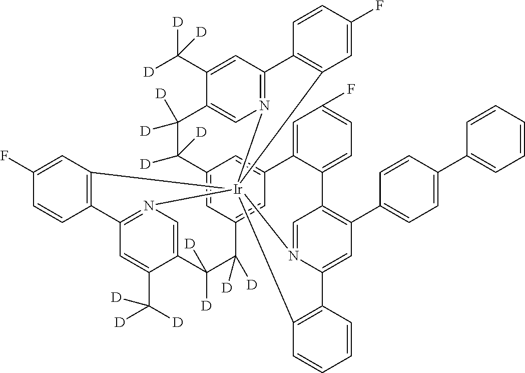

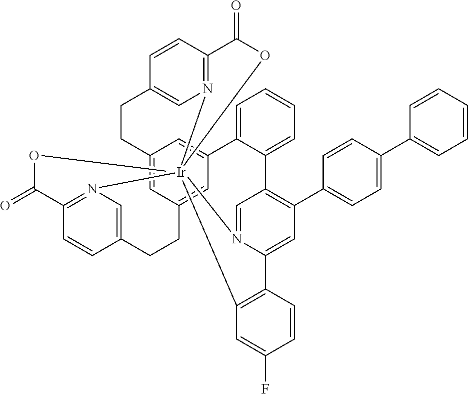

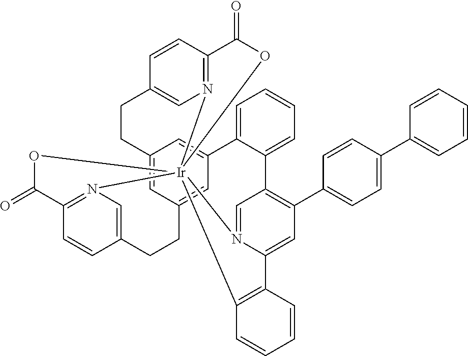

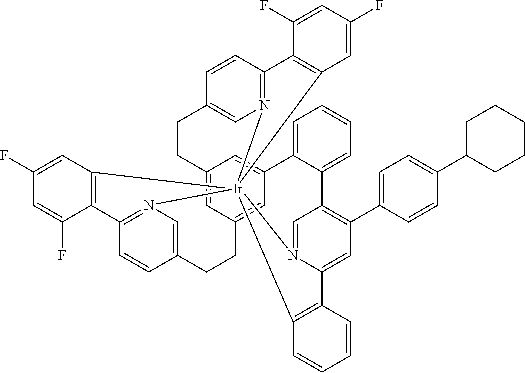

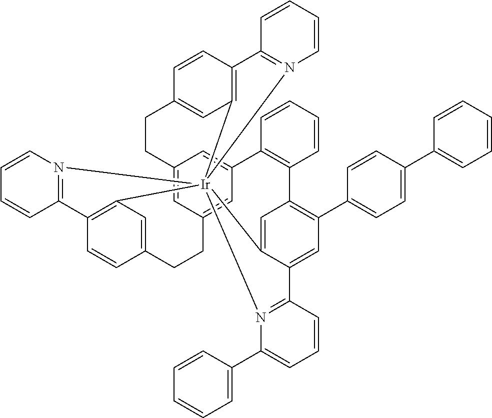

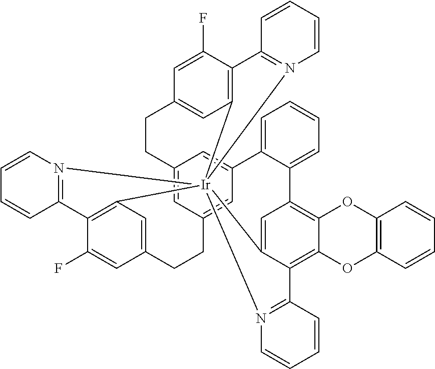

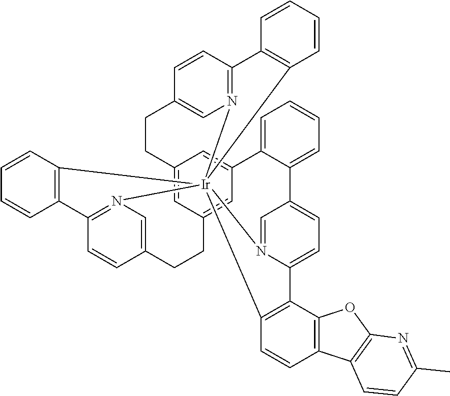

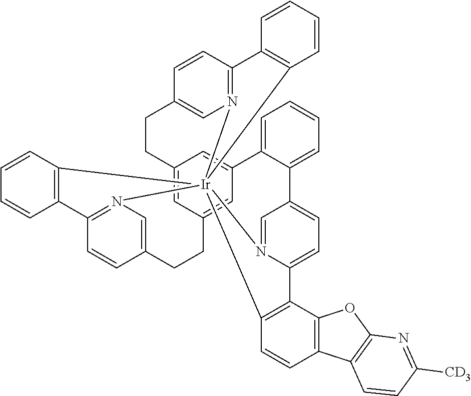

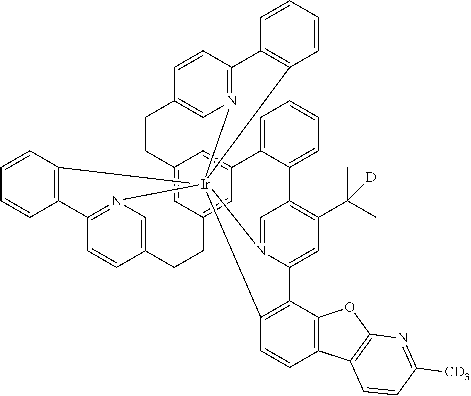

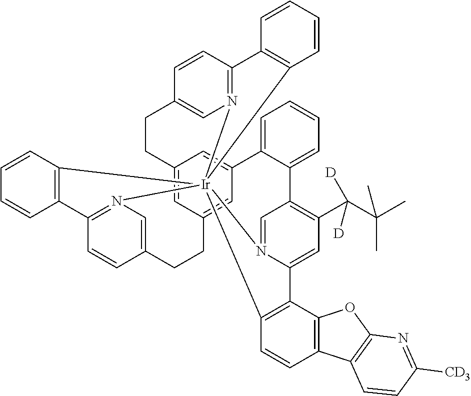

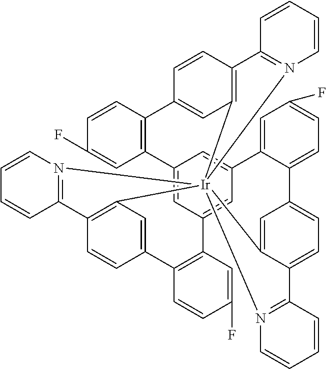

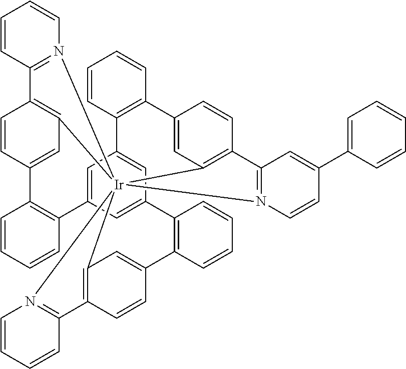

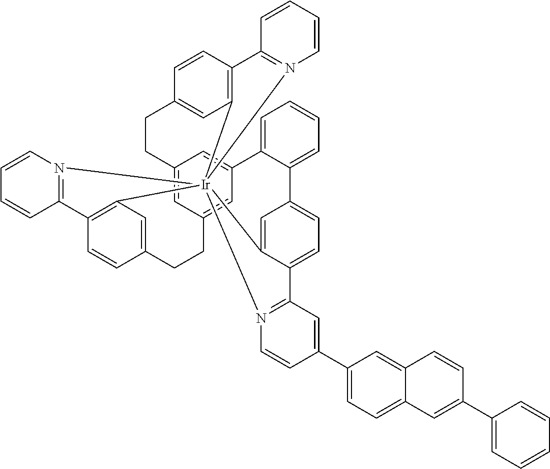

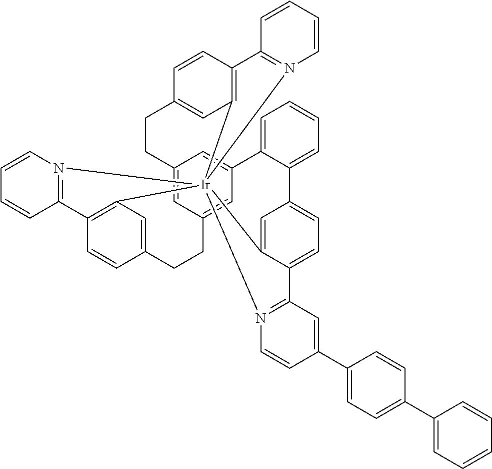

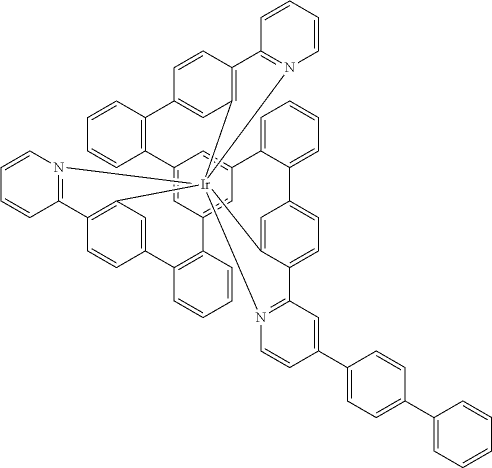

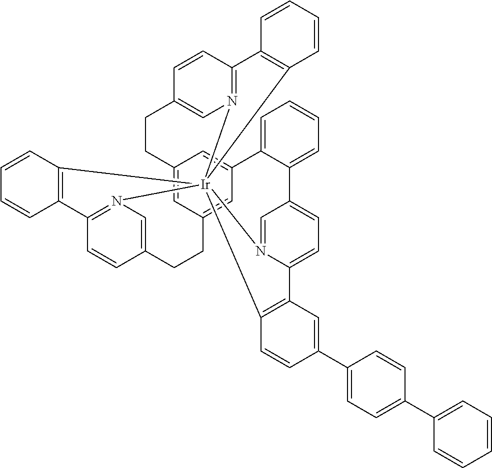

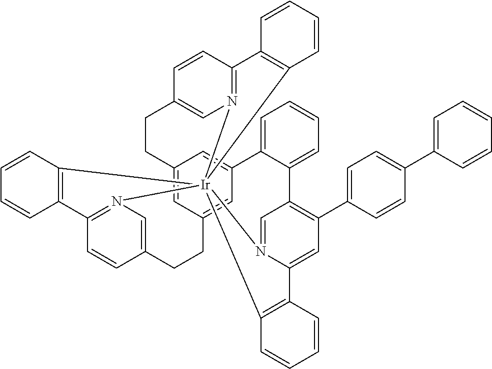

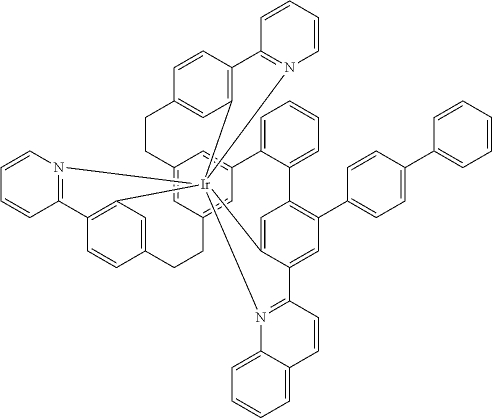

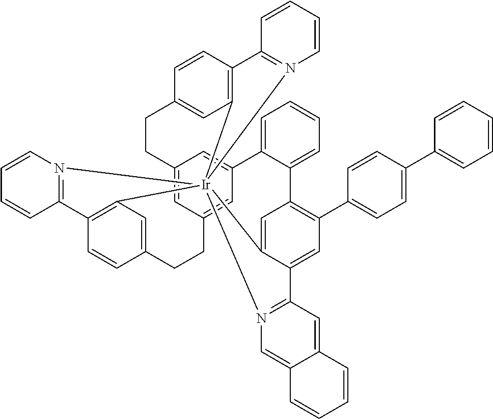

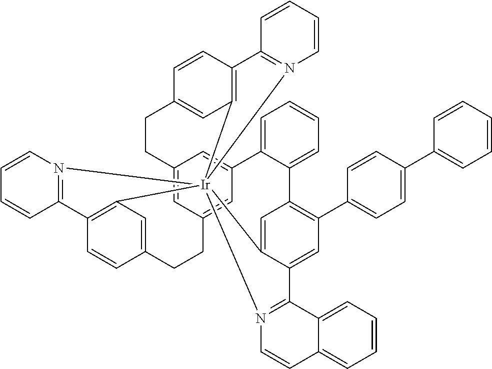

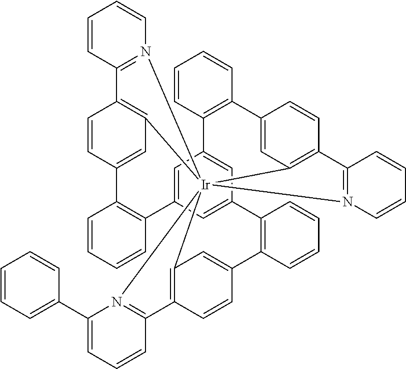

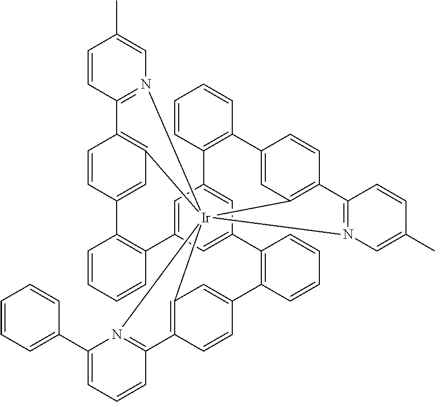

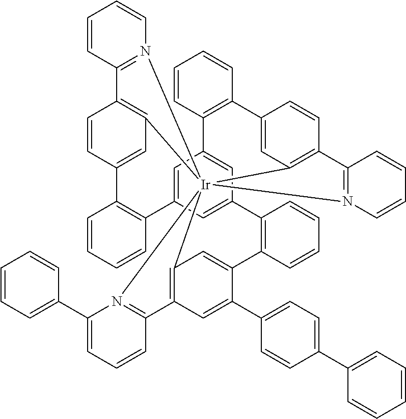

The compounds of the invention are especially also suitable as matrix materials for phosphorescent emitters in organic electroluminescent devices, as described, for example, in WO 98/24271, US 2011/0248247 and US 2012/0223633. In these multicolor display components, an additional blue emission layer is applied by vapor deposition over the full area to all pixels, including those having a color other than blue.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds comprising at least one structure of formula (Ia) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

Formulations for application of a compound comprising at least one structure of formula (I) or the preferred embodiments thereof or the preferred embodiments thereof set out above are novel. The present invention therefore further provides a formulation comprising at least one solvent and a compound comprising at least one structure of formula (I) or the preferred embodiments thereof that are set out above. The present invention further provides a formulation comprising at least one solvent and a compound comprising at least one structure of formula (Ia) or the preferred embodiments thereof that are set out above, and a compound of at least one of the formulae (H-1), (H-2), (H-3), (H-4) or (H-5).

In addition, hybrid methods are possible, in which, for example, one or more layers are applied from solution and one or more further layers are applied by vapor deposition.

Those skilled in the art are generally aware of these methods and are able to apply them without exercising inventive skill to organic electroluminescent devices comprising the compounds of the invention.

The compounds of the invention and the organic electroluminescent devices of the invention have the particular feature of an improved lifetime over the prior art. At the same time, the further electronic properties of the electroluminescent devices, such as efficiency or operating voltage, remain at least equally good. In a further variant, the compounds of the invention and the organic electroluminescent devices of the invention especially feature improved efficiency and/or operating voltage and higher lifetime compared to the prior art.

The electronic devices of the invention, especially organic electroluminescent devices, are notable for one or more of the following surprising advantages over the prior art:
1. Electronic devices, especially organic electroluminescent devices, comprising compounds comprising at least one structure of formula (Ia) or the preferred embodiments recited above and hereinafter, especially as matrix material or as electron-conducting materials, have a very good lifetime. In this context, these compounds especially bring about low roll-off, i.e. a small drop in power efficiency of the device at high luminances.
2. Electronic devices, especially organic electroluminescent devices, comprising compounds comprising at least one structure of formula (Ia) or the preferred embodiments recited above and hereinafter, as electron-conducting materials and/or matrix materials, have excellent efficiency. In this context, inventive compounds comprising at least one structure of formula (I) or the preferred embodiments recited above and hereinafter bring about a low operating voltage when used in electronic devices.
3. The inventive compounds comprising at least one structure of formula (I) or the preferred embodiments recited above and hereinafter exhibit very high stability and lifetime.
4. With compounds comprising at least one structure of formula (I) or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
5. Compounds comprising at least one structure of formula (I) or the preferred embodiments recited above and hereinafter have excellent glass film formation.
6. Compounds comprising at least one structure of formula (I) or the preferred embodiments recited above and hereinafter form very good films from solutions.
7. Electronic devices, especially organic electroluminescent devices comprising compounds comprising at least one structure of formula (Ia) or the preferred embodiments detailed above and hereinafter, in combination with host materials of one or more of the formulae (H-1) to (H-5), especially as matrix material, have an improved lifetime and higher efficiency.
8. The compounds comprising at least one structure of formula (I) or the preferred embodiments recited above and hereinafter have a low triplet level Ti which may, for example, be in the range of −2.22 eV to −2.9 eV.

These abovementioned advantages are not accompanied by an inordinately high deterioration in the further electronic properties.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

It should also be pointed out that many of the features, and especially those of the preferred embodiments of the present invention, should themselves be regarded as inventive and not merely as some of the embodiments of the present invention. For these features, independent protection may be sought in addition to or as an alternative to any currently claimed invention.

The technical teaching disclosed with the present invention may be abstracted and combined with other examples.

The invention is illustrated in detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

The syntheses which follow, unless stated otherwise, are conducted under a protective gas atmosphere in dried solvents. The solvents and reagents can be purchased, for example, from Sigma-ALDRICH or ABCR. For the compounds known from the literature, the corresponding CAS numbers are also reported in each case.

### Synthesis Examples

### a) 2-Bromo-6-phenylquinoxalino[2,1-b]quinazolin-12-one

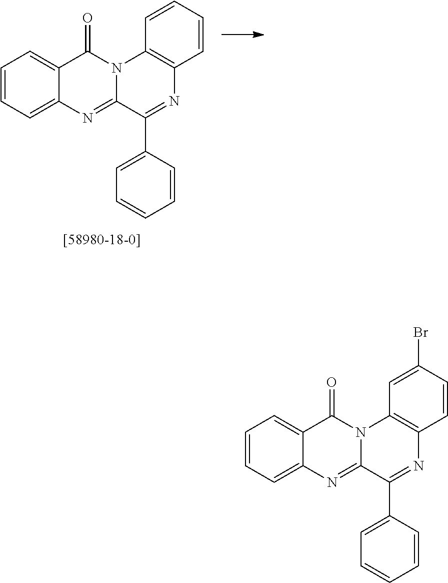 [SMILES: O=c1c2ccccc2nc2c(-c3ccccc3)nc3ccccc3n12 ; O=c1c2ccccc2nc2c(-c3ccccc3)nc3ccc(Br)cc3n12]

To a solution of 23.2 g (75 mmol) of 6-phenylquinoxalino[2,1-b]quinazolin-12-one in chloroform (400 ml) is added N-bromosuccinimide (13.3 g, 75 mmol) in portions at 0° C. in the dark, and the mixture is stirred at this temperature for 2 h. The reaction is ended by addition of sodium sulfite solution and the mixture is stirred at room temperature for a further 30 min. After phase separation, the organic phase is washed with water and the aqueous phase is extracted with dichloromethane. The combined organic phases are dried over sodium sulfate and concentrated under reduced pressure. The residue is dissolved in toluene and filtered through silica gel. Subsequently, the crude product is recrystallized from toluene/heptane.

Yield: 21 g (52 mmol), 70% of theory, colorless solid.

The following compounds are prepared in an analogous manner:

| Ex. | Reactant 1 | Product 1 | Yield |
|---|---|---|---|
| 1a | 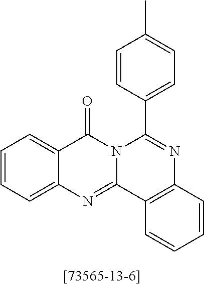 [SMILES: Cc1ccc(-c2nc3ccccc3c3nc4ccccc4c(=O)n23)cc1] | 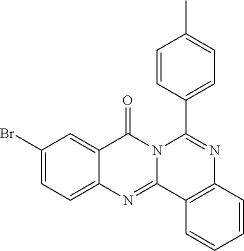 [SMILES: Cc1ccc(-c2nc3ccccc3c3nc4ccc(Br)cc4c(=O)n23)cc1] | 46% |
| 2a | 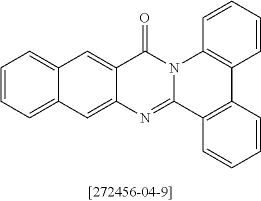 [SMILES: O=c1c2cc3ccccc3cc2nc2c3ccccc3c3ccccc3n12] | 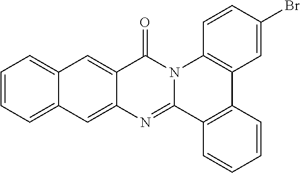 [SMILES: O=c1c2cc3ccccc3cc2nc2c3ccccc3c3cc(Br)ccc3n12] | 36% |

### b) 2-Dibenzofuran-1-yl-6-phenylquinoxalino[2,1-b]quinazolin-12-one

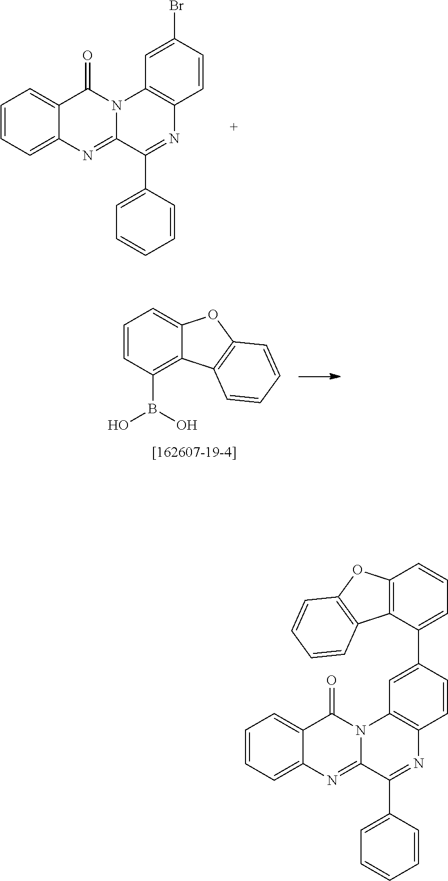 [SMILES: O=c1c2ccccc2nc2c(-c3ccccc3)nc3ccc(Br)cc3n12 ; OB(O)c1cccc2oc3ccccc3c12 ; O=c1c2ccccc2nc2c(-c3ccccc3)nc3ccc(-c4cccc5oc6ccccc6c45)cc3n12]

63 g (158 mmol) of 2-bromo-6-phenylquinoxalino[2,1-b]quinazolin-12-one, 36 g (170 mmol) of dibenzofuran-1-boronic acid and 36 g (340 mmol) of sodium carbonate are suspended in 1000 ml of ethylene glycol diamine ether and 280 ml of water. To this suspension is added 1.8 g (1.5 mmol) of tetrakis(triphenylphosphine)palladium(0), and the reaction mixture is heated under reflux for 14 h. After cooling, the organic phase is removed, filtered through silica gel and then concentrated to dryness. The product is purified via column chromatography on silica gel with toluene/heptane (1:3) and finally sublimed under high vacuum (p=5×10⁻⁷ mbar) (99.9% purity). The yield is 53 g (109 mmol), corresponding to 70% of theory.

The following compounds are prepared in an analogous manner:

| | Reactant 1 | Reactant 2 | Product | Yield |
|---|---|---|---|---|
| 1b | 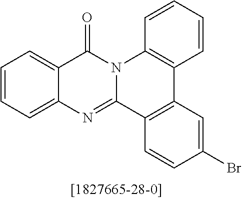 [SMILES: O=c1c2ccccc2nc2c3ccc(Br)cc3c3ccccc3n12] | 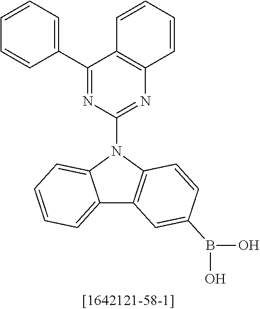 [SMILES: OB(O)c1ccc2c(c1)c1ccccc1n2-c1nc(-c2ccccc2)c2ccccc2n1] | 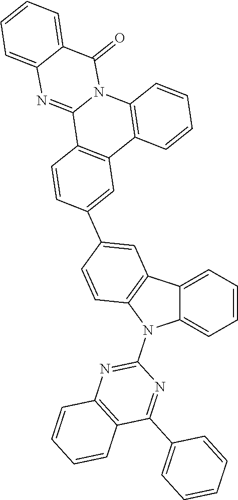 [SMILES: O=c1c2ccccc2nc2c3ccc(-c4ccc5c(c4)c4ccccc4n5-c4nc(-c5ccccc5)c5ccccc5n4)cc3c3ccccc3n12] | 70% |
| 2b | 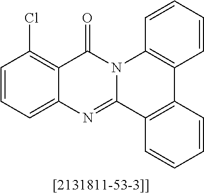 [SMILES: O=c1c2c(Cl)cccc2nc2c3ccccc3c3ccccc3n12] | 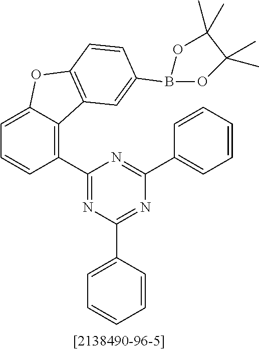 [SMILES: CC1(C)OB(c2ccc3oc4cccc(-c5nc(-c6ccccc6)nc(-c6ccccc6)n5)c4c3c2)OC1(C)C] | 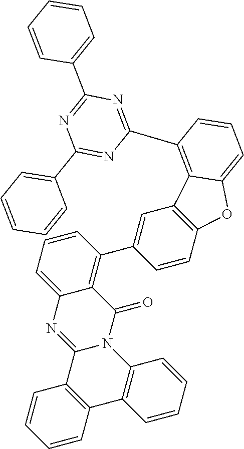 [SMILES: O=c1c2c(-c3ccc4oc5cccc(-c6nc(-c7ccccc7)nc(-c7ccccc7)n6)c5c4c3)cccc2nc2c3ccccc3c3ccccc3n12] | 71% |
| 3b | 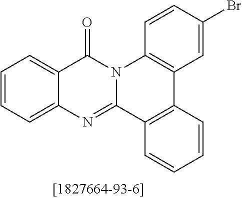 [SMILES: O=c1c2ccccc2nc2c3ccccc3c3cc(Br)ccc3n12] | 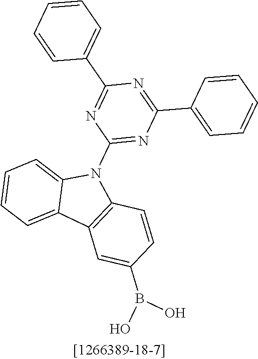 [SMILES: OB(O)c1ccc2c(c1)c1ccccc1n2-c1nc(-c2ccccc2)nc(-c2ccccc2)n1] | 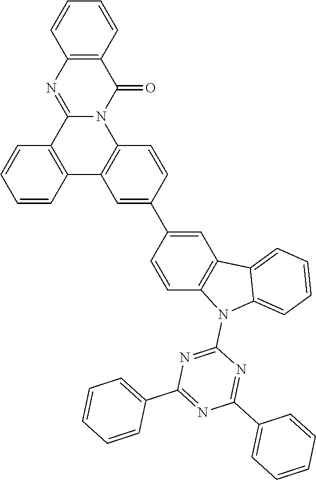 [SMILES: O=c1c2ccccc2nc2c3ccccc3c3cc(-c4ccc5c(c4)c4ccccc4n5-c4nc(-c5ccccc5)nc(-c5ccccc5)n4)ccc3n12] | 73% |
| 4b | 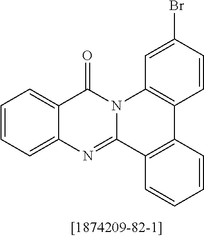 [SMILES: O=c1c2ccccc2nc2c3ccccc3c3ccc(Br)cc3n12] | 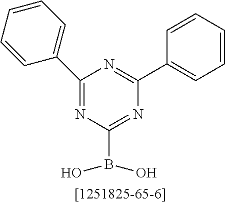 [SMILES: OB(O)c1nc(-c2ccccc2)nc(-c2ccccc2)n1] | 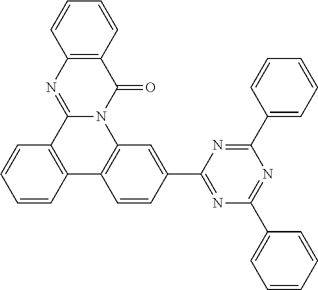 [SMILES: O=c1c2ccccc2nc2c3ccccc3c3ccc(-c4nc(-c5ccccc5)nc(-c5ccccc5)n4)cc3n12] | 80% |
| 5b | 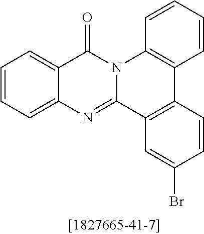 [SMILES: O=c1c2ccccc2nc2c3cc(Br)ccc3c3ccccc3n12] | 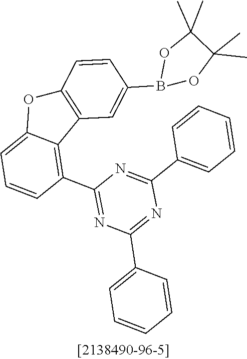 [SMILES: CC1(C)OB(c2ccc3oc4cccc(-c5nc(-c6ccccc6)nc(-c6ccccc6)n5)c4c3c2)OC1(C)C] | 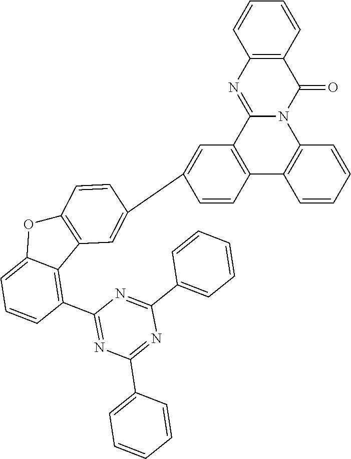 [SMILES: O=c1c2ccccc2nc2c3cc(-c4ccc5oc6cccc(-c7nc(-c8ccccc8)nc(-c8ccccc8)n7)c6c5c4)ccc3c3ccccc3n12] | 72% |
| 6b | 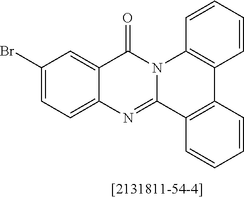 [SMILES: O=c1c2cc(Br)ccc2nc2c3ccccc3c3ccccc3n12] | 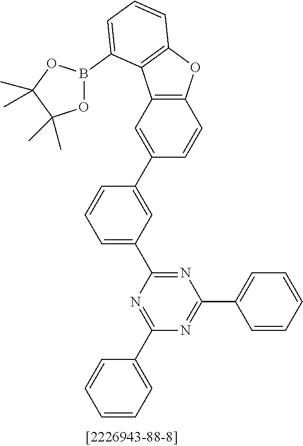 [SMILES: CC1(C)OB(c2cccc3oc4ccc(-c5cccc(-c6nc(-c7ccccc7)nc(-c7ccccc7)n6)c5)cc4c23)OC1(C)C] | 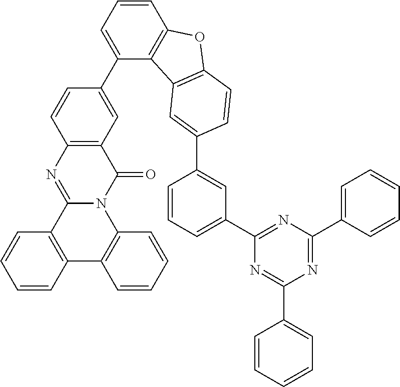 [SMILES: O=c1c2cc(-c3cccc4oc5ccc(-c6cccc(-c7nc(-c8ccccc8)nc(-c8ccccc8)n7)c6)cc5c34)ccc2nc2c3ccccc3c3ccccc3n12] | 70% |
| 7b | 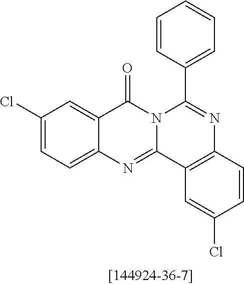 [SMILES: O=c1c2cc(Cl)ccc2nc2c3cc(Cl)ccc3nc(-c3ccccc3)n12] | 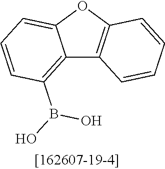 [SMILES: OB(O)c1cccc2oc3ccccc3c12] | 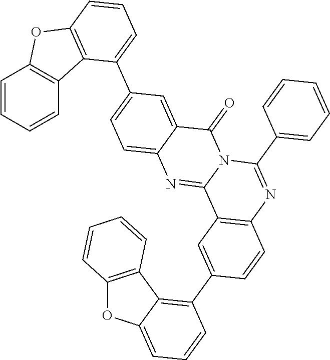 [SMILES: O=c1c2cc(-c3cccc4oc5ccccc5c34)ccc2nc2c3cc(-c4cccc5oc6ccccc6c45)ccc3nc(-c3ccccc3)n12] | 83% |
| 8b | 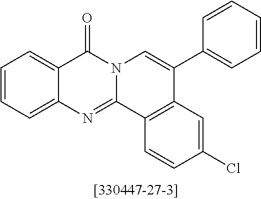 [SMILES: O=c1c2ccccc2nc2c3ccc(Cl)cc3c(-c3ccccc3)cn12] | 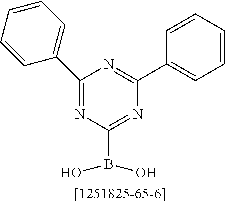 [SMILES: OB(O)c1nc(-c2ccccc2)nc(-c2ccccc2)n1] | 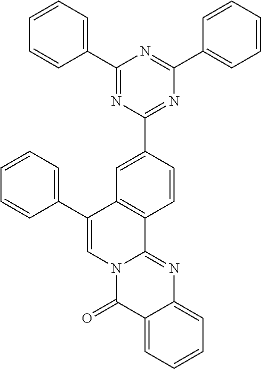 [SMILES: O=c1c2ccccc2nc2c3ccc(-c4nc(-c5ccccc5)nc(-c5ccccc5)n4)cc3c(-c3ccccc3)cn12] | 85% |
| 9b | 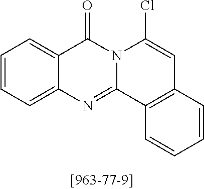 [SMILES: O=c1c2ccccc2nc2c3ccccc3cc(Cl)n12] | 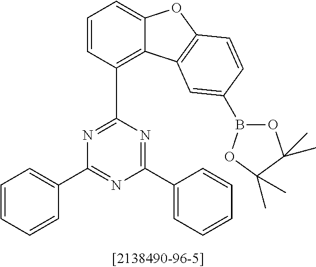 [SMILES: CC1(C)OB(c2ccc3oc4cccc(-c5nc(-c6ccccc6)nc(-c6ccccc6)n5)c4c3c2)OC1(C)C] | 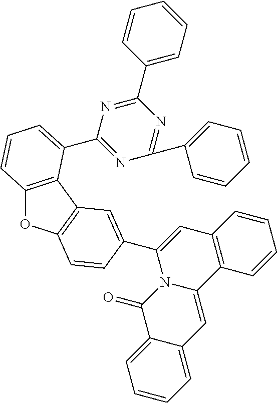 [SMILES: O=c1c2ccccc2cc2c3ccccc3cc(-c3ccc4oc5cccc(-c6nc(-c7ccccc7)nc(-c7ccccc7)n6)c5c4c3)n12] | 81% |
| 10b | 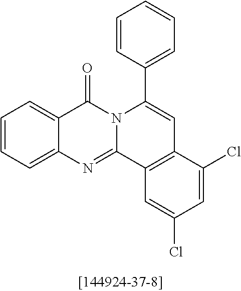 [SMILES: O=c1c2ccccc2nc2c3cc(Cl)cc(Cl)c3cc(-c3ccccc3)n12] | 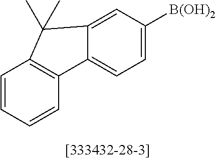 [SMILES: CC1(C)c2ccccc2-c2ccc(B(O)O)cc21] | 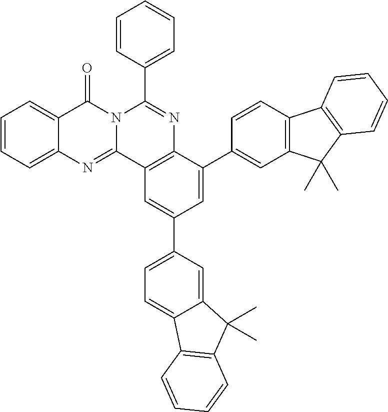 [SMILES: CC1(C)c2ccccc2-c2ccc(-c3cc(-c4ccc5c(c4)C(C)(C)c4ccccc4-5)c4nc(-c5ccccc5)n5c(=O)c6ccccc6nc5c4c3)cc21] | 84% |
| 11b | 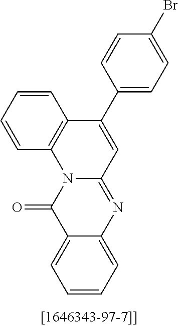 [SMILES: O=c1c2ccccc2nc2cc(-c3ccc(Br)cc3)c3ccccc3n12] | 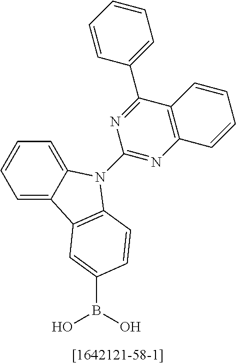 [SMILES: OB(O)c1ccc2c(c1)c1ccccc1n2-c1nc(-c2ccccc2)c2ccccc2n1] | 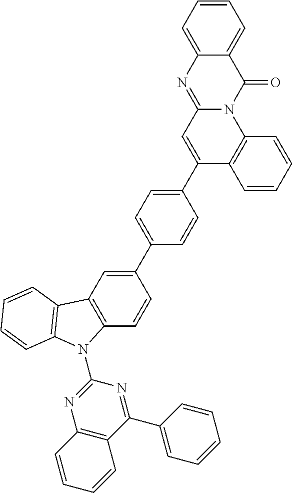 [SMILES: O=c1c2ccccc2nc2cc(-c3ccc(-c4ccc5c(c4)c4ccccc4n5-c4nc(-c5ccccc5)c5ccccc5n4)cc3)c3ccccc3n12] | 69% |
| 12b | 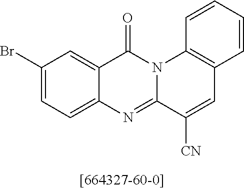 [SMILES: N#Cc1cc2ccccc2n2c(=O)c3cc(Br)ccc3nc12] | 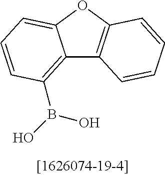 [SMILES: OB(O)c1cccc2oc3ccccc3c12] | 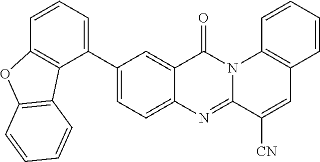 [SMILES: N#Cc1cc2ccccc2n2c(=O)c3cc(-c4cccc5oc6ccccc6c45)ccc3nc12] | 70% |
| 13b | 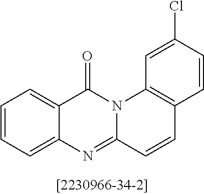 [SMILES: O=c1c2ccccc2nc2ccc3ccc(Cl)cc3n12] | 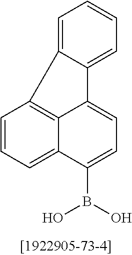 [SMILES: OB(O)c1ccc2c3c(cccc13)-c1ccccc1-2] | 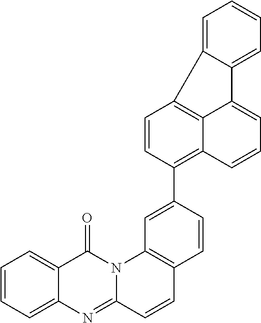 [SMILES: O=c1c2ccccc2nc2ccc3ccc(-c4ccc5c6c(cccc46)-c4ccccc4-5)cc3n12] | 81% |
| 14b | 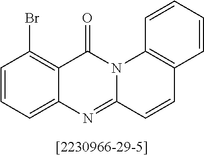 [SMILES: O=c1c2c(Br)cccc2nc2ccc3ccccc3n12] | 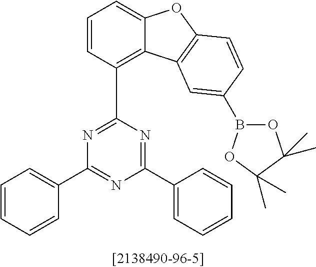 [SMILES: CC1(C)OB(c2ccc3oc4cccc(-c5nc(-c6ccccc6)nc(-c6ccccc6)n5)c4c3c2)OC1(C)C] | 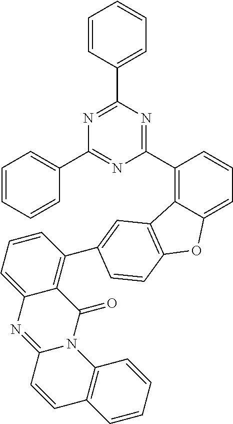 [SMILES: O=c1c2c(-c3ccc4oc5cccc(-c6nc(-c7ccccc7)nc(-c7ccccc7)n6)c5c4c3)cccc2nc2ccc3ccccc3n12] | 83% |
| 15b | 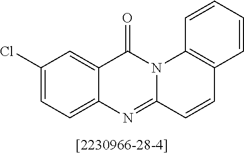 [SMILES: O=c1c2cc(Cl)ccc2nc2ccc3ccccc3n12] | 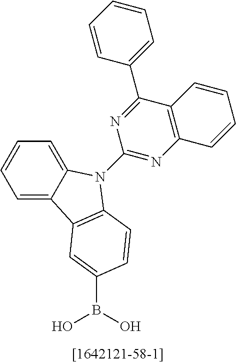 [SMILES: OB(O)c1ccc2c(c1)c1ccccc1n2-c1nc(-c2ccccc2)c2ccccc2n1] | 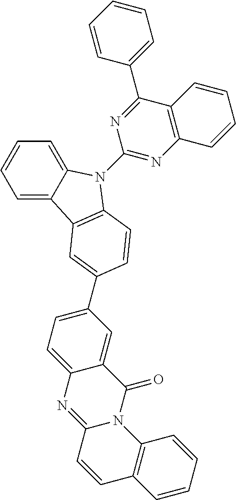 [SMILES: O=c1c2cc(-c3ccc4c(c3)c3ccccc3n4-c3nc(-c4ccccc4)c4ccccc4n3)ccc2nc2ccc3ccccc3n12] | 83% |
| 16b | 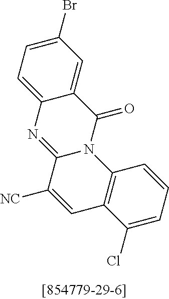 [SMILES: N#Cc1cc2c(Cl)cccc2n2c(=O)c3cc(Br)ccc3nc12] | 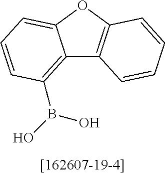 [SMILES: OB(O)c1cccc2oc3ccccc3c12] | 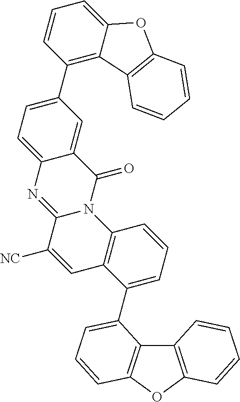 [SMILES: N#Cc1cc2c(-c3cccc4oc5ccccc5c34)cccc2n2c(=O)c3cc(-c4cccc5oc6ccccc6c45)ccc3nc12] | 87% |
| 17b | 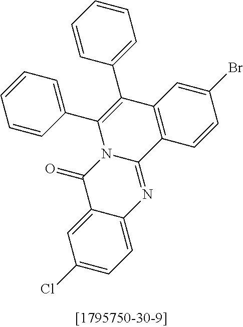 [SMILES: O=c1c2cc(Cl)ccc2nc2c3ccc(Br)cc3c(-c3ccccc3)c(-c3ccccc3)n12] | 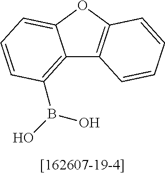 [SMILES: OB(O)c1cccc2oc3ccccc3c12] | 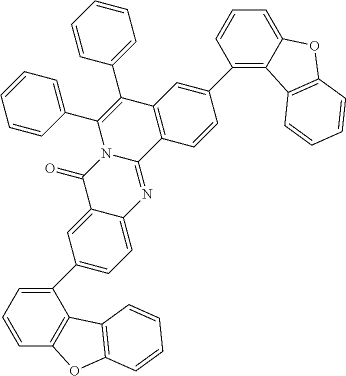 [SMILES: O=c1c2cc(-c3cccc4oc5ccccc5c34)ccc2nc2c3ccc(-c4cccc5oc6ccccc6c45)cc3c(-c3ccccc3)c(-c3ccccc3)n12] | 75% |
| 18b | 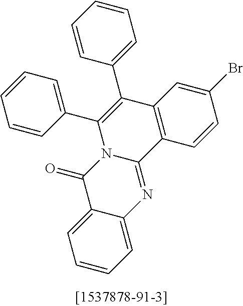 [SMILES: O=c1c2ccccc2nc2c3ccc(Br)cc3c(-c3ccccc3)c(-c3ccccc3)n12] | 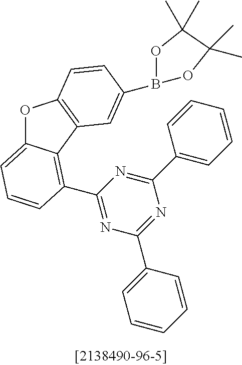 [SMILES: CC1(C)OB(c2ccc3oc4cccc(-c5nc(-c6ccccc6)nc(-c6ccccc6)n5)c4c3c2)OC1(C)C] | 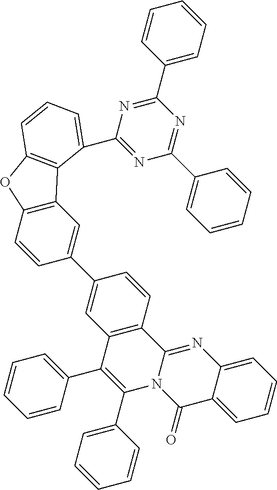 [SMILES: O=c1c2ccccc2nc2c3ccc(-c4ccc5oc6cccc(-c7nc(-c8ccccc8)nc(-c8ccccc8)n7)c6c5c4)cc3c(-c3ccccc3)c(-c3ccccc3)n12] | 76% |
| 19b | 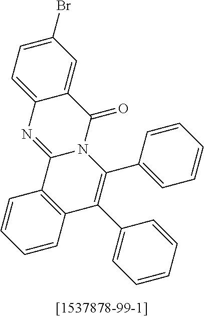 [SMILES: O=c1c2cc(Br)ccc2nc2c3ccccc3c(-c3ccccc3)c(-c3ccccc3)n12] | 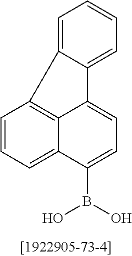 [SMILES: OB(O)c1ccc2c3c(cccc13)-c1ccccc1-2] | 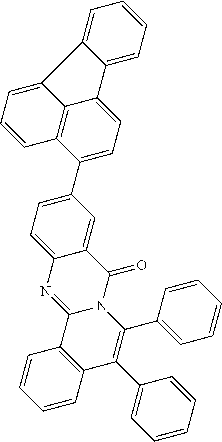 [SMILES: O=c1c2cc(-c3ccc4c5c(cccc35)-c3ccccc3-4)ccc2nc2c3ccccc3c(-c3ccccc3)c(-c3ccccc3)n12] | 66% |
| 20b | 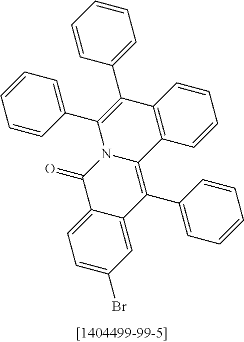 [SMILES: O=c1c2ccc(Br)cc2c(-c2ccccc2)c2c3ccccc3c(-c3ccccc3)c(-c3ccccc3)n12] | 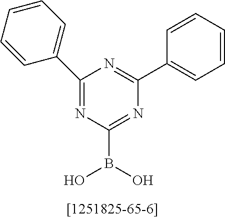 [SMILES: OB(O)c1nc(-c2ccccc2)nc(-c2ccccc2)n1] | 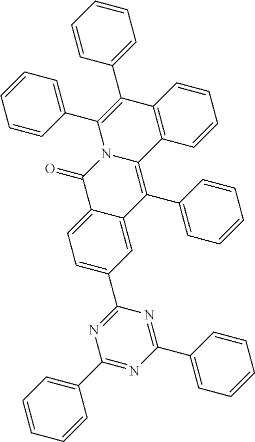 [SMILES: O=c1c2ccc(-c3nc(-c4ccccc4)nc(-c4ccccc4)n3)cc2c(-c2ccccc2)c2c3ccccc3c(-c3ccccc3)c(-c3ccccc3)n12] | 71% |
| 21b | 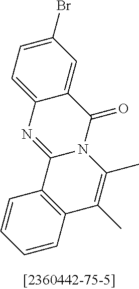 [SMILES: Cc1c(C)n2c(=O)c3cc(Br)ccc3nc2c2ccccc12] | 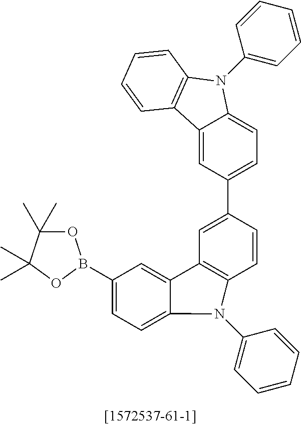 [SMILES: CC1(C)OB(c2ccc3c(c2)c2cc(-c4ccc5c(c4)c4ccccc4n5-c4ccccc4)ccc2n3-c2ccccc2)OC1(C)C] | 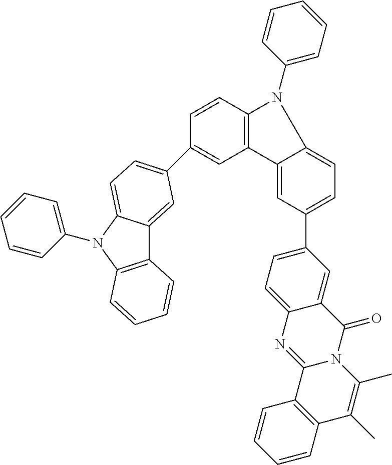 [SMILES: Cc1c(C)n2c(=O)c3cc(-c4ccc5c(c4)c4cc(-c6ccc7c(c6)c6ccccc6n7-c6ccccc6)ccc4n5-c4ccccc4)ccc3nc2c2ccccc12] | 62% |
| 22b | 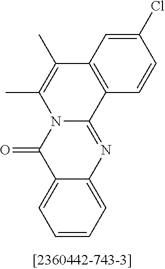 [SMILES: Cc1c(C)n2c(=O)c3ccccc3nc2c2ccc(Cl)cc12] | 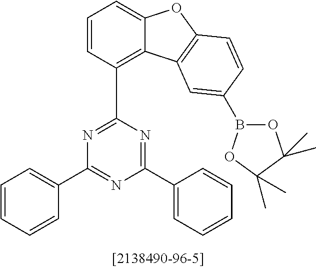 [SMILES: CC1(C)OB(c2ccc3oc4cccc(-c5nc(-c6ccccc6)nc(-c6ccccc6)n5)c4c3c2)OC1(C)C] | 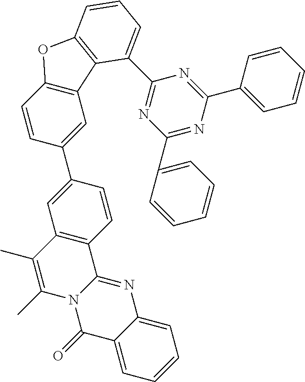 [SMILES: Cc1c(C)n2c(=O)c3ccccc3nc2c2ccc(-c3ccc4oc5cccc(-c6nc(-c7ccccc7)nc(-c7ccccc7)n6)c5c4c3)cc12] | 67% |
| 23b | 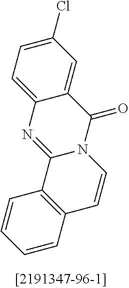 [SMILES: O=c1c2cc(Cl)ccc2nc2c3ccccc3ccn12] | 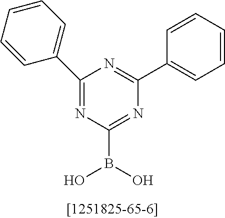 [SMILES: OB(O)c1nc(-c2ccccc2)nc(-c2ccccc2)n1] | 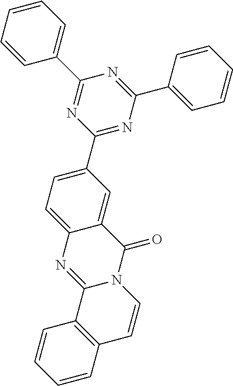 [SMILES: O=c1c2cc(-c3nc(-c4ccccc4)nc(-c4ccccc4)n3)ccc2nc2c3ccccc3ccn12] | 68% |
| 24b | 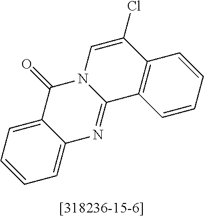 [SMILES: O=c1c2ccccc2nc2c3ccccc3c(Cl)cn12] | 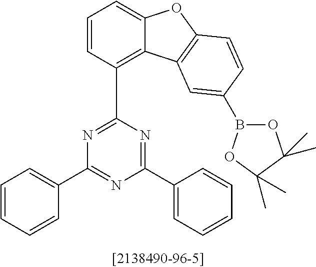 [SMILES: CC1(C)OB(c2ccc3oc4cccc(-c5nc(-c6ccccc6)nc(-c6ccccc6)n5)c4c3c2)OC1(C)C] | 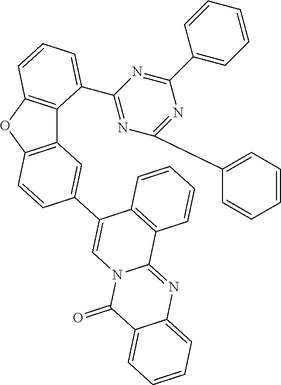 [SMILES: O=c1c2ccccc2nc2c3ccccc3c(-c3ccc4oc5cccc(-c6nc(-c7ccccc7)nc(-c7ccccc7)n6)c5c4c3)cn12] | 69% |
| 25b | 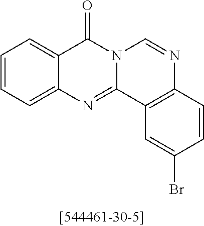 [SMILES: O=c1c2ccccc2nc2c3cc(Br)ccc3ncn12] | 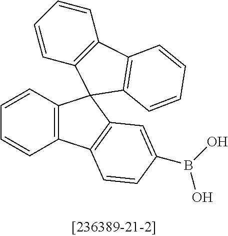 [SMILES: OB(O)c1ccc2c(c1)C1(c3ccccc3-c3ccccc31)c1ccccc1-2] | 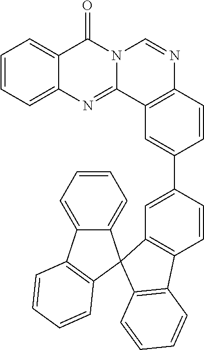 [SMILES: O=c1c2ccccc2nc2c3cc(-c4ccc5c(c4)C4(c6ccccc6-c6ccccc64)c4ccccc4-5)ccc3ncn12] | 63% |
| 26b | 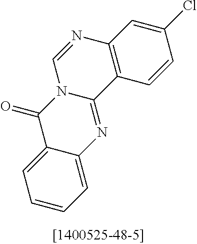 [SMILES: O=c1c2ccccc2nc2c3ccc(Cl)cc3ncn12] | 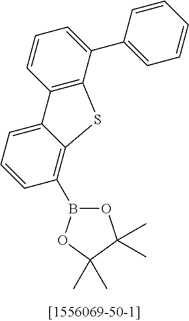 [SMILES: CC1(C)OB(c2cccc3c2sc2c(-c4ccccc4)cccc23)OC1(C)C] | 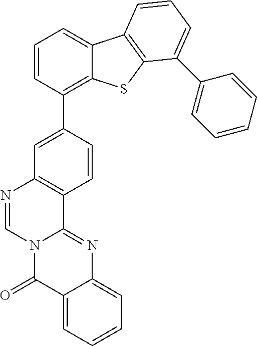 [SMILES: O=c1c2ccccc2nc2c3ccc(-c4cccc5c4sc4c(-c6ccccc6)cccc45)cc3ncn12] | 64% |
| 27b | 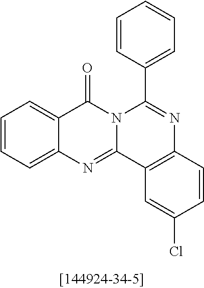 [SMILES: O=c1c2ccccc2nc2c3cc(Cl)ccc3nc(-c3ccccc3)n12] | 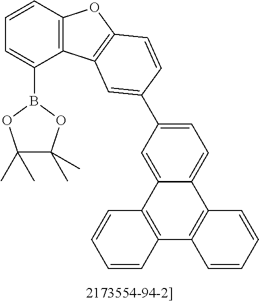 [SMILES: CC1(C)OB(c2cccc3oc4ccc(-c5ccc6c7ccccc7c7ccccc7c6c5)cc4c23)OC1(C)C] | 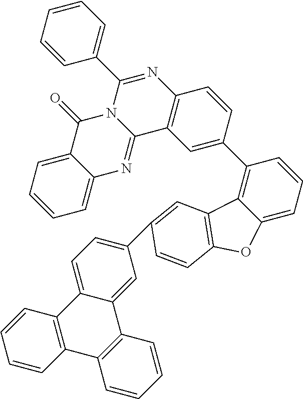 [SMILES: O=c1c2ccccc2nc2c3cc(-c4cccc5oc6ccc(-c7ccc8c9ccccc9c9ccccc9c8c7)cc6c45)ccc3nc(-c3ccccc3)n12] | 67% |
| 28b | 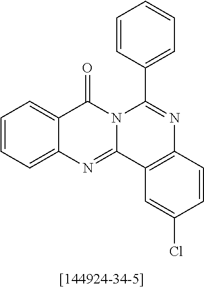 [SMILES: O=c1c2ccccc2nc2c3cc(Cl)ccc3nc(-c3ccccc3)n12] | 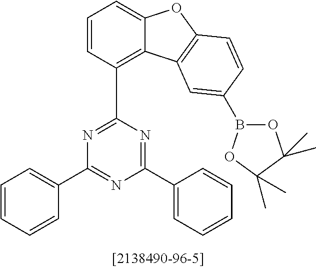 [SMILES: CC1(C)OB(c2ccc3oc4cccc(-c5nc(-c6ccccc6)nc(-c6ccccc6)n5)c4c3c2)OC1(C)C] | 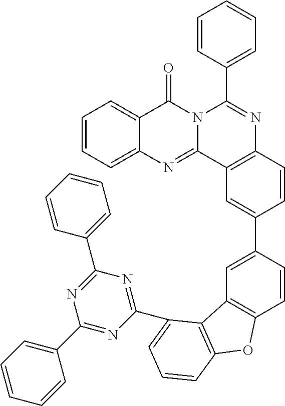 [SMILES: O=c1c2ccccc2nc2c3cc(-c4ccc5oc6cccc(-c7nc(-c8ccccc8)nc(-c8ccccc8)n7)c6c5c4)ccc3nc(-c3ccccc3)n12] | 65% |
| 29b | 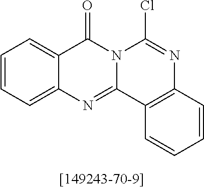 [SMILES: O=c1c2ccccc2nc2c3ccccc3nc(Cl)n12] | 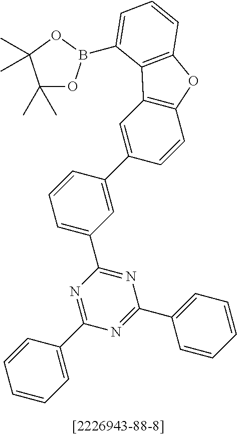 [SMILES: CC1(C)OB(c2cccc3oc4ccc(-c5cccc(-c6nc(-c7ccccc7)nc(-c7ccccc7)n6)c5)cc4c23)OC1(C)C] | 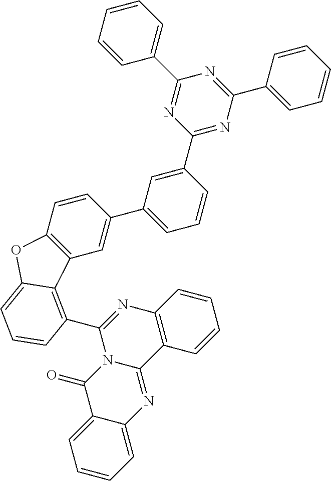 [SMILES: O=c1c2ccccc2nc2c3ccccc3nc(-c3cccc4oc5ccc(-c6cccc(-c7nc(-c8ccccc8)nc(-c8ccccc8)n7)c6)cc5c34)n12] | 71% |
| 30b | 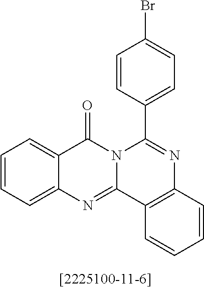 [SMILES: O=c1c2ccccc2nc2c3ccccc3nc(-c3ccc(Br)cc3)n12] | 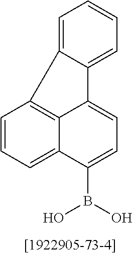 [SMILES: OB(O)c1ccc2c3c(cccc13)-c1ccccc1-2] | 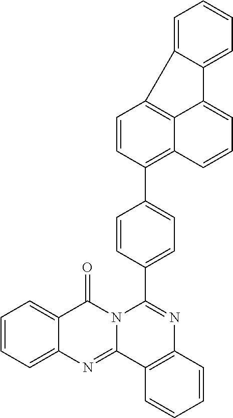 [SMILES: O=c1c2ccccc2nc2c3ccccc3nc(-c3ccc(-c4ccc5c6c(cccc46)-c4ccccc4-5)cc3)n12] | 75% |
| 31b | 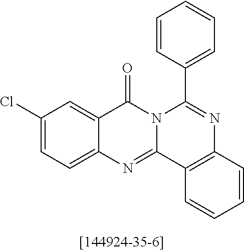 [SMILES: O=c1c2cc(Cl)ccc2nc2c3ccccc3nc(-c3ccccc3)n12] | 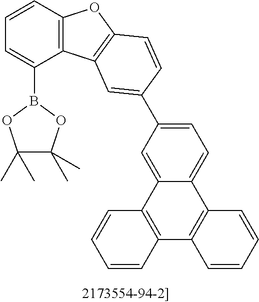 [SMILES: CC1(C)OB(c2cccc3oc4ccc(-c5ccc6c7ccccc7c7ccccc7c6c5)cc4c23)OC1(C)C] | 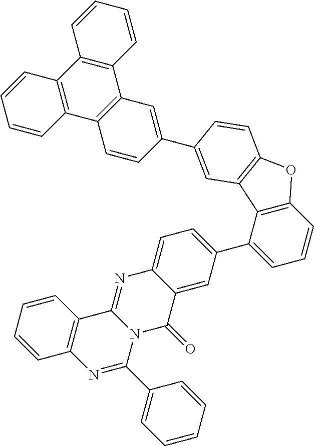 [SMILES: O=c1c2cc(-c3cccc4oc5ccc(-c6ccc7c8ccccc8c8ccccc8c7c6)cc5c34)ccc2nc2c3ccccc3nc(-c3ccccc3)n12] | 69% |
| 32b | 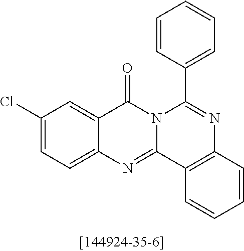 [SMILES: O=c1c2cc(Cl)ccc2nc2c3ccccc3nc(-c3ccccc3)n12] | 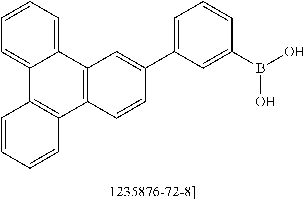 [SMILES: OB(O)c1cccc(-c2ccc3c4ccccc4c4ccccc4c3c2)c1] | 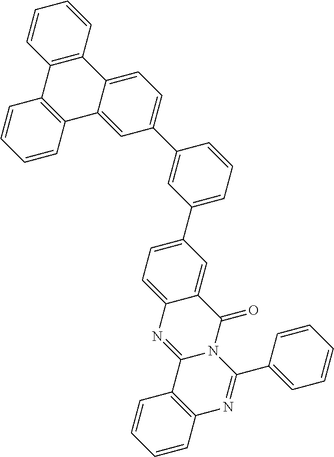 [SMILES: O=c1c2cc(-c3cccc(-c4ccc5c6ccccc6c6ccccc6c5c4)c3)ccc2nc2c3ccccc3nc(-c3ccccc3)n12] | 53% |
| 33b | 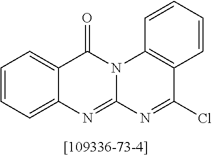 [SMILES: O=c1c2ccccc2nc2nc(Cl)c3ccccc3n12] | 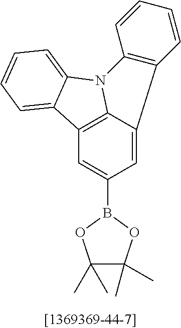 [SMILES: CC1(C)OB(c2cc3c4ccccc4n4c5ccccc5c(c2)c34)OC1(C)C] | 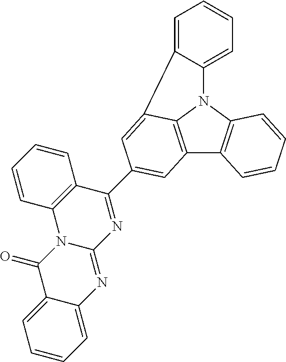 [SMILES: O=c1c2ccccc2nc2nc(-c3cc4c5ccccc5n5c6ccccc6c(c3)c45)c3ccccc3n12] | 60% |
| 34b | 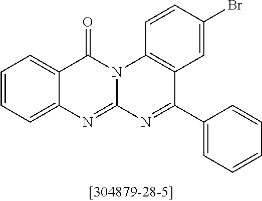 [SMILES: O=c1c2ccccc2nc2nc(-c3ccccc3)c3cc(Br)ccc3n12] | 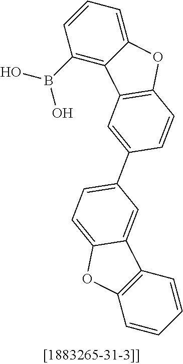 [SMILES: OB(O)c1cccc2oc3ccc(-c4ccc5oc6ccccc6c5c4)cc3c12] | 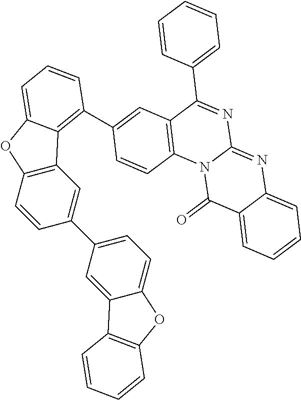 [SMILES: O=c1c2ccccc2nc2nc(-c3ccccc3)c3cc(-c4cccc5oc6ccc(-c7ccc8oc9ccccc9c8c7)cc6c45)ccc3n12] | 71% |
| 35b | 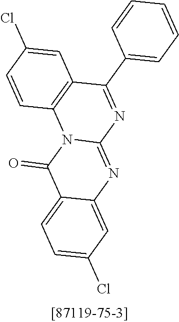 [SMILES: O=c1c2ccc(Cl)cc2nc2nc(-c3ccccc3)c3cc(Cl)ccc3n12] | 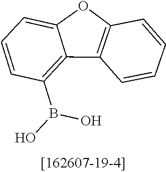 [SMILES: OB(O)c1cccc2oc3ccccc3c12] | 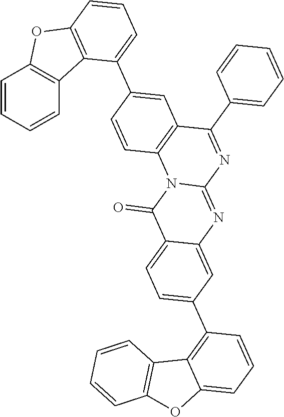 [SMILES: O=c1c2ccc(-c3cccc4oc5ccccc5c34)cc2nc2nc(-c3ccccc3)c3cc(-c4cccc5oc6ccccc6c45)ccc3n12] | 70% |
| 36b | 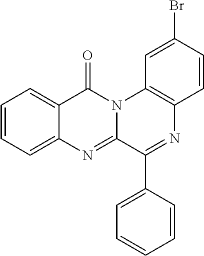 [SMILES: O=c1c2ccccc2nc2c(-c3ccccc3)nc3ccc(Br)cc3n12] | 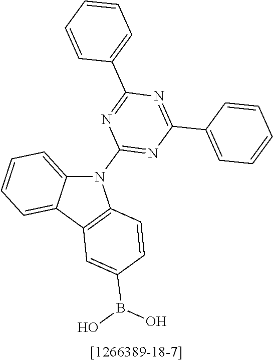 [SMILES: OB(O)c1ccc2c(c1)c1ccccc1n2-c1nc(-c2ccccc2)nc(-c2ccccc2)n1] | 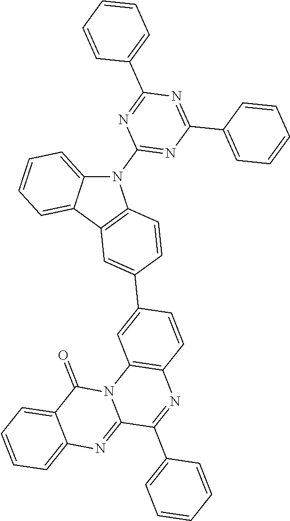 [SMILES: O=c1c2ccccc2nc2c(-c3ccccc3)nc3ccc(-c4ccc5c(c4)c4ccccc4n5-c4nc(-c5ccccc5)nc(-c5ccccc5)n4)cc3n12] | 71% |
| 37b | 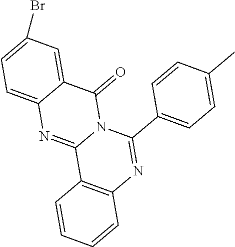 [SMILES: Cc1ccc(-c2nc3ccccc3c3nc4ccc(Br)cc4c(=O)n23)cc1] | 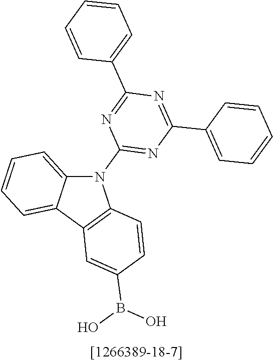 [SMILES: OB(O)c1ccc2c(c1)c1ccccc1n2-c1nc(-c2ccccc2)nc(-c2ccccc2)n1] | 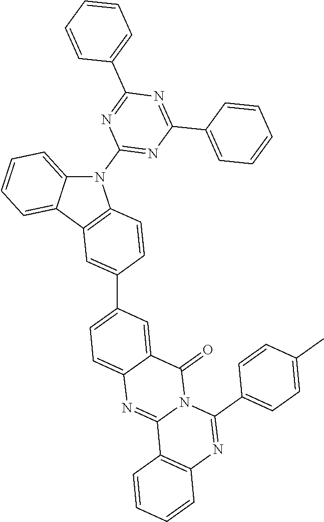 [SMILES: Cc1ccc(-c2nc3ccccc3c3nc4ccc(-c5ccc6c(c5)c5ccccc5n6-c5nc(-c6ccccc6)nc(-c6ccccc6)n5)cc4c(=O)n23)cc1] | 73% |
| 38b | 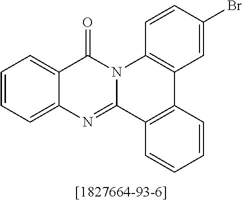 [SMILES: O=c1c2ccccc2nc2c3ccccc3c3cc(Br)ccc3n12] | 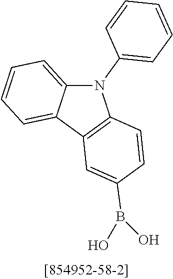 [SMILES: OB(O)c1ccc2c(c1)c1ccccc1n2-c1ccccc1] | 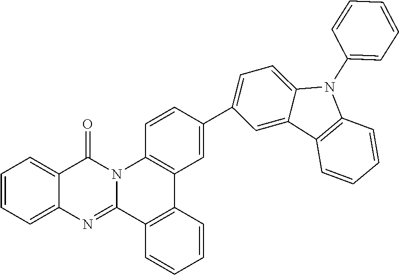 [SMILES: O=c1c2ccccc2nc2c3ccccc3c3cc(-c4ccc5c(c4)c4ccccc4n5-c4ccccc4)ccc3n12] | 70% |
| 39b | 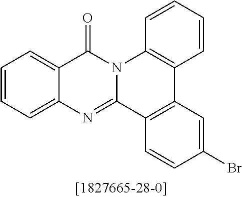 [SMILES: O=c1c2ccccc2nc2c3ccc(Br)cc3c3ccccc3n12] | 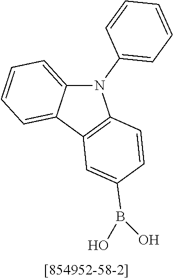 [SMILES: OB(O)c1ccc2c(c1)c1ccccc1n2-c1ccccc1] | 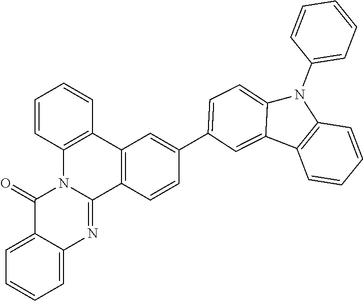 [SMILES: O=c1c2ccccc2nc2c3ccc(-c4ccc5c(c4)c4ccccc4n5-c4ccccc4)cc3c3ccccc3n12] | 69% |
| 40b | 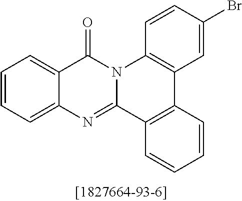 [SMILES: O=c1c2ccccc2nc2c3ccccc3c3cc(Br)ccc3n12] | 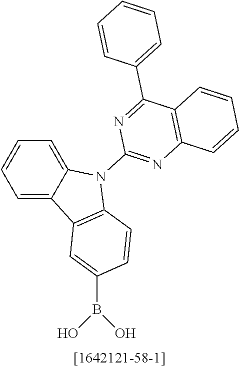 [SMILES: OB(O)c1ccc2c(c1)c1ccccc1n2-c1nc(-c2ccccc2)c2ccccc2n1] | 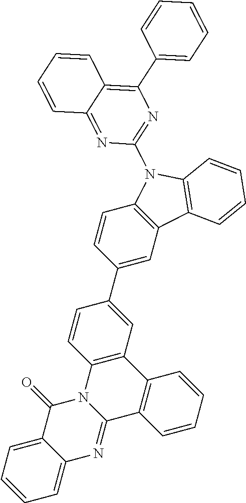 [SMILES: O=c1c2ccccc2nc2c3ccccc3c3cc(-c4ccc5c(c4)c4ccccc4n5-c4nc(-c5ccccc5)c5ccccc5n4)ccc3n12] | 73% |
| 41b | 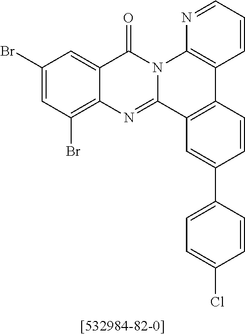 [SMILES: O=c1c2cc(Br)cc(Br)c2nc2c3cc(-c4ccc(Cl)cc4)ccc3c3cccnc3n12] | 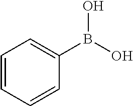 | 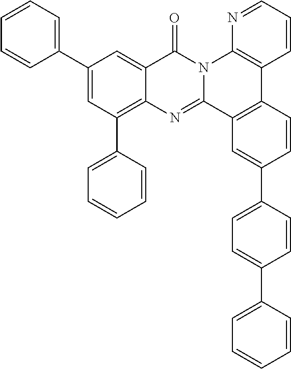 [SMILES: O=c1c2cc(-c3ccccc3)cc(-c3ccccc3)c2nc2c3cc(-c4ccc(-c5ccccc5)cc4)ccc3c3cccnc3n12] | 78% |
| 42b | 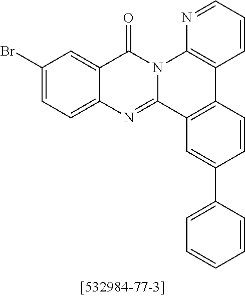 [SMILES: O=c1c2cc(Br)ccc2nc2c3cc(-c4ccccc4)ccc3c3cccnc3n12] | 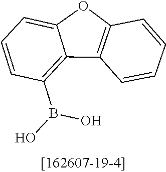 [SMILES: OB(O)c1cccc2oc3ccccc3c12] | 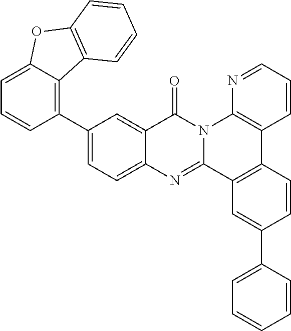 [SMILES: O=c1c2cc(-c3cccc4oc5ccccc5c34)ccc2nc2c3cc(-c4ccccc4)ccc3c3cccnc3n12] | 80% |
| 43b | 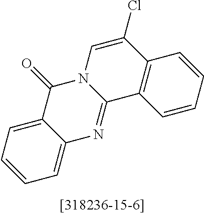 [SMILES: O=c1c2ccccc2nc2c3ccccc3c(Cl)cn12] | 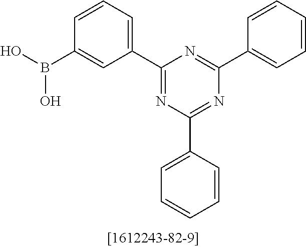 [SMILES: OB(O)c1cccc(-c2nc(-c3ccccc3)nc(-c3ccccc3)n2)c1] | 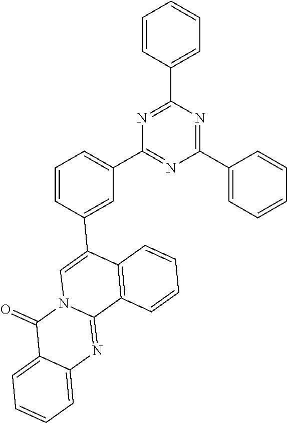 [SMILES: O=c1c2ccccc2nc2c3ccccc3c(-c3cccc(-c4nc(-c5ccccc5)nc(-c5ccccc5)n4)c3)cn12] | 74% |
| 44b | 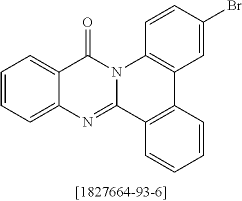 [SMILES: O=c1c2ccccc2nc2c3ccccc3c3cc(Br)ccc3n12] | 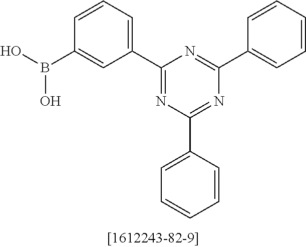 [SMILES: OB(O)c1cccc(-c2nc(-c3ccccc3)nc(-c3ccccc3)n2)c1] | 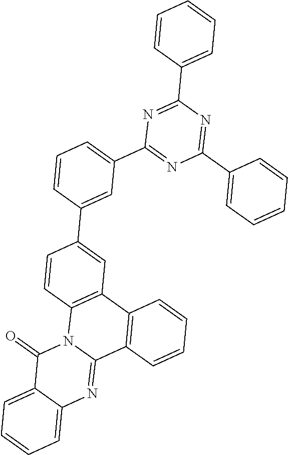 [SMILES: O=c1c2ccccc2nc2c3ccccc3c3cc(-c4cccc(-c5nc(-c6ccccc6)nc(-c6ccccc6)n5)c4)ccc3n12] | 76% |
| 45b | 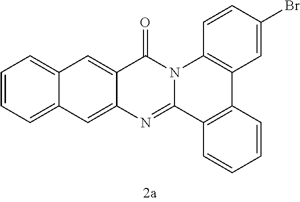 [SMILES: O=c1c2cc3ccccc3cc2nc2c3ccccc3c3cc(Br)ccc3n12] | 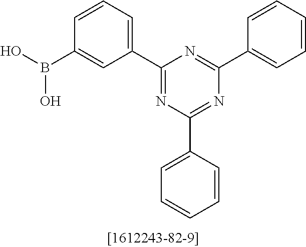 [SMILES: OB(O)c1cccc(-c2nc(-c3ccccc3)nc(-c3ccccc3)n2)c1] | 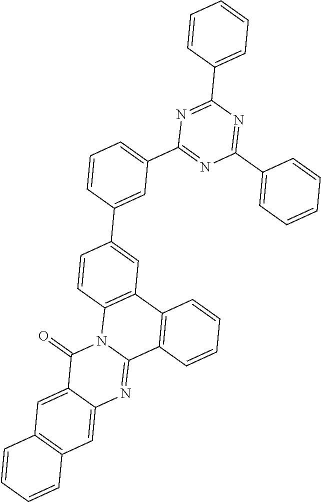 [SMILES: O=c1c2cc3ccccc3cc2nc2c3ccccc3c3cc(-c4cccc(-c5nc(-c6ccccc6)nc(-c6ccccc6)n5)c4)ccc3n12] | 63% |
| 46b | 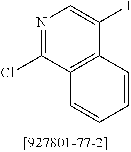 [SMILES: Clc1ncc(I)c2ccccc12] | 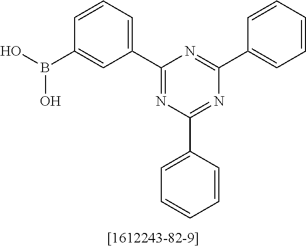 [SMILES: OB(O)c1cccc(-c2nc(-c3ccccc3)nc(-c3ccccc3)n2)c1] | 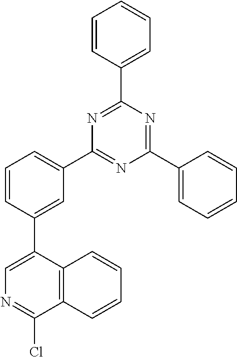 [SMILES: Clc1ncc(-c2cccc(-c3nc(-c4ccccc4)nc(-c4ccccc4)n3)c2)c2ccccc12] | 63% |

### c) 5-Phenyl-3-[3-(9-phenylcarbazol-3-yl)carbazol-9-yl]quinazolino[2,1-b]quinazolin-12-one

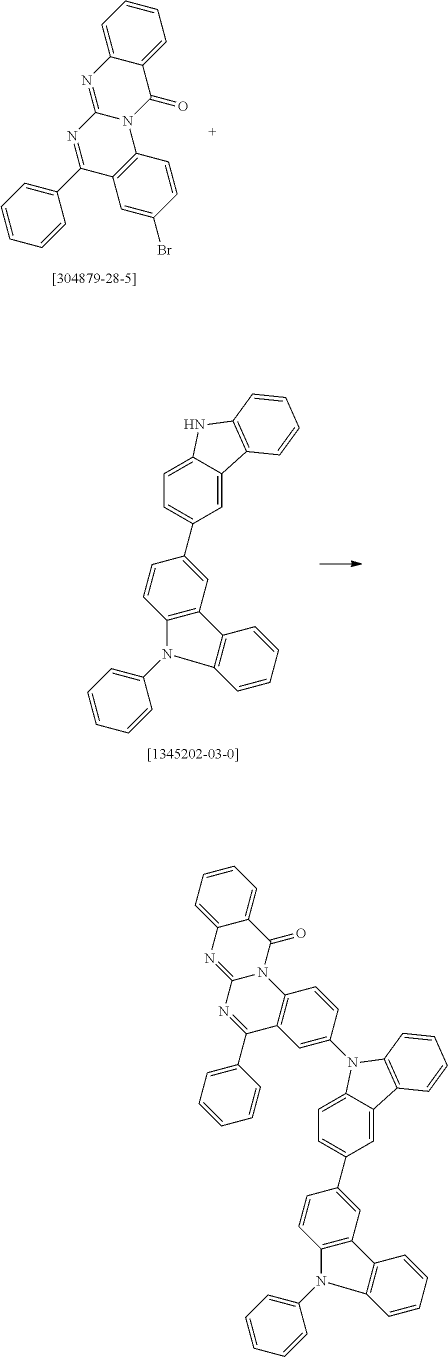 [SMILES: O=c1c2ccccc2nc2nc(-c3ccccc3)c3cc(Br)ccc3n12 ; c1ccc(-n2c3ccccc3c3cc(-c4ccc5[nH]c6ccccc6c5c4)ccc32)cc1 ; O=c1c2ccccc2nc2nc(-c3ccccc3)c3cc(-n4c5ccccc5c5cc(-c6ccc7c(c6)c6ccccc6n7-c6ccccc6)ccc54)ccc3n12]

20.4 g (50 mmol) of 9-phenyl-3,3′-bi-9H-carbazole and 24 g (60 mmol) of 3-bromo-5-phenylquinazolino[3,2-a]quinazolin-12-one are dissolved in 400 ml of toluene under an argon atmosphere. 1.0 g (5 mmol) of tri-tert-butylphosphine is added to the flask and the mixture is stirred under an argon atmosphere. 0.6 g (2 mmol) of Pd(OAc)₂ is added to the flask and the mixture is stirred under an argon atmosphere, and then 9.5 g (99 mmol) of sodium tert-butoxide is added to the flask. The reaction mixture is stirred under reflux for 24 h. After cooling, the organic phase is separated, washed three times with 200 ml of water, dried over MgSO₄ and filtered, and the solvent is removed under reduced pressure. The residue is purified by column chromatography using silica gel (eluent: DCM/heptane (1:3)). The residue is subjected to hot extraction with toluene and recrystallized from toluene/n-heptane and finally sublimed under high vacuum.

The yield is 29 g (40 mmol), corresponding to 84% of theory.

The following compounds can be prepared analogously:

| Ex. | Reactant 1 | Reactant 2 |
|---|---|---|
| 1c | 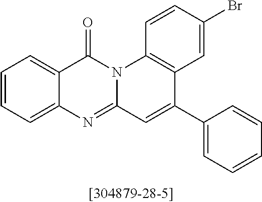 [SMILES: O=c1c2ccccc2nc2cc(-c3ccccc3)c3cc(Br)ccc3n12] | 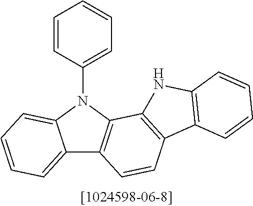 [SMILES: c1ccc(-n2c3ccccc3c3ccc4c5ccccc5[nH]c4c32)cc1] |
| 2c | 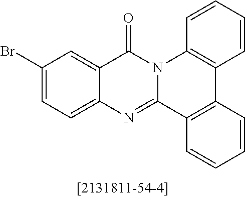 [SMILES: O=c1c2cc(Br)ccc2nc2c3ccccc3c3ccccc3n12] | 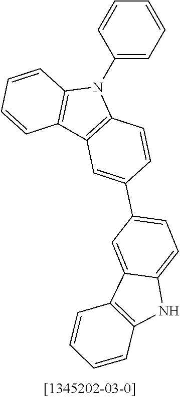 [SMILES: c1ccc(-n2c3ccccc3c3cc(-c4ccc5[nH]c6ccccc6c5c4)ccc32)cc1] |
| 3c | 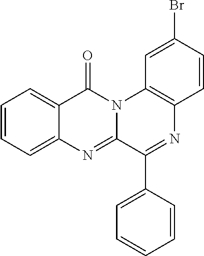 [SMILES: O=c1c2ccccc2nc2c(-c3ccccc3)nc3ccc(Br)cc3n12] | 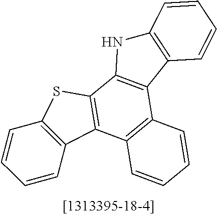 [SMILES: c1ccc2c(c1)[nH]c1c3sc4ccccc4c3c3ccccc3c21] |
| 4c | 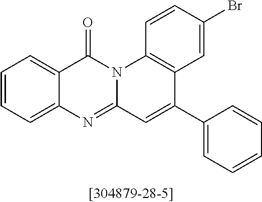 [SMILES: O=c1c2ccccc2nc2cc(-c3ccccc3)c3cc(Br)ccc3n12] | 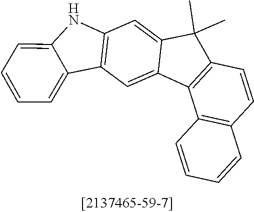 [SMILES: CC1(C)c2cc3[nH]c4ccccc4c3cc2-c2c1ccc1ccccc21] |
| 5c | 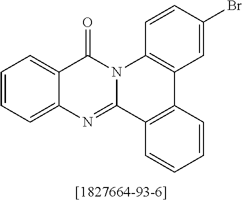 [SMILES: O=c1c2ccccc2nc2c3ccccc3c3cc(Br)ccc3n12] | 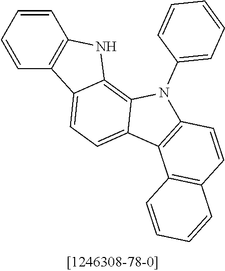 [SMILES: c1ccc(-n2c3ccc4ccccc4c3c3ccc4c5ccccc5[nH]c4c32)cc1] |
| 6c | 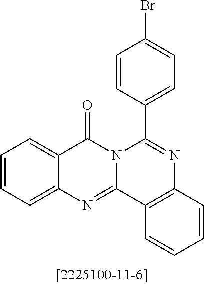 [SMILES: O=c1c2ccccc2nc2c3ccccc3nc(-c3ccc(Br)cc3)n12] | 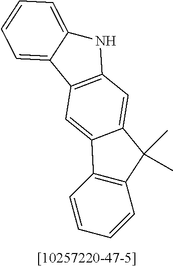 [SMILES: CC1(C)c2ccccc2-c2cc3c(cc21)[nH]c1ccccc13] |
| 7c | 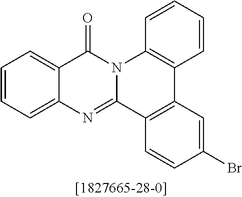 [SMILES: O=c1c2ccccc2nc2c3ccc(Br)cc3c3ccccc3n12] | 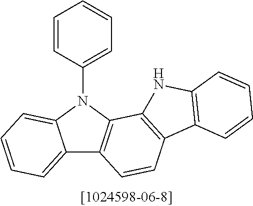 [SMILES: c1ccc(-n2c3ccccc3c3ccc4c5ccccc5[nH]c4c32)cc1] |
| 8c | 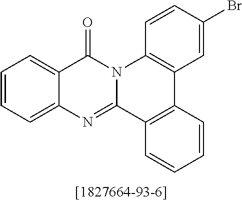 [SMILES: O=c1c2ccccc2nc2c3ccccc3c3cc(Br)ccc3n12] | 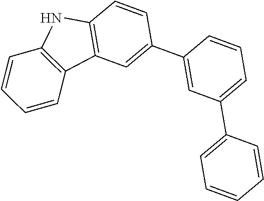 [SMILES: c1ccc(-c2cccc(-c3ccc4[nH]c5ccccc5c4c3)c2)cc1] |
| 9c | 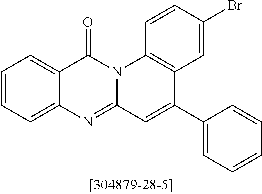 [SMILES: O=c1c2ccccc2nc2cc(-c3ccccc3)c3cc(Br)ccc3n12] | 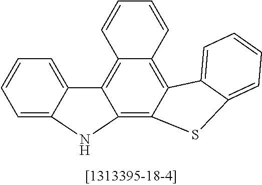 [SMILES: c1ccc2c(c1)[nH]c1c3sc4ccccc4c3c3ccccc3c21] |
| 10c | 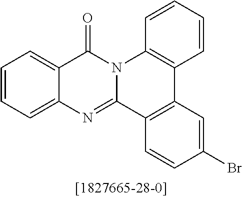 [SMILES: O=c1c2ccccc2nc2c3ccc(Br)cc3c3ccccc3n12] | 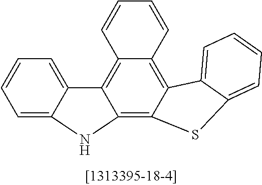 [SMILES: c1ccc2c(c1)[nH]c1c3sc4ccccc4c3c3ccccc3c21] |
| 11c | 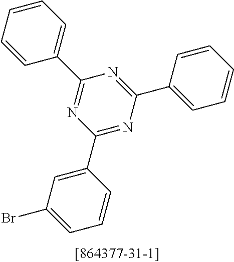 [SMILES: Brc1cccc(-c2nc(-c3ccccc3)nc(-c3ccccc3)n2)c1] | 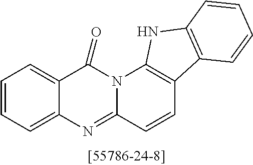 [SMILES: O=c1c2ccccc2nc2ccc3c4ccccc4[nH]c3n12] |
| 12c | 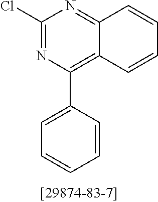 [SMILES: Clc1nc(-c2ccccc2)c2ccccc2n1] | 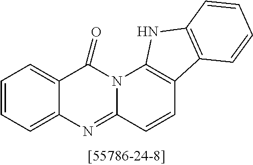 [SMILES: O=c1c2ccccc2nc2ccc3c4ccccc4[nH]c3n12] |
| Ex. | Product | Yield |
| 1c | 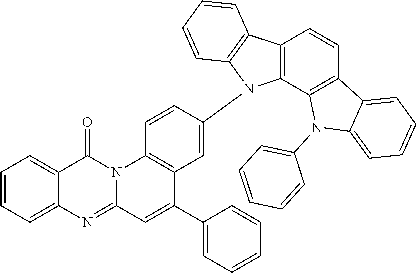 [SMILES: O=c1c2ccccc2nc2cc(-c3ccccc3)c3cc(-n4c5ccccc5c5ccc6c7ccccc7n(-c7ccccc7)c6c54)ccc3n12] | 73% |
| 2c | 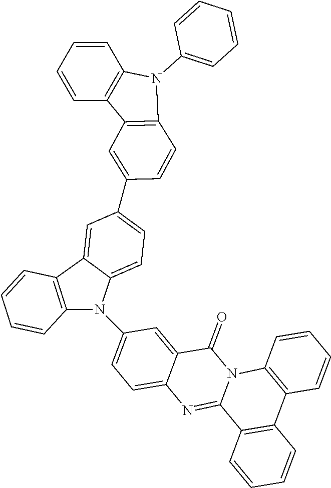 [SMILES: O=c1c2cc(-n3c4ccccc4c4cc(-c5ccc6c(c5)c5ccccc5n6-c5ccccc5)ccc43)ccc2nc2c3ccccc3c3ccccc3n12] | 70% |
| 3c | 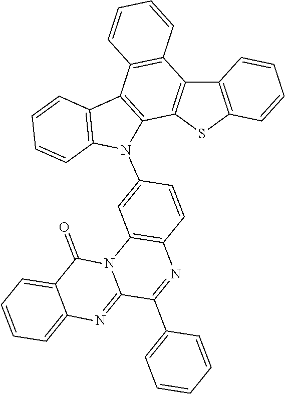 [SMILES: O=c1c2ccccc2nc2c(-c3ccccc3)nc3ccc(-n4c5ccccc5c5c6ccccc6c6c7ccccc7sc6c54)cc3n12] | 68% |
| 4c | 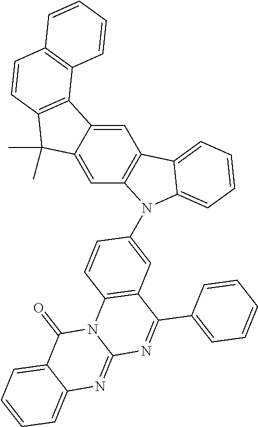 [SMILES: CC1(C)c2cc3c(cc2-c2c1ccc1ccccc21)c1ccccc1n3-c1ccc2c(c1)c(-c1ccccc1)nc1nc3ccccc3c(=O)n12] | 73% |
| 5c | 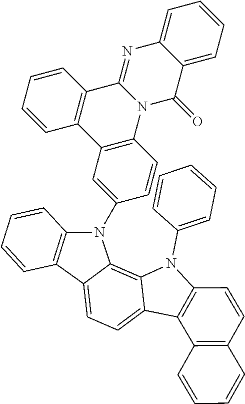 [SMILES: O=c1c2ccccc2nc2c3ccccc3c3cc(-n4c5ccccc5c5ccc6c7c8ccccc8ccc7n(-c7ccccc7)c6c54)ccc3n12] | 84% |
| 6c | 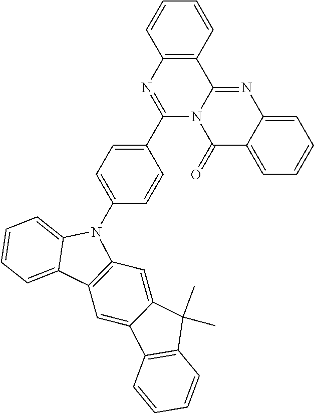 [SMILES: CC1(C)c2ccccc2-c2cc3c4ccccc4n(-c4ccc(-c5nc6ccccc6c6nc7ccccc7c(=O)n56)cc4)c3cc21] | 82% |
| 7c | 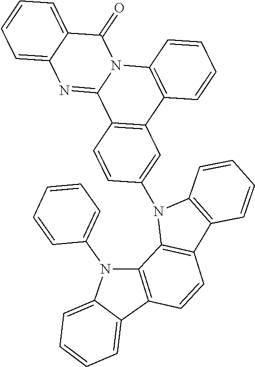 [SMILES: O=c1c2ccccc2nc2c3ccc(-n4c5ccccc5c5ccc6c7ccccc7n(-c7ccccc7)c6c54)cc3c3ccccc3n12] | 67% |
| 8c | 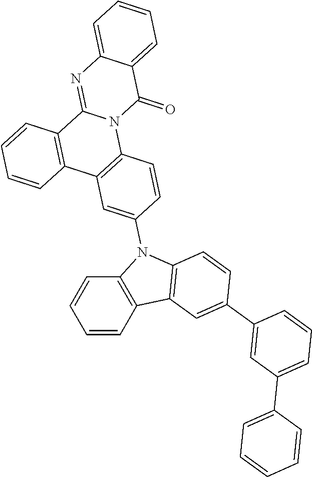 [SMILES: O=c1c2ccccc2nc2c3ccccc3c3cc(-n4c5ccccc5c5cc(-c6cccc(-c7ccccc7)c6)ccc54)ccc3n12] | 62% |
| 9c | 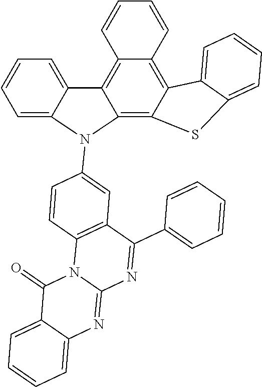 [SMILES: O=c1c2ccccc2nc2nc(-c3ccccc3)c3cc(-n4c5ccccc5c5c6ccccc6c6c7ccccc7sc6c54)ccc3n12] | 60% |
| 10c | 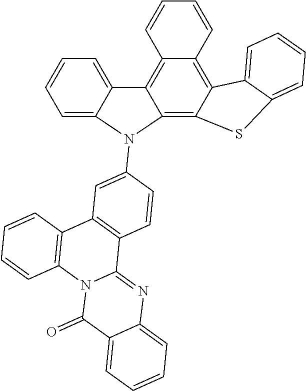 [SMILES: O=c1c2ccccc2nc2c3ccc(-n4c5ccccc5c5c6ccccc6c6c7ccccc7sc6c54)cc3c3ccccc3n12] | 78% |
| 11c | 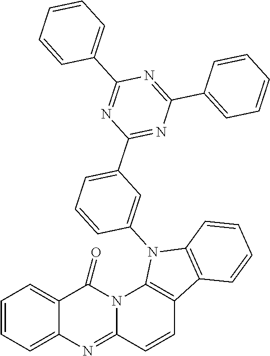 [SMILES: O=c1c2ccccc2nc2ccc3c4ccccc4n(-c4cccc(-c5nc(-c6ccccc6)nc(-c6ccccc6)n5)c4)c3n12] | 65% |
| 12c | 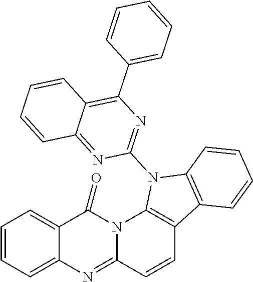 [SMILES: O=c1c2ccccc2nc2ccc3c4ccccc4n(-c4nc(-c5ccccc5)c5ccccc5n4)c3n12] | 58% |

### d) Synthesis of Comparative Compounds

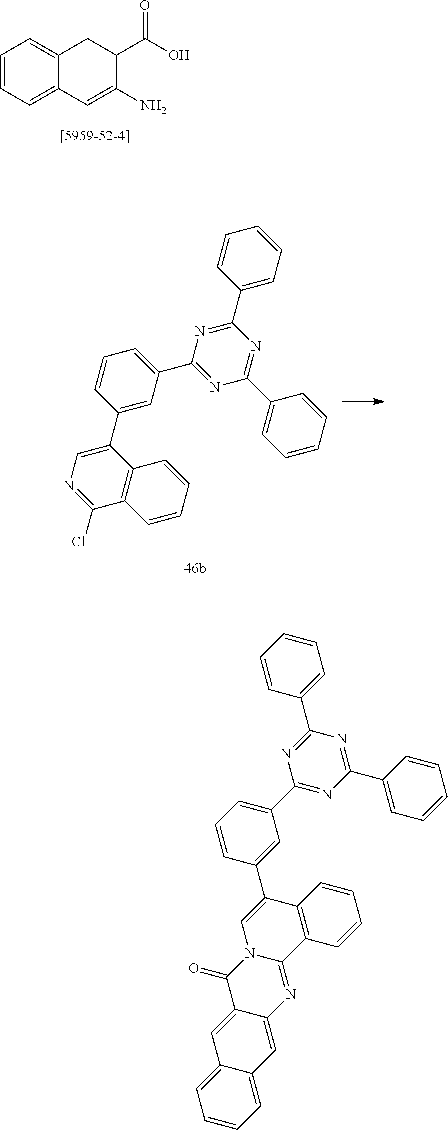 [SMILES: NC1=Cc2ccccc2CC1C(=O)O ; Clc1ncc(-c2cccc(-c3nc(-c4ccccc4)nc(-c4ccccc4)n3)c2)c2ccccc12 ; O=c1c2cc3ccccc3cc2nc2c3ccccc3c(-c3cccc(-c4nc(-c5ccccc5)nc(-c5ccccc5)n4)c3)cn12]

A mixture of 3-aminonaphthalene-2-carboxylic acid, 30 g (160 mmol), 1-chloro-4-[3-(4,6-diphenyl-1,3,5-triazin-2-yl)phenyl]isoquinoline (46b), 75 g (160 mmol), and 700 ml of ethanol and 15 ml of concentrated HCl is heated under reflux for 24 h. The mixture is cooled down to 0° C. and the resulting solid precipitate is collected by filtration. The corresponding hydrochloride is recrystallized twice from pyridine.

The yield is 48.8 g (88 mmol), corresponding to 55% of theory.

Production of the Electroluminescent Devices

Pretreatment for examples E1-E28: Glass plates coated with structured ITO (indium tin oxide) of thickness 50 nm are treated prior to coating with an oxygen plasma, followed by an argon plasma. These plasma-treated glass plates form the substrates to which the OLEDs are applied.

The OLEDs basically have the following layer structure: substrate/optional interlayer (IL)/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 1. The materials required for production of the OLEDs are shown in table 2. The data of the OLEDs are listed in tables 3 and 4.

All materials are applied by thermal vapor deposition in a vacuum chamber. In this case, the emission layer always consists of at least one matrix material (host material) and an emitting dopant (emitter) which is added to the matrix material(s) in a particular proportion by volume by co-evaporation. Details given in such a form as EG1:IC2:TER5 (55%:35%:10%) mean here that the material EG1 is present in the layer in a proportion by volume of 55%, IC2 in a proportion of 35% and TER5 in a proportion of 10%. Analogously, the electron transport layer may also consist of a mixture of two materials.

The OLEDs are characterized in a standard manner. For this purpose, the electroluminescence spectra, the current efficiency (CE, measured in cd/A) and the external quantum efficiency (EQE, measured in %) are determined as a function of luminance, calculated from current-voltage-luminance characteristics assuming Lambertian emission characteristics, as is the lifetime. Electroluminescence spectra are determined at a luminance of 1000 cd/m², and these are used to calculate the CIE 1931 x and y color coordinates. The parameter U1000 refers to the voltage which is required for a luminance of 1000 cd/m². CE1000 and EQE1000 respectively denote the current efficiency and external quantum efficiency that are attained at 1000 cd/m².

The lifetime LT is defined as the time after which the luminance drops from the starting luminance to a certain proportion L1 in the course of operation with constant current density j₀. A figure of L1=95% means that the lifetime reported in the LT column corresponds to the time after which the luminance falls to 95% of its starting value.

Use and Benefit of the Materials of the Invention in OLEDs

A mixture of two host materials is typically used in the emission layer of OLEDs in order to achieve optimal charge balance and hence very good performance data of the OLED. With regard to simplified production of OLEDs, a reduction in the materials to be used is desirable. The use of just one host material in the emission layer is thus advantageous.

By the use of the inventive compounds EG1 to EG4 in examples E1 to E6 as matrix material in the emission layer of red-phosphorescing OLEDs, it is possible to show that the use as a single material (E1 and E2) and particularly in a mixture with a second host material, for example IC2 (E3 to E6), gives very good performance data of the OLEDs, particularly with regard to lifetime and efficiency.

By the use of the inventive compounds EG5 to EG15 in examples E7 to E18 as matrix material in the emission layer of green-phosphorescing OLEDs (as single material and in a mixture with a second host material, for example IC1 or IC3), it is possible to show that these give very good performance data of the OLEDs, particularly with regard to lifetime and efficiency.

The use of the inventive compounds EG5 and EG6 as electron transport material is shown in examples E19 to E22. This gives low operating voltage and very good efficiency and lifetime of the OLEDs.

Comparison of Inventive Compounds with Reference Compounds Having an Enlarged Ring System

Compounds EG14 and EG15 are each compared with comparative compounds EG16 and EG17 in otherwise identical OLED stacks. Compounds EG16 and EG17 have an enlarged ring system on the C═O side of the ring system that contains the C═O—N unit. The comparison shows that the inventive compounds, i.e. the compounds without the enlarged ring system on the side mentioned, have much better performance data (comparison of E18 containing EG15 with V1 containing EG16 and comparison of E17 containing EG14 with V2 containing EG17).

TABLE 1
Structure of the electroluminescent devices

| | | HIL | HTL | EBL | EML | HBL | ETL | EIL |
|---|---|---|---|---|---|---|---|---|
| Ex. | IL | thickness | thickness | thickness | thickness | thickness | thickness | thickness |
| E1 | — | HATCN | SpMA1 | SpMA3 | EG2:TER5 | ST2 | ST2:LiQ | LiQ |
| | | 5 nm | 125 nm | 10 nm | (97%:3%) | 10 nm | (50%:50%) | 1 nm |
| | | | | | 35 nm | | 30 nm | |
| E2 | — | HATCN | SpMA1 | SpMA3 | EG3:TER5 | ST2 | ST2:LiQ | LiQ |
| | | 5 nm | 125 nm | 10 nm | (97%:3%) | 10 nm | (50%:50%) | 1 nm |
| | | | | | 35 nm | | 30 nm | |
| E3 | — | HATCN | SpMA1 | SpMA3 | EG1:IC2:TER5 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E4 | — | HATCN | SpMA1 | SpMA3 | EG2:IC2:TER5 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E5 | — | HATCN | SpMA1 | SpMA3 | EG3:IC2:TER5 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E6 | — | HATCN | SpMA1 | SpMA3 | EG4:IC2:TER5 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E7 | — | HATCN | SpMA1 | SpMA3 | EG6:TEG1 | ST2 | ST2:LiQ | LiQ |
| | | 5 nm | 125 nm | 10 nm | (97%:3%) | 10 nm | (50%:50%) | 1 nm |
| | | | | | 35 nm | | 30 nm | |
| E8 | — | HATCN | SpMA1 | SpMA3 | EG6:IC1:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E9 | — | HATCN | SpMA1 | SpMA3 | EG5:IC3:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E10 | — | HATCN | SpMA1 | SpMA3 | EG7:IC3:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E11 | — | HATCN | SpMA1 | SpMA3 | EG8:IC1:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E12 | — | HATCN | SpMA1 | SpMA3 | EG9:IC1:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E13 | — | HATCN | SpMA1 | SpMA3 | EG10:IC1:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E14 | — | HATCN | SpMA1 | SpMA3 | EG11:IC1:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E15 | — | HATCN | SpMA1 | SpMA3 | EG12:IC1:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E16 | — | HATCN | SpMA1 | SpMA3 | EG13:IC3:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E17 | | HATCN | SpMA1 | SpMA3 | EG14:IC1:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E18 | | HATCN | SpMA1 | SpMA3 | EG15:IC1:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| V1 | | HATCN | SpMA1 | SpMA3 | EG16:IC1:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| V2 | | HATCN | SpMA1 | SpMA3 | EG17:IC1:TEG1 | ST2 | ST2:LiQ | LiQ |
| | | 5 nm | 125 nm | 10 nm | (44%:44%:12%) | 10 nm | (50%:50%) | 1 nm |
| | | | | | 30 nm | | 30 nm | |
| E19 | SpA1 | — | HATCN | SpMA1 | M2:SEB | — | EG5:LiQ | — |
| | 140 nm | | 5 nm | 20 nm | (95%:5%) | | (50%:50%) | |
| | | | | | 20 nm | | 30 nm | |
| E20 | SpA1 | — | HATCN | SpMA1 | IC1:TEG1 | IC1 | EG5:LiQ | — |
| | 70 nm | | 5 nm | 90 nm | (90%:10%) | 10 nm | (50%:50%) | |
| | | | | | 30 nm | | 30 nm | |
| E21 | SpA1 | — | HATCN | SpMA1 | M2:SEB | — | EG6:LiQ | — |
| | 140 nm | | 5 nm | 20 nm | (95%:5%) | | (50%:50%) | |
| | | | | | 20 nm | | 30 nm | |
| E22 | SpA1 | — | HATCN | SpMA1 | IC1:TEG1 | IC1 | EG6:LiQ | — |
| | 70 nm | | 5 nm | 90 nm | (90%:10%) | 10 nm | (50%:50%) | |
| | | | | | 30 nm | | 30 nm | |

TABLE 2
Structural formulae of the materials for the
electroluminescent devices

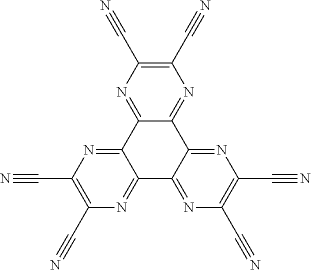 [SMILES: N#Cc1nc2c3nc(C#N)c(C#N)nc3c3nc(C#N)c(C#N)nc3c2nc1C#N]

HATCN

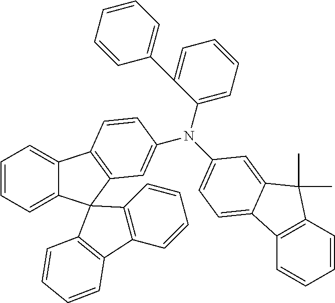 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

SpMA1

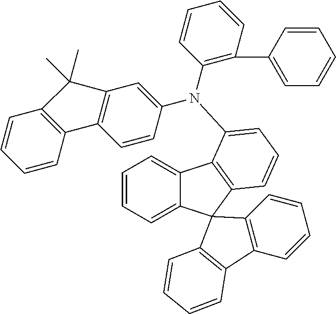 [SMILES: CC1(C)c2ccccc2-c2ccc(N(c3ccccc3-c3ccccc3)c3cccc4c3-c3ccccc3C43c4ccccc4-c4ccccc43)cc21]

SpMA3

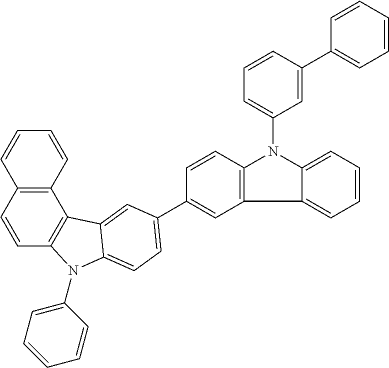 [SMILES: c1ccc(-c2cccc(-n3c4ccccc4c4cc(-c5ccc6c(c5)c5c7ccccc7ccc5n6-c5ccccc5)ccc43)c2)cc1]

IC2

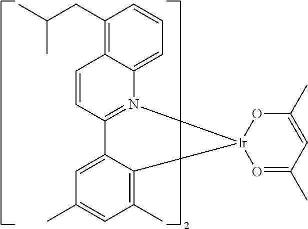

TER5

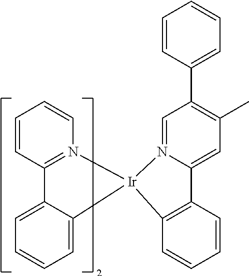

TEG1

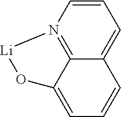

LiQ

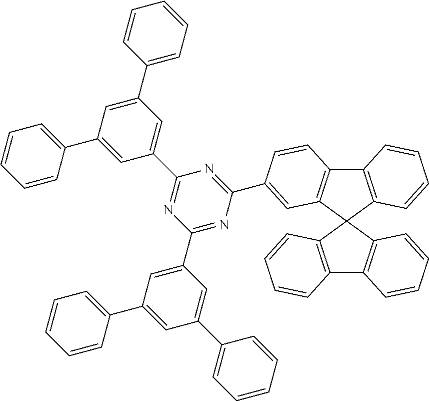 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ST2

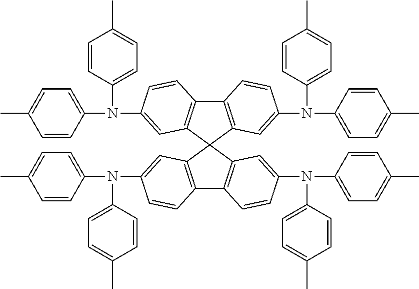

SpA1

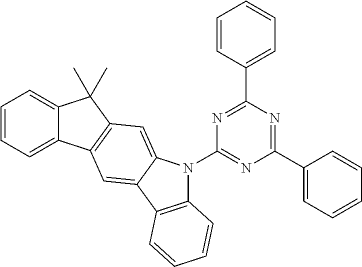 [SMILES: CC1(C)c2ccccc2-c2cc3c4ccccc4n(-c4nc(-c5ccccc5)nc(-c5ccccc5)n4)c3cc21]

IC1

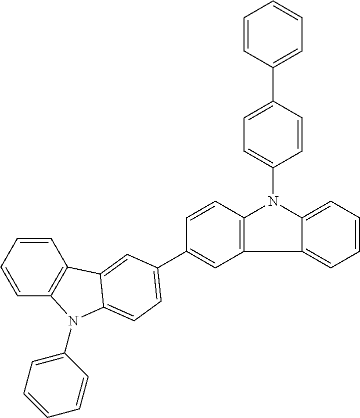 [SMILES: c1ccc(-c2ccc(-n3c4ccccc4c4cc(-c5ccc6c(c5)c5ccccc5n6-c5ccccc5)ccc43)cc2)cc1]

IC3

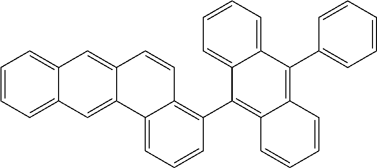 [SMILES: c1ccc(-c2c3ccccc3c(-c3cccc4c3ccc3cc5ccccc5cc34)c3ccccc23)cc1]

M2

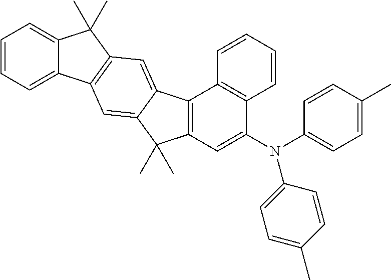 [SMILES: Cc1ccc(N(c2ccc(C)cc2)c2cc3c(c4ccccc24)-c2cc4c(cc2C3(C)C)-c2ccccc2C4(C)C)cc1]

SEB

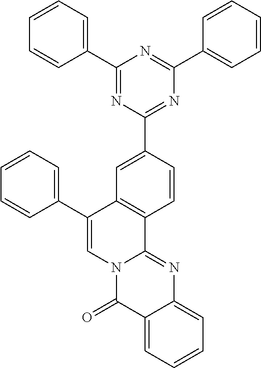 [SMILES: O=c1c2ccccc2nc2c3ccc(-c4nc(-c5ccccc5)nc(-c5ccccc5)n4)cc3c(-c3ccccc3)cn12]

EG1(8b)

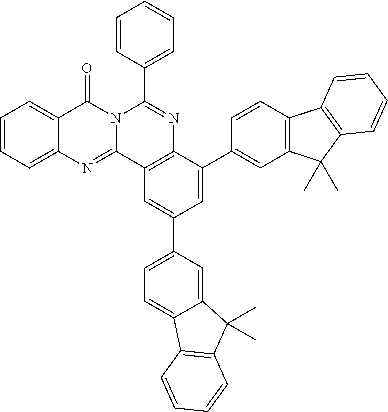 [SMILES: CC1(C)c2ccccc2-c2ccc(-c3cc(-c4ccc5c(c4)C(C)(C)c4ccccc4-5)c4nc(-c5ccccc5)n5c(=O)c6ccccc6nc5c4c3)cc21]

EG2 (10b)

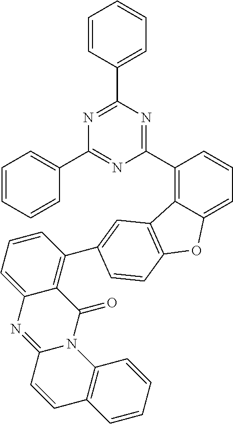 [SMILES: O=c1c2c(-c3ccc4oc5cccc(-c6nc(-c7ccccc7)nc(-c7ccccc7)n6)c5c4c3)cccc2nc2ccc3ccccc3n12]

EG3 (14b)

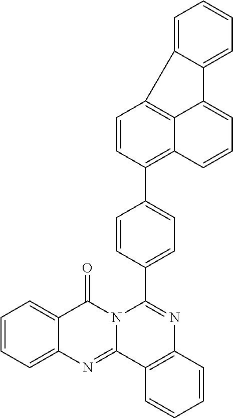 [SMILES: O=c1c2ccccc2nc2c3ccccc3nc(-c3ccc(-c4ccc5c6c(cccc46)-c4ccccc4-5)cc3)n12]

EG4(30b)

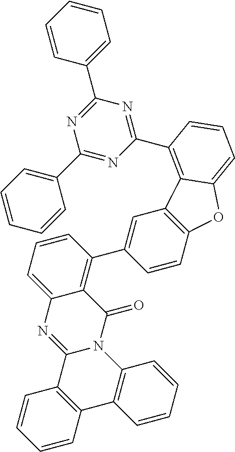 [SMILES: O=c1c2c(-c3ccc4oc5cccc(-c6nc(-c7ccccc7)nc(-c7ccccc7)n6)c5c4c3)cccc2nc2c3ccccc3c3ccccc3n12]

EG5 (2b)

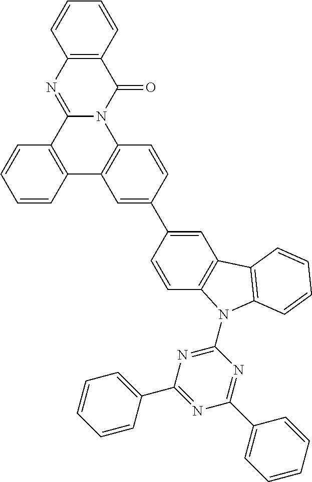 [SMILES: O=c1c2ccccc2nc2c3ccccc3c3cc(-c4ccc5c(c4)c4ccccc4n5-c4nc(-c5ccccc5)nc(-c5ccccc5)n4)ccc3n12]

EG6 (3b)

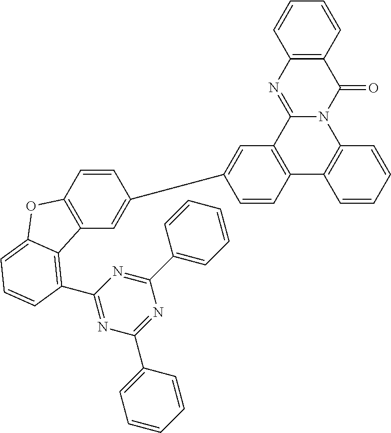 [SMILES: O=c1c2ccccc2nc2c3cc(-c4ccc5oc6cccc(-c7nc(-c8ccccc8)nc(-c8ccccc8)n7)c6c5c4)ccc3c3ccccc3n12]

EG7 (5b)

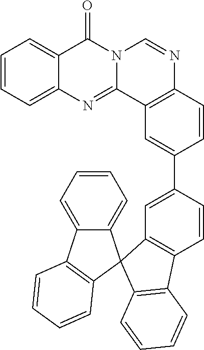 [SMILES: O=c1c2ccccc2nc2c3cc(-c4ccc5c(c4)C4(c6ccccc6-c6ccccc64)c4ccccc4-5)ccc3ncn12]

EG8 (25b)

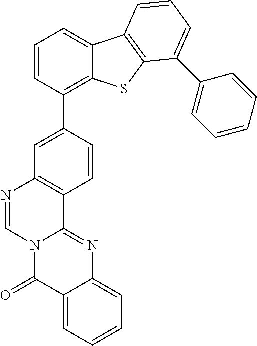 [SMILES: O=c1c2ccccc2nc2c3ccc(-c4cccc5c4sc4c(-c6ccccc6)cccc45)cc3ncn12]

EG9 (26b)

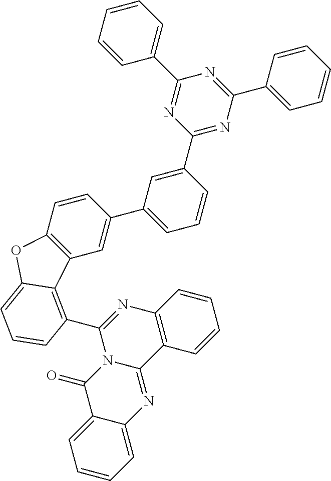 [SMILES: O=c1c2ccccc2nc2c3ccccc3nc(-c3cccc4oc5ccc(-c6cccc(-c7nc(-c8ccccc8)nc(-c8ccccc8)n7)c6)cc5c34)n12]

EG10 (29b)

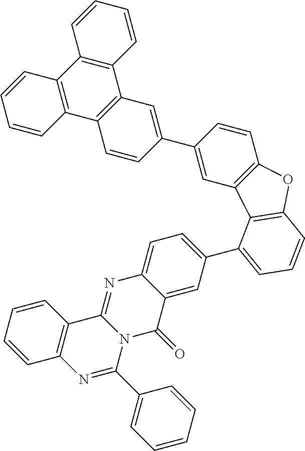 [SMILES: O=c1c2cc(-c3cccc4oc5ccc(-c6ccc7c8ccccc8c8ccccc8c7c6)cc5c34)ccc2nc2c3ccccc3nc(-c3ccccc3)n12]

EG11 (31b)

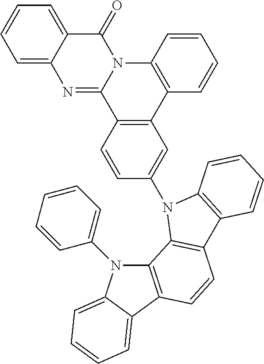 [SMILES: O=c1c2ccccc2nc2c3ccc(-n4c5ccccc5c5ccc6c7ccccc7n(-c7ccccc7)c6c54)cc3c3ccccc3n12]

EG12 (7c)

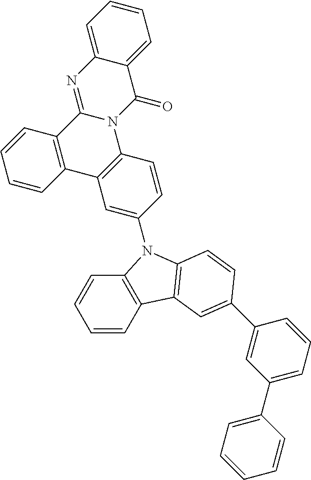 [SMILES: O=c1c2ccccc2nc2c3ccccc3c3cc(-n4c5ccccc5c5cc(-c6cccc(-c7ccccc7)c6)ccc54)ccc3n12]

EG13 (8c)

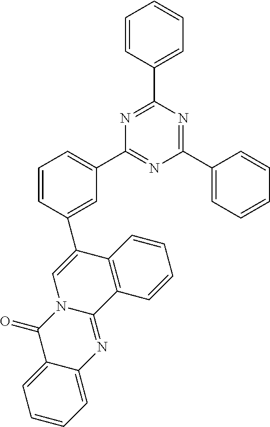 [SMILES: O=c1c2ccccc2nc2c3ccccc3c(-c3cccc(-c4nc(-c5ccccc5)nc(-c5ccccc5)n4)c3)cn12]

EG14 (43b)

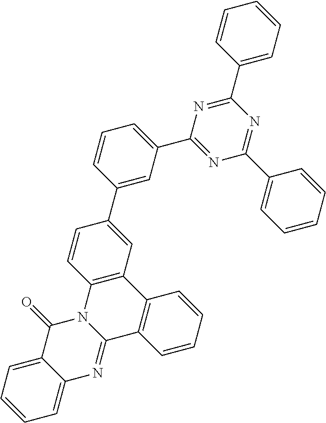 [SMILES: O=c1c2ccccc2nc2c3ccccc3c3cc(-c4cccc(-c5nc(-c6ccccc6)nc(-c6ccccc6)n5)c4)ccc3n12]

EG15 (44b)

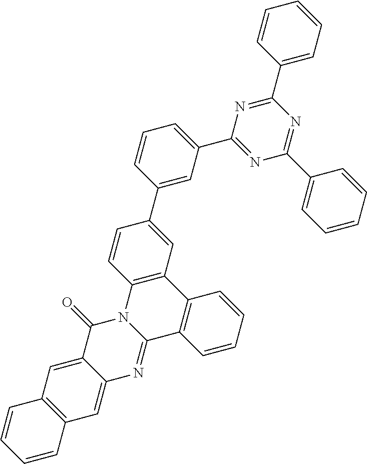 [SMILES: O=c1c2cc3ccccc3cc2nc2c3ccccc3c3cc(-c4cccc(-c5nc(-c6ccccc6)nc(-c6ccccc6)n5)c4)ccc3n12]

EG16 (comparative compound 45b)

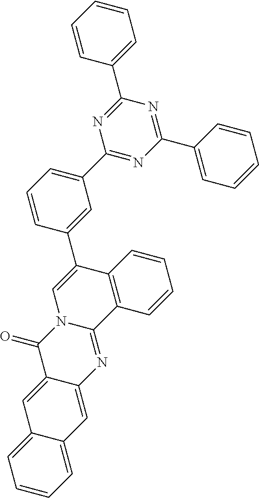 [SMILES: O=c1c2cc3ccccc3cc2nc2c3ccccc3c(-c3cccc(-c4nc(-c5ccccc5)nc(-c5ccccc5)n4)c3)cn12]

EG17 (comparative compound d)

The figure between brackets for the respective compound in table 2 relates to the synthesis example.

TABLE 3
Performance data of the electroluminescent
devices of examples E1-E18, V1 and V2

| | | | EQE | CIE x/y at | | | |
|---|---|---|---|---|---|---|---|
| | U1000 | SE1000 | 1000 | 1000 | j₀ | L1 | LT |
| Ex. | (V) | (cd/A) | (%) | cd/m² | (mA/cm²) | (%) | (h) |
| E1 | 4.3 | 23 | 15 | 0.67/0.33 | 20 | 95 | 510 |
| E2 | 4.4 | 23 | 14 | 0.66/0.34 | 20 | 95 | 550 |
| E3 | 4.2 | 23 | 16 | 0.66/0.34 | 20 | 95 | 830 |
| E4 | 3.4 | 24 | 16 | 0.67/0.33 | 20 | 95 | 845 |
| E5 | 3.9 | 23 | 18 | 0.66/0.33 | 20 | 95 | 760 |
| E6 | 3.4 | 24 | 17 | 0.67/0.34 | 20 | 95 | 825 |
| E7 | 4.5 | 70 | 14.5 | 0.32/0.64 | 20 | 95 | 430 |
| E8 | 3.2 | 67 | 19.1 | 0.33/0.63 | 20 | 95 | 920 |
| E9 | 3.4 | 70 | 18.5 | 0.32/0.64 | 20 | 95 | 944 |
| E10 | 3.2 | 67 | 19.1 | 0.33/0.63 | 20 | 95 | 940 |
| E11 | 3.3 | 69 | 17.3 | 0.32/0.64 | 20 | 95 | 540 |
| E12 | 3.2 | 74 | 16.5 | 0.32/0.63 | 20 | 95 | 760 |
| E13 | 3.9 | 71 | 19.0 | 0.32/0.64 | 20 | 95 | 880 |
| E14 | 3.8 | 77 | 16.2 | 0.33/0.63 | 20 | 95 | 562 |
| E15 | 3.3 | 69 | 18.2 | 0.32/0.63 | 20 | 80 | 770 |
| E16 | 3.2 | 74 | 18.0 | 0.32/0.63 | 20 | 80 | 810 |
| E17 | 3.4 | 24 | 16 | 0.67/0.34 | 20 | 80 | 650 |
| E18 | 3.3 | 24 | 15.5 | 0.67/0.34 | 20 | 80 | 610 |
| V1 | 3.6 | 24 | 13 | 0.67/0.34 | 20 | 80 | 290 |
| V2 | 3.7 | 24 | 12 | 0.67/0.34 | 20 | 80 | 275 |

TABLE 4
Performance data of the electroluminescent devices, examples E19-E22

| | U1000 | SE1000 | PE1000 | EQE | CIE x/y at 1000 | | L1 | LT |
|---|---|---|---|---|---|---|---|---|
| Ex. | (V) | (cd/A) | (lm/W) | 1000 | cd/m² | L₀; j₀ | % | (h) |
| E19 | 4.2 | 9 | 5 | 8.4% | 0.13/0.14 | 6000 | 80 | 49 |
| | | | | | | cd/m² | | |
| E20 | 3.5 | 63 | 55 | 17.7% | 0.31/0.64 | 20 | 80 | 151 |
| | | | | | | mA/cm² | | |
| E21 | 3.3 | 7 | 7 | 7.5% | 0.14/0.13 | 6000 | 80 | 55 |
| | | | | | | cd/m² | | |
| E22 | 3.4 | 65 | 52 | 17.2% | 0.34/0.62 | 20 | 80 | 142 |
| | | | | | | mA/cm² | | |

1.-18. (canceled)

19. A compound comprising at least one structure of the formula (I): 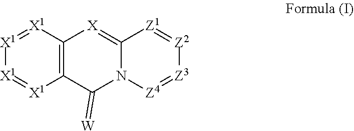
where the symbols and indices used are as follows:
W is O or S;
Z¹ is X², or the Z¹ group together with the Z² group forms a fused ring A¹ bonded via two adjacent carbon atoms to the further groups of the 6-membered ring;
Z² is X², or the Z² group together with the Z¹ group forms a fused ring A¹ bonded via two adjacent carbon atoms to the further groups of the 6-membered ring, or the Z² group together with the Z³ group forms a fused ring A² bonded via two adjacent carbon atoms to the further groups of the 6-membered ring;
Z³ is X², or the Z³ group together with the Z² group forms a fused ring A² bonded via two adjacent carbon atoms to the further groups of the 6-membered ring, or the Z³ group together with the Z⁴ group forms a fused ring A³ bonded via two adjacent carbon atoms to the further groups of the 6-membered ring;
Z⁴ is X², or the Z⁴ group together with the Z³ group forms a fused ring A³ bonded via two adjacent carbon atoms to the further groups of the 6-membered ring;
X is the same or different at each instance and is N or CR;
X¹ is the same or different at each instance and is N or CR¹, with the proviso that not more than two of the X¹ groups in one cycle are N;
X² is the same or different at each instance and is N or CR²;
A¹, A², A³ is the same or different at each instance and is an aromatic or heteroaromatic ring system which has 5 to 40 aromatic ring atoms and has R³ radicals, where the aromatic or heteroaromatic ring system is bonded via two connected carbon atoms to the further groups in the structure shown in formula (I), where two adjacent A¹, A², A³ groups together may form a fused ring system;
R is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is possible here for an R radical together with a further radical;
R¹ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R¹ radicals together to form an aliphatic or heteroaliphatic ring system;
R² is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R² radicals or one R² radical together with a further radical, an R, R³ group, to form an aromatic, heteroaromatic, aliphatic or heteroaliphatic ring system; the R² radicals;
R³ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R³ radicals or one R³ radical together with a further radical, an R, R² group, an aromatic, heteroaromatic, aliphatic or heteroaliphatic ring system;
Ar′ is the same or different at each instance and is an aromatic or heteroaromatic ring system which has 5 to 40 aromatic ring atoms and has R⁴ radicals;
R⁴ is the same or different at each instance and is H, D, F, Cl, Br, I, R⁵C═C(R⁵)₂, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁵ radicals, where one or more nonadjacent CH₂ groups may be replaced by R⁵C═CR⁵, C≡C, Si(R⁵)₂, C═O, C═S, C═Se, C═NR⁵, —C(═O)O—, —C(═O)NR⁵—, NR⁵, P(═O)(R⁵), —O—, —S—, SO or SO₂, or an aromatic or heteroaromatic ring system which has 5 to 40 aromatic ring atoms and in each case has R⁵ radicals, or an aryloxy or heteroaryloxy group which has 5 to 40 aromatic ring atoms and in each case has R⁵ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁵ radicals; it is possible here for two or more R⁴ radicals together to form an aromatic, heteroaromatic, aliphatic or heteroaliphatic ring system;
R⁵ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F; it is possible here for two or more R⁵ radicals together to form a ring system;
wherein
the structure of formula (I) has at least one ring A¹, A², A³ which is formed by at least two adjacent groups selected from the Z¹, Z², Z³ and Z⁴ groups, and
at least one of the R, R¹, R², R³ radicals is selected from the group consisting of a heteroaromatic ring system which has 6 to 60 aromatic ring atoms and has R⁴ radicals, an aromatic ring system which has 10 to 60 aromatic ring atoms and has R⁴ radicals, an aryloxy group having 10 to 60 aromatic ring atoms or heteroaryloxy group having 6 to 40 aromatic ring atoms, each of which has R⁴ radicals, a diarylamino group having 6 to 60 aromatic ring atoms in the respective aromatic radical, an arylheteroarylamino group having 6 to 60 aromatic ring atoms in the respective aromatic or heteroaromatic radical, and a diheteroarylamino group having 6 to 60 aromatic ring atoms in the respective heteroaromatic radical, where the diarylamino, arylheteroarylamino, diheteroarylamino group has R⁴ radicals.

20. The compound as claimed in claim 19, comprising at least one structure of the formulae (II-1) to (II-7): 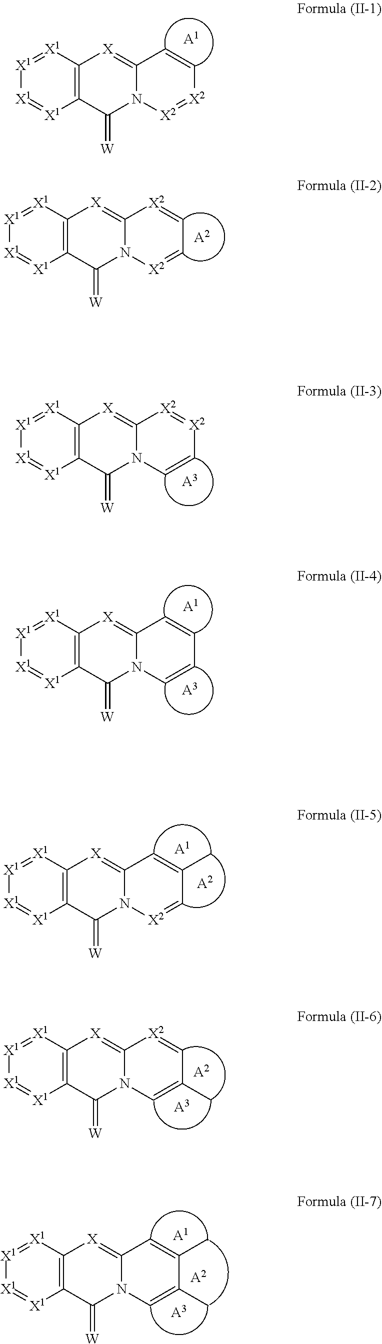
where the symbols W, X, X¹, X², A¹, A² and A³ have the definition given in claim 19.

21. The compound as claimed in claim 19, wherein the rings of the formulae A¹, A² and A³ are represented by structures of the formulae (KAr-1) or (KAr-2) 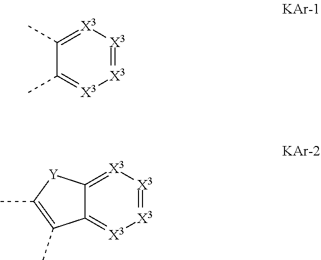
where the symbol X³ is N or CR³, with the proviso that not more than two of the X³ groups in one cycle are N, Y is O, S, NR³, NAr′ or C(R³)₂, where Ar′ and R³ have the definitions set out in claim 19 and the dotted bonds represent the sites of attachment of the ring to the further groups of the structure shown in formula (I) or (II-1) to (II-7), where the carbon atoms bonded to the dotted bonds represent the Z¹, Z², Z³, Z⁴ groups shown in the structure of the formula (I).

22. The compound as claimed in claim 19, wherein at least one X² group is N, and the Z³ group is N.

23. The compound as claimed in claim 19, wherein the X¹ group is N and one of the X² groups is N, one of the Z¹, Z², Z³ groups is N.

24. The compound as claimed in claim 19, comprising at least one structure of the formulae (III-1) to (III-11): 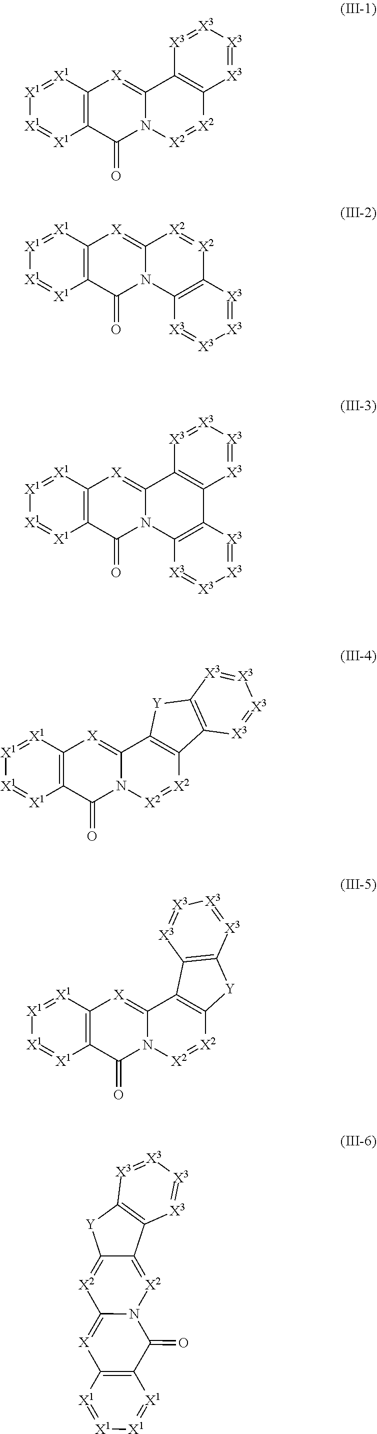 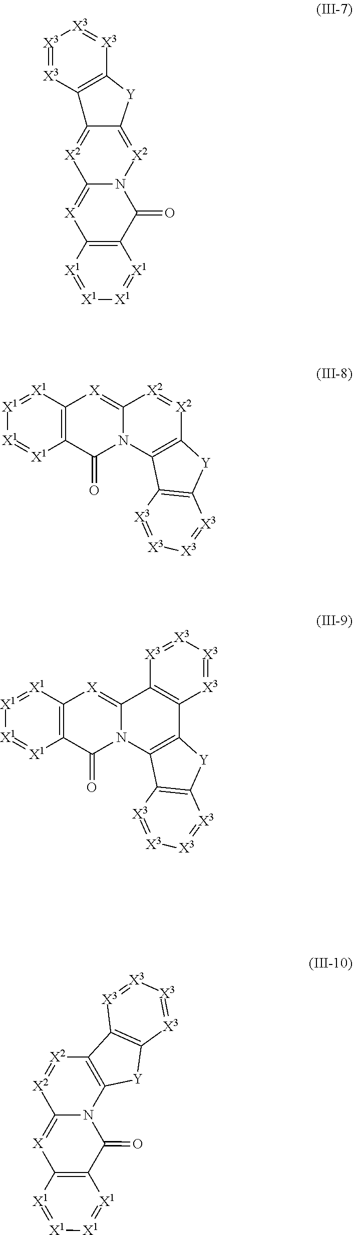 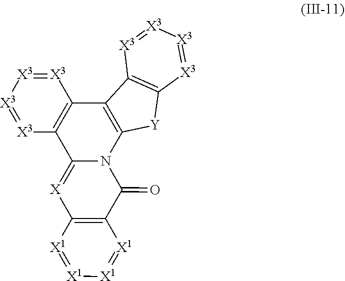
where the symbols X, X¹ and X² have the definitions given in claim 19,
where the symbol X³ is N or CR³, with the proviso that not more than two of the X³ groups in one cycle are N, Y is O, S, NR³, NAr′ or C(R³)₂, where Ar′ and R³ have the definitions set out in claim 19.

25. The compound as claimed in claim 19, comprising at least one structure of the formulae (IV-1) to (IV-11): 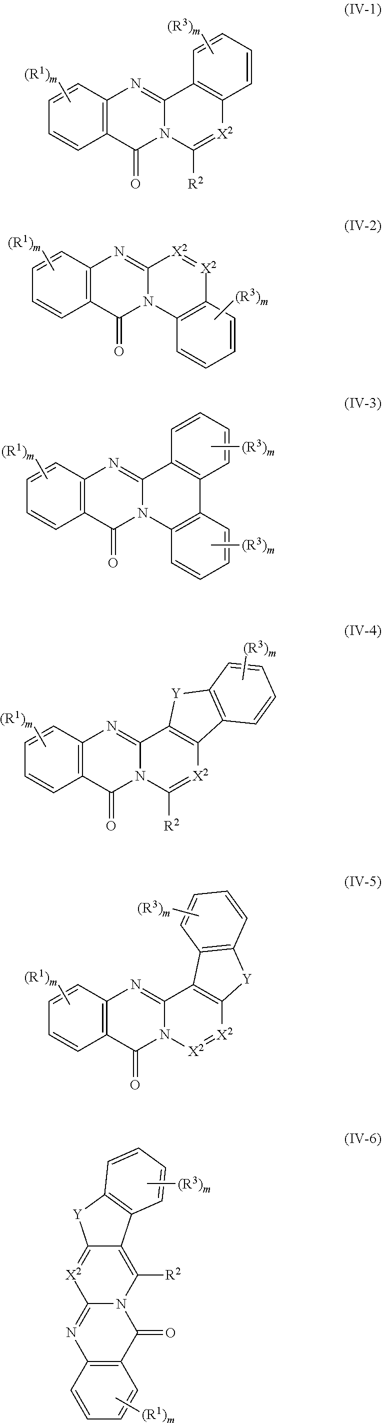 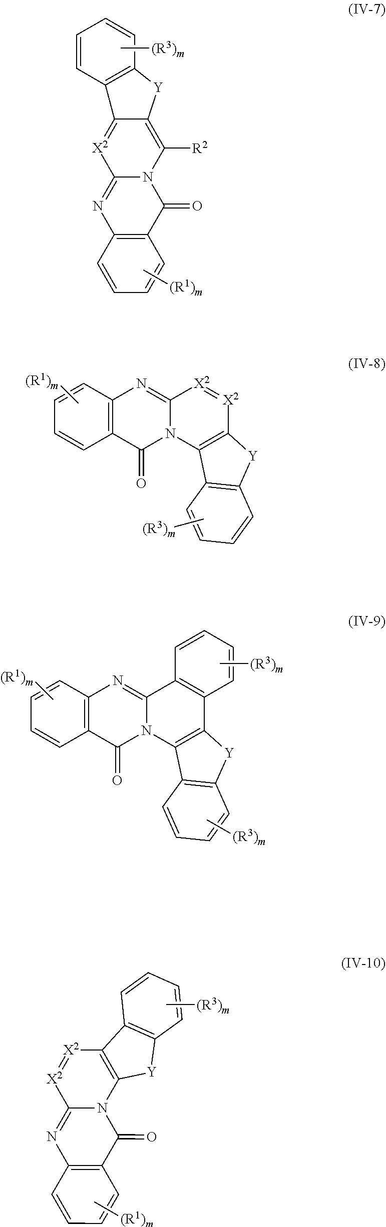 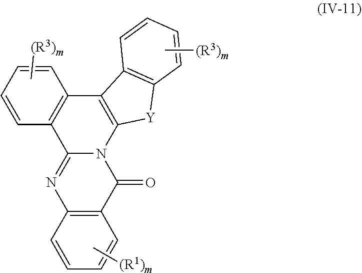
where the symbols R¹, R², R³ and X² have the definitions given in claim 19,
Y is O, S, NR³, NAr′ or C(R³)₂, where Ar′ and R³ have the definitions set out in claim 19,
the index m is 0, 1, 2, 3 or 4, where at least one X² group is N.

26. The compound as claimed in claim 19, comprising at least one structure of the formulae (V-1) to (V-5): 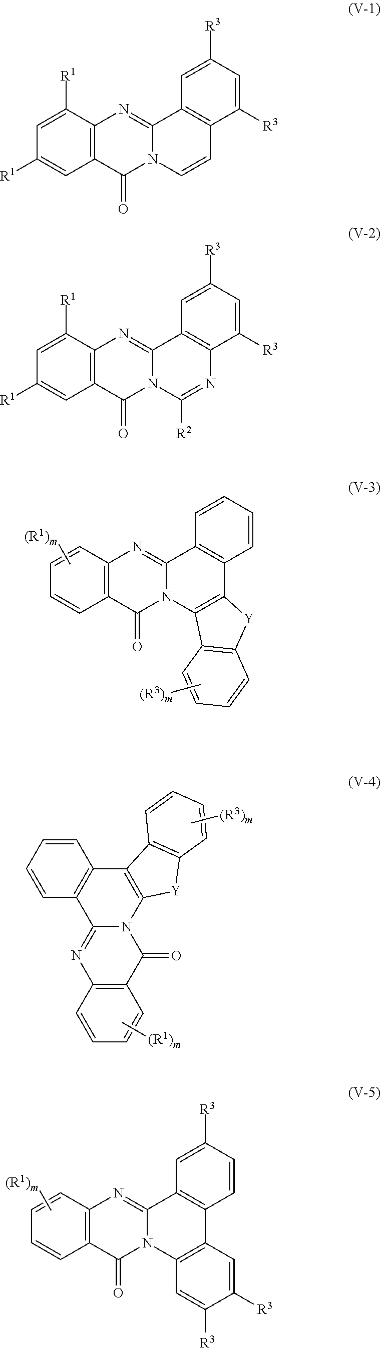
where R¹, R² and R³ have the definitions given in claim 19, the index m is 0, 1, 2, 3 or 4.

27. The compound as claimed in claim 19, wherein at least one R, R¹, R² and/or R³ group is the same or different and is selected from the radicals of the following formulae SAr-1 to SAr-18: 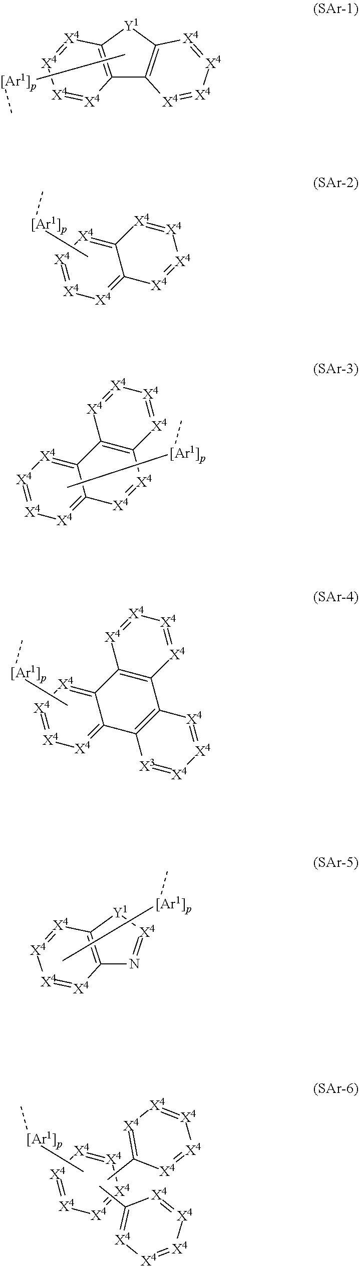 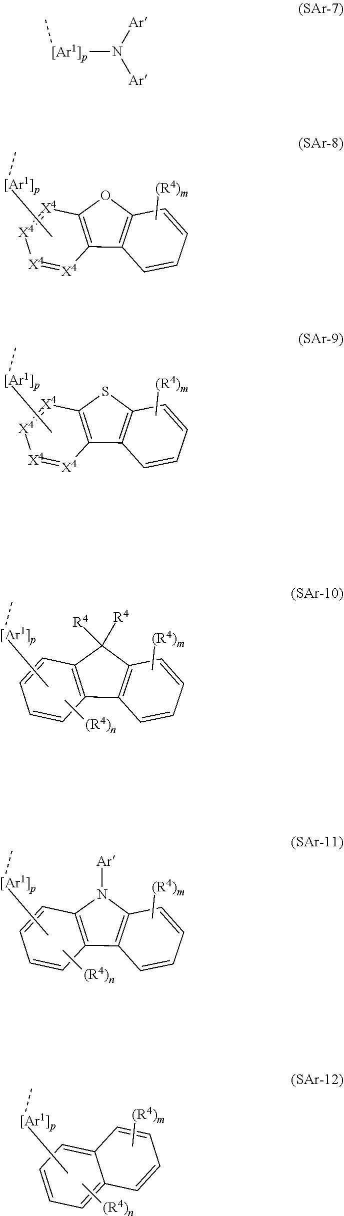 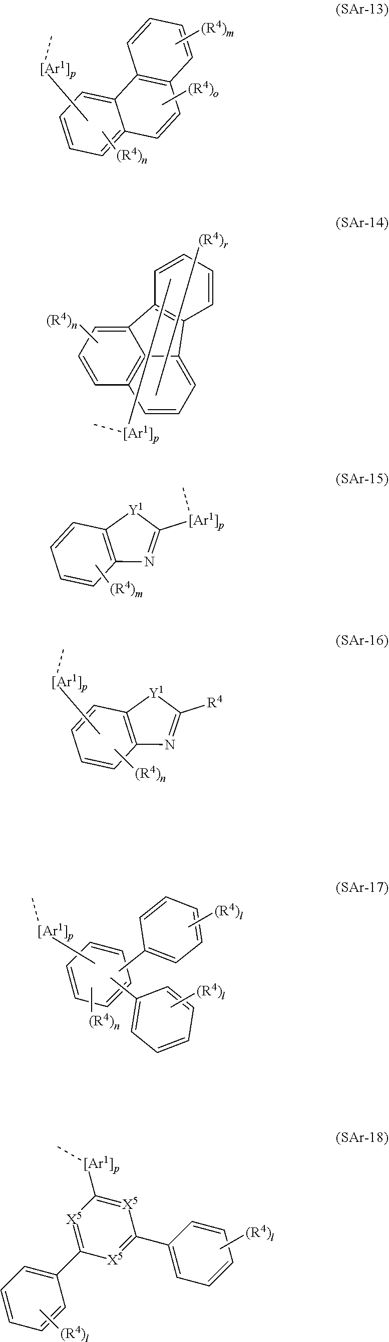
where R⁴ and Ar′ have the definitions given in claim 19 and the further symbols and indices are as follows:
X⁴ is the same or different at each instance and is CR⁴, N, or C if the [Ar¹]ₚ group binds thereto;
X⁵ is the same or different at each instance and is CR⁴ or N;
Ar¹ is the same or different at each instance and is a bivalent aromatic or heteroaromatic ring system which has 6 to 18 aromatic ring atoms and in each case has R⁴ radicals;
Y¹ is the same or different at each instance and is C(R⁴)₂, NR⁴, O, S, or, if the [Ar¹]ₚ group binds thereto, is C(R⁴) or N;
p is 0 or 1, where p=0 means that the Ar¹ group is absent and that the corresponding aromatic or heteroaromatic group is bonded directly to the corresponding radical;
n is 0, 1, 2 or 3;
m is 0, 1, 2, 3 or 4;
1 is 0, 1, 2, 3, 4 or 5;
r is 0, 1, 2, 3, 4, 5 or 6

28. The compound as claimed in claim 19, wherein R, R¹, R² and/or R³ are the same or different at each instance and are selected from the group consisting of H, D or an aromatic or heteroaromatic ring system selected from the groups of the following formulae Ar-1 to Ar-79; at least one R, R¹, R² and/or R³ group is the same or different and is selected from the groups of the following formulae Ar-1 to Ar-79 and/or the Ar′ group is the same or different at each instance and is selected from the groups of the following formulae Ar-1 to Ar-79: 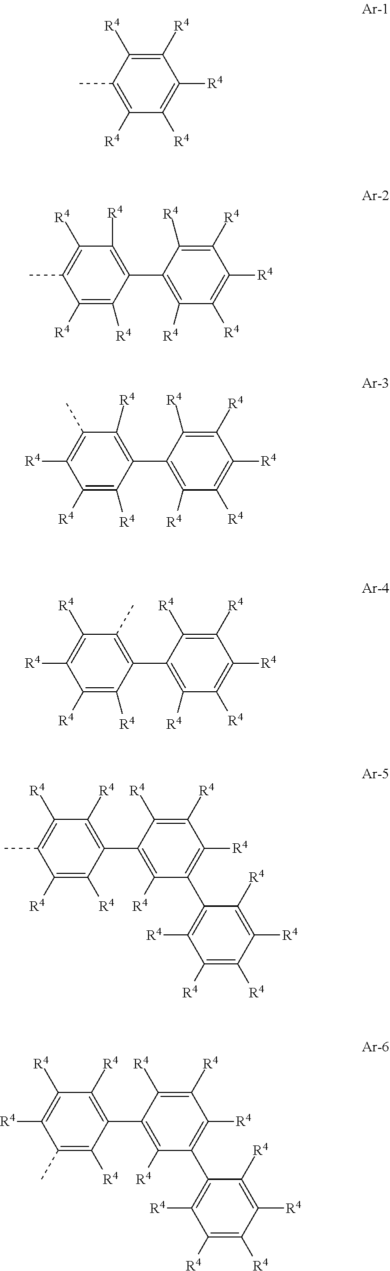 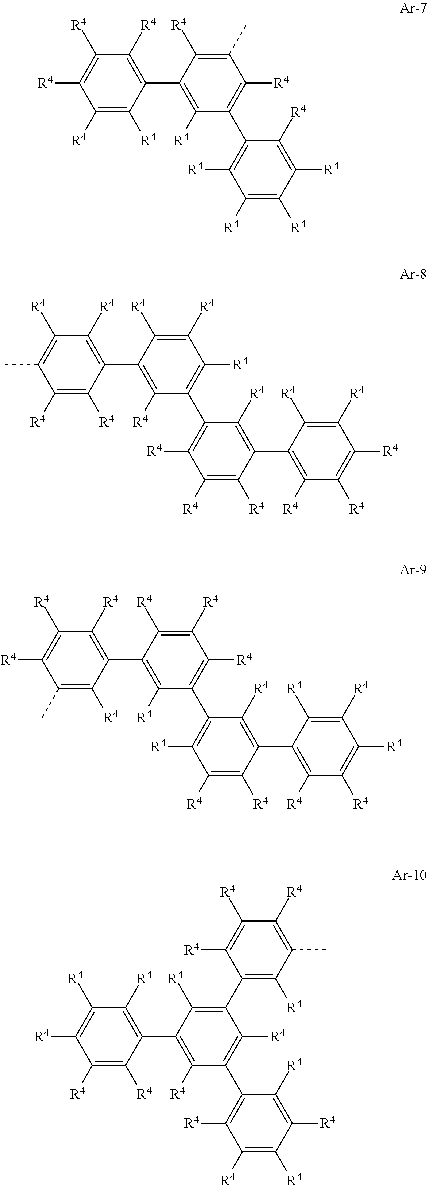 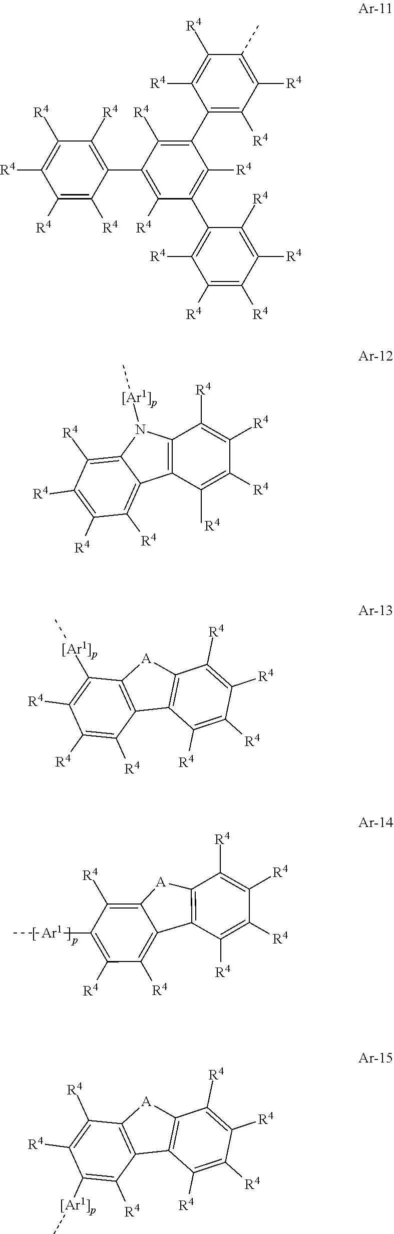 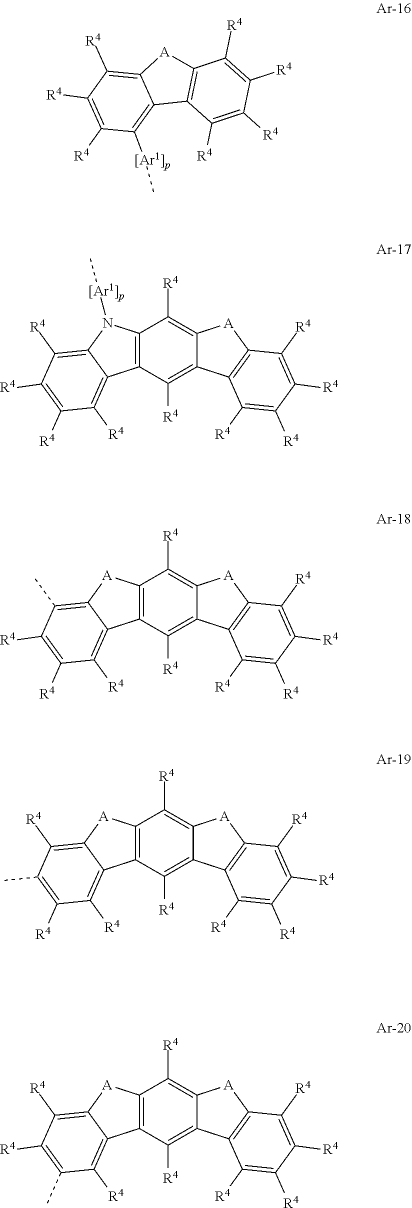 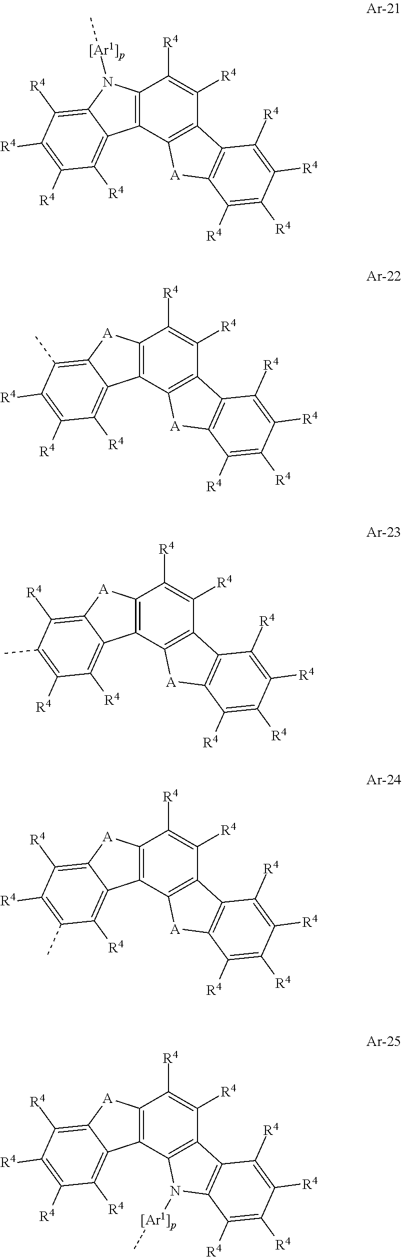 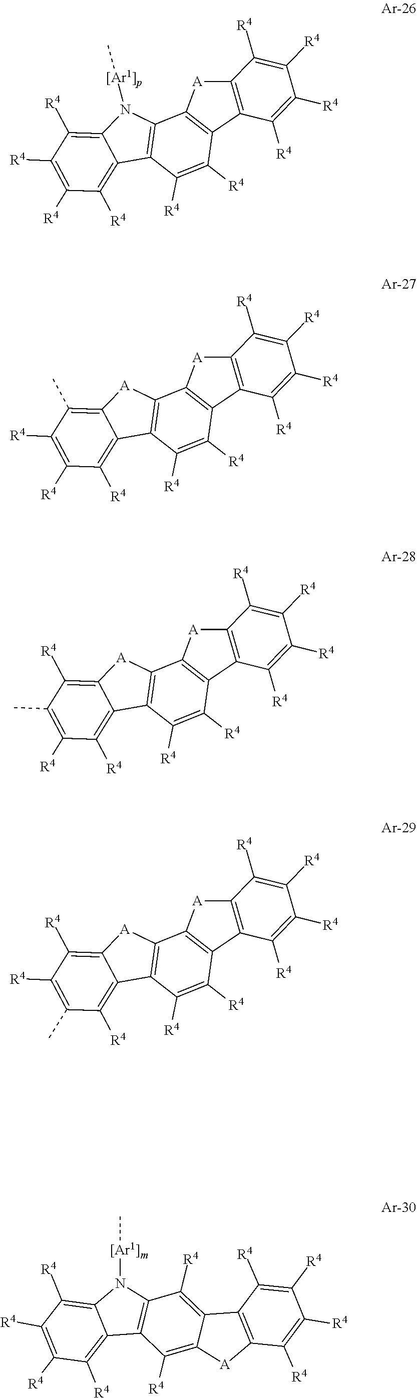 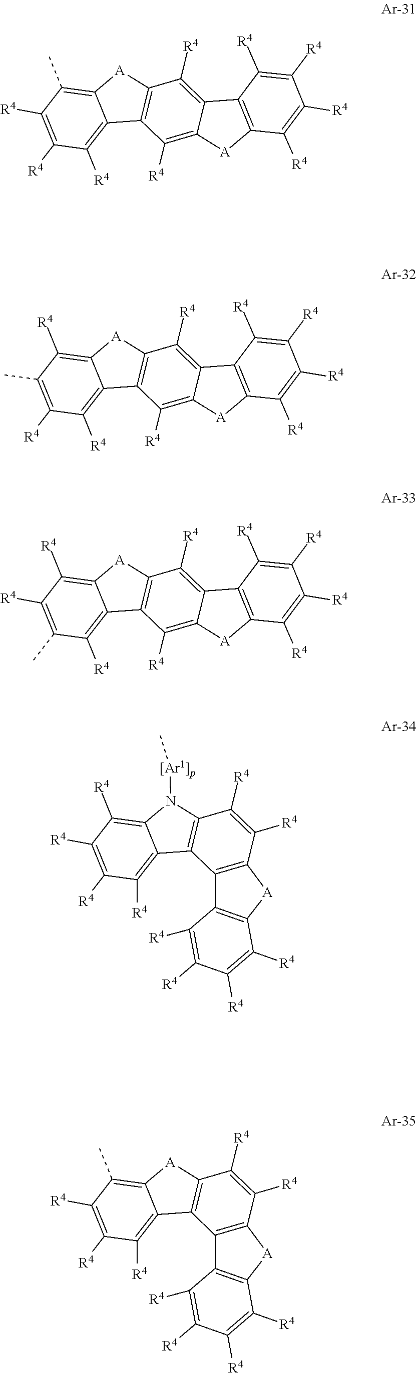 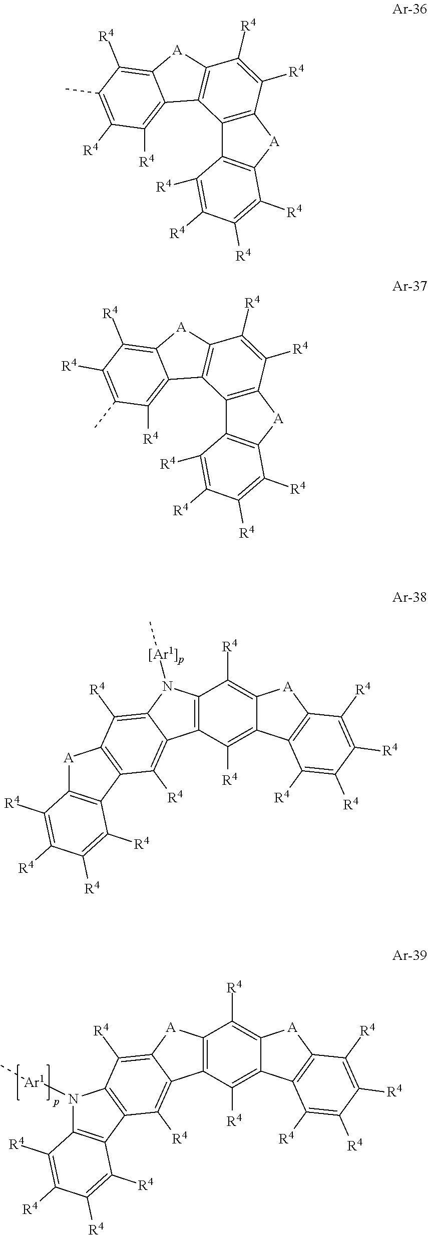 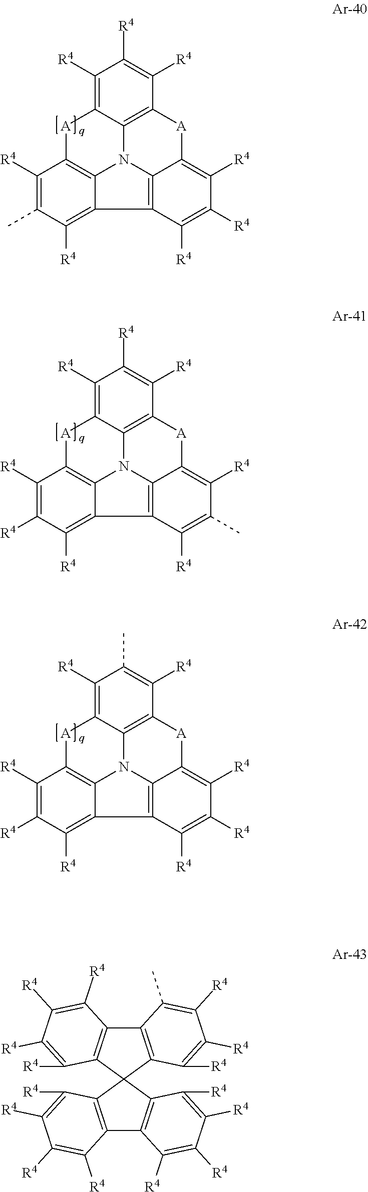 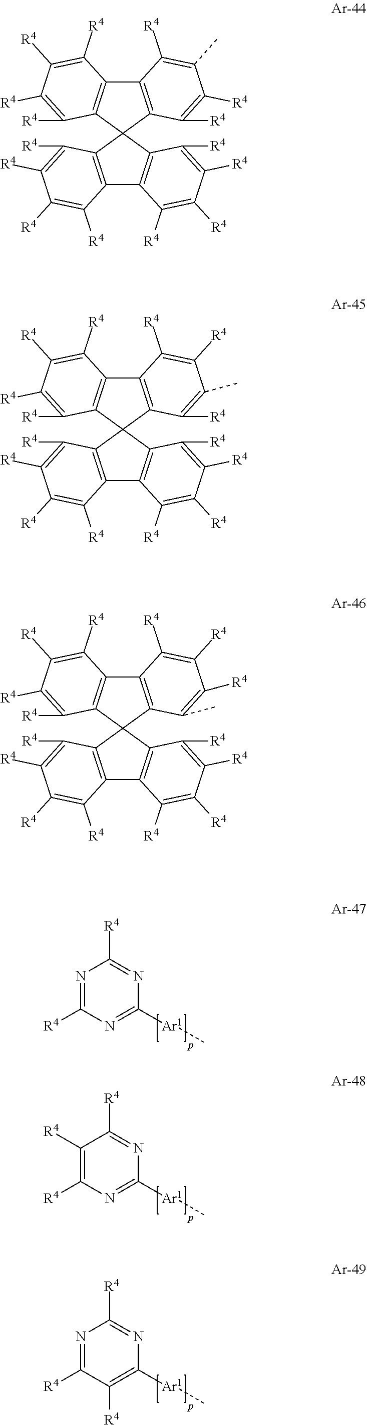 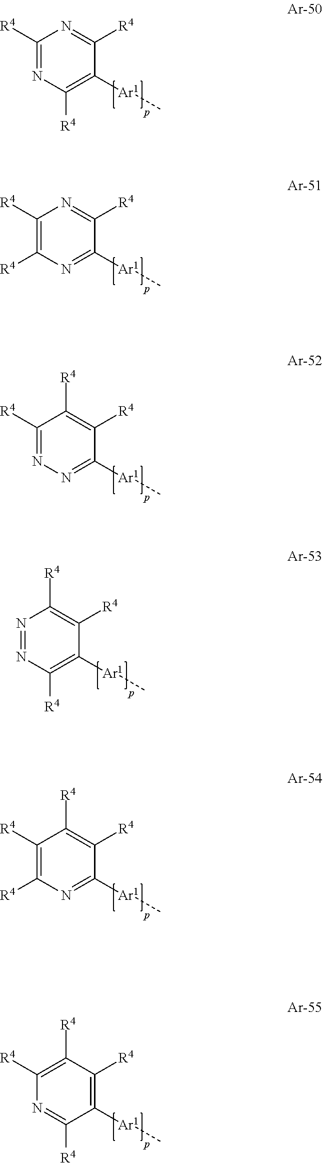 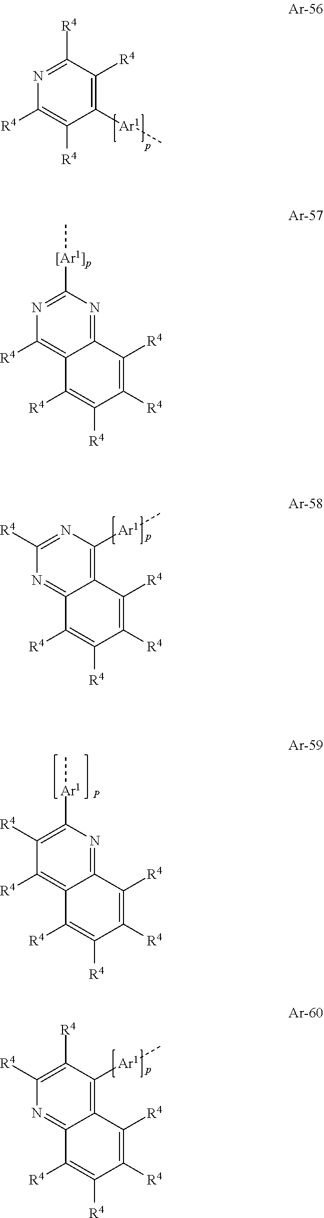 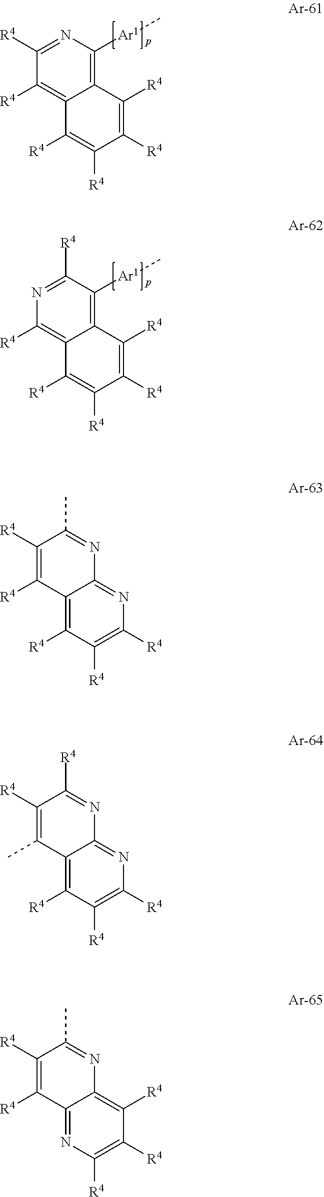 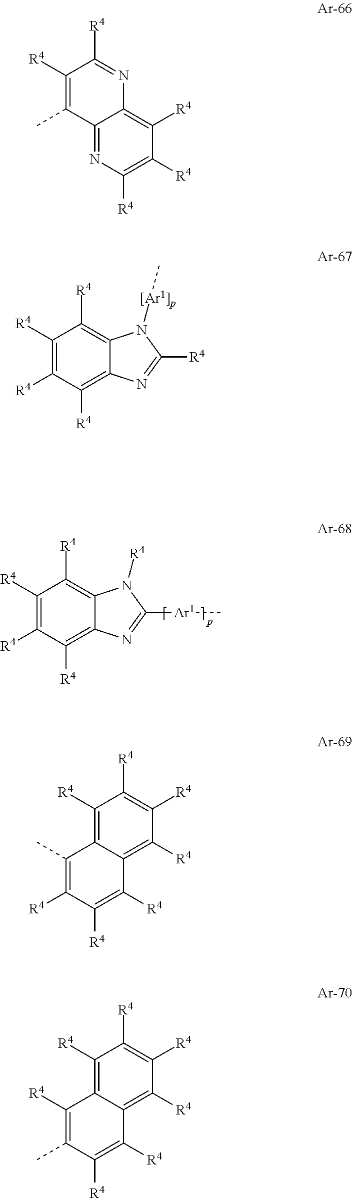 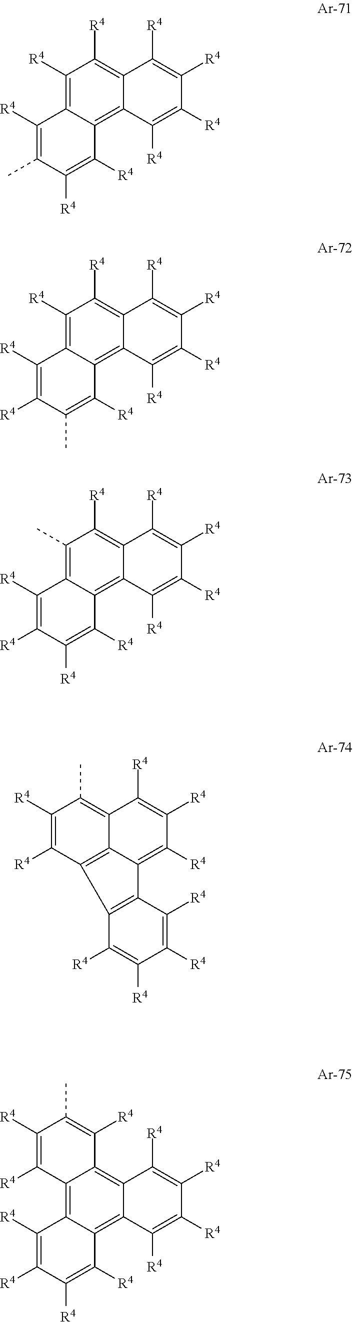 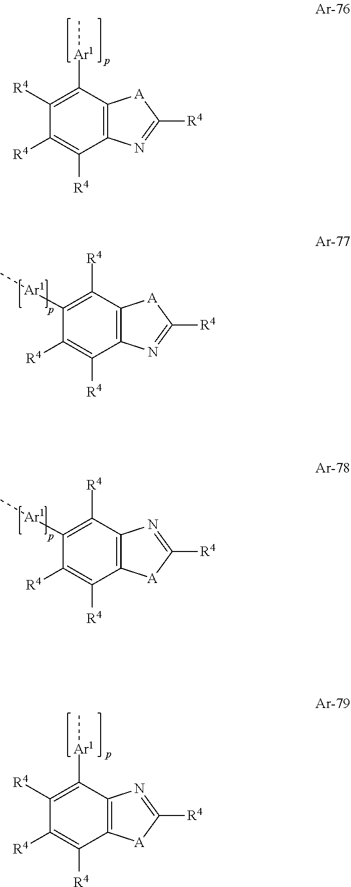
where R⁴ has the definitions given above, the dotted bond represents the bond to the corresponding group and in addition:
Ar¹ is the same or different at each instance and is a bivalent aromatic or heteroaromatic ring system which has 6 to 18 aromatic ring atoms and in each case has R⁴ radicals;
A is the same or different at each instance and is C(R⁴)₂, NR⁴, O or S;
p is 0 or 1, where p=0 means that the Ar¹ group is absent and that the corresponding aromatic or heteroaromatic group is bonded directly to the corresponding radical;
q is 0 or 1, where q=0 means that no A group is bonded at this position and R⁴ radicals are bonded to the corresponding carbon atoms instead.

29. A process for preparing the compound as claimed in claim 19, which comprises reacting a nitrogen-containing aromatic or heteroaromatic compound in a ring-forming reaction.

30. A composition containing at least one compound comprising at least one structure of the formula (Ia): 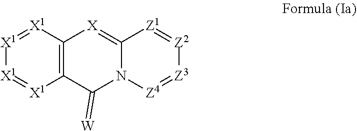
where the symbols and indices used are as follows:
W is O or S;
Z¹ is X², or the Z¹ group together with the Z² group forms a fused ring A¹ bonded via two adjacent carbon atoms to the further groups of the 6-membered ring;
Z² is X², or the Z² group together with the Z¹ group forms a fused ring A¹ bonded via two adjacent carbon atoms to the further groups of the 6-membered ring, or the Z² group together with the Z³ group forms a fused ring A² bonded via two adjacent carbon atoms to the further groups of the 6-membered ring;
Z³ is X², or the Z³ group together with the Z² group forms a fused ring A² bonded via two adjacent carbon atoms to the further groups of the 6-membered ring, or the Z³ group together with the Z⁴ group forms a fused ring A³ bonded via two adjacent carbon atoms to the further groups of the 6-membered ring;
Z⁴ is X², or the Z⁴ group together with the Z³ group forms a fused ring A³ bonded via two adjacent carbon atoms to the further groups of the 6-membered ring;
X is the same or different at each instance and is N or CR;
X¹ is the same or different at each instance and is N or CR¹, with the proviso that not more than two of the X¹ groups in one cycle are N;
X² is the same or different at each instance and is N or CR²;
A¹, A², A³ is the same or different at each instance and is an aromatic or heteroaromatic ring system which has 5 to 40 aromatic ring atoms and has R³ radicals, where the aromatic or heteroaromatic ring system is bonded via two connected carbon atoms to the further groups in the structure shown in formula (I), where two adjacent A¹, A², A³ groups together may form a fused ring system;
R is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is possible here for an R radical together with a further radical;
R¹ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R¹ radicals together to form an aliphatic or heteroaliphatic ring system; the R¹ radicals; with exclusion of the formation of an aromatic or heteroaromatic ring system by two R¹ radicals;
R² is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R² radicals or one R² radical together with a further radical, form an aromatic, heteroaromatic, aliphatic or heteroaliphatic ring system;
R³ is the same or different at each instance and is H, D, F, Cl, Br, I, R⁴C═C(R⁴)₂, N(R⁴)₂, N(Ar′)₂, CN, NO₂, OR⁴, OAr′, SR⁴, SAr′, C(═O)OR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁴ radicals and where one or more nonadjacent CH₂ groups may be replaced by R⁴C═CR⁴, C≡C, Si(R⁴)₂, C═O, C═S, C═Se, C═NR⁴, —C(═O)O—, —C(═O)NR⁴—, NR⁴, P(═O)(R⁴), —O—, —S—, SO or SO₂, or an aromatic or heteroaromatic ring system which has 5 to 60 aromatic ring atoms, and in each case has R⁴ radicals, or an aryloxy or heteroaryloxy group which has 5 to 40 aromatic ring atoms and in each case has R⁴ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁴ radicals; it is also possible here for two R³ radicals or one R³ radical together with a further radical, form an aromatic, heteroaromatic, aliphatic or heteroaliphatic ring system; the R³ radicals;
Ar′ is the same or different at each instance and is an aromatic or heteroaromatic ring system which has 5 to 40 aromatic ring atoms and has R⁴ radicals;
R⁴ is the same or different at each instance and is H, D, F, Cl, Br, I, R⁵C═C(R⁵)₂, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl, alkoxy or thioalkyl group having 1 to 20 carbon atoms or an alkenyl or alkynyl group having 2 to 20 carbon atoms or a branched or cyclic alkyl, alkoxy or thioalkyl group having 3 to 20 carbon atoms, where each alkyl, alkoxy or thioalkyl, alkenyl or alkynyl group has R⁵ radicals, where one or more nonadjacent CH₂ groups may be replaced by R⁵C═CR⁵, C≡C, Si(R⁵)₂, C═O, C═S, C═Se, C═NR⁵, —C(═O)O—, —C(═O)NR⁵—, NR⁵, P(═O)(R⁵), —O—, —S—, SO or SO₂, or an aromatic or heteroaromatic ring system which has 5 to 40 aromatic ring atoms and in each case has R⁵ radicals, or an aryloxy or heteroaryloxy group which has 5 to 40 aromatic ring atoms and in each case has R⁵ radicals, or a diarylamino, arylheteroarylamino, diheteroarylamino group having 5 to 60 aromatic ring atoms in the respective aromatic or heteroaromatic radical, where the diarylamino, arylheteroarylamino, diheteroarylamino group has R⁵ radicals; it is possible here for two or more R⁴ radicals together to form an aromatic, heteroaromatic, aliphatic or heteroaliphatic ring system;
R⁵ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F; it is possible here for two or more R⁵ radicals together to form a ring system;
where the structure of formula (Ia) has at least one ring A¹, A², A³ which is formed by at least two adjacent groups selected from the Z¹, Z², Z³ and Z⁴ groups;
and
at least one further matrix material, where the further matrix material is selected from compounds of one of the formulae (H-1), (H-2), (H-3), (H-4) or (H-5): 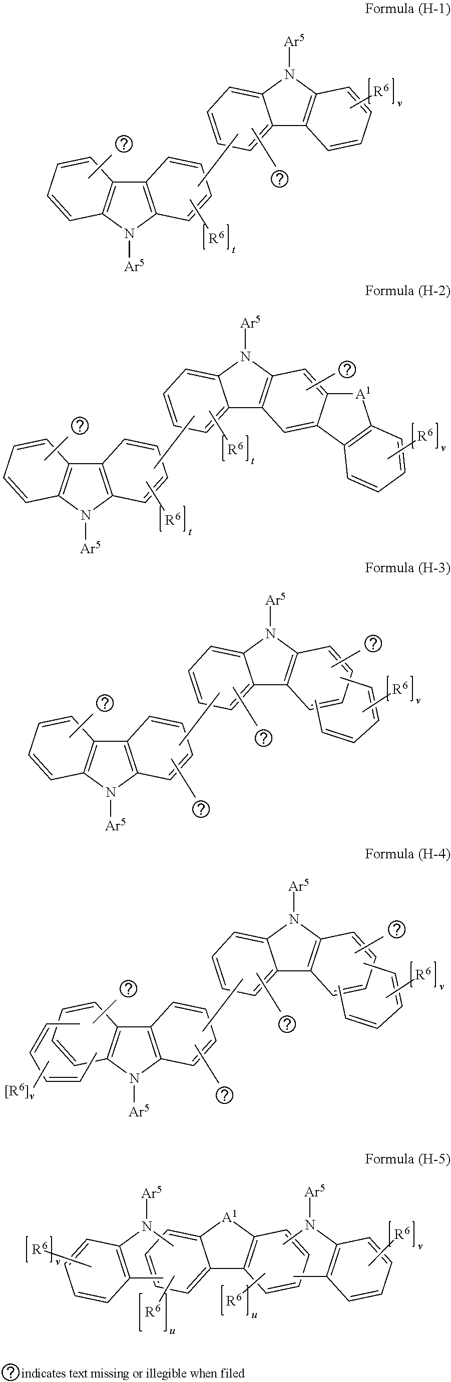
where the symbols and indices used are as follows:
R⁶ is the same or different at each instance and is H, D, F, Cl, Br, I, N(R⁷)₂, N(Ar″)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R⁷ radicals and where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, and in each case has R⁷ radicals; at the same time, two R⁶ radicals together may also form an aromatic, heteroaromatic, aliphatic or heteroaliphatic ring system;
Ar″ is the same or different at each instance and is an aromatic or heteroaromatic ring system which has 5 to 40 aromatic ring atoms and has R⁷ radicals;
A¹ is C(R⁷)₂, NR⁷, O or S;
Ar⁵ is the same or different at each instance and is an aromatic or heteroaromatic ring system which has 5 to 40 aromatic ring atoms and has R⁷ radicals;
R⁷ is the same or different at each instance and is H, D, F, Cl, Br, I, N(R⁸)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R¹ radicals, where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and in each case has R¹ radicals; at the same time, two or more R⁷ radicals together may form an aromatic, heteroaromatic, aliphatic or heteroaliphatic ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
v is the same or different at each instance and is 0, 1, 2, 3 or 4;
t is the same or different at each instance and is 0, 1, 2 or 3;
u is the same or different at each instance and is 0, 1 or 2.

31. The composition as claimed in claim 30, wherein the compounds of one of the formulae (H-1), (H-2), (H-3), (H-4) or (H-5) have a proportion by mass in the composition in the range from 5% by weight to 90% by weight, based on the overall composition.

32. A formulation comprising at least one compound as claimed in claim 19 and at least one further compound.

33. A formulation comprising at least one composition as claimed in claim 30 and at least one further compound.

34. An electronic device containing at least one compound comprising at least one structure of the formula (Ia): 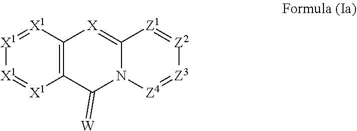
where the symbols and indices used have the definition given in claim 30;
where the structure of formula (Ia) has at least one ring A¹, A², A³ which is formed by at least two adjacent groups selected from the Z¹, Z², Z³ and Z⁴ groups

35. The electronic device as claimed in claim 34 which is an organic electroluminescent device, wherein the compound comprising at least one structure of the formula (Ia) is used as matrix material in an emitting layer and/or in an electron transport layer and/or in a hole blocker layer.

36. The electronic device as claimed in claim 34, wherein the compound comprising at least one structure of the formula (Ia) is used as matrix material for phosphorescent emitters in combination with a further matrix material, where the further matrix material is selected from compounds of one of the formulae (H-1), (H-2), (H-3), (H-4) or (H-5): 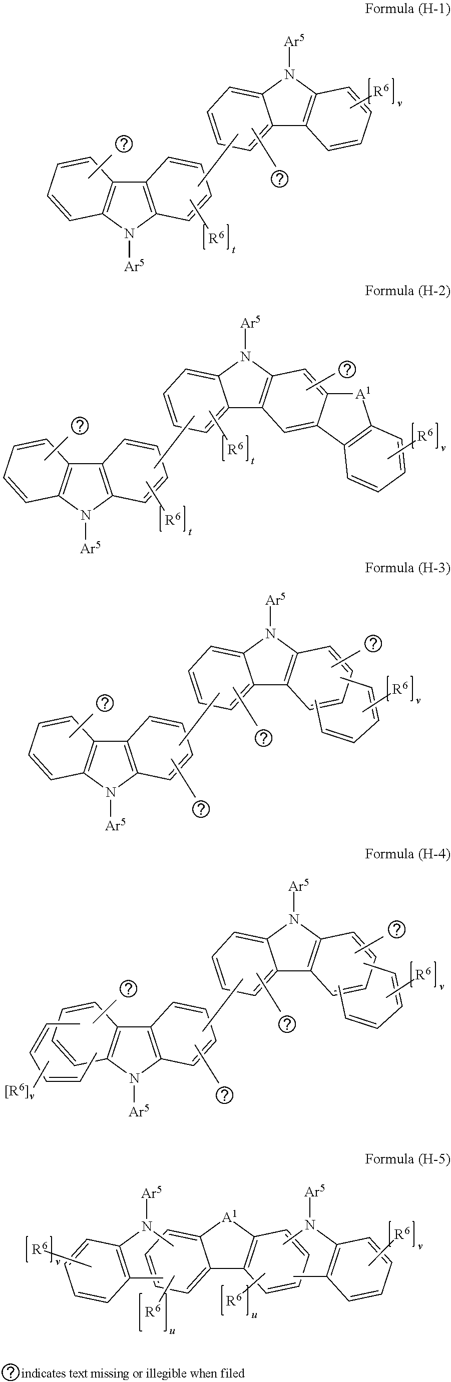
where
R⁶ is the same or different at each instance and is H, D, F, Cl, Br, I, N(R⁷)₂, N(Ar″)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R⁷ radicals and where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, and in each case has R⁷ radicals; at the same time, two R⁶ radicals together may also form an aromatic, heteroaromatic, aliphatic or heteroaliphatic ring system;
Ar″ is the same or different at each instance and is an aromatic or heteroaromatic ring system which has 5 to 40 aromatic ring atoms and has R⁷ radicals;
A¹ is C(R⁷)₂, NR⁷, O or S;
Ar⁵ is the same or different at each instance and is an aromatic or heteroaromatic ring system which has 5 to 40 aromatic ring atoms and has R⁷ radicals;
R⁷ is the same or different at each instance and is H, D, F, Cl, Br, I, N(R⁸)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where each alkyl, alkenyl or alkynyl group has R¹ radicals, where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and in each case has R¹ radicals; at the same time, two or more R⁷ radicals together may form an aromatic, heteroaromatic, aliphatic or heteroaliphatic ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
v is the same or different at each instance and is 0, 1, 2, 3 or 4;
t is the same or different at each instance and is 0, 1, 2 or 3; and
u is the same or different at each instance and is 0, 1 or 2.
